SEQUENCE LISTING (500 sequences)

SEQ ID NO: 1 - protein, 594 aa, synthetic construct
  REGION 1..594: note = Description of sequence: CV1-KR/G12C-A0201
MSRSVALAVLALLSLSGLEAKLVVVGACGVGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKVATNFSLLKQAGDVEENPGPMKWVTFISLLFLFSSAYSILMQYIKANSKFIGIPMGLPQSIALSSLMVAQPMGLPAKFVAAWTLKAAA

SEQ ID NO: 2 - DNA, 1788 bp, synthetic construct
  misc_feature 1..1788: note = Description of sequence: CV1-KR/G12C-A0201
  source 1..1788: mol_type = other DNA
atgtctcgctccgtggccttagctgtgctcgcgctactctctctttctggcctggaggctaaacttgtggtagttggagcttgcggcgtaggctgcggcgcctctggcggcggaggatccggaggcggaggaagcatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggcggaggcggctctggcggaggaggcagcggcggtggcggcagcggcggcggcggctctggcagcggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctgctacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgtcttcccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtggctaccaacttcagcctgctgaagcaggctggcgatgtggaagaaaaccctggcccaatgaagtgggtaacctttatttcccttctttttctctttagctcggcttattccatactaatgcaatacataaaagcaaacagcaaattcataggaataccaatgggactaccacaaagcatagcactaagcagcctaatggtagcacaaccaatgggactaccagcaaaattcgtagcagcatggacactaaaagcagcagcatgatag

SEQ ID NO: 3 - protein, 593 aa, synthetic construct
  REGION 1..593: note = Description of sequence: CV2-KR/G12C-A0301
MSRSVALAVLALLSLSGLEAVVGACGVGKGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSGSHSMRYFFTSVSRPGRGEPRFIAVGYVDDTQFVRFDSDAASQRMEPRAPWIEQEGPEYWDQETRNVKAQSQTDRVDLGTLRGCYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKVATNFSLLKQAGDVEENPGPMKWVTFISLLFLFSSAYSILMQYIKANSKFIGIPMGLPQSIALSSLMVAQPMGLPAKFVAAWTLKAAA

SEQ ID NO: 4 - DNA, 1785 bp, synthetic construct
  misc_feature 1..1785: note = Description of sequence: CV2-KR/G12C-A0301
  source 1..1785: mol_type = other DNA
atgtctcgctccgtggccttagctgtgctcgcgctactctctctttctggcctggaggctgtagttggagcttgtggcgtaggcaagggctgcggcgcctctggcggcggaggatccggaggcggaggaagcatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggcggaggcggctctggcggaggaggcagcggcggtggcggcagcggcggcggcggctctggcagcggctcccactccatgaggtatttcttcacatccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccaggagacacggaatgtgaaggcccagtcacagactgaccgagtggacctggggaccctgcgcggctgctacaaccagagcgaggccggttctcacaccatccagataatgtatggctgcgacgtggggtcggacgggcgcttcctccgcgggtaccggcaggacgcctacgacggcaaggattacatcgccctgaacgaggacctgcgctcttggaccgcggcggacatggcggctcagatcaccaagcgcaagtgggaggcggcccatgaggcggagcagttgagagcctacctggatggcacgtgcgtggagtggctccgcagatacctggagaacgggaaggagacgctgcagcgcacggacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatgggagctgtcttcccagcccaccatccccatcgtgggcatcattgctggcctggttctccttggagctgtgatcactggagctgtggtcgctgccgtgatgtggaggaggaagagctcagatagaaaaggagggagttacactcaggctgcaagcagtgacagtgcccagggctctgatgtgtccctcacagcttgtaaagtggctaccaacttcagcctgctgaagcaggctggcgatgtggaagaaaaccctggcccaatgaagtgggtaacctttatttcccttctttttctctttagctcggcttattccatactaatgcaatacataaaagcaaacagcaaattcataggaataccaatgggactaccacaaagcatagcactaagcagcctaatggtagcacaaccaatgggactaccagcaaaattcgtagcagcatggacactaaaagcagcagcatgatag

SEQ ID NO: 5 - protein, 593 aa, synthetic construct
  REGION 1..593: note = Description of sequence: CV3-KR/G12C-A1101
MSRSVALAVLALLSLSGLEAVVGACGVGKGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRLLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKVATNFSLLKQAGDVEENPGPMKWVTFISLLFLFSSAYSILMQYIKANSKFIGIPMGLPQSIALSSLMVAQPMGLPAKFVAAWTLKAAA

SEQ ID NO: 6 - DNA, 1785 bp, synthetic construct
  misc_feature 1..1785: note = Description of sequence: CV3-KR/G12C-A1101
  source 1..1785: mol_type = other DNA
atgtctcgctccgtggccttagctgtgctcgcgctactctctctttctggcctggaggctgtagttggagcttgtggcgtaggcaagggctgcggcgcctctggcggcggaggatccggaggcggaggaagcatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggcggaggcggctctggcggaggaggcagcggcggtggcggcagcggcggcggcggctctggcagcggctcccactccatgaggtatttctacacctccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccaggagacacggaatgtgaaggcccagtcacagactgaccgagtggacctggggaccctgcgcggctgctacaaccagagcgaggacggttctcacaccatccagataatgtatggctgcgacgtggggccggacgggcgcttactccgcgggtaccggcaggacgcctacgacggcaaggattacatcgccctgaacgaggacctgcgctcttggaccgcggcggacatggcagctcagatcaccaagcgcaagtgggaggcggcccatgcggcggagcagcagagagcctacctggagggccggtgcgtggagtggctccgcagatacctggagaacgggaaggagacgctgcagcgcacggacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatgggagctgtcttcccagcccaccatccccatcgtgggcatcattgctggcctggttctccttggagctgtgatcactggagctgtggtcgctgccgtgatgtggaggaggaagagctcagatagaaaaggagggagttacactcaggctgcaagcagtgacagtgcccagggctctgatgtgtctctcacagcttgtaaagtggctaccaacttcagcctgctgaagcaggctggcgatgtggaagaaaaccctggcccaatgaagtgggtaacctttatttcccttctttttctctttagctcggcttattccatactaatgcaatacataaaagcaaacagcaaattcataggaataccaatgggactaccacaaagcatagcactaagcagcctaatggtagcacaaccaatgggactaccagcaaaattcgtagcagcatggacactaaaagcagcagcatgatag

SEQ ID NO: 7 - protein, 590 aa, synthetic construct
  REGION 1..590: note = Description of sequence: CV4-KR/G12C-B0702
MSRSVALAVLALLSLSGLEAGACGVGKSAGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSGSHSMRYFYTSVSRPGRGEPRFISVGYVDDTQFVRFDSDAASPREEPRAPWIEQEGPEYWDRNTQIYKAQAQTDRESLRNLRGCYNQSEAGSHTLQSMYGCDVGPDGRLLRGHDQLAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTAATNFSLLKQAGDVEENPGPMKWVTFISLLFLFSSAYSILMQYIKANSKFIGIPMGLPQSIALSSLMVAQPMGLPAKFVAAWTLKAAA

SEQ ID NO: 8 - DNA, 1776 bp, synthetic construct
  misc_feature 1..1776: note = Description of sequence: CV4-KR/G12C-B0702
  source 1..1776: mol_type = other DNA
atgtctcgctccgtggccttagctgtgctcgcgctactctctctttctggcctggaggctggagcttgcggcgtaggcaagagtgccggctgcggcgcctctggcggcggaggatccggaggcggaggaagcatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggcggaggcggctctggcggaggaggcagcggcggtggcggcagcggcggcggcggctctggcagcggctcccactccatgaggtatttctacacctccgtgtcccggcccggccgcggggagccccgcttcatctcagtgggctacgtggacgacacccagttcgtgaggttcgacagcgacgccgcgagtccgagagaggagccgcgggcgccgtggatagagcaggaggggccggagtattgggaccggaacacacagatctacaaggcccaggcacagactgaccgagagagcctgcggaacctgcgcggctgctacaaccagagcgaggccgggtctcacaccctccagagcatgtacggctgcgacgtggggccggacgggcgcctcctccgcgggcatgaccagttagcctacgacggcaaggattacatcgccctgaacgaggacctgcgctcctggaccgccgcggacacggcggctcagatcacccagcgcaagtgggaggcggcccgtgaggcggagcagcggagagcctacctggagggcgagtgcgtggagtggctccgcagatacctggagaacgggaaggacaagctggagcgcgctgaccccccaaagacacacgtgacccaccaccccatctctgaccatgaggccaccctgaggtgctgggccctgggtttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccgtcttcccagtccaccgtccccatcgtgggcattgttgctggcctggctgtcctagcagttgtggtcatcggagctgtggtcgctgctgtgatgtgtaggaggaagagttcaggtggaaaaggagggagctactctcaggctgcgtgcagcgacagtgcccagggctctgatgtgtctctcacagctgctaccaacttcagcctgctgaagcaggctggcgatgtggaagaaaaccctggcccaatgaagtgggtaacctttatttcccttctttttctctttagctcggcttattccatactaatgcaatacataaaagcaaacagcaaattcataggaataccaatgggactaccacaaagcatagcactaagcagcctaatggtagcacaaccaatgggactaccagcaaaattcgtagcagcatggacactaaaagcagcagcatgatag

SEQ ID NO: 9 - protein, 594 aa, synthetic construct
  REGION 1..594: note = Description of sequence: CV5-KR/G12D-A0201
MSRSVALAVLALLSLSGLEAKLVVVGADGVGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKVATNFSLLKQAGDVEENPGPMKWVTFISLLFLFSSAYSILMQYIKANSKFIGIPMGLPQSIALSSLMVAQPMGLPAKFVAAWTLKAAA

SEQ ID NO: 10 - DNA, 1788 bp, synthetic construct
  misc_feature 1..1788: note = Description of sequence: CV5-KR/G12D-A0201
  source 1..1788: mol_type = other DNA
atgtctcgctccgtggccttagctgtgctcgcgctactctctctttctggcctggaggctaaacttgtggtagttggagctgacggcgtaggctgcggcgcctctggcggcggaggatccggaggcggaggaagcatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggcggaggcggctctggcggaggaggcagcggcggtggcggcagcggcggcggcggctctggcagcggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctgctacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgtcttcccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtggctaccaacttcagcctgctgaagcaggctggcgatgtggaagaaaaccctggcccaatgaagtgggtaacctttatttcccttctttttctctttagctcggcttattccatactaatgcaatacataaaagcaaacagcaaattcataggaataccaatgggactaccacaaagcatagcactaagcagcctaatggtagcacaaccaatgggactaccagcaaaattcgtagcagcatggacactaaaagcagcagcatgatag

SEQ ID NO: 11 - protein, 593 aa, synthetic construct
  REGION 1..593: note = Description of sequence: CV6-KR/G12D-A0301
MSRSVALAVLALLSLSGLEAVVGADGVGKGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSGSHSMRYFFTSVSRPGRGEPRFIAVGYVDDTQFVRFDSDAASQRMEPRAPWIEQEGPEYWDQETRNVKAQSQTDRVDLGTLRGCYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKVATNFSLLKQAGDVEENPGPMKWVTFISLLFLFSSAYSILMQYIKANSKFIGIPMGLPQSIALSSLMVAQPMGLPAKFVAAWTLKAAA

SEQ ID NO: 12 - DNA, 1785 bp, synthetic construct
  misc_feature 1..1785: note = Description of sequence: CV6-KR/G12D-A0301
  source 1..1785: mol_type = other DNA
atgtctcgctccgtggccttagctgtgctcgcgctactctctctttctggcctggaggctgtagttggagctgatggcgtaggcaagggctgcggcgcctctggcggcggaggatccggaggcggaggaagcatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggcggaggcggctctggcggaggaggcagcggcggtggcggcagcggcggcggcggctctggcagcggctcccactccatgaggtatttcttcacatccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccaggagacacggaatgtgaaggcccagtcacagactgaccgagtggacctggggaccctgcgcggctgctacaaccagagcgaggccggttctcacaccatccagataatgtatggctgcgacgtggggtcggacgggcgcttcctccgcgggtaccggcaggacgcctacgacggcaaggattacatcgccctgaacgaggacctgcgctcttggaccgcggcggacatggcggctcagatcaccaagcgcaagtgggaggcggcccatgaggcggagcagttgagagcctacctggatggcacgtgcgtggagtggctccgcagatacctggagaacgggaaggagacgctgcagcgcacggacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatgggagctgtcttcccagcccaccatccccatcgtgggcatcattgctggcctggttctccttggagctgtgatcactggagctgtggtcgctgccgtgatgtggaggaggaagagctcagatagaaaaggagggagttacactcaggctgcaagcagtgacagtgcccagggctctgatgtgtccctcacagcttgtaaagtggctaccaacttcagcctgctgaagcaggctggcgatgtggaagaaaaccctggcccaatgaagtgggtaacctttatttcccttctttttctctttagctcggcttattccatactaatgcaatacataaaagcaaacagcaaattcataggaataccaatgggactaccacaaagcatagcactaagcagcctaatggtagcacaaccaatgggactaccagcaaaattcgtagcagcatggacactaaaagcagcagcatgatag

SEQ ID NO: 13 - protein, 593 aa, synthetic construct
  REGION 1..593: note = Description of sequence: CV7-KR/G12D-A1101
MSRSVALAVLALLSLSGLEAVVGADGVGKGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRLLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKVATNFSLLKQAGDVEENPGPMKWVTFISLLFLFSSAYSILMQYIKANSKFIGIPMGLPQSIALSSLMVAQPMGLPAKFVAAWTLKAAA

SEQ ID NO: 14 - DNA, 1785 bp, synthetic construct
  misc_feature 1..1785: note = Description of sequence: CV7-KR/G12D-A1101
  source 1..1785: mol_type = other DNA
atgtctcgctccgtggccttagctgtgctcgcgctactctctctttctggcctggaggctgtagttggagctgatggcgtaggcaagggctgcggcgcctctggcggcggaggatccggaggcggaggaagcatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggcggaggcggctctggcggaggaggcagcggcggtggcggcagcggcggcggcggctctggcagcggctcccactccatgaggtatttctacacctccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccaggagacacggaatgtgaaggcccagtcacagactgaccgagtggacctggggaccctgcgcggctgctacaaccagagcgaggacggttctcacaccatccagataatgtatggctgcgacgtggggccggacgggcgcttactccgcgggtaccggcaggacgcctacgacggcaaggattacatcgccctgaacgaggacctgcgctcttggaccgcggcggacatggcagctcagatcaccaagcgcaagtgggaggcggcccatgcggcggagcagcagagagcctacctggagggccggtgcgtggagtggctccgcagatacctggagaacgggaaggagacgctgcagcgcacggacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatgggagctgtcttcccagcccaccatccccatcgtgggcatcattgctggcctggttctccttggagctgtgatcactggagctgtggtcgctgccgtgatgtggaggaggaagagctcagatagaaaaggagggagttacactcaggctgcaagcagtgacagtgcccagggctctgatgtgtctctcacagcttgtaaagtggctaccaacttcagcctgctgaagcaggctggcgatgtggaagaaaaccctggcccaatgaagtgggtaacctttatttcccttctttttctctttagctcggcttattccatactaatgcaatacataaaagcaaacagcaaattcataggaataccaatgggactaccacaaagcatagcactaagcagcctaatggtagcacaaccaatgggactaccagcaaaattcgtagcagcatggacactaaaagcagcagcatgatag

SEQ ID NO: 15 - protein, 590 aa, synthetic construct
  REGION 1..590: note = Description of sequence: CV8-KR/G12D-B0702
MSRSVALAVLALLSLSGLEAGADGVGKSAGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSGSHSMRYFYTSVSRPGRGEPRFISVGYVDDTQFVRFDSDAASPREEPRAPWIEQEGPEYWDRNTQIYKAQAQTDRESLRNLRGCYNQSEAGSHTLQSMYGCDVGPDGRLLRGHDQLAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTAATNFSLLKQAGDVEENPGPMKWVTFISLLFLFSSAYSILMQYIKANSKFIGIPMGLPQSIALSSLMVAQPMGLPAKFVAAWTLKAAA

SEQ ID NO: 16 - DNA, 1776 bp, synthetic construct
  misc_feature 1..1776: note = Description of sequence: CV8-KR/G12D-B0702
  source 1..1776: mol_type = other DNA
atgtctcgctccgtggccttagctgtgctcgcgctactctctctttctggcctggaggctggagctgatggcgtaggcaagagtgccggctgcggcgcctctggcggcggaggatccggaggcggaggaagcatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggcggaggcggctctggcggaggaggcagcggcggtggcggcagcggcggcggcggctctggcagcggctcccactccatgaggtatttctacacctccgtgtcccggcccggccgcggggagccccgcttcatctcagtgggctacgtggacgacacccagttcgtgaggttcgacagcgacgccgcgagtccgagagaggagccgcgggcgccgtggatagagcaggaggggccggagtattgggaccggaacacacagatctacaaggcccaggcacagactgaccgagagagcctgcggaacctgcgcggctgctacaaccagagcgaggccgggtctcacaccctccagagcatgtacggctgcgacgtggggccggacgggcgcctcctccgcgggcatgaccagttagcctacgacggcaaggattacatcgccctgaacgaggacctgcgctcctggaccgccgcggacacggcggctcagatcacccagcgcaagtgggaggcggcccgtgaggcggagcagcggagagcctacctggagggcgagtgcgtggagtggctccgcagatacctggagaacgggaaggacaagctggagcgcgctgaccccccaaagacacacgtgacccaccaccccatctctgaccatgaggccaccctgaggtgctgggccctgggtttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccgtcttcccagtccaccgtccccatcgtgggcattgttgctggcctggctgtcctagcagttgtggtcatcggagctgtggtcgctgctgtgatgtgtaggaggaagagttcaggtggaaaaggagggagctactctcaggctgcgtgcagcgacagtgcccagggctctgatgtgtctctcacagctgctaccaacttcagcctgctgaagcaggctggcgatgtggaagaaaaccctggcccaatgaagtgggtaacctttatttcccttctttttctctttagctcggcttattccatactaatgcaatacataaaagcaaacagcaaattcataggaataccaatgggactaccacaaagcatagcactaagcagcctaatggtagcacaaccaatgggactaccagcaaaattcgtagcagcatggacactaaaagcagcagcatgatag

SEQ ID NO: 17 - protein, 594 aa, synthetic construct
  REGION 1..594: note = Description of sequence: CV9-HPV/E6-29- A0201
MSRSVALAVLALLSLSGLEATIHDIILECVGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKVATNFSLLKQAGDVEENPGPMKWVTFISLLFLFSSAYSILMQYIKANSKFIGIPMGLPQSIALSSLMVAQPMGLPAKFVAAWTLKAAA

SEQ ID NO: 18 - DNA, 1524 bp, synthetic construct
  misc_feature 1..1524: note = Description of sequence: CV9-HPV/E6-29- A0201
  source 1..1524: mol_type = other DNA
atgagcagaagcgtggccctggccgtgctggccctgctgagcctgagcggcctggaggccaccatccacgacatcatcctggaatgcgtgggatgcggcgctagcggcgggggcggaagcggtggcggaggtagcattcagagaacccccaagatccaagtgtacagcagacaccctgccgaaaacggaaagagcaacttcctgaactgctacgtgagcggcttccaccctagcgacatcgaggtggacctgctgaagaacggcgagagaatcgagaaggtggagcacagcgacctgagcttcagcaaggactggagcttctacctgctgtactacaccgagttcacccccaccgagaaggacgagtacgcctgcagagtgaaccacgtgaccctgtctcagcccaagatcgtgaagtgggaccgggacatggggggcggaggctcgggtggcggtggcagcggcggtgggggtagtgggggtggcggcagcggaagtggtagccacagcatgagatacttcttcacaagcgtgagcagacccggcagaggcgagcctagattcatcgccgtgggctacgtggacgacacacagttcgtgagattcgacagcgacgccgcttctcagagaatggagcctagagccccctggatcgagcaagagggccccgagtactgggacggcgagacaagaaaggtgaaggcccactctcagacccacagagtggacctgggcaccctgagaggctgctacaatcagagcgaggccggcagccacaccgtgcagagaatgtacggctgcgacgtgggcagcgactggagattcctgagaggctaccatcagtacgcctacgacggcaaggactacatcgccctgaaggaggacctgagaagctggaccgccgccgacatggccgcccaaaccaccaaacacaagtgggaggccgcccacgtggccgagcagctgagagcctacctggagggcacctgcgtggagtggctgagaagatacctggaaaacggcaaagagacgctgcagagaaccgacgcccccaagacccacatgacccaccacgccgtgagcgaccacgaggccaccctgagatgctgggccctgagcttctaccccgcagagatcacgctgacctggcagagagacggcgaggatcagacccaagacaccgagctggtggagacaagaccggccggcgatggcacatttcagaaatgggccgccgttgtggtgcctagcggccaagagcagagatacacctgccacgtgcagcacgagggcctgcccaagcccctgaccctgagatgggagcctagctctcagcccaccatccccatcgtgggcatcatcgccggcctggtgctgttcggcgccgtgatcaccggcgccgtggtggccgccgtgatgtggagaagaaagagcagcgaccgcaaagggggcagctatagccaagcagctagctctgacagcgcccaaggcagcgacgtgagcctgaccgcctgcaaggtgtag

SEQ ID NO: 19 - protein, 593 aa, synthetic construct
  REGION 1..593: note = Description of sequence: CV10-HPV/E7-11- A0201
MSRSVALAVLALLSLSGLEAYMLDLQPETGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKVATNFSLLKQAGDVEENPGPMKWVTFISLLFLFSSAYSILMQYIKANSKFIGIPMGLPQSIALSSLMVAQPMGLPAKFVAAWTLKAAA

SEQ ID NO: 20 - DNA, 1785 bp, synthetic construct
  misc_feature 1..1785: note = Description of sequence: CV10-HPV/E7-11- A0201
  source 1..1785: mol_type = other DNA
atgtctcgctccgtggccttagctgtgctcgcgctactctctctttctggcctggaggcttatatgttagatttgcaaccagagacaggctgcggcgcctctggcggcggaggatccggaggcggaggaagcatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggcggaggcggctctggcggaggaggcagcggcggtggcggcagcggcggcggcggctctggcagcggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctgctacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgtcttcccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtggctaccaacttcagcctgctgaagcaggctggcgatgtggaagaaaaccctggcccaatgaagtgggtaacctttatttcccttctttttctctttagctcggcttattccatactaatgcaatacataaaagcaaacagcaaattcataggaataccaatgggactaccacaaagcatagcactaagcagcctaatggtagcacaaccaatgggactaccagcaaaattcgtagcagcatggacactaaaagcagcagcatgatag

SEQ ID NO: 21 - protein, 15 aa, synthetic construct
  REGION 1..15: note = Description of sequence: Universal CD4 T epitopes -Tetanus
ILMQYIKANSKFIGI

SEQ ID NO: 22 - DNA, 45 bp, synthetic construct
  misc_feature 1..45: note = Description of sequence: Universal CD4 T epitopes -Tetanus
  source 1..45: mol_type = other DNA
atactaatgcaatacataaaagcaaacagcaaattcataggaata

SEQ ID NO: 23 - protein, 12 aa, synthetic construct
  REGION 1..12: note = Description of sequence: Universal CD4 T epitopes -Diphtheria
QSIALSSLMVAQ

SEQ ID NO: 24 - DNA, 36 bp, synthetic construct
  misc_feature 1..36: note = Description of sequence: Universal CD4 T epitopes -Diphtheria
  source 1..36: mol_type = other DNA
caaagcatagcactaagcagcctaatggtagcacaa

SEQ ID NO: 25 - protein, 13 aa, synthetic construct
  REGION 1..13: note = Description of sequence: Universal CD4 T epitopes - PADRE peptide
AKFVAAWTLKAAA

SEQ ID NO: 26 - DNA, 39 bp, synthetic construct
  misc_feature 1..39: note = Description of sequence: Universal CD4 T epitopes - PADRE peptide
  source 1..39: mol_type = other DNA
gcaaaattcgtagcagcatggacactaaaagcagcagca

SEQ ID NO: 27 - protein, 10 aa, synthetic construct
  REGION 1..10: note = Description of sequence: Gene: KRAS; mutation: G12D; HLA type: A*02:01
KLVVVGADGV

SEQ ID NO: 28 - protein, 10 aa, synthetic construct
  REGION 1..10: note = Description of sequence: Gene: KRAS; mutation: G12C; HLA type: A*02:01
KLVVVGACGV

SEQ ID NO: 29 - protein, 9 aa, synthetic construct
  REGION 1..9: note = Description of sequence: Gene: KRAS; mutation: G12D; HLA type: B*07:02/C*08:02
GADGVGKSA

SEQ ID NO: 30 - protein, 10 aa, synthetic construct
  REGION 1..10: note = Description of sequence: Gene: KRAS; mutation: G12D; HLA type: B*07:02/C*08:02
GADGVGKSAL

SEQ ID NO: 31 - protein, 9 aa, synthetic construct
  REGION 1..9: note = Description of sequence: Gene: KRAS; mutation: G12C; HLA type: B*07:02/C*08:02
GACGVGKSA

SEQ ID NO: 32 - protein, 10 aa, synthetic construct
  REGION 1..10: note = Description of sequence: Gene: KRAS; mutation: G12C; HLA type: B*07:02/C*08:02
GACGVGKSAL

SEQ ID NO: 33 - protein, 9 aa, synthetic construct
  REGION 1..9: note = Description of sequence: Gene: KRAS; mutation: G12R; HLA type: B*07:02/C*08:02
GARGVGKSA

SEQ ID NO: 34 - protein, 10 aa, synthetic construct
  REGION 1..10: note = Description of sequence: Gene: KRAS; mutation: G12R; HLA type: B*07:02/C*08:02
GARGVGKSAL

SEQ ID NO: 35 - protein, 9 aa, synthetic construct
  REGION 1..9: note = Description of sequence: Gene: KRAS; mutation: G12V; HLA type: B*07:02/C*08:02
GAVGVGKSA

SEQ ID NO: 36 - protein, 10 aa, synthetic construct
  REGION 1..10: note = Description of sequence: Gene: KRAS; mutation: G12V; HLA type: B*07:02/C*08:02
GAVGVGKSAL

SEQ ID NO: 37 - protein, 9 aa, synthetic construct
  REGION 1..9: note = Description of sequence: Gene: KRAS; mutation: G12D; HLA type: A*11:01/ A*03:01
VVGADGVGK

SEQ ID NO: 38 - protein, 10 aa, synthetic construct
  REGION 1..10: note = Description of sequence: Gene: KRAS; mutation: G12D; HLA type: A*11:01/ A*03:01
VVVGADGVGK

SEQ ID NO: 39 - protein, 9 aa, synthetic construct
  REGION 1..9: note = Description of sequence: Gene: KRAS; mutation: G12V; HLA type: A*11:01/ A*03:01
VVGAVGVGK

SEQ ID NO: 40 - protein, 10 aa, synthetic construct
  REGION 1..10: note = Description of sequence: Gene: KRAS; mutation: G12V; HLA type: A*11:01/ A*03:01
VVVGAVGVGK

SEQ ID NO: 41 - protein, 9 aa, synthetic construct
  REGION 1..9: note = Description of sequence: Gene: KRAS; mutation: G12R; HLA type: A*11:01/ A*03:01
VVGARGVGK

SEQ ID NO: 42 - protein, 10 aa, synthetic construct
  REGION 1..10: note = Description of sequence: Gene: KRAS; mutation: G12R; HLA type: A*11:01/ A*03:01
VVVGARGVGK

SEQ ID NO: 43 - protein, 9 aa, synthetic construct
  REGION 1..9: note = Description of sequence: Gene: KRAS; mutation: G12C; HLA type: A*11:01/ A*03:01
VVGACGVGK

SEQ ID NO: 44 - protein, 10 aa, synthetic construct
  REGION 1..10: note = Description of sequence: Gene: KRAS; mutation: G12C; HLA type: A*11:01/ A*03:01
VVVGACGVGK

SEQ ID NO: 45 - protein, 10 aa, synthetic construct
  REGION 1..10: note = Description of sequence: Gene: KRAS; mutation: G12V; HLA type: A*02:01
KLVVVGAVGV

SEQ ID NO: 46 - protein, 10 aa, synthetic construct
  REGION 1..10: note = Description of sequence: Gene: KRAS; mutation: G12R; HLA type: A*02:01
KLVVVGARGV

SEQ ID NO: 47 - protein, 12 aa, synthetic construct
  REGION 1..12: note = Description of sequence: Gene: KRAS; mutation: G13D; HLA type: A*02:01
GAGDVGKSALTI

SEQ ID NO: 48 - protein, 10 aa, synthetic construct
  REGION 1..10: note = Description of sequence: Gene: KRAS; mutation: Q61R; HLA type: A01:01
ILDTAGREEY

SEQ ID NO: 49 - protein, 10 aa, synthetic construct
  REGION 1..10: note = Description of sequence: Gene: BRAF; mutation: V600E; HLA type: A*02:01
LATEKSRWSG

SEQ ID NO: 50 - protein, 9 aa, synthetic construct
  REGION 1..9: note = Description of sequence: Gene: BRAF; mutation: V600E; HLA type: A*02:01
LATEKSRWS

SEQ ID NO: 51 - protein, 9 aa, synthetic construct
  REGION 1..9: note = Description of sequence: Gene: PIK3CA; mutation: H1047L; HLA type: A*03:01
ALHGGWTTK

SEQ ID NO: 52 - protein, 9 aa, synthetic construct
  REGION 1..9: note = Description of sequence: Gene: PIK3CA; mutation: H1047L; HLA type: B*15:18
LHGGWTTKM

SEQ ID NO: 53 - protein, 10 aa, synthetic construct
  REGION 1..10: note = Description of sequence: Gene: PIK3CA; mutation: H1047R; HLA type: C*07:01/ C*07:02
ARHGGWTTKM

SEQ ID NO: 54 - protein, 11 aa, synthetic construct
  REGION 1..11: note = Description of sequence: Gene: PIK3CA; mutation: E545K; HLA type: A*03:01
STRDPLSEITK

SEQ ID NO: 55 - protein, 10 aa, synthetic construct
  REGION 1..10: note = Description of sequence: Gene: IDH1; mutation: R132H; HLA type: A*02:01
GWVKPIIIGH

SEQ ID NO: 56 - protein, 9 aa, synthetic construct
  REGION 1..9: note = Description of sequence: Gene: TP53; mutation: R175H; HLA type: A*02:01
HMTEVVRHC

SEQ ID NO: 57 - protein, 10 aa, synthetic construct
  REGION 1..10: note = Description of sequence: Gene: TP53; mutation: R248W; HLA type: A*68:01
SSCMGGMNWR

SEQ ID NO: 58 - protein, 9 aa, synthetic construct
  REGION 1..9: note = Description of sequence: Gene: TP53; mutation: Y220C; HLA type: A*02:01
VVPCEPPEV

SEQ ID NO: 59 - protein, 9 aa, synthetic construct
  REGION 1..9: note = Description of sequence: Gene: ERBB3 ; mutation: V104M; HLA type: C*07:02
LRMVRGTQV

SEQ ID NO: 60 - protein, 10 aa, synthetic construct
  REGION 1..10: note = Description of sequence: Gene: ERBB3 ; mutation: V104M; HLA type: C*07:02
LRMVRGTQVY

SEQ ID NO: 61 - protein, 9 aa, synthetic construct
  REGION 1..9: note = Description of sequence: Gene: ERBB3 ; mutation: V104M; HLA type: B*15:18
RMVRGTQVY

SEQ ID NO: 62 - protein, 11 aa, synthetic construct
  REGION 1..11: note = Description of sequence: Gene: FBXW7; mutation: R505G; HLA type: A*02:01
LHVLMGHVAAV

SEQ ID NO: 63 - protein, 10 aa, synthetic construct
  REGION 1..10: note = Description of sequence: Gene: FBXW7; mutation: R505G; HLA type: A*02:01
HVLMGHVAAV

SEQ ID NO: 64 - protein, 9 aa, synthetic construct
  REGION 1..9: note = Description of sequence: Gene: FBXW7; mutation: R505G; HLA type: A*02:01
VLMGHVAAV

SEQ ID NO: 65 - protein, 9 aa, synthetic construct
  REGION 1..9: note = Description of sequence: Gene: KIAA1429; mutation: D1358E; HLA type: A*02: 01
FLAEHEYGL

SEQ ID NO: 66 - protein, 9 aa, synthetic construct
  REGION 1..9: note = Description of sequence: Gene: MAPK13; mutation: G137V; HLA type: A*02: 01
YLVYQMLKV

SEQ ID NO: 67 - protein, 9 aa, synthetic construct
  REGION 1..9: note = Description of sequence: Gene: DSG3 ; mutation: I444M ; HLA type: B*67
MIDSKTAEM

SEQ ID NO: 68 - protein, 10 aa, synthetic construct
  REGION 1..10: note = Description of sequence: Gene: C3orf70 ; mutation: S6L ; HLA type: B*40:01
ALPASERGWK

SEQ ID NO: 69 - protein, 9 aa, synthetic construct
  REGION 1..9: note = Description of sequence: Gene: SH2D3C ; mutation: Q129R ; HLA type: A*34:01
SYTHIQYLF

SEQ ID NO: 70 - protein, 10 aa, synthetic construct
  REGION 1..10: note = Description of sequence: Gene: ZNF468 ; mutation: Q19E ; HLA type: C*12:03
LTFRDVAIEF

SEQ ID NO: 71 - protein, 9 aa, synthetic construct
  REGION 1..9: note = Description of sequence: Gene: ADAM29 ; mutation: R399H ; HLA type: A*34:01
HTKDIFNVK

SEQ ID NO: 72 - protein, 9 aa, synthetic construct
  REGION 1..9: note = Description of sequence: Gene: LUZP4 ; mutation: T8M ; HLA type: B*67:01
MASFRKLML

SEQ ID NO: 73 - protein, 9 aa, synthetic construct
  REGION 1..9: note = Description of sequence: Gene: MAGEC3 ; mutation: R9H ; HLA type: C*07:02
FPNLPHLSF

SEQ ID NO: 74 - protein, 9 aa, synthetic construct
  REGION 1..9: note = Description of sequence: Gene: MAGEC3 ; mutation: R9H ; HLA type: B*67:01
FPNLPHLSF

SEQ ID NO: 75 - protein, 10 aa, synthetic construct
  REGION 1..10: note = Description of sequence: Gene: MAGEC3 ; mutation: R9H ; HLA type: B*15:18
MPLFPNLPHL

SEQ ID NO: 76 - protein, 9 aa, synthetic construct
  REGION 1..9: note = Description of sequence: Gene: OTOA ; mutation: T195M ; HLA type: A*34:01
SPIEIGLFI

SEQ ID NO: 77 - protein, 9 aa, synthetic construct
  REGION 1..9: note = Description of sequence: Gene: OTOA ; mutation: T195M ; HLA type: C*07:02
SRHNKALKL

SEQ ID NO: 78 - protein, 9 aa, synthetic construct
  REGION 1..9: note = Description of sequence: Gene: PRSS55 ; mutation: A236V ; HLA type: B*15:18
FPKLTKNML

SEQ ID NO: 79 - protein, 9 aa, synthetic construct
  REGION 1..9: note = Description of sequence: Gene: PRSS55 ; mutation: A236V ; HLA type: B*15:18
MFPKLTKNM

SEQ ID NO: 80 - protein, 10 aa, synthetic construct
  REGION 1..10: note = Description of sequence: Gene: PRSS55 ; mutation: A236V ; HLA type: A*34:01
MFPKLTKNML

SEQ ID NO: 81 - protein, 9 aa, synthetic construct
  REGION 1..9: note = Description of sequence: Gene: SAGE1 ; mutation: M193L ; HLA type: A*26:01
IPALSARDL

SEQ ID NO: 82 - protein, 9 aa, synthetic construct
  REGION 1..9: note = Description of sequence: Gene: SAGE1 ; mutation: M193L ; HLA type: B*15:18
LINMAATPI

SEQ ID NO: 83 - protein, 9 aa, synthetic construct
  REGION 1..9: note = Description of sequence: Gene: SAGE1 ; mutation: M193L ; HLA type: B*15:18
MAATPIPAL

SEQ ID NO: 84 - protein, 9 aa, synthetic construct
  REGION 1..9: note = Description of sequence: Gene: SAGE1 ; mutation: M193L ; HLA type: B*15:18
SARDLYATV

SEQ ID NO: 85 - protein, 11 aa, synthetic construct
  REGION 1..11: note = Description of sequence: Gene: SAGE1 ; mutation: M193L ; HLA type: A*26:01
TPIPALSARDL

SEQ ID NO: 86 - protein, 9 aa, synthetic construct
  REGION 1..9: note = Description of sequence: Gene: SPAG9 ; mutation: E283K ; HLA type: B*40:01
IVKNDLIAK

SEQ ID NO: 87 - protein, 9 aa, synthetic construct
  REGION 1..9: note = Description of sequence: Gene: SPAG9 ; mutation: E283K ; HLA type: A*03:01
KVDKLTCEK

SEQ ID NO: 88 - protein, 10 aa, synthetic construct
  REGION 1..10: note = Description of sequence: Gene: AKAP4 ; mutation: A712V ; HLA type: A*02:01
SVMKLCLIMV

SEQ ID NO: 89 - protein, 9 aa, synthetic construct
  REGION 1..9: note = Description of sequence: Gene: ARX ; mutation: V508M ; HLA type: B*35:01
TPAMEGAVA

SEQ ID NO: 90 - protein, 9 aa, synthetic construct
  REGION 1..9: note = Description of sequence: Gene: ARX ; mutation: V508M ; HLA type: C*03:03
LLRQPTPAM

SEQ ID NO: 91 - protein, 10 aa, synthetic construct
  REGION 1..10: note = Description of sequence: Gene: CRISP2 ; mutation: P5L ; HLA type: A*02:01
MALLLVLFLV

SEQ ID NO: 92 - protein, 10 aa, synthetic construct
  REGION 1..10: note = Description of sequence: Gene: CRISP2 ; mutation: P5L ; HLA type: A*02:01
LLLVLFLVTV

SEQ ID NO: 93 - protein, 9 aa, synthetic construct
  REGION 1..9: note = Description of sequence: Gene: CRISP2 ; mutation: P5L ; HLA type: A*02:01
ALLLVLFLV

SEQ ID NO: 94 - protein, 9 aa, synthetic construct
  REGION 1..9: note = Description of sequence: Gene: CRISP2 ; mutation: P5L ; HLA type: A*02:01
LLVLFLVTV

SEQ ID NO: 95 - protein, 9 aa, synthetic construct
  REGION 1..9: note = Description of sequence: Gene: CRISP2 ; mutation: P5L ; HLA type: A*02:01
VLFLVTVLL

SEQ ID NO: 96 - protein, 9 aa, synthetic construct
  REGION 1..9: note = Description of sequence: Gene: CRISP2 ; mutation: P5L ; HLA type: A*02:01
FLVTVLLPS

SEQ ID NO: 97 - protein, 9 aa, synthetic construct
  REGION 1..9: note = Description of sequence: Gene: CRISP2 ; mutation: P5L ; HLA type: C*03:03
MALLLVLFL

SEQ ID NO: 98 - protein, 9 aa, synthetic construct
  REGION 1..9: note = Description of sequence: Gene: CRISP2 ; mutation: P5L ; HLA type: C*03:03
LVLFLVTVL

SEQ ID NO: 99 - protein, 9 aa, synthetic construct
  REGION 1..9: note = Description of sequence: Gene: MAGEC2 ; mutation: R287Q ; HLA type: A*02:01
KVWVQGHYL

SEQ ID NO: 100 - protein, 9 aa, synthetic construct
  REGION 1..9: note = Description of sequence: Gene: MAGEC2 ; mutation: R287Q ; HLA type: B*35:01
WVQGHYLEY

SEQ ID NO: 101 - protein, 9 aa, synthetic construct
  REGION 1..9: note = Description of sequence: Gene: MAGEC2 ; mutation: R287Q ; HLA type: B*35:01
EVPHSSPPY

SEQ ID NO: 102 - protein, 9 aa, synthetic construct
  REGION 1..9: note = Description of sequence: Gene: MAGEC2 ; mutation: R287Q ; HLA type: B*35:01
VPHSSPPYY

SEQ ID NO: 103 - protein, 9 aa, synthetic construct
  REGION 1..9: note = Description of sequence: Gene: AASDH; mutation: S589F; HLA type: A*02: 01
FLNSGGDFL

SEQ ID NO: 104 - protein, 9 aa, synthetic construct
  REGION 1..9: note = Description of sequence: Gene: ABCA2; mutation: S715F; HLA type: A*02: 01
MVIFWVYSV

SEQ ID NO: 105 - protein, 9 aa, synthetic construct
  REGION 1..9: note = Description of sequence: Gene: ACOT8; mutation: M252L; HLA type: A*02: 01
FLVSLDHSM

SEQ ID NO: 106 - protein, 10 aa, synthetic construct
  REGION 1..10: note = Description of sequence: Gene: ACP2; mutation: F393L; HLA type: A*02: 01
LLLIVLLLTV

SEQ ID NO: 107 - protein, 10 aa, synthetic construct
  REGION 1..10: note = Description of sequence: Gene: ADCY9; mutation: S938F; HLA type: A*02: 01
YVFLCPDSSV

SEQ ID NO: 108 - protein, 9 aa, synthetic construct
  REGION 1..9: note = Description of sequence: Gene: AKR1C3; mutation: P119S; HLA type: A*02: 01
YLIHSSMSL

SEQ ID NO: 109 - protein, 10 aa, synthetic construct
  REGION 1..10: note = Description of sequence: Gene: AKT2; mutation: S34F; HLA type: A*02: 01
FLLKSDGFFI

SEQ ID NO: 110 - protein, 10 aa, synthetic construct
  REGION 1..10: note = Description of sequence: Gene: ALKBH1; mutation: P354S; HLA type: A*02: 01
VLATDQNFSL

SEQ ID NO: 111 - protein, 9 aa, synthetic construct
  REGION 1..9: note = Description of sequence: Gene: ALMS1; mutation: R4120W; HLA type: A*02: 01
WIYEQLPEV

SEQ ID NO: 112 - protein, 9 aa, synthetic construct
  REGION 1..9: note = Description of sequence: Gene: ARID1B; mutation: S2118F; HLA type: A*02: 01
FMALLSNLA

SEQ ID NO: 113 - protein, 10 aa, synthetic construct
  REGION 1..10: note = Description of sequence: Gene: ATG2B; mutation: E1609K; HLA type: A*02: 01
FLMKIQLSKV

SEQ ID NO: 114 - protein, 10 aa, synthetic construct
  REGION 1..10: note = Description of sequence: Gene: ATP6V0A2; mutation: R755K; HLA type: A*02: 01
KLWALSLAHA

SEQ ID NO: 115 - protein, 9 aa, synthetic construct
  REGION 1..9: note = Description of sequence: Gene: ATXN10; mutation: L159F; HLA type: A*02: 01
FLIITDFFL

SEQ ID NO: 116 - protein, 9 aa, synthetic construct
  REGION 1..9: note = Description of sequence: Gene: BCAR1; mutation: R255Q; HLA type: A*02: 01
ALAQLQQGV

SEQ ID NO: 117 - protein, 9 aa, synthetic construct
  REGION 1..9: note = Description of sequence: Gene: C11orf74; mutation: P63S; HLA type: A*02: 01
SISSCIPFV

SEQ ID NO: 118 - protein, 9 aa, synthetic construct
  REGION 1..9: note = Description of sequence: Gene: CDCA2; mutation: P815L; HLA type: A*02: 01
KLMESSSVV

SEQ ID NO: 119 - protein, 10 aa, synthetic construct
  REGION 1..10: note = Description of sequence: Gene: CEPT1; mutation: Q199R; HLA type: A*02: 01
FMFYCAHWRT

SEQ ID NO: 120 - protein, 9 aa, synthetic construct
  REGION 1..9: note = Description of sequence: Gene: CHMP2B; mutation: E132K; HLA type: A*02: 01
VLDEIGIKI

SEQ ID NO: 121 - protein, 9 aa, synthetic construct
  REGION 1..9: note = Description of sequence: Gene: CHST2; mutation: P150L; HLA type: A*02: 01
GMAGVAAPL

SEQ ID NO: 122 - protein, 9 aa, synthetic construct
  REGION 1..9: note = Description of sequence: Gene: CLMP; mutation: S2F; HLA type: A*02: 01
FLLLLLLLV

SEQ ID NO: 123 - protein, 10 aa, synthetic construct
  REGION 1..10: note = Description of sequence: Gene: COL12A1; mutation: T1055R; HLA type: A*02: 01
YQIGWDRFCV

SEQ ID NO: 124 - protein, 9 aa, synthetic construct
  REGION 1..9: note = Description of sequence: Gene: COPZ1; mutation: G97R; HLA type: A*02: 01
ALLENMERL

SEQ ID NO: 125 - protein, 10 aa, synthetic construct
  REGION 1..10: note = Description of sequence: Gene: CRYBG3; mutation: A2862T; HLA type: A*02: 01
SLADTRTTSV

SEQ ID NO: 126 - protein, 9 aa, synthetic construct
  REGION 1..9: note = Description of sequence: Gene: CTNS; mutation: P73S; HLA type: A*02: 01
TILELSDEV

SEQ ID NO: 127 - protein, 9 aa, synthetic construct
  REGION 1..9: note = Description of sequence: Gene: CYP2R1; mutation: E212K; HLA type: A*02: 01
KLFSENVEL

SEQ ID NO: 128 - protein, 10 aa, synthetic construct
  REGION 1..10: note = Description of sequence: Gene: DLST; mutation: P25S; HLA type: A*02: 01
SLGRRSLPGV

SEQ ID NO: 129 - protein, 9 aa, synthetic construct
  REGION 1..9: note = Description of sequence: Gene: DNPH1; mutation: E159K; HLA type: A*02: 01
ALLDRYFKA

SEQ ID NO: 130 - protein, 10 aa, synthetic construct
  REGION 1..10: note = Description of sequence: Gene: DOHH; mutation: E208K; HLA type: A*02: 01
ALFRHKVGYV

SEQ ID NO: 131 - protein, 9 aa, synthetic construct
  REGION 1..9: note = Description of sequence: Gene: DPY19L3; mutation: V424I; HLA type: A*02: 01
FILSITVIV

SEQ ID NO: 132 - protein, 10 aa, synthetic construct
  REGION 1..10: note = Description of sequence: Gene: DSP; mutation: S2077F; HLA type: A*02: 01
SLQDAVFQGV

SEQ ID NO: 133 - protein, 10 aa, synthetic construct
  REGION 1..10: note = Description of sequence: Gene: ECE1; mutation: R524Q; HLA type: A*02: 01
MQFFNFSWRV

SEQ ID NO: 134 - protein, 9 aa, synthetic construct
  REGION 1..9: note = Description of sequence: Gene: EPB41L1; mutation: P115L; HLA type: A*02: 01
FAFTVKFYL

SEQ ID NO: 135 - protein, 9 aa, synthetic construct
  REGION 1..9: note = Description of sequence: Gene: FAM171B; mutation: P645S; HLA type: A*02: 01
VMTSFSSEL

SEQ ID NO: 136 - protein, 9 aa, synthetic construct
  REGION 1..9: note = Description of sequence: Gene: FANCG; mutation: P243L; HLA type: A*02: 01
GLCPRLVLV

SEQ ID NO: 137 - protein, 9 aa, synthetic construct
  REGION 1..9: note = Description of sequence: Gene: FARS2; mutation: S368L; HLA type: A*02: 01
VINDILFWL

SEQ ID NO: 138 - protein, 9 aa, synthetic construct
  REGION 1..9: note = Description of sequence: Gene: FAT1; mutation: P2085L; HLA type: A*02: 01
LLYYAVVKV

SEQ ID NO: 139 - protein, 9 aa, synthetic construct
  REGION 1..9: note = Description of sequence: Gene: FURIN; mutation: M434I; HLA type: A*02: 01
GLLDAGAIV

SEQ ID NO: 140 - protein, 10 aa, synthetic construct
  REGION 1..10: note = Description of sequence: Gene: FZD6; mutation: S238F; HLA type: A*02: 01
IIYYFVCYSI

SEQ ID NO: 141 - protein, 11 aa, synthetic construct
  REGION 1..11: note = Description of sequence: Gene: GAK; mutation: R578K; HLA type: A*02: 01
ILVKAVVMTPV

SEQ ID NO: 142 - protein, 10 aa, synthetic construct
  REGION 1..10: note = Description of sequence: Gene: HSPH1; mutation: S393F; HLA type: A*02: 01
FVTDAVPFPI

SEQ ID NO: 143 - protein, 10 aa, synthetic construct
  REGION 1..10: note = Description of sequence: Gene: KLF4; mutation: P195L; HLA type: A*02: 01
SLSGGFVAEL

SEQ ID NO: 144 - protein, 9 aa, synthetic construct
  REGION 1..9: note = Description of sequence: Gene: LTN1; mutation: K1279N; HLA type: A*02: 01
FLNYCSSPL

SEQ ID NO: 145 - protein, 10 aa, synthetic construct
  REGION 1..10: note = Description of sequence: Gene: MAP4K1; mutation: S108F; HLA type: A*02: 01
FLSELQISYV

SEQ ID NO: 146 - protein, 10 aa, synthetic construct
  REGION 1..10: note = Description of sequence: Gene: MED13; mutation: P1679L; HLA type: A*02: 01
FLEMVQTLPL

SEQ ID NO: 147 - protein, 9 aa, synthetic construct
  REGION 1..9: note = Description of sequence: Gene: MED14; mutation: P1344L; HLA type: A*02: 01
VQFCLTIPL

SEQ ID NO: 148 - protein, 10 aa, synthetic construct
  REGION 1..10: note = Description of sequence: Gene: MFSD10; mutation: P199L; HLA type: A*02: 01
LLLEMAPWFA

SEQ ID NO: 149 - protein, 9 aa, synthetic construct
  REGION 1..9: note = Description of sequence: Gene: MLXIP; mutation: P270L; HLA type: A*02: 01
LLLDTDMLM

SEQ ID NO: 150 - protein, 10 aa, synthetic construct
  REGION 1..10: note = Description of sequence: Gene: MRGBP; mutation: I111N; HLA type: A*02: 01
VLPEENIQEV

SEQ ID NO: 151 - protein, 9 aa, synthetic construct
  REGION 1..9: note = Description of sequence: Gene: MTOR; mutation: P896S; HLA type: A*02: 01
ALDSYKHKV

SEQ ID NO: 152 - protein, 9 aa, synthetic construct
  REGION 1..9: note = Description of sequence: Gene: MYCBP2; mutation: S4018F; HLA type: A*02: 01
YCFELLFMV

SEQ ID NO: 153 - protein, 9 aa, synthetic construct
  REGION 1..9: note = Description of sequence: Gene: MYCBPAP; mutation: E606K; HLA type: A*02: 01
VLQKLLMGV

SEQ ID NO: 154 - protein, 9 aa, synthetic construct
  REGION 1..9: note = Description of sequence: Gene: MYO1B; mutation: M14I; HLA type: A*02: 01
SLLDNIIGV

SEQ ID NO: 155 - protein, 9 aa, synthetic construct
  REGION 1..9: note = Description of sequence: Gene: NCOA6; mutation: P657S; HLA type: A*02: 01
LMSQGQMMV

SEQ ID NO: 156 - protein, 9 aa, synthetic construct
  REGION 1..9: note = Description of sequence: Gene: NRBP2; mutation: F365L; HLA type: A*02: 01
SLMELDKFL

SEQ ID NO: 157 - protein, 9 aa, synthetic construct
  REGION 1..9: note = Description of sequence: Gene: NUMB; mutation: P40L; HLA type: A*02: 01
FLVKYLGHV

SEQ ID NO: 158 - protein, 9 aa, synthetic construct
  REGION 1..9: note = Description of sequence: Gene: NVL; mutation: S19F; HLA type: A*02: 01
HMNSSLLFL

SEQ ID NO: 159 - protein, 9 aa, synthetic construct
  REGION 1..9: note = Description of sequence: Gene: AMPD2; mutation: E494K; HLA type: A*02: 01
KLHLFLEHV

SEQ ID NO: 160 - protein, 9 aa, synthetic construct
  REGION 1..9: note = Description of sequence: Gene: ATN1; mutation: P356S; HLA type: A*02: 01
TLAPSSHSL

SEQ ID NO: 161 - protein, 9 aa, synthetic construct
  REGION 1..9: note = Description of sequence: Gene: BEND3; mutation: P583L; HLA type: A*02: 01
LLVHLFLEL

SEQ ID NO: 162 - protein, 9 aa, synthetic construct
  REGION 1..9: note = Description of sequence: Gene: BRPF1; mutation: F292Y; HLA type: A*02: 01
ILYCDMCNL

SEQ ID NO: 163 - protein, 10 aa, synthetic construct
  REGION 1..10: note = Description of sequence: Gene: C14orf93; mutation: L303F; HLA type: A*02: 01
FVLSKLVHNV

SEQ ID NO: 164 - protein, 9 aa, synthetic construct
  REGION 1..9: note = Description of sequence: Gene: C17orf58; mutation: P8L; HLA type: A*02: 01
TLDGFFFRV

SEQ ID NO: 165 - protein, 10 aa, synthetic construct
  REGION 1..10: note = Description of sequence: Gene: CAD; mutation: S1928Y; HLA type: A*02: 01
SLVGQHILYV

SEQ ID NO: 166 - protein, 10 aa, synthetic construct
  REGION 1..10: note = Description of sequence: Gene: CCDC28A; mutation: P19S; HLA type: A*02: 01
SLGAWRLYLL

SEQ ID NO: 167 - protein, 9 aa, synthetic construct
  REGION 1..9: note = Description of sequence: Gene: CDK5R1; mutation: S96F; HLA type: A*02: 01
FTFAQPPPA

SEQ ID NO: 168 - protein, 9 aa, synthetic construct
  REGION 1..9: note = Description of sequence: Gene: CHST15; mutation: F468C; HLA type: A*02: 01
YLLDWLSVC

SEQ ID NO: 169 - protein, 9 aa, synthetic construct
  REGION 1..9: note = Description of sequence: Gene: DALRD3; mutation: P427S; HLA type: A*02: 01
GLYSTFPPV

SEQ ID NO: 170 - protein, 11 aa, synthetic construct
  REGION 1..11: note = Description of sequence: Gene: DDX19B; mutation: S129F; HLA type: A*02: 01
LLFSATFEDFV

SEQ ID NO: 171 - protein, 10 aa, synthetic construct
  REGION 1..10: note = Description of sequence: Gene: ELMSAN1; mutation: S625F; HLA type: A*02: 01
FIAPPVYFNI

SEQ ID NO: 172 - protein, 9 aa, synthetic construct
  REGION 1..9: note = Description of sequence: Gene: ESRP2; mutation: P655L; HLA type: A*02: 01
ALASALTSV

SEQ ID NO: 173 - protein, 9 aa, synthetic construct
  REGION 1..9: note = Description of sequence: Gene: FADS2; mutation: S429F; HLA type: A*02: 01
ALLDIIRFL

SEQ ID NO: 174 - protein, 9 aa, synthetic construct
  REGION 1..9: note = Description of sequence: Gene: FKBP15; mutation: H413Y; HLA type: A*02: 01
SLYPAHPAL

SEQ ID NO: 175 - protein, 9 aa, synthetic construct
  REGION 1..9: note = Description of sequence: Gene: FLII; mutation: A42V; HLA type: A*02: 01
YLPEELVAL

SEQ ID NO: 176 - protein, 9 aa, synthetic construct
  REGION 1..9: note = Description of sequence: Gene: FMO4; mutation: G519R; HLA type: A*02: 01
YLKAWRAPV

SEQ ID NO: 177 - protein, 9 aa, synthetic construct
  REGION 1..9: note = Description of sequence: Gene: IQGAP3; mutation: P560S; HLA type: A*02: 01
GLDDVSLSV

SEQ ID NO: 178 - protein, 9 aa, synthetic construct
  REGION 1..9: note = Description of sequence: Gene: ITGB1; mutation: S277F; HLA type: A*02: 01
FTDAGFHFA

SEQ ID NO: 179 - protein, 9 aa, synthetic construct
  REGION 1..9: note = Description of sequence: Gene: RAPGEF2; mutation: S806L; HLA type: A*02: 01
LMFAIISGL

SEQ ID NO: 180 - protein, 9 aa, synthetic construct
  REGION 1..9: note = Description of sequence: Gene: RBBP5; mutation: I313F; HLA type: A*02: 01
IIASFSSGV

SEQ ID NO: 181 - protein, 9 aa, synthetic construct
  REGION 1..9: note = Description of sequence: Gene: SEC23IP; mutation: P436L; HLA type: A*02: 01
ILDGEMPQV

SEQ ID NO: 182 - protein, 9 aa, synthetic construct
  REGION 1..9: note = Description of sequence: Gene: SLC25A30; mutation: P274S; HLA type: A*02: 01
GSWNIIFFV

SEQ ID NO: 183 - protein, 10 aa, synthetic construct
  REGION 1..10: note = Description of sequence: Gene: SMTN; mutation: R665W; HLA type: A*02: 01
KLIWAALREL

SEQ ID NO: 184 - protein, 10 aa, synthetic construct
  REGION 1..10: note = Description of sequence: Gene: SNX19; mutation: L387F; HLA type: A*02: 01
IMFMTPGSFL

SEQ ID NO: 185 - protein, 10 aa, synthetic construct
  REGION 1..10: note = Description of sequence: Gene: STRN3; mutation: P406S; HLA type: A*02: 01
ALAFHSVEPV

SEQ ID NO: 186 - protein, 10 aa, synthetic construct
  REGION 1..10: note = Description of sequence: Gene: TBL2; mutation: R162C; HLA type: A*02: 01
WLANGDTLCV

SEQ ID NO: 187 - protein, 10 aa, synthetic construct
  REGION 1..10: note = Description of sequence: Gene: TMCO1; mutation: I65V; HLA type: A*02: 01
FADTLLIVFV

SEQ ID NO: 188 - protein, 10 aa, synthetic construct
  REGION 1..10: note = Description of sequence: Gene: TMEM125; mutation: V126I; HLA type: A*02: 01
VLLSGLVLLI

SEQ ID NO: 189 - protein, 11 aa, synthetic construct
  REGION 1..11: note = Description of sequence: Gene: TRAPPC9; mutation: E639K; HLA type: A*02: 01
SLPAKSGLYPV

SEQ ID NO: 190 - protein, 9 aa, synthetic construct
  REGION 1..9: note = Description of sequence: Gene: TRIAP1; mutation: P58L; HLA type: A*02: 01
ILIEGLEFM

SEQ ID NO: 191 - protein, 9 aa, synthetic construct
  REGION 1..9: note = Description of sequence: Gene: TRRAP; mutation: S702F; HLA type: A*02: 01
RLPEMGFNV

SEQ ID NO: 192 - protein, 10 aa, synthetic construct
  REGION 1..10: note = Description of sequence: Gene: UBE3C; mutation: P991L; HLA type: A*02: 01
HLVIKVFWRV

SEQ ID NO: 193 - protein, 10 aa, synthetic construct
  REGION 1..10: note = Description of sequence: Gene: URGCP; mutation: P310L; HLA type: A*02: 01
GLVEISWFFL

SEQ ID NO: 194 - protein, 10 aa, synthetic construct
  REGION 1..10: note = Description of sequence: Gene: USP15; mutation: P800S; HLA type: A*02: 01
KLDLWSLPSV

SEQ ID NO: 195 - protein, 9 aa, synthetic construct
  REGION 1..9: note = Description of sequence: Gene: WSB2; mutation: S194L; HLA type: A*02: 01
VLLGHLQWV

SEQ ID NO: 196 - protein, 9 aa, synthetic construct
  REGION 1..9: note = Description of sequence: Gene: ZNF33A; mutation: S288F; HLA type: A*02: 01
FTLSKPHGV

SEQ ID NO: 197 - protein, 10 aa, synthetic construct
  REGION 1..10: note = Description of sequence: Gene: KIAA1958; mutation: P400S; HLA type: A*02: 01
KLNKFSVFNI

SEQ ID NO: 198 - protein, 9 aa, synthetic construct
  REGION 1..9: note = Description of sequence: Gene: KIF23; mutation: S334F; HLA type: A*02: 01
ITISQLFLV

SEQ ID NO: 199 - protein, 10 aa, synthetic construct
  REGION 1..10: note = Description of sequence: Gene: LIG3; mutation: G76R; HLA type: A*02: 01
YLVFLPRLHV

SEQ ID NO: 200 - protein, 9 aa, synthetic construct
  REGION 1..9: note = Description of sequence: Gene: LNPEP; mutation: S349F; HLA type: A*02: 01
VQDEFFESV

SEQ ID NO: 201 - protein, 9 aa, synthetic construct
  REGION 1..9: note = Description of sequence: Gene: LPHN3; mutation: H1049Y; HLA type: A*02: 01
KMFHYTAIL

SEQ ID NO: 202 - protein, 10 aa, synthetic construct
  REGION 1..10: note = Description of sequence: Gene: MROH7; mutation: S214F; HLA type: A*02: 01
SLDLDFNPLL

SEQ ID NO: 203 - protein, 9 aa, synthetic construct
  REGION 1..9: note = Description of sequence: Gene: MTHFD1L; mutation: V418I; HLA type: A*02: 01
VLIAGITPT

SEQ ID NO: 204 - protein, 9 aa, synthetic construct
  REGION 1..9: note = Description of sequence: Gene: MYADM; mutation: S68F; HLA type: A*02: 01
FMFTWCFCF

SEQ ID NO: 205 - protein, 9 aa, synthetic construct
  REGION 1..9: note = Description of sequence: Gene: NID1; mutation: G495E; HLA type: A*02: 01
GIIEWMFAV

SEQ ID NO: 206 - protein, 9 aa, synthetic construct
  REGION 1..9: note = Description of sequence: Gene: NLRP1; mutation: P614L; HLA type: A*02: 01
ILQEHLIPL

SEQ ID NO: 207 - protein, 10 aa, synthetic construct
  REGION 1..10: note = Description of sequence: Gene: NPHP4; mutation: P159L; HLA type: A*02: 01
RLYHGTLRAL

SEQ ID NO: 208 - protein, 9 aa, synthetic construct
  REGION 1..9: note = Description of sequence: Gene: NUP214; mutation: P1463S; HLA type: A*02: 01
SLPTSFPTL

SEQ ID NO: 209 - protein, 9 aa, synthetic construct
  REGION 1..9: note = Description of sequence: Gene: PADI1; mutation: S118F; HLA type: A*02: 01
YLTGVDIFL

SEQ ID NO: 210 - protein, 10 aa, synthetic construct
  REGION 1..10: note = Description of sequence: Gene: PAQR7; mutation: S119F; HLA type: A*02: 01
YLFFSALAHL

SEQ ID NO: 211 - protein, 10 aa, synthetic construct
  REGION 1..10: note = Description of sequence: Gene: PKP2; mutation: F491L; HLA type: A*02: 01
TLTENIIIPL

SEQ ID NO: 212 - protein, 9 aa, synthetic construct
  REGION 1..9: note = Description of sequence: Gene: PLEKHG4; mutation: S635F; HLA type: A*02: 01
ALPQAFPTV

SEQ ID NO: 213 - protein, 11 aa, synthetic construct
  REGION 1..11: note = Description of sequence: Gene: PTK7; mutation: D463N; HLA type: A*02: 01
MLISENSRFEV

SEQ ID NO: 214 - protein, 10 aa, synthetic construct
  REGION 1..10: note = Description of sequence: Virus: HPV16; location: E6 29-38; HLA: A0201
TIHDIILECV

SEQ ID NO: 215 - protein, 9 aa, synthetic construct
  REGION 1..9: note = Description of sequence: Virus: HPV16; location: E7 11-19; HLA: A0201
YMLDLQPET

SEQ ID NO: 216 - protein, 8 aa, synthetic construct
  REGION 1..8: note = Description of sequence: Virus: HPV16; location: E6 31-38; HLA: B4002
HDIILECV

SEQ ID NO: 217 - protein, 10 aa, synthetic construct
  REGION 1..10: note = Description of sequence: Virus: HPV; location: E7 11-20; HLA: A0201
YMLDLQPETT

SEQ ID NO: 218 - protein, 8 aa, synthetic construct
  REGION 1..8: note = Description of sequence: Virus: HPV; location: E7 86-93; HLA: A0201
TLGIVCPI

SEQ ID NO: 219 - protein, 9 aa, synthetic construct
  REGION 1..9: note = Description of sequence: Virus: HPV-16; location: E6 (aa 11-19); HLA: A*02
KLPQLCTEV

SEQ ID NO: 220 - protein, 9 aa, synthetic construct
  REGION 1..9: note = Description of sequence: Virus: HPV-16; location: E6 (aa 29-37); HLA: B*48
TIHDIILEC

SEQ ID NO: 221 - protein, 9 aa, synthetic construct
  REGION 1..9: note = Description of sequence: Virus: HPV-16; location: E6 (aa 72-80); HLA: A*02
KISEYRHYC

SEQ ID NO: 222 - protein, 10 aa, synthetic construct
  REGION 1..10: note = Description of sequence: Virus: HPV-16; location: E6 (aa 90-99); HLA: A*02
QLYNKPLCDV

SEQ ID NO: 223 - protein, 9 aa, synthetic construct
  REGION 1..9: note = Description of sequence: Virus: HPV-16; location: E7 (aa 7-15); HLA: A*02/B*48
TLHEYMLDL

SEQ ID NO: 224 - protein, 9 aa, synthetic construct
  REGION 1..9: note = Description of sequence: Virus: HPV-16; location: E7 (aa 61-69); HLA: A*24
CDSTLRLCV

SEQ ID NO: 225 - protein, 10 aa, synthetic construct
  REGION 1..10: note = Description of sequence: Virus: HPV-16; location: E7 (aa 67-76); HLA: A*24
LCVQSTHVDI

SEQ ID NO: 226 - protein, 10 aa, synthetic construct
  REGION 1..10: note = Description of sequence: Virus: HPV-16; location: E7 (aa 77-86); HLA: A*02
RTLEDLLMGV

SEQ ID NO: 227 - protein, 9 aa, synthetic construct
  REGION 1..9: note = Description of sequence: Virus: HPV-16; location: E7 (aa 79-87); HLA: B*60
LEDLLMGTL

SEQ ID NO: 228 - protein, 9 aa, synthetic construct
  REGION 1..9: note = Description of sequence: Virus: HPV-16; location: E7 (aa 82-90); HLA: A*02
LLMGTLGIV

SEQ ID NO: 229 - protein, 9 aa, synthetic construct
  REGION 1..9: note = Description of sequence: Virus: HPV16; location: L1 443-452; HLA: A*24:02/A*23:01
KYTFWEVNL

SEQ ID NO: 230 - protein, 9 aa, synthetic construct
  REGION 1..9: note = Description of sequence: Virus: HPV16; location: L1 444-453; HLA: A*68:01/A*11:01
YTFWEVNLK

SEQ ID NO: 231 - protein, 9 aa, synthetic construct
  REGION 1..9: note = Description of sequence: Virus: HPV16; location: L1 398-408; HLA: A*02:01/A*02:06
ILEDWNFGL

SEQ ID NO: 232 - protein, 9 aa, synthetic construct
  REGION 1..9: note = Description of sequence: Virus: HPV16; location: L1 244-253; HLA: A*33:01
DSLFFYLRR

SEQ ID NO: 233 - protein, 9 aa, synthetic construct
  REGION 1..9: note = Description of sequence: Virus: HPV16; location: L1 186-195; HLA: B*51:01
PPLELINTV

SEQ ID NO: 234 - protein, 9 aa, synthetic construct
  REGION 1..9: note = Description of sequence: Virus: HPV16; location: L1 27-36; HLA: A*26:01/A*01:01/A*30:02/B*35:01/B*15:01
YVARTNIYY

SEQ ID NO: 235 - protein, 9 aa, synthetic construct
  REGION 1..9: note = Description of sequence: Virus: HPV16; location: L1 45-54; HLA: A*30:01
VGHPYFPIK

SEQ ID NO: 236 - protein, 9 aa, synthetic construct
  REGION 1..9: note = Description of sequence: Virus: HPV16; location: L1 330-339; HLA: A*68:01
FVTVVDTTR

SEQ ID NO: 237 - protein, 9 aa, synthetic construct
  REGION 1..9: note = Description of sequence: Virus: HPV16; location: L1 41-50; HLA: A*32:01/B*15:01/A*30:02
RLLAVGHPY

SEQ ID NO: 238 - protein, 9 aa, synthetic construct
  REGION 1..9: note = Description of sequence: Virus: HPV16; location: L1 133-142; HLA: A*68:02
SAYAANAGV

SEQ ID NO: 239 - protein, 9 aa, synthetic construct
  REGION 1..9: note = Description of sequence: Virus: HPV16; location: L1 42-51; HLA: B*15:01
LLAVGHPYF

SEQ ID NO: 240 - protein, 9 aa, synthetic construct
  REGION 1..9: note = Description of sequence: Virus: HPV16; location: L1 247-256; HLA: B*08:01
FFYLRREQM

SEQ ID NO: 241 - protein, 9 aa, synthetic construct
  REGION 1..9: note = Description of sequence: Virus: HPV16; location: L1 368-397; HLA: B*44:03/B*44:02/B*40:01
EEYDLQFIF

SEQ ID NO: 242 - protein, 9 aa, synthetic construct
  REGION 1..9: note = Description of sequence: Virus: HPV16; location: L1 82-91; HLA: A*23:01/A*24:02
KFGFPDTSF

SEQ ID NO: 243 - protein, 9 aa, synthetic construct
  REGION 1..9: note = Description of sequence: Virus: HPV16; location: L1 422-431; HLA: A*11:01
TSQAIACQK

SEQ ID NO: 244 - protein, 9 aa, synthetic construct
  REGION 1..9: note = Description of sequence: Virus: HPV16; location: L1 105-114; HLA: B*40:01
VEVGRGQPL

SEQ ID NO: 245 - protein, 9 aa, synthetic construct
  REGION 1..9: note = Description of sequence: Virus: HPV16; location: L1 83-92; HLA: B*35:01/A*30:02/A*01:01
FGFPDTSFY

SEQ ID NO: 246 - protein, 9 aa, synthetic construct
  REGION 1..9: note = Description of sequence: Virus: HPV16; location: L1 348-357; HLA: A*11:01
ISTSETTYK

SEQ ID NO: 247 - protein, 9 aa, synthetic construct
  REGION 1..9: note = Description of sequence: Virus: HPV16; location: E5 71-80; HLA: A*33:01
LFLIHTHAR

SEQ ID NO: 248 - protein, 9 aa, synthetic construct
  REGION 1..9: note = Description of sequence: Virus: HPV16; location: E5 72-81; HLA: B*15:01
FLIHTHARF

SEQ ID NO: 249 - protein, 9 aa, synthetic construct
  REGION 1..9: note = Description of sequence: Virus: HPV16; location: E5 38-47; HLA: A*24:02/ A*23:01
TYTSLIILV

SEQ ID NO: 250 - protein, 9 aa, synthetic construct
  REGION 1..9: note = Description of sequence: Virus: HPV16; location: E6 134-143; HLA: A*33:01
NIRGRWTGR

SEQ ID NO: 251 - protein, 9 aa, synthetic construct
  REGION 1..9: note = Description of sequence: Virus: HPV16; location: E6 53-62; HLA: A*30:02
AFRDLCIVY

SEQ ID NO: 252 - protein, 9 aa, synthetic construct
  REGION 1..9: note = Description of sequence: Virus: HPV16; location: E6 80-89; HLA: A*01:01
ISEYRHYCY

SEQ ID NO: 253 - protein, 9 aa, synthetic construct
  REGION 1..9: note = Description of sequence: Virus: HPV16; location: E6 11-20; HLA: B*08:01/ B*51:01
DPQERPRKL

SEQ ID NO: 254 - protein, 9 aa, synthetic construct
  REGION 1..9: note = Description of sequence: Virus: HPV16; location: E6 87-96; HLA: A*24:02
CYSLYGTTL

SEQ ID NO: 255 - protein, 9 aa, synthetic construct
  REGION 1..9: note = Description of sequence: Virus: HPV16; location: E7 15-24; HLA: B*15:01
LQPETTDLY

SEQ ID NO: 256 - protein, 9 aa, synthetic construct
  REGION 1..9: note = Description of sequence: Virus: HPV16; location: E7 49-58; HLA: A*32:01/B*15:01/B*58:01/B*57:01/B*35:01
RAHYNIVTF

SEQ ID NO: 257 - protein, 9 aa, synthetic construct
  REGION 1..9: note = Description of sequence: Virus: HPV16; location: E7 46-55; HLA: B*51:01/ B*53:01
EPDRAHYNI

SEQ ID NO: 258 - protein, 9 aa, synthetic construct
  REGION 1..9: note = Description of sequence: Virus: HPV16; location: E7 82-91; HLA: A*02:03
LLMGTLGIV

SEQ ID NO: 259 - protein, 9 aa, synthetic construct
  REGION 1..9: note = Description of sequence: Virus: HPV 18; location: E5 (aa61-70); HLA: A*02:01/A*02:03/A*02:06/B*08:01
MLLLHIHAI

SEQ ID NO: 260 - protein, 9 aa, synthetic construct
  REGION 1..9: note = Description of sequence: Virus: HPV 18; location: E5 (aa31-40); HLA: B*35:01
YAWVLVFVY

SEQ ID NO: 261 - protein, 9 aa, synthetic construct
  REGION 1..9: note = Description of sequence: Virus: HPV 18; location: E6 (aa 13-21); HLA: A*02
KLPDLCTEL

SEQ ID NO: 262 - protein, 9 aa, synthetic construct
  REGION 1..9: note = Description of sequence: Virus: HPV 18; location: E6 (aa 36-44); HLA: A*02
KTVLELTEV

SEQ ID NO: 263 - protein, 9 aa, synthetic construct
  REGION 1..9: note = Description of sequence: Virus: HPV 18; location: E6 (aa 37-45); HLA: B*35:01/B*53:01
TVLELTEVF

SEQ ID NO: 264 - protein, 9 aa, synthetic construct
  REGION 1..9: note = Description of sequence: Virus: HPV 18; location: E6 (aa 39-47); HLA: B*44:03/B*44:02
LELTEVFEF

SEQ ID NO: 265 - protein, 9 aa, synthetic construct
  REGION 1..9: note = Description of sequence: Virus: HPV 18; location: E6 (aa 40-49); HLA: A*68:02
ELTEVFEFA

SEQ ID NO: 266 - protein, 9 aa, synthetic construct
  REGION 1..9: note = Description of sequence: Virus: HPV 18; location: E6 (aa 45-51); HLA: B*40:01/B*44:03
FEFAFKDLF

SEQ ID NO: 267 - protein, 9 aa, synthetic construct
  REGION 1..9: note = Description of sequence: Virus: HPV 18; location: E6 (aa 50-58); HLA: A*02
ELTEVFEFA

SEQ ID NO: 268 - protein, 10 aa, synthetic construct
  REGION 1..10: note = Description of sequence: Virus: HPV18; location: E6 52-61; HLA: B*35/B*57
FAFRDLCIVY

SEQ ID NO: 269 - protein, 9 aa, synthetic construct
  REGION 1..9: note = Description of sequence: Virus: HPV 18; location: E6 (aa 54-62); HLA: A*11
VVYRDSIPH

SEQ ID NO: 270 - protein, 9 aa, synthetic construct
  REGION 1..9: note = Description of sequence: Virus: HPV 18; location: E6 (aa 67-75); HLA: A*02
KCIDFYSRI

SEQ ID NO: 271 - protein, 9 aa, synthetic construct
  REGION 1..9: note = Description of sequence: Virus: HPV 18; location: E6 (aa 70-79); HLA: B*08:01
DFYSRIREL

SEQ ID NO: 272 - protein, 9 aa, synthetic construct
  REGION 1..9: note = Description of sequence: Virus: HPV 18; location: E6 (aa 84-92); HLA: A*11
SVYGDTLEK

SEQ ID NO: 273 - protein, 9 aa, synthetic construct
  REGION 1..9: note = Description of sequence: Virus: HPV 18; location: E6 (aa 90-99); HLA: B*40:01
LEKLTNTGL

SEQ ID NO: 274 - protein, 9 aa, synthetic construct
  REGION 1..9: note = Description of sequence: Virus: HPV 18; location: E6 (aa 126-135); HLA: A*30:02
RFHNIAGHY

SEQ ID NO: 275 - protein, 9 aa, synthetic construct
  REGION 1..9: note = Description of sequence: Virus: HPV 18; location: E7 (aa 7-15); HLA: A*02
TLQDIVLHL

SEQ ID NO: 276 - protein, 15 aa, synthetic construct
  REGION 1..15: note = Description of sequence: Virus: HPV 18; location: E7 (aa 81-95); HLA: A*02/A*11/A*24/A*33
DDLRAFQQLFLNTLS

SEQ ID NO: 277 - protein, 9 aa, synthetic construct
  REGION 1..9: note = Description of sequence: Virus: HPV 18; location: E7 (aa 86-94); HLA: A*02
FQQLFLNTL

SEQ ID NO: 278 - protein, 10 aa, synthetic construct
  REGION 1..10: note = Description of sequence: Virus: HPV 18; location: E7 (aa 88-97); HLA: A*02
QLFLNTLSFV

SEQ ID NO: 279 - protein, 15 aa, synthetic construct
  REGION 1..15: note = Description of sequence: Virus: HPV 18; location: E7 (aa 89-103); HLA: A*02/A*11/A*24/A*33
LFLNTLSFVCPWCAS

SEQ ID NO: 280 - skipped

SEQ ID NO: 281 - protein, 9 aa, synthetic construct
  REGION 1..9: note = Description of sequence: Virus: CMV; location: pp65 495-503; HLA: A0201
NLVPMVATV

SEQ ID NO: 282 - protein, 9 aa, synthetic construct
  REGION 1..9: note = Description of sequence: Virus: CMV; location: pp65 417-425; HLA: B0702
TPRVTGGGA

SEQ ID NO: 283 - protein, 9 aa, synthetic construct
  REGION 1..9: note = Description of sequence: Virus: CMV; location: pp65 418-426; HLA: B0702
PRVTGGGAM

SEQ ID NO: 284 - protein, 9 aa, synthetic construct
  REGION 1..9: note = Description of sequence: Virus: CMV; location: pp65 188-196; HLA: A6801
FPTKDVALR

SEQ ID NO: 285 - protein, 11 aa, synthetic construct
  REGION 1..11: note = Description of sequence: Virus: CMV; location: GB: 619-628 ; HLA: A2
FIAGNSAYEYV

SEQ ID NO: 286 - protein, 12 aa, synthetic construct
  REGION 1..12: note = Description of sequence: Virus: CMV; location: IE1: 378-389 ; HLA: B18
SDEEEAIVAYTL

SEQ ID NO: 287 - protein, 9 aa, synthetic construct
  REGION 1..9: note = Description of sequence: Virus: CMV; location: US8 74-82; HLA: A*02
GVLDAVWRV

SEQ ID NO: 288 - protein, 10 aa, synthetic construct
  REGION 1..10: note = Description of sequence: Virus: CMV; location: UL150A 152-161; HLA: A*02
ALWDVALLEV

SEQ ID NO: 289 - protein, 9 aa, synthetic construct
  REGION 1..9: note = Description of sequence: Virus: CMV; location: UL100 200-208; HLA: A*02
TLIVNLVEV

SEQ ID NO: 290 - protein, 9 aa, synthetic construct
  REGION 1..9: note = Description of sequence: Virus: CMV; location: UL44 259-267; HLA: A*02
GLFAVENFL

SEQ ID NO: 291 - protein, 9 aa, synthetic construct
  REGION 1..9: note = Description of sequence: Virus: CMV; location: UL16 162-170; HLA: B*07
YPRPPGSGL

SEQ ID NO: 292 - protein, 9 aa, synthetic construct
  REGION 1..9: note = Description of sequence: Virus: CMV; location: TRS1 166-174; HLA: B*07
SPRDAWIVL

SEQ ID NO: 293 - protein, 10 aa, synthetic construct
  REGION 1..10: note = Description of sequence: Virus: CMV; location: UL52 349-357; HLA: B*07
SPSRDRFVQL

SEQ ID NO: 294 - protein, 9 aa, synthetic construct
  REGION 1..9: note = Description of sequence: Virus: CMV; location: UL23 22-30; HLA: B*07
RPWKPGQRV

SEQ ID NO: 295 - protein, 9 aa, synthetic construct
  REGION 1..9: note = Description of sequence: Virus: CMV; location: UL46 76-84; HLA: B*07
SPRHLYISL

SEQ ID NO: 296 - protein, 10 aa, synthetic construct
  REGION 1..10: note = Description of sequence: Virus: CMV; location: UL112/UL113 125-134; HLA: B*44
SENGNLQVTY

SEQ ID NO: 297 - protein, 9 aa, synthetic construct
  REGION 1..9: note = Description of sequence: Virus: CMV; location: UL117 358-366; HLA: B*44
HETGVYQMW

SEQ ID NO: 298 - protein, 9 aa, synthetic construct
  REGION 1..9: note = Description of sequence: Virus: CMV; location: UL105 715-723; HLA: A*01
YADPFFLKY

SEQ ID NO: 299 - protein, 9 aa, synthetic construct
  REGION 1..9: note = Description of sequence: Virus: CMV; location: UL44 245-253; HLA: A*01
VTEHDTLLY

SEQ ID NO: 300 - protein, 10 aa, synthetic construct
  REGION 1..10: note = Description of sequence: Virus: CMV; location: UL69 569-578; HLA: A*01
RTDPATLTAY

SEQ ID NO: 301 - protein, 9 aa, synthetic construct
  REGION 1..9: note = Description of sequence: Virus: CMV; location: US28 122-130; HLA: A*01
ITEIALDRY

SEQ ID NO: 302 - protein, 10 aa, synthetic construct
  REGION 1..10: note = Description of sequence: Virus: CMV; location: UL55 657-665; HLA: A*01
NTDFRVLELY

SEQ ID NO: 303 - protein, 9 aa, synthetic construct
  REGION 1..9: note = Description of sequence: Virus: CMV; location: UL77 228-236; HLA: A*03
GLYTQPRWK

SEQ ID NO: 304 - protein, 9 aa, synthetic construct
  REGION 1..9: note = Description of sequence: Virus: CMV; location: UL57 790-798; HLA: A*03
RVKNRPIYR

SEQ ID NO: 305 - protein, 9 aa, synthetic construct
  REGION 1..9: note = Description of sequence: Virus: CMV; location: UL34 180-188; HLA: B*08
LPHERHREL

SEQ ID NO: 306 - protein, 9 aa, synthetic construct
  REGION 1..9: note = Description of sequence: Virus: CMV; location: UL26 61-69; HLA: B*08
LPYPRGYTL

SEQ ID NO: 307 - protein, 9 aa, synthetic construct
  REGION 1..9: note = Description of sequence: Virus: CMV; location: UL13 465-473; HLA: B*08
YLVRRPMTI

SEQ ID NO: 308 - protein, 8 aa, synthetic construct
  REGION 1..8: note = Description of sequence: Virus: CMV; location: UL83 116-123; HLA: B*51
LPLKMLNI

SEQ ID NO: 309 - protein, 9 aa, synthetic construct
  REGION 1..9: note = Description of sequence: Virus: CMV; location: UL38 156-164; HLA: B*51
LPYPRGYTL

SEQ ID NO: 310 - protein, 9 aa, synthetic construct
  REGION 1..9: note = Description of sequence: Virus: CMV; location: UL26 61-69; HLA: B*51
FPVEVRSHV

SEQ ID NO: 311 - protein, 9 aa, synthetic construct
  REGION 1..9: note = Description of sequence: Virus: CMV; location: UL56 503-511; HLA: B*51
DARSRIHNV

SEQ ID NO: 312 - protein, 9 aa, synthetic construct
  REGION 1..9: note = Description of sequence: Virus: CMV; location: IE-1; HLA: A*0201
VLEETSVML

SEQ ID NO: 313 - protein, 9 aa, synthetic construct
  REGION 1..9: note = Description of sequence: Virus: CMV; location: IE-1; HLA: A*0301
KLGGALQAK

SEQ ID NO: 314 - protein, 9 aa, synthetic construct
  REGION 1..9: note = Description of sequence: Virus: CMV; location: pp65; HLA: A*2301
QYDPVAALF

SEQ ID NO: 315 - protein, 9 aa, synthetic construct
  REGION 1..9: note = Description of sequence: Virus: CMV; location: pp65; HLA: A*2301
AYAQKIFKI

SEQ ID NO: 316 - protein, 9 aa, synthetic construct
  REGION 1..9: note = Description of sequence: Virus: CMV; location: pp65; HLA: A*2402
VYALPLKML

SEQ ID NO: 317 - protein, 11 aa, synthetic construct
  REGION 1..11: note = Description of sequence: Virus: CMV; location: pp65; HLA: B*0702
RPHERNGFTVL

SEQ ID NO: 318 - protein, 10 aa, synthetic construct
  REGION 1..10: note = Description of sequence: Virus: CMV; location: pp65; HLA: B*0702
TPRVTGGGAM

SEQ ID NO: 319 - protein, 9 aa, synthetic construct
  REGION 1..9: note = Description of sequence: Virus: CMV; location: IE-1; HLA: B*0801
ELKRKMIYM

SEQ ID NO: 320 - protein, 9 aa, synthetic construct
  REGION 1..9: note = Description of sequence: Virus: CMV; location: IE-1; HLA: B*0801
ELNRKMIYM

SEQ ID NO: 321 - protein, 9 aa, synthetic construct
  REGION 1..9: note = Description of sequence: Virus: CMV; location: IE-1; HLA: B*0801
ELRRKMMYM

SEQ ID NO: 322 - protein, 9 aa, synthetic construct
  REGION 1..9: note = Description of sequence: Virus: CMV; location: IE-1; HLA: B*0801
QIKVRVDMV

SEQ ID NO: 323 - protein, 9 aa, synthetic construct
  REGION 1..9: note = Description of sequence: Virus: CMV; location: pp65; HLA: B*3501
IPSINVHHY

SEQ ID NO: 324 - protein, 10 aa, synthetic construct
  REGION 1..10: note = Description of sequence: Virus: EBV; location: EBNA 2; HLA: A*02
DTPLIPLTIF

SEQ ID NO: 325 - protein, 9 aa, synthetic construct
  REGION 1..9: note = Description of sequence: Virus: EBV; location: EBNA 3a; HLA: B8
FLRGRAYGL

SEQ ID NO: 326 - protein, 10 aa, synthetic construct
  REGION 1..10: note = Description of sequence: Virus: EBV; location: EBNA 3c; HLA: B44
EENLLDFVRF

SEQ ID NO: 327 - protein, 9 aa, synthetic construct
  REGION 1..9: note = Description of sequence: Virus: EBV; location: EBNA 3 A: 379-387; HLA: B7
RPPIFIRRL

SEQ ID NO: 328 - protein, 9 aa, synthetic construct
  REGION 1..9: note = Description of sequence: Virus: EBV; location: EBNA 3 A: 603-611; HLA: A3
RLRAEAQVK

SEQ ID NO: 329 - protein, 9 aa, synthetic construct
  REGION 1..9: note = Description of sequence: Virus: EBV; location: EBNA 3 B: 416-424; HLA: A11
IVTDFSVIK

SEQ ID NO: 330 - protein, 9 aa, synthetic construct
  REGION 1..9: note = Description of sequence: Virus: EBV; location: EBNA 3 C: 163-171; HLA: B44
EGGVGWRHW

SEQ ID NO: 331 - protein, 9 aa, synthetic construct
  REGION 1..9: note = Description of sequence: Virus: EBV; location: EBNA 3 C: 881-891; HLA: B7
QPRAPIRPI

SEQ ID NO: 332 - protein, 9 aa, synthetic construct
  REGION 1..9: note = Description of sequence: Virus: EBV; location: EBNA 3 A: 325-333; HLA: B8
FRLGRAYGL

SEQ ID NO: 333 - protein, 9 aa, synthetic construct
  REGION 1..9: note = Description of sequence: Virus: EBV; location: EBNA 3 A: 458-466; HLA: B35
YPLHEQHGM

SEQ ID NO: 334 - protein, 9 aa, synthetic construct
  REGION 1..9: note = Description of sequence: Virus: EBV; location: LMP-2: 426-434; HLA: A2
CLGGLLTMV

SEQ ID NO: 335 - protein, 9 aa, synthetic construct
  REGION 1..9: note = Description of sequence: Virus: EBV; location: BMLF-1: 280-288; HLA: A2
GLCTLVAML

SEQ ID NO: 336 - protein, 8 aa, synthetic construct
  REGION 1..8: note = Description of sequence: Virus: EBV; location: BZLF-1: 190-197; HLA: B8
RAKFKQLL

SEQ ID NO: 337 - protein, 15 aa, synthetic construct
  REGION 1..15: note = Description of sequence: Virus: EBV; location: EBNA 2; HLA: B18
PRSPTVFYNIPPMPL

SEQ ID NO: 338 - protein, 15 aa, synthetic construct
  REGION 1..15: note = Description of sequence: Virus: EBV; location: EBNA 3c; HLA: A24
RGIKEHVIQNAFRKA

SEQ ID NO: 339 - protein, 9 aa, synthetic construct
  REGION 1..9: note = Description of sequence: Virus: EBV; location: BRLF1; HLA: A2.1
YVLDHLIVV

SEQ ID NO: 340 - protein, 9 aa, synthetic construct
  REGION 1..9: note = Description of sequence: Virus: EBV; location: BMRF1; HLA: A2.1
TLDYKPLSV

SEQ ID NO: 341 - protein, 9 aa, synthetic construct
  REGION 1..9: note = Description of sequence: Virus: EBV; location: EBNA3A; HLA: B8
QAKWRLQTL

SEQ ID NO: 342 - protein, 9 aa, synthetic construct
  REGION 1..9: note = Description of sequence: Virus: EBV; location: BALF4; HLA: A*0201
FLDKGTYTL

SEQ ID NO: 343 - protein, 9 aa, synthetic construct
  REGION 1..9: note = Description of sequence: Virus: EBV; location: BMLF1259-267; HLA: A*0201
GLCTLVAML

SEQ ID NO: 344 - protein, 9 aa, synthetic construct
  REGION 1..9: note = Description of sequence: Virus: EBV; location: BMRF1; HLA: A*0201
TLDYKPLSV

SEQ ID NO: 345 - protein, 9 aa, synthetic construct
  REGION 1..9: note = Description of sequence: Virus: EBV; location: BRLF1109- 117; HLA: A*0201
YVLDHLIVV

SEQ ID NO: 346 - protein, 10 aa, synthetic construct
  REGION 1..10: note = Description of sequence: Virus: EBV; location: EBNA 3B; HLA: A*0201
LLDFVRFMGV

SEQ ID NO: 347 - protein, 9 aa, synthetic construct
  REGION 1..9: note = Description of sequence: Virus: EBV; location: LMP-1; HLA: A*0201
YLLEMLWRL

SEQ ID NO: 348 - protein, 9 aa, synthetic construct
  REGION 1..9: note = Description of sequence: Virus: EBV; location: LMP1159-167; HLA: A*0201
YLQQNWWTL

SEQ ID NO: 349 - protein, 9 aa, synthetic construct
  REGION 1..9: note = Description of sequence: Virus: EBV; location: LMP-2A; HLA: A*0201
CLGGLLTMV

SEQ ID NO: 350 - protein, 9 aa, synthetic construct
  REGION 1..9: note = Description of sequence: Virus: EBV; location: LMP-2A; HLA: A*0201
FLYALALLL

SEQ ID NO: 351 - protein, 9 aa, synthetic construct
  REGION 1..9: note = Description of sequence: Virus: EBV; location: EMNA 3A; HLA: A*0301
RLRAEAQVK

SEQ ID NO: 352 - protein, 10 aa, synthetic construct
  REGION 1..10: note = Description of sequence: Virus: EBV; location: EBNA 3B; HLA: A*1101
AVFDRKSDAK

SEQ ID NO: 353 - protein, 9 aa, synthetic construct
  REGION 1..9: note = Description of sequence: Virus: EBV; location: EBNA 3B; HLA: A*1101
IVTDFSVIK

SEQ ID NO: 354 - protein, 10 aa, synthetic construct
  REGION 1..10: note = Description of sequence: Virus: EBV; location: BRLF1; HLA: A*2402
DYCNVLNKEF

SEQ ID NO: 355 - protein, 9 aa, synthetic construct
  REGION 1..9: note = Description of sequence: Virus: EBV; location: LMP-2; HLA: A*2402
TYGPVFMCL

SEQ ID NO: 356 - protein, 9 aa, synthetic construct
  REGION 1..9: note = Description of sequence: Virus: EBV; location: BRLF1; HLA: A*2902
IACPIVMRY

SEQ ID NO: 357 - protein, 9 aa, synthetic construct
  REGION 1..9: note = Description of sequence: Virus: EBV; location: EBNA 3B; HLA: A*6801
IVTDFSVIK

SEQ ID NO: 358 - protein, 13 aa, synthetic construct
  REGION 1..13: note = Description of sequence: Virus: EBV; location: BMRF1; HLA: B*0702
RPQGGSRPEFVKL

SEQ ID NO: 359 - protein, 9 aa, synthetic construct
  REGION 1..9: note = Description of sequence: Virus: EBV; location: EBNA 3A; HLA: B*0702
RPPIFIRRL

SEQ ID NO: 360 - protein, 9 aa, synthetic construct
  REGION 1..9: note = Description of sequence: Virus: EBV; location: EBNA 6; HLA: B*0702
QPRAPIRPI

SEQ ID NO: 361 - protein, 8 aa, synthetic construct
  REGION 1..8: note = Description of sequence: Virus: EBV; location: BZLF1; HLA: B*0801
RAKFKQLL

SEQ ID NO: 362 - protein, 9 aa, synthetic construct
  REGION 1..9: note = Description of sequence: Virus: EBV; location: EBNA 3A; HLA: B*0801
FLRGRAYGL

SEQ ID NO: 363 - protein, 11 aa, synthetic construct
  REGION 1..11: note = Description of sequence: Virus: EBV; location: BZLF1; HLA: B*3501
EPLPQGQLTAY

SEQ ID NO: 364 - protein, 11 aa, synthetic construct
  REGION 1..11: note = Description of sequence: Virus: EBV; location: EBNA 1; HLA: B*3501
HPVAEADYFEY

SEQ ID NO: 365 - protein, 11 aa, synthetic construct
  REGION 1..11: note = Description of sequence: Virus: EBV; location: EBNA 1; HLA: B*3501
HPVGDADYFEY

SEQ ID NO: 366 - protein, 11 aa, synthetic construct
  REGION 1..11: note = Description of sequence: Virus: EBV; location: EBNA 1; HLA: B*3501
HPVGEADYFEY

SEQ ID NO: 367 - protein, 11 aa, synthetic construct
  REGION 1..11: note = Description of sequence: Virus: EBV; location: EBNA 1; HLA: B*3501
HPVGQADYFEY

SEQ ID NO: 368 - protein, 9 aa, synthetic construct
  REGION 1..9: note = Description of sequence: Virus: EBV; location: EBNA 3A; HLA: B*3501
YPLHEQHGM

SEQ ID NO: 369 - protein, 9 aa, synthetic construct
  REGION 1..9: note = Description of sequence: Virus: HTLV; location: bZIP factor; HLA: A*0201
AVLDGLLSL

SEQ ID NO: 370 - protein, 9 aa, synthetic construct
  REGION 1..9: note = Description of sequence: Virus: HTLV; location: bZIP factor; HLA: A*0201
GLLSLEEEL

SEQ ID NO: 371 - protein, 9 aa, synthetic construct
  REGION 1..9: note = Description of sequence: Virus: HTLV; location: Tax; HLA: A*0201
LLFGYPVYV

SEQ ID NO: 372 - protein, 9 aa, synthetic construct
  REGION 1..9: note = Description of sequence: Virus: HTLV; location: tax; HLA: A*2402
SFHSLHLLF

SEQ ID NO: 373 - protein, 9 aa, synthetic construct
  REGION 1..9: note = Description of sequence: Virus: HTLV; location: bZIP factor; HLA: B*0702
LPVSCPEDL

SEQ ID NO: 374 - protein, 9 aa, synthetic construct
  REGION 1..9: note = Description of sequence: Virus: HBV; location: HBX 133-141; HLA: A*02:01
VLGGCRHKL

SEQ ID NO: 375 - protein, 9 aa, synthetic construct
  REGION 1..9: note = Description of sequence: Virus: HBV; location: HBX 52-60; HLA: A*02:01
HLSLRGLPV

SEQ ID NO: 376 - protein, 9 aa, synthetic construct
  REGION 1..9: note = Description of sequence: Virus: HBV; location: HBX 135-143; HLA: A*11:01
GGCRHKLVC

SEQ ID NO: 377 - protein, 9 aa, synthetic construct
  REGION 1..9: note = Description of sequence: Virus: HBV; location: HBX 92-100; HLA: A*02:01
VLHKRTLGL

SEQ ID NO: 378 - protein, 10 aa, synthetic construct
  REGION 1..10: note = Description of sequence: Virus: HBV; location: HBX 91-100; HLA: A*02:01
KVLHKRTLGL

SEQ ID NO: 379 - protein, 10 aa, synthetic construct
  REGION 1..10: note = Description of sequence: Virus: HBV; location: HBX 99-108; HLA: A*02
GLSAMSTTDL

SEQ ID NO: 380 - protein, 9 aa, synthetic construct
  REGION 1..9: note = Description of sequence: Virus: HBV; location: HBX 102-110; HLA: A*02:01
AMSTTDLEA

SEQ ID NO: 381 - protein, 9 aa, synthetic construct
  REGION 1..9: note = Description of sequence: Virus: HBV; location: HBX 15-23; HLA: A*02:01
VLCLRPVGA

SEQ ID NO: 382 - protein, 9 aa, synthetic construct
  REGION 1..9: note = Description of sequence: Virus: HBV; location: HBX 8-16; HLA: A*02:01
QLDPARDVL

SEQ ID NO: 383 - protein, 9 aa, synthetic construct
  REGION 1..9: note = Description of sequence: Virus: HBV; location: HBX 115-123; HLA: A*02:01
CLFKDWEEL

SEQ ID NO: 384 - protein, 9 aa, synthetic construct
  REGION 1..9: note = Description of sequence: Virus: HBV; location: HBX 84-92; HLA: A*02:01
NAHQVLPKV

SEQ ID NO: 385 - protein, 9 aa, synthetic construct
  REGION 1..9: note = Description of sequence: Virus: HBV; location: HBX 97-105; HLA: A*02:01
TLGLAAMST

SEQ ID NO: 386 - protein, 9 aa, synthetic construct
  REGION 1..9: note = Description of sequence: Virus: HBV; location: HBX 140-148; HLA: A*02:01
KLVCSPAPC

SEQ ID NO: 387 - protein, 10 aa, synthetic construct
  REGION 1..10: note = Description of sequence: Virus: HBV; location: HBX 44-53; HLA: A*02:01
VVPTDHGAHL

SEQ ID NO: 388 - protein, 9 aa, synthetic construct
  REGION 1..9: note = Description of sequence: Virus: polyomaviruses (JCV/BKV); location: JCV-VP1-p36-44; HLA: A*02:01
SITEVECFL

SEQ ID NO: 389 - protein, 9 aa, synthetic construct
  REGION 1..9: note = Description of sequence: Virus: polyomaviruses (JCV/BKV); location: JCV-VP1-p100-108; HLA: A*02:01
ILMWEAVTL

SEQ ID NO: 390 - protein, 9 aa, synthetic construct
  REGION 1..9: note = Description of sequence: Virus: polyomaviruses (JCV/BKV); location: BKV-VP1-p44-52; HLA: A*02:01
AITEVECFL

SEQ ID NO: 391 - protein, 9 aa, synthetic construct
  REGION 1..9: note = Description of sequence: Virus: polyomaviruses (JCV/BKV); location: BKV-VP1-p108-116; HLA: A*02:01
LLMWEAVTV

SEQ ID NO: 392 - protein, 9 aa, synthetic construct
  REGION 1..9: note = Description of sequence: Virus: polyomaviruses (JCV/BKV); location: OP50; HLA: A*02:01
IGVTSLMNV

SEQ ID NO: 393 - protein, 9 aa, synthetic construct
  REGION 1..9: note = Description of sequence: Virus: polyomaviruses (JCV/BKV); location: OP20; HLA: A*02:01
VARIPLPNL

SEQ ID NO: 394 - protein, 9 aa, synthetic construct
  REGION 1..9: note = Description of sequence: Virus: polyomaviruses (JCV/BKV); location: OP28; HLA: A*02:01
TEVIGVTSL

SEQ ID NO: 395 - protein, 9 aa, synthetic construct
  REGION 1..9: note = Description of sequence: Virus: polyomaviruses (JCV/BKV); location: OP52; HLA: A*02:01
WVPDPTRNE

SEQ ID NO: 396 - protein, 9 aa, synthetic construct
  REGION 1..9: note = Description of sequence: Virus: polyomaviruses (JCV/BKV); location: OP52; HLA: A*02:01
ENTRYFGTL

SEQ ID NO: 397 - protein, 9 aa, synthetic construct
  REGION 1..9: note = Description of sequence: Virus: polyomaviruses (JCV/BKV); location: OP65; HLA: A*02:01
GMFTNRSGS

SEQ ID NO: 398 - protein, 9 aa, synthetic construct
  REGION 1..9: note = Description of sequence: Virus: polyomaviruses (JCV/BKV); location: OP72; HLA: A*02:01
KNPYPISFL

SEQ ID NO: 399 - protein, 9 aa, synthetic construct
  REGION 1..9: note = Description of sequence: Virus: polyomaviruses (JCV/BKV); location: OP73; HLA: A*02:01
PISFLLTDL

SEQ ID NO: 400 - protein, 9 aa, synthetic construct
  REGION 1..9: note = Description of sequence: Virus: polyomaviruses (JCV/BKV); location: OP78; HLA: A*02:01
PMYGMDAQV

SEQ ID NO: 401 - protein, 9 aa, synthetic construct
  REGION 1..9: note = Description of sequence: Virus: polyomaviruses (JCV/BKV); location: OP79; HLA: A*02:01
GMDAQVEEV

SEQ ID NO: 402 - protein, 9 aa, synthetic construct
  REGION 1..9: note = Description of sequence: Virus: polyomaviruses (JCV/BKV); location: OP83; HLA: A*02:01
ELPGDPDMM

SEQ ID NO: 403 - protein, 9 aa, synthetic construct
  REGION 1..9: note = Description of sequence: Virus: polyomaviruses (JCV/BKV); location: p100; HLA: A*02:01
ILMWEAVTL

SEQ ID NO: 404 - protein, 9 aa, synthetic construct
  REGION 1..9: note = Description of sequence: Virus: polyomaviruses (JCV/BKV); location: OP12; HLA: B7
DPDEHLRGF

SEQ ID NO: 405 - protein, 9 aa, synthetic construct
  REGION 1..9: note = Description of sequence: Virus: polyomaviruses (JCV/BKV); location: OP13; HLA: B7
DPDEHLRGF

SEQ ID NO: 406 - protein, 9 aa, synthetic construct
  REGION 1..9: note = Description of sequence: Virus: polyomaviruses (JCV/BKV); location: OP18; HLA: B7
SDSPNRDML

SEQ ID NO: 407 - protein, 9 aa, synthetic construct
  REGION 1..9: note = Description of sequence: Virus: polyomaviruses (JCV/BKV); location: OP23; HLA: B7
EDLTCGNIL

SEQ ID NO: 408 - protein, 9 aa, synthetic construct
  REGION 1..9: note = Description of sequence: Virus: polyomaviruses (JCV/BKV); location: OP40; HLA: B7
RTKYPDGTI

SEQ ID NO: 409 - protein, 9 aa, synthetic construct
  REGION 1..9: note = Description of sequence: Virus: polyomaviruses (JCV/BKV); location: OP53; HLA: B7
ENTRYFGTL

SEQ ID NO: 410 - protein, 9 aa, synthetic construct
  REGION 1..9: note = Description of sequence: Virus: polyomaviruses (JCV/BKV); location: OP57; HLA: B7
ITNTATTVL

SEQ ID NO: 411 - protein, 9 aa, synthetic construct
  REGION 1..9: note = Description of sequence: Virus: polyomaviruses (JCV/BKV); location: p244; HLA: B7
GPLCKGDNL

SEQ ID NO: 412 - protein, 9 aa, synthetic construct
  REGION 1..9: note = Description of sequence: Virus: polyomaviruses (JCV/BKV); location: OP76; HLA: B7
TPRVDGQPM

SEQ ID NO: 413 - protein, 9 aa, synthetic construct
  REGION 1..9: note = Description of sequence: Virus: polyomaviruses (JCV/BKV); location: OP72; HLA: B7
NPYPISFLL

SEQ ID NO: 414 - protein, 9 aa, synthetic construct
  REGION 1..9: note = Description of sequence: Virus: polyomaviruses (JCV/BKV); location: OP72; HLA: B7
NPYPISFLL

SEQ ID NO: 415 - protein, 13 aa, synthetic construct
  REGION 1..13: note = Description of sequence: Gene: PADRE peptide; HLA type: pan DR epitope
AKFVAAWTLKAAA

SEQ ID NO: 416 - protein, 15 aa, synthetic construct
  REGION 1..15: note = Description of sequence: Gene: S816-830; origin: SARS-CoV-2; HLA type: broadly recognized and present by common HLA II alleles
SFIEDLLFNKVTLAD

SEQ ID NO: 417 - protein, 21 aa, synthetic construct
  REGION 1..21: note = Description of sequence: Gene: S811-831; origin: SARS-CoV-2; HLA type: broadly recognized and present by common HLA II alleles
KPSKRSFIEDLLFNKVTLADA

SEQ ID NO: 418 - protein, 21 aa, synthetic construct
  REGION 1..21: note = Description of sequence: Gene: S946-966; origin: SARS-CoV-2; HLA type: broadly recognized and present by common HLA II alleles
GKLQDVVNQNAQALNTLVKQL

SEQ ID NO: 419 - protein, 21 aa, synthetic construct
  REGION 1..21: note = Description of sequence: Gene: S986-1006; origin: SARS-CoV-2; HLA type: broadly recognized and present by common HLA II alleles
KVEAEVQIDRLITGRLQSLQT

SEQ ID NO: 420 - protein, 17 aa, synthetic construct
  REGION 1..17: note = Description of sequence: Gene: S442-458 ; origin: SARS-CoV-2; HLA type: DRB1*11:01
DSKVGGNYNYLYRLFRK

SEQ ID NO: 421 - protein, 17 aa, synthetic construct
  REGION 1..17: note = Description of sequence: Gene: S813-829 ; origin: SARS-CoV-2; HLA type: DPA1*01:03/DPB1*04: 01
SKRSFIEDLLFNKVTLA

SEQ ID NO: 422 - protein, 17 aa, synthetic construct
  REGION 1..17: note = Description of sequence: Gene: S372-388; origin: SARS-CoV-2; HLA type: DRB1*15:02
ASFSTFKCYGVSPTKLN

SEQ ID NO: 423 - protein, 17 aa, synthetic construct
  REGION 1..17: note = Description of sequence: Gene: S729-745; origin: SARS-CoV-2; HLA type: DQA1*02:01/DQB1*02: 02
VSMTKTSVDCTMYICGD

SEQ ID NO: 424 - protein, 17 aa, synthetic construct
  REGION 1..17: note = Description of sequence: Gene: S729-745; origin: SARS-CoV-2; HLA type: DPA1*01:03/DPB1*04: 01
SKRSFIEDLLFNKVTLA

SEQ ID NO: 425 - protein, 17 aa, synthetic construct
  REGION 1..17: note = Description of sequence: Gene: S813-829; origin: SARS-CoV-2; HLA type: DPA1*01:03/DPB1*04: 01
SKRSFIEDLLFNKVTLA

SEQ ID NO: 426 - protein, 17 aa, synthetic construct
  REGION 1..17: note = Description of sequence: Gene: S190-206; origin: SARS-CoV-2; HLA type: DRB5*01:01
REFVFKNIDGYFKIYSK

SEQ ID NO: 427 - protein, 17 aa, synthetic construct
  REGION 1..17: note = Description of sequence: Gene: S750-766 ; origin: SARS-CoV-2; HLA type: DRB1*15:01
SNLLLQYGSFCTQLNRA

SEQ ID NO: 428 - protein, 17 aa, synthetic construct
  REGION 1..17: note = Description of sequence: Gene: S813-829; origin: SARS-CoV-2; HLA type: DPA1*02:01/DPB1
SKRSFIEDLLFNKVTLA

SEQ ID NO: 429 - protein, 17 aa, synthetic construct
  REGION 1..17: note = Description of sequence: Gene: S351-367; origin: SARS-CoV-2; HLA type: DRB3*02:02
YAWNRKRISNCVADYSV

SEQ ID NO: 430 - protein, 17 aa, synthetic construct
  REGION 1..17: note = Description of sequence: Gene: S610-626; origin: SARS-CoV-2; HLA type: DRB1*13:01
VLYQDVNCTEVPVAIHA

SEQ ID NO: 431 - protein, 17 aa, synthetic construct
  REGION 1..17: note = Description of sequence: Gene: S652-668; origin: SARS-CoV-2; HLA type: DQA1*02:01/DQB1*02:
GAEHVNNSYECDIPIGA

SEQ ID NO: 432 - protein, 17 aa, synthetic construct
  REGION 1..17: note = Description of sequence: Gene: S680-696; origin: SARS-CoV-2; HLA type: DQA1*01:03/DQB1*06:
SPRRARSVASQSIIAYT

SEQ ID NO: 433 - protein, 17 aa, synthetic construct
  REGION 1..17: note = Description of sequence: Gene: S85-101 ; origin: SARS-CoV-2; HLA type: DRB3*02:02
PFNDGVYFASTEKSNII

SEQ ID NO: 434 - protein, 17 aa, synthetic construct
  REGION 1..17: note = Description of sequence: Gene: S99-115 ; origin: SARS-CoV-2; HLA type: DPA1*01:03/ DPB1*06:01
NIIRGWIFGTTLDSKTQ

SEQ ID NO: 435 - protein, 17 aa, synthetic construct
  REGION 1..17: note = Description of sequence: Gene: S120-136 ; origin: SARS-CoV-2; HLA type: DRB3*02:02
VNNATNVVIKVCEFQFC

SEQ ID NO: 436 - protein, 17 aa, synthetic construct
  REGION 1..17: note = Description of sequence: Gene: S134-150; origin: SARS-CoV-2; HLA type: DRB1*11:01
QFCNDPFLGVYYHKNNK

SEQ ID NO: 437 - protein, 17 aa, synthetic construct
  REGION 1..17: note = Description of sequence: Gene: S351-367 ; origin: SARS-CoV-2; HLA type: DRB3*02:02{ DRB4*01:03
YAWNRKRISNCVADYSV

SEQ ID NO: 438 - protein, 17 aa, synthetic construct
  REGION 1..17: note = Description of sequence: Gene: S372-388 ; origin: SARS-CoV-2; HLA type: DPA1*01:03/DPB1*06: 01
ASFSTFKCYGVSPTKLN

SEQ ID NO: 439 - protein, 17 aa, synthetic construct
  REGION 1..17: note = Description of sequence: Gene: S386-402 ; origin: SARS-CoV-2; HLA type: DPA1*02:01/DPB1*14:
KLNDLCFTNVYADSFVI

SEQ ID NO: 440 - protein, 17 aa, synthetic construct
  REGION 1..17: note = Description of sequence: Gene: S400-416; origin: SARS-CoV-2; HLA type: DQA1*05:05/DQB1*03:
FVIRGDEVRQIAPGQTG

SEQ ID NO: 441 - protein, 17 aa, synthetic construct
  REGION 1..17: note = Description of sequence: Gene: S918-934; origin: SARS-CoV-2; HLA type: DRB3*02:02 16
ENQKLIANQFNSAIGKI

SEQ ID NO: 442 - protein, 17 aa, synthetic construct
  REGION 1..17: note = Description of sequence: Gene: S960-976; origin: SARS-CoV-2; HLA type: DRB3*02:02
NTLVKQLSSNFGAISSV

SEQ ID NO: 443 - protein, 17 aa, synthetic construct
  REGION 1..17: note = Description of sequence: Gene: S8--24; origin: SARS-CoV-2; HLA type: DRB4*01:01
LPLVSSQCVNLTTRTQL

SEQ ID NO: 444 - protein, 17 aa, synthetic construct
  REGION 1..17: note = Description of sequence: Gene: S36-52; origin: SARS-CoV-2; HLA type: DPA1*01:03/DPB1*04:
VYYPDKVFRSSVLHSTQ

SEQ ID NO: 445 - protein, 17 aa, synthetic construct
  REGION 1..17: note = Description of sequence: Gene: S162-178 ; origin: SARS-CoV-2; HLA type: DPA1*01:03/DPB1*04:
SANNCTFEYVSQPFLMD

SEQ ID NO: 446 - protein, 17 aa, synthetic construct
  REGION 1..17: note = Description of sequence: Gene: S190-206 ; origin: SARS-CoV-2; HLA type: DQA1*01:01/DQB1*05:
REFVFKNIDGYFKIYSK

SEQ ID NO: 447 - protein, 17 aa, synthetic construct
  REGION 1..17: note = Description of sequence: Gene: S715-731 ; origin: SARS-CoV-2; HLA type: DRB1*07:01
PTNFTISVTTEILPVSM

SEQ ID NO: 448 - protein, 17 aa, synthetic construct
  REGION 1..17: note = Description of sequence: Gene: S729-745 ; origin: SARS-CoV-2; HLA type: DQA1*02:01/DQB1*02:
VSMTKTSVDCTMYICGD

SEQ ID NO: 449 - protein, 17 aa, synthetic construct
  REGION 1..17: note = Description of sequence: Gene: S750-766; origin: SARS-CoV-2; HLA type: DRB1*15:01
SNLLLQYGSFCTQLNRA

SEQ ID NO: 450 - protein, 17 aa, synthetic construct
  REGION 1..17: note = Description of sequence: Gene: S813-829; origin: SARS-CoV-2; HLA type: DPA1*01:03/DPB1*04: 01
SKRSFIEDLLFNKVTLA

SEQ ID NO: 451 - protein, 17 aa, synthetic construct
  REGION 1..17: note = Description of sequence: Gene: S750-766 ; origin: SARS-CoV-2; HLA type: HLA-DRB1*15:01
SNLLLQYGSFCTQLNRA

SEQ ID NO: 452 - protein, 17 aa, synthetic construct
  REGION 1..17: note = Description of sequence: Gene: S813-829 ; origin: SARS-CoV-2; HLA type: DPA1*01:03/DPB1*04: 01
SKRSFIEDLLFNKVTLA

SEQ ID NO: 453 - protein, 17 aa, synthetic construct
  REGION 1..17: note = Description of sequence: Gene: S141-157 ; origin: SARS-CoV-2; HLA type: DRB3*02:02
LGVYYHKNNKSWMESEF

SEQ ID NO: 454 - protein, 17 aa, synthetic construct
  REGION 1..17: note = Description of sequence: Gene: S680-696 ; origin: SARS-CoV-2; HLA type: DQA1*02:01/DQB1*03: 03
SPRRARSVASQSIIAYT

SEQ ID NO: 455 - protein, 17 aa, synthetic construct
  REGION 1..17: note = Description of sequence: Gene: S729-745 ; origin: SARS-CoV-2; HLA type: DQA1*02:01/DQB1*03: 03
VSMTKTSVDCTMYICGD

SEQ ID NO: 456 - protein, 15 aa, synthetic construct
  REGION 1..15: note = Description of sequence: Gene: S165-179; origin: SARS-CoV-2; HLA type: DRB1*04:05/DQA1*02:01/DQB1*02:02/DQA 1*03:02/DQB1*02:02/DQA1*05:01/DQB1*02:01
NCTFEYVSQPFLMDL

SEQ ID NO: 457 - protein, 15 aa, synthetic construct
  REGION 1..15: note = Description of sequence: Gene: S801-815; origin: SARS-CoV-2; HLA type: DRB1*03:01/DRB1*04:01
NFSQILPDPSKPSKR

SEQ ID NO: 458 - protein, 13 aa, synthetic construct
  REGION 1..13: note = Description of sequence: Gene: S815-827; origin: SARS-CoV-2; HLA type: DPA1*01:03/DPB1*04:01
RSFIEDLLFNKVT

SEQ ID NO: 459 - protein, 15 aa, synthetic construct
  REGION 1..15: note = Description of sequence: Gene: S1101-1115; origin: SARS-CoV-2; HLA type: DRB1*04:05/DQA1*01:01/DQB1*05:01
HWFVTQRNFYEPQII

SEQ ID NO: 460 - protein, 15 aa, synthetic construct
  REGION 1..15: note = Description of sequence: Gene: N217-231; origin: SARS-CoV-2; HLA type: DRB1*03:01/DRB1*11:01/DRB1*13:01/DQA1*01:01/DQB1*05:01
AALALLLLDRLNQLE

SEQ ID NO: 461 - protein, 20 aa, synthetic construct
  REGION 1..20: note = Description of sequence: Gene: VP2P2; origin: Rhinovirus; HLA type: broadly recognized and present by common HLA II alleles
RGDSTITSQDVANAVVGYGV

SEQ ID NO: 462 - protein, 20 aa, synthetic construct
  REGION 1..20: note = Description of sequence: Gene: VP2P2 ; origin: Rhinovirus; HLA type: broadly recognized and present by common HLA II alleles
SDRIIQITRGDSTITSQDVA

SEQ ID NO: 463 - protein, 20 aa, synthetic construct
  REGION 1..20: note = Description of sequence: Gene: VP1P23; origin: Rhinovirus; HLA type: broadly recognized and present by common HLA II alleles
PRFSLPFLSIASAYYMFYDG

SEQ ID NO: 464 - protein, 20 aa, synthetic construct
  REGION 1..20: note = Description of sequence: Gene: VP2P26 ; origin: Rhinovirus; HLA type: broadly recognized and present by common HLA II alleles
VPYVNAVPMDSMVRHNNWSL

SEQ ID NO: 465 - protein, 20 aa, synthetic construct
  REGION 1..20: note = Description of sequence: Gene: VP2P10; origin: Rhinovirus; HLA type: broadly recognized and present by common HLA II alleles
TSKGWWWKLPDALKDMGIFG

SEQ ID NO: 466 - protein, 20 aa, synthetic construct
  REGION 1..20: note = Description of sequence: Gene: VP2P24; origin: Rhinovirus; HLA type: broadly recognized and present by common HLA II alleles
PHQFINLRSNNSATLIVPYV

SEQ ID NO: 467 - protein, 20 aa, synthetic construct
  REGION 1..20: note = Description of sequence: Gene: VP2P25; origin: Rhinovirus; HLA type: broadly recognized and present by common HLA II alleles
SNNSATLIVPYVNAVPMDSM

SEQ ID NO: 468 - protein, 20 aa, synthetic construct
  REGION 1..20: note = Description of sequence: Gene: VP1P21 ; origin: Rhinovirus; HLA type: broadly recognized and present by common HLA II alleles
QSGTNASVFWQHGQPFPRFS

SEQ ID NO: 469 - protein, 20 aa, synthetic construct
  REGION 1..20: note = Description of sequence: Gene: VP2P21; origin: Rhinovirus; HLA type: broadly recognized and present by common HLA II alleles
NEKQPSDDNWLNFDGTLLGN

SEQ ID NO: 470 - protein, 20 aa, synthetic construct
  REGION 1..20: note = Description of sequence: Gene: VP1P18 ; origin: Rhinovirus; HLA type: broadly recognized and present by common HLA II alleles
HIVMQYMYVPPGAPIPTTRD

SEQ ID NO: 471 - protein, 13 aa, synthetic construct
  REGION 1..13: note = Description of sequence: Gene: HA306-318; origin: Influenza A; HLA type: HLA-DRA1?01:01/HLA-DRB1?04:01/HLA-D RA1?01:01/HLA-DRB1?01:01 (HLA-DR1)
PKYVKQNTLKLAT

SEQ ID NO: 472 - protein, 14 aa, synthetic construct
  REGION 1..14: note = Description of sequence: Gene: M1; origin: Influenza A; HLA type: broadly recognized and present by common HLA II alleles
SGPLKAEIAQRLED

SEQ ID NO: 473 - protein, 14 aa, synthetic construct
  REGION 1..14: note = Description of sequence: Gene: M1; origin: Influenza A; HLA type: broadly recognized and present by common HLA II alleles
GLIYNRMGAVTTEV

SEQ ID NO: 474 - protein, 16 aa, synthetic construct
  REGION 1..16: note = Description of sequence: Gene: M1; origin: Influenza A; HLA type: broadly recognized and present by common HLA II alleles
QARQMVQAMRTIGTHP

SEQ ID NO: 475 - protein, 13 aa, synthetic construct
  REGION 1..13: note = Description of sequence: Gene: NP; origin: Influenza A; HLA type: broadly recognized and present by common HLA II alleles
DPFRLLQNSQVFS

SEQ ID NO: 476 - protein, 13 aa, synthetic construct
  REGION 1..13: note = Description of sequence: Gene: NP; origin: Influenza A; HLA type: broadly recognized and present by common HLA II alleles
LPFERATIMAAFT

SEQ ID NO: 477 - protein, 13 aa, synthetic construct
  REGION 1..13: note = Description of sequence: Gene: NP; origin: Influenza A; HLA type: broadly recognized and present by common HLA II alleles
PTFSVQRNLPFER

SEQ ID NO: 478 - protein, 13 aa, synthetic construct
  REGION 1..13: note = Description of sequence: Gene: PB1; origin: Influenza A; HLA type: broadly recognized and present by common HLA II alleles
NVVRKMMTNSQDT

SEQ ID NO: 479 - protein, 13 aa, synthetic construct
  REGION 1..13: note = Description of sequence: Gene: PB1; origin: Influenza A; HLA type: broadly recognized and present by common HLA II alleles
IMFSNKMARLGKG

SEQ ID NO: 480 - protein, 13 aa, synthetic construct
  REGION 1..13: note = Description of sequence: Gene: PB1; origin: Influenza A; HLA type: broadly recognized and present by common HLA II alleles
GMFNMLSTVLGVS

SEQ ID NO: 481 - protein, 15 aa, synthetic construct
  REGION 1..15: note = Description of sequence: Gene: L1 93-107; origin: HPV18; HLA type: HLA-DRB1*09:01/HLA-DRB1*07:01
SIFYHAGSSRLLTVG

SEQ ID NO: 482 - protein, 18 aa, synthetic construct
  REGION 1..18: note = Description of sequence: Gene: L1 524-541; origin: HPV18; HLA type: HLA-DRB5*01:01
YPLGRKFLVQAGLRRKPT

SEQ ID NO: 483 - protein, 16 aa, synthetic construct
  REGION 1..16: note = Description of sequence: Gene: E5 47-62; origin: HPV18; HLA type: HLA-DPA1*01:03/DPB1*02:01
ATAFTVYVFCFLLPML

SEQ ID NO: 484 - protein, 15 aa, synthetic construct
  REGION 1..15: note = Description of sequence: Gene: L1 30-44; origin: HPV16; HLA type: HLA-DRB1*09:01
RTNIYYHAGTSRLLA

SEQ ID NO: 485 - protein, 17 aa, synthetic construct
  REGION 1..17: note = Description of sequence: Gene: L1 385-401; origin: HPV16; HLA type: HLA-DRB1*04:05/HLA-DRB1*09:01/HLA-DRB3*02:02
ADVMTYIHSMNSTILED

SEQ ID NO: 486 - protein, 17 aa, synthetic construct
  REGION 1..17: note = Description of sequence: Gene: E5 67-83; origin: HPV16; HLA type: HLA-DRB1*07:01
VYIPLFLIHTHARFLIT

SEQ ID NO: 487 - protein, 20 aa, synthetic construct
  REGION 1..20: note = Description of sequence: Gene: 632-651; origin: TTD; HLA type: broadly recognized and present by common HLA II alleles
IDKISDVSTIVPYIGPALNI

SEQ ID NO: 488 - protein, 15 aa, synthetic construct
  REGION 1..15: note = Description of sequence: Gene: 827-841; origin: TTD; HLA type: broadly recognized and present by common HLA II alleles
ILMQYIKANSKFIGI

SEQ ID NO: 489 - protein, 21 aa, synthetic construct
  REGION 1..21: note = Description of sequence: Gene: 950-969; origin: TTD; HLA type: broadly recognized and present by common HLA II alleles
NNFTVSFWLRVPKVSASHLET

SEQ ID NO: 490 - protein, 19 aa, synthetic construct
  REGION 1..19: note = Description of sequence: Gene: 271-290; origin: DTD; HLA type: broadly recognized and present by common HLA II alleles
PVFAGANYAAWAVNVAQVI

SEQ ID NO: 491 - protein, 20 aa, synthetic construct
  REGION 1..20: note = Description of sequence: Gene: 321-340; origin: DTD; HLA type: broadly recognized and present by common HLA II alleles
VHHNTEEIVAQSIALSSLMV

SEQ ID NO: 492 - protein, 20 aa, synthetic construct
  REGION 1..20: note = Description of sequence: Gene: 331-350; origin: DTD; HLA type: broadly recognized and present by common HLA II alleles
QSIALSSLMVAQAIPLVGEL

SEQ ID NO: 493 - protein, 12 aa, synthetic construct
  REGION 1..12: note = Description of sequence: Gene: 331-342; origin: DTD; HLA type: broadly recognized and present by common HLA II alleles
QSIALSSLMVAQ

SEQ ID NO: 494 - protein, 20 aa, synthetic construct
  REGION 1..20: note = Description of sequence: Gene: 351-370; origin: DTD; HLA type: broadly recognized and present by common HLA II alleles
VDIGFAAYNFVESIINLFQV

SEQ ID NO: 495 - protein, 20 aa, synthetic construct
  REGION 1..20: note = Description of sequence: Gene: 411-430; origin: DTD; HLA type: broadly recognized and present by common HLA II alleles
QGESGHDIKITAENTPLPIA

SEQ ID NO: 496 - protein, 20 aa, synthetic construct
  REGION 1..20: note = Description of sequence: Gene: 431-450; origin: DTD; HLA type: broadly recognized and present by common HLA II alleles
GVLLPTIPGKLDVNKSKTHI

SEQ ID NO: 497 - protein, 452 aa, synthetic construct
  REGION 1..452: note = Description of sequence: CV11-HPV/E6-E7-S2
MHQKRTAMVQDPQERPRKLPQLCTELQTTIHDIILECVYCKQQLLRREVYDFAFRDLCIVYRDGNPYAVGDKCLKFYSKISEYRHYCYSLYGTTLEQQYNKPLCDLLIRCINCQKPLCPEEKQRHLDKKQRFHNTRGRWTGRCMSCCRSSRTRRETQLMPGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKPLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQ

SEQ ID NO: 498 - DNA, 1359 bp, synthetic construct
  misc_feature 1..1359: note = Description of sequence: CV11-HPV/E6-E7-S2
  source 1..1359: mol_type = other DNA
atgcaccagaaacggacggccatggtccaggatccgcaggagaggcctaggaagcttccacaactctgtactgagttacagacaaccatccatgacataatactggaatgtgtatactgcaagcagcagctactgagacgggaagtctacgacttcgcttttcgggacctatgtatcgtgtatagagacggtaacccctacgcggtgggagataagtgcctgaagttctattcgaagatcagtgagtaccgtcactactgttacagcctctacggtacaaccctggaacagcaatacaataaacccctgtgtgaccttctcatccggtgcatcaattgccagaagcctctctgtccagaggaaaaacagcgacatctggacaagaaacaaaggttccacaatactcgagggcgctggacggggagatgcatgtcatgctgcagatcatcccgcactagacgcgaaacccagctcatgcccggcgacacccctactttgcacgagtatatgctggatttacaacctgagacaacagatctttatgggtacggacaactgaacgactctagcgaggaagaagatgagattgatggcccagccggccaggccgagccggatagggcacattataacattgttaccttttgctgtaaatgtgacagtacactgcgcttgtgcgtgcagagcacccacgtggatatcaggacattggaggacctcctgatgggcaccctgggaattgtctgccccatttgttcccagaaaccactcggcgacatagccgcccgggacctgatctgcgcccagaagttcaacggcctgaccgtgctgcctcccctgctgaccgacgagatgatcgcccagtacaccagcgccctgttagccggaacaatcaccagcggctggactttcggcgctggagccgctctgcagatccccttcgctatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcaggacgtggtgaaccagaacgcccaggccctgaacaccctggtgaagcagctgagcagcaacttcggcgccatcagcagcgtgctgaacgacatcctgagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagtaa

SEQ ID NO: 499 - protein, 497 aa, synthetic construct
  REGION 1..497: note = Description of sequence: CV12-HLA A0201
MSRSVALAVLALLSLSGLEAGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 500 - DNA, 1494 bp, synthetic construct
  misc_feature 1..1494: note = Description of sequence: CV12-HLA A0201
  source 1..1494: mol_type = other DNA
atgtctcgctccgtggccttagctgtgctcgcgctactctctctttctggcctggaggctggctgcggcgcctctggcggcggaggatccggaggcggaggaagcatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgagttcacccccactgaaaaagatgagtatgcctgccgtgtgaaccatgtgactttgtcacagcccaagatagttaagtgggatcgagacatgggcggaggcggctctggcggaggaggcagcggcggtggcggcagcggcggcggcggctctggcagcggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctgctacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgtcttcccagcccaccatccccatcgtgggcatcattgctggcctggttctctttggagctgtgatcactggagctgtggtcgctgctgtgatgtggaggaggaaaagctcagatagaaaaggagggagctactctcaggctgcaagcagtgacagtgcccagggctctgatgtgtctctcacagcttgtaaagtgtga

(19) United States
(12) Patent Application Publication
(10) Pub. No.: US 2026/0199448 A1
(43) Pub. Date: Jul. 16, 2026

(54) NUCLEIC ACID BASED CANCER VACCINE AND METHODS THEREOF

(71) Applicant: ADVANCED RNA VACCINE (ARV) TECHNOLOGIES, INC., North Bethesda, MD (US)

(72) Inventors: Huabin ZHU, Evans, GA (US); Jiangsheng XU, Gaithersburgh, MD (US); Renhuan XU, North Potomac, MD (US)

(21) Appl. No.: 19/137,183

(22) PCT Filed: Dec. 11, 2023
(86) PCT No.: PCT/US2023/083348

§ 371 Date: Jun. 9, 2025

Related U.S. Application Data
(60) Provisional application No. 63/431,571, filed on Dec. 9, 2022.

(51) Int. Cl. A61K 39/12; A61K 9/51; A61K 31/7105; A61K 39/00; A61P 31/12; C07K 14/005; C07K 14/195; C07K 14/47; C07K 14/74; C12N 5/0781; C12N 5/0784; C12N 5/0786; C12N 15/62

(52) U.S. Cl. CPC A61K 39/12; A61K 9/5123; A61K 31/7105; A61P 31/12; C07K 14/005; C07K 14/195; C07K 14/4748; C07K 14/70539; C12N 5/0635; C12N 5/0639; C12N 5/0645; C12N 15/62; A61K 2039/53; A61K 2039/54; A61K 2039/545; A61K 2039/572; C07K 2319/00; C12N 2710/20034

(57) ABSTRACT

Described herein are composition and methods of using thereof. The compositions described herein can include a single chain trimer nucleic acid encoding a first T cell epitope, a β2-microglobulin, and a MHC class I heavy chain sequence. The methods described herein can be used to activate and/or expand an antigen-presenting cell. The methods described herein can also be used to treat or prevent a viral infection, bacterial infection, parasitic infection and/or a cancer in a subject.

### CROSS-REFERENCE TO RELATED APPLICATION

This application claims priority to, and the benefit of U.S. Provisional Application 63/431,571, filed on Dec. 9, 2022, the contents of which is hereby incorporated in its entirety.

### REFERENCE TO SEQUENCE LISTING

The Sequence Listing submitted Dec. 11, 2023, as a text filed named “11538-006WO1_ST26.xml” created Dec. 8, 2023, and having a file size of 679,893 bytes is hereby incorporated by reference pursuant to 37 C.F.R. § 1.52(e)(5).

### BACKGROUND

Tumor Specific Antigens (TSAs) or Tumor Associated Antigens (TAAs) are essential targets for cancer vaccines that activate host anti-tumor CD8 T cells to specifically attack and destroy cancer cells that overexpress the same antigens. However, cancer vaccines have not achieved the anticipated clinical results and the main reasons for their ineffectiveness are at least the following:
1) The MHC binding affinity and/or abundance of these tumor antigen-derived peptides is low and they must compete with a large number of endogenous self-antigen-derived or other pathogen-derived peptides to bind MHC I molecules. Since MHC I does not distinguish between self- and non-self-derived peptides, therefore the processing and MHC I presentation efficiency of these tumor peptides is usually inefficient, which results in a clinically ineffective anti-tumor CD8 T cell response.
2) Most cancer vaccines are designed to activate antigen-specific CD8 T cell responses, but more evidence supports a critical role for CD4 T cells not only in cellular immunity, but also providing necessary help for tumor-specific cytotoxic T lymphocytes (CTL), including activation of antigen-presenting cells, promotion of CD8 T cell homing to tumor tissue and generation of effective memory CD8 T cells.
3) Vaccine delivery platforms are not sufficient to express tumor antigens or cannot deliver antigens to antigen-presenting cells (APCs), which are specialized cells that effectively activate T cells.

Accordingly, there is still a need for a compositions and methods of treating and preventing bacterial infections, viral infection, parasitic infections, and/or cancer.

The compositions and methods disclosed herein address these and other needs.

### SUMMARY

Provided herein are single chain nucleic acid trimer, compositions including a single chain trimer nucleic acid described herein and methods of using thereof.

Described herein are single chain trimer nucleic acid encoding a first T cell epitope, a β2-microglobulin, and a MHC class I heavy chain sequence. In some embodiments, the compositions described herein include a single chain trimer nucleic acid encoding a first T cell epitope, a β2-microglobulin, and a MHC class I heavy chain sequence.

In some embodiments, the single chain trimer nucleic acid can further include a second T cell epitope. In some embodiments, the second T cell epitope can be a MHC class II restricted epitope. In some embodiments, the second T cell epitope can include SEQ ID NO: 21, SEQ ID NO: 22, SEQ ID NO: 23, SEQ ID NO: 24, SEQ ID NO: 25, SEQ ID NO: 26, or an epitope listed in Table 5.

In some embodiments, the single chain trimer nucleic acid can include a nucleic acid encoding an amino to carboxy terminal order T cell epitope, the β2 microglobulin, and the MHC heavy chain sequence.

In some embodiments, the single chain trimer nucleic acid can further include a flexible first linker between the first T cell epitope and the β2 microglobulin and second linker between the β2 microglobulin and the MHC class I heavy chain sequence.

In some embodiments, the compositions can further include a pharmaceutically acceptable carrier. In some aspects, disclosed herein is a pharmaceutical composition can be a single chain trimer nucleic acid (e.g., mRNA) encoding a first T cell epitope, a β2-microglobulin, and a MHC heavy chain sequence; and a pharmaceutically acceptable carrier. In some embodiments, the composition can be a nanoparticle, a lipid nanoparticle dispersion, a liposomal formulation, a lipid emulsion, vaccine, vector, or any combination thereof.

In some embodiments, the pharmaceutical composition can include the lipid nanoparticle including a single chain trimer nucleic acid (e.g., mRNA) encoding a first T cell epitope, a β2-microglobulin, and a MHC class I heavy chain sequence.

Described herein are also cells including compositions described herein. In some embodiments, the cell can include a dendritic cell, B cell, macrophage, or other cells derived from a subject that can be used to for immunotherapy.

Described herein are also methods of activating and/or expanding an antigen-presenting cell the method including co-culturing an antigen-presenting cell and a cell including the single chain trimer nucleic acid described herein, the composition described herein, the lipid nanoparticle described herein, or the pharmaceutical composition described herein. In some embodiments, the antigen-presenting cell can be a dendritic cell, B cell, macrophage, or other cells derived from a subject that can be used to for immunotherapy.

Described herein are also methods of treating a subject with a viral infection, bacterial infection, parasitic infection and/or a cancer said method including administering to the subject a therapeutically effective amount of the single chain trimer nucleic acid described herein, the composition described herein, the lipid nanoparticle described herein, the cell described herein, or the pharmaceutical composition described herein.

Described herein are also methods of treating a subject with a viral infection, bacterial infection, parasitic infection and/or a cancer said method including obtaining an antigen-presenting cell from a subject contacting the antigen-presenting cell from the subject with the single chain trimer nucleic acid described herein, the composition described herein, the lipid nanoparticle described herein, or the pharmaceutical composition described herein thereby activating the antigen-presenting cells, and administering the activated antigen-presenting cells to the subject. In some embodiments, the antigen-presenting cell can include a dendritic cell, B cell, macrophage, or other cells derived from a subject that can be used to for immunotherapy.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIGS. 1A-1F. show a design of Kb-E6-SCT mRNA and in vitro potency. (FIG. 1A), shows the design of mRNA of SCT-E6. (FIG. 1B) shows the linearized plasmid template, and (FIG. 1C) shows the in vitro transcription of mRNA of SCT-E6. (FIG. 1D-1E) shows physicochemical characterization of lipid nanoparticles (LNP) formulation and encapsulation efficacy of L2M6 (L2M6 denotes LNP formulation of Kb-E6-SCT mRNA using an ionizable lipid ARV-T1). FIG. 1D shows particle size and polydispersity index. FIG. 1E shows zeta potential and encapsulation efficiency. (FIG. 1F) shows expression of SCT-E6 in 293T cell after transfection.

FIGS. 2A-2C show in vivo validation. C57BL/6 mice were immunized i.m. with 1 ug mRNA of indicated LNP-formulated vaccine twice with two week apart, 7 days after the last immunization, E6-CD8 T cell peptide was used to determine the E6 specific T cell population by ELISPOT (FIG. 2A-2B) (2A) image and (2B) graph; and intracellular IFN-gamma staining (FIG. 2C-2D).

FIGS. 3A-3C show In vivo antitumor efficacy of prophylactic vaccination of mRNA-L2M6. FIG. 3A, is an illustration of 5 C57BL/c mice immunized with 1 μg of mRNA/SCT-E6 intramuscular on day 1 and day 14, on day 21, 1×10\hat{ }5 TC1 tumor cells were injected subcutaneously. Tumor growth (FIG. 3B) and survival (FIG. 3C) were monitored.

FIGS. 4A-4C. show In vivo efficacy of therapeutic administration of mRNA-L2M6 vaccine. FIG. 4A is an experimental scheme of 10 female C57BL/6 mice injected with 2×10\hat{ }5 TC-1 tumor cells on the frank s.c., day 1, 8 and 15 after tumor challenge, mice received three doses of immunization of L2M6 or PBS. FIG. 4B is a graph of tumor progression. FIG. 4C is a graph of long-term survival of each experimental group.

FIG. 5 shows synthesization of HPV mRNA vaccine in vitro. HPV mRNA 1-3 were synthesized with T7 RNA polymerase in vitro transcription and run on 0.8% MOPS agarose gel.

FIGS. 6A-6C show validation of HPV mRNA vaccines in vitro. HPV mRNA 1-4 were transfected into 293T cells and detected expression on 48 h. 6A-6B, The expression level of HPV mRNA1 (6A) and mRNA 2-4 (6B) were detected by flow cytometry with anti-Sars-S2 (6A) and anti-HLA-A2 (6B) antibodies, respectively. (6C) The expression of HPV mRNA-1 were detected by western blot with E7 antibody.

FIGS. 7A-7I show HPV mRNA vaccine elicits robust T cell responses and strong antibody responses in hHLA-A2 transgenic mice. (7C-7D) Splenocytes were isolated from mouse spleen and performed for ELISPOT assay with stimulation of a E6 mix peptides pool, E7 mix peptides pool and E6 single peptide pool. 7C representative image of ELISpot plate readout, and the IFNγ-producing T lymphocytes were quantified as shown in 7D. (7E-7I) Splenocytes were isolated from mouse spleen and performed flow cytometry intracellular staining with stimulation of a E6+E7 mix peptides pool (7F-7G) or E6 single peptide pool (7H-7I).

FIGS. 8A-8C show HPV mRNA induced a strong cell mediated immune response. hHLA-A2 transgenic mice were intramuscularly immunized on DO and D14 with 5 ug of LNP formulated mRNA vaccines (HPV mRNA1+3). Mouse spleens were collected on D28. Splenocytes were isolated from mouse spleen. (8A-8B) mouse CD8 T cells were isolated with kit and detected by flow cytometry with anti-mouse CD8 T cells antibody. (8C) T cell mediated immune response were evaluated by Cytotoxic T lymphocyte assay (CTL assay). Tumor target cells (T), TA2-Luc stable cells (hHLA-A2 and Luciferase overexpressed TC-1 cell line), were seed on 96-well plate with the cell suspension 2×10⁴ cells/well on DO. Assuming that the cancer cells doubled overnight. Isolated CD8 T cells from A as the effector cells (E) were added to the tumor target cells with 1×10⁵/well (E:T=2.5:1) on D1. Cells were performed for flow cytometry based cytotoxicity assessment with annexin V and Helix NP on D3.

Like reference symbols in the various drawings indicate like elements.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

### General Definitions

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. A range may be construed to include the start and the end of the range. For example, a range of 10% to 20% (i.e., range of 10%-20%) can includes 10% and also includes 20%, and includes percentages in between 10% and 20%, unless explicitly stated otherwise herein.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

“Administration” to a subject includes any route of introducing or delivering to a subject an agent. Administration can be carried out by any suitable route, including oral, topical, transcutaneous, transdermal, intra-joint, intra-arteriole, intradermal, intraventricular, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inactivate”, “inactivating” and “inactivation” means to decrease or eliminate an activity, response, condition, disease, or other biological parameter due to a chemical (covalent bond formation) between the ligand and a its biological target.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective’ amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n-COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician. In some embodiments, the subject is a mammal. In some embodiments, the mammal is a human. In some embodiments, the subject is a veterinary patient. Administration of the therapeutic agents can be carried out at dosages and for periods of time effective for treatment of a subject. In some embodiments, the subject is a human.

### Genetic Definitions

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers. The polynucleotide sequence may be modified, for example, to enhance efficacy and/or to reduce immune responsivity, by using, for example, base modifications or end-capping. In other embodiments, an unmodified polynucleotide sequence is used. For example, the polynucleotide can be an RNA sequence or a DNA sequence. In some embodiments, the mRNA can include an optimized codon. By codon optimizing, the formation of secondary structures can be reduced and translational efficiency improved. In certain embodiments, the codon optimization includes GC enrichment of the coding region. In certain embodiments, the codon optimization includes codon quality enrichment of the coding region. In certain aspects, the mRNA can include one or more regions or parts, which act or function as an untranslated region (UTRs) of a gene. UTRs are transcribed but not translated. In mRNA, the 5′ UTR starts at the transcription start site and continues to the start codon but does not include the start codon. The 3′ UTR starts immediately following the stop codon and continues until the transcriptional termination signal. The use of human-derived UTRs may facilitate the expression of the polypeptide in cells. In some embodiments, the polynucleotide comprises at least one chemically modified nucleotide. In some embodiments, the at least one chemically modified nucleotide comprises a chemically modified nucleobase, a chemically modified ribose, a chemically modified phosphodiester linkage, or a combination thereof. In some embodiments, the polynucleotide sequence as used comprise modified nucleosides such as 5-methylcystonsine or psudouridine.

As used herein “modified” refers to a changed state or structure of a molecule of the invention. Molecules may be modified in many ways including chemically, structurally, and functionally. In one embodiment, the polynucleotides of the present invention are “chemically modified” by the introduction of non-natural nucleosides and/or nucleotides, e.g., as it relates to the natural ribonucleotides A, U, G, and C. Modifications of the nucleosides and/or nucleotides as used in the present invention may be naturally occurring (i.e. comprise a nucleotide and/or nucleoside other than the natural ribonucleotides A, U, G, and C) or may be artificial. Non-canonical nucleotides such as the cap structures are not considered “modified” although they differ from the chemical structure of A, G, C, and U ribonucleotides. As used herein, a “structural” modification is one in which two or more linked nucleosides are inserted, deleted, duplicated, inverted or randomized in a polynucleotide without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. When the polynucleotides of the present invention are chemically and/or structurally modified, the polynucleotides may be referred to as “modified nucleotides”.

In some embodiments, the nucleic acids disclosed herein can include at least one chemically modified nucleotide. In some embodiments, the at least one chemically modified nucleotide comprises a chemically modified nucleobase, a chemically modified ribose, a chemically modified phosphodiester linkage, or a combination thereof.

In one embodiment, the at least one chemically modified nucleotide is a chemically modified nucleobase.

In one embodiment, the chemically modified nucleobase is selected from 5-formylcytidine (5fC), 5-methylcytidine (5meC), 5-methoxycytidine (5moC), 5-hydroxycytidine (5hoC), 5-hydroxymethylcytidine (5hmC), 5-formyluridine (5fU), 5-methyluridine (5-meU), 5-methoxyuridine (5moU), 5-carboxymethylesteruridine (5camU), pseudouridine (Ψ), N¹-methylpseudouridine (me¹Ψ), N⁶-methyladenosine (me⁶A), or thienoguanosine (^{th}G).

In some embodiments, the chemically modified nucleobase is 5-methoxyuridine (5moU). In some embodiments, the chemically modified nucleobase is pseudouridine (Ψ). In some embodiments, the chemically modified nucleobase is N¹-methylpseudouridine (me¹Ψ).

The structures of these modified nucleobases are shown below:

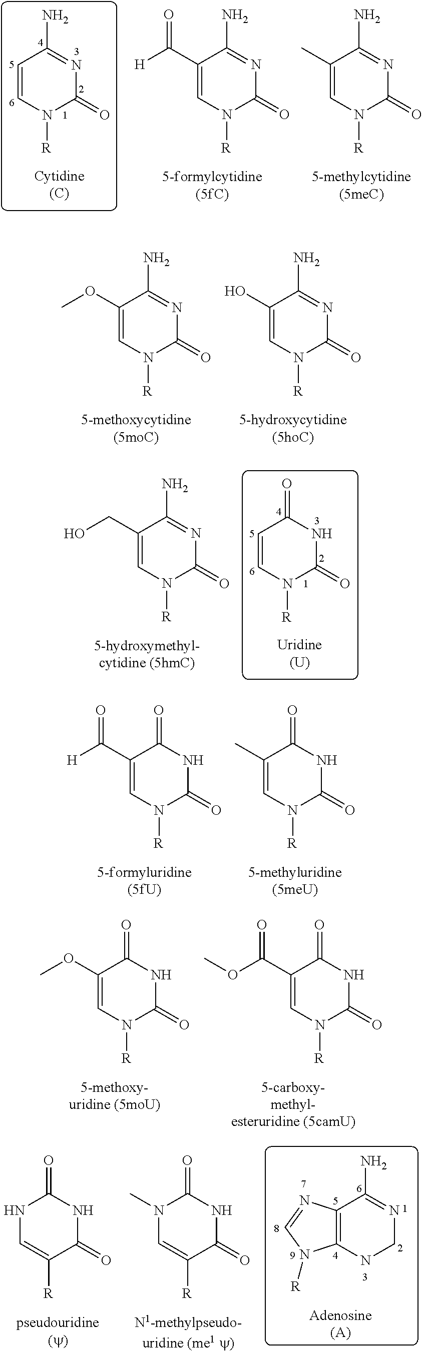

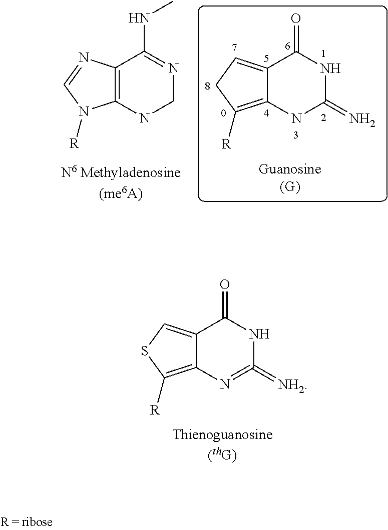

In one embodiment, the at least one chemically modified nucleotide is a chemically modified ribose.

In one embodiment, the chemically modified ribose is selected from 2′-O-methyl(2′-O-Me), 2′-Fluoro (2′-F), 2′-deoxy-2′-fluoro-beta-D-arabino-nucleic acid (2′F-ANA), 4′-S, 4′-SFANA, 2′-azido, UNA, 2′-O-methoxy-ethyl(2′-O-ME), 2′-O-Allyl, 2′-O-Ethylamine, 2′-O-Cyanoethyl, Locked nucleic acid (LAN), Methylene-cLAN, N-MeO-amino BNA, or N-MeO-aminooxy BNA. In one embodiment, the chemically modified ribose is 2′-O-methyl(2′-O-Me). In one embodiment, the chemically modified ribose is 2′-Fluoro (2′-F).

The structures of these modified riboses are shown below:

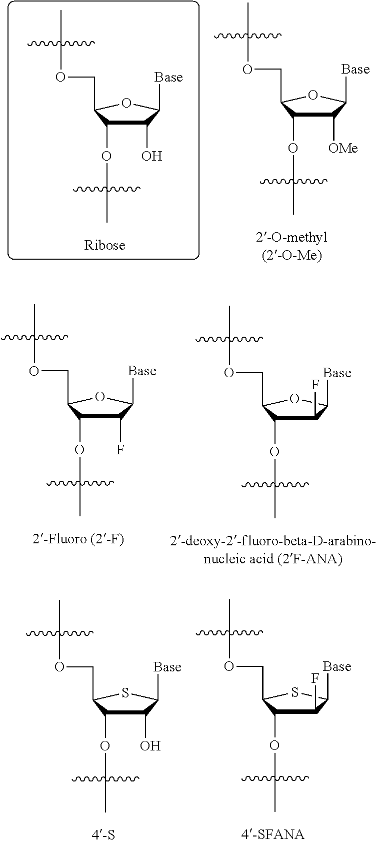

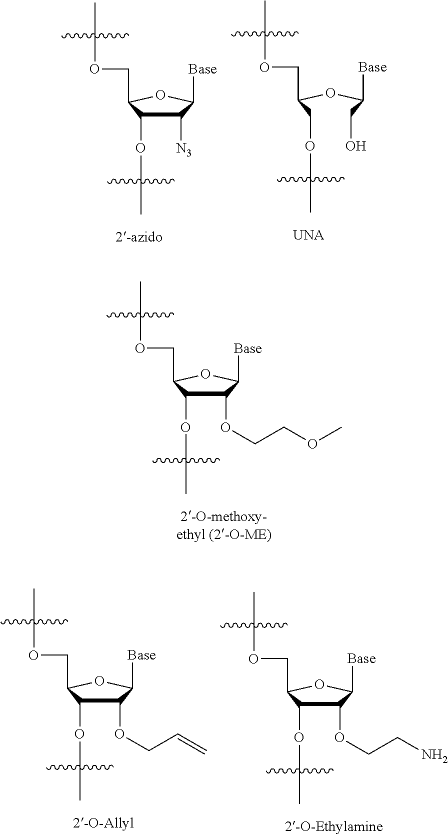

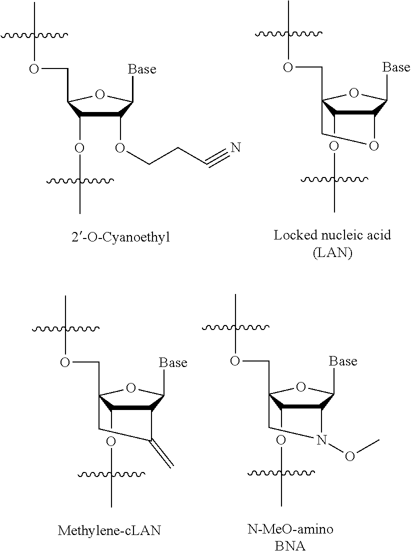

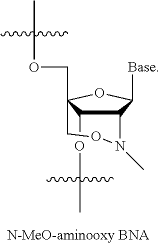

In one embodiment, the at least one chemically modified nucleotide is a chemically modified phosphodiester linkage.

In one embodiment, the chemically modified phosphodiester linkage is selected from phosphorothioate (PS), boranophosphate, phosphodithioate (PS2), 3′,5′-amide, N3′-phosphoramidate (NP), Phosphodiester (PO), or 2′,5′-phosphodiester (2′,5′-PO). In one embodiment, the chemically modified phosphodiester linkage is phosphorothioate.

The structures of these modified phosphodiester linkages are shown below:

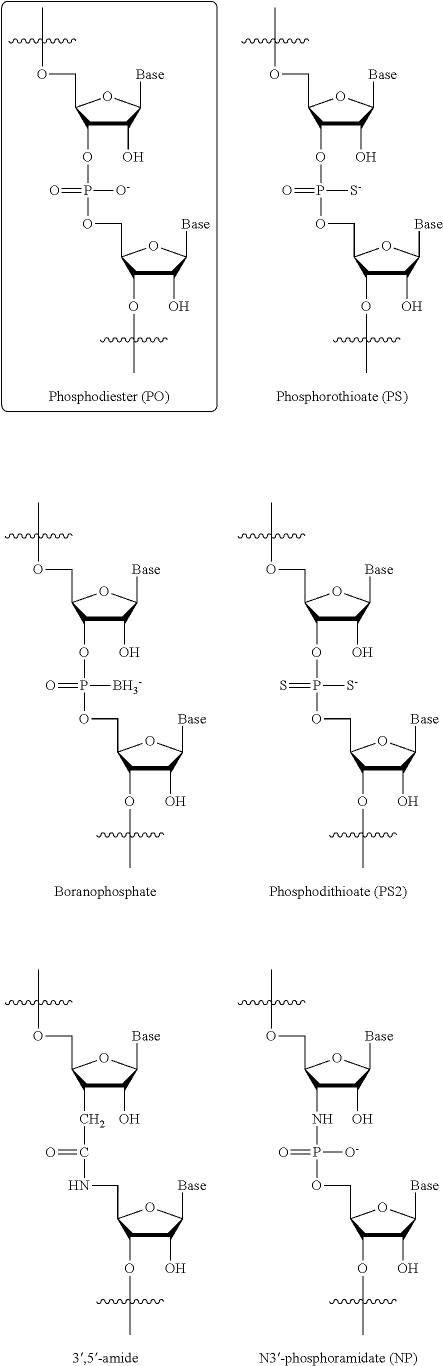

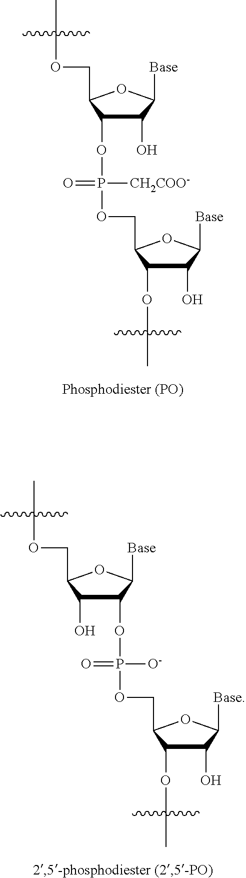

In some embodiments, the mRNA can include a heterologous 5′ untranslated region (5′UTR). In some embodiments, the mRNA can include a heterologous 3′ untranslated region (3′UTR).

The term “polypeptide” refers to a compound made up of a single chain of D- or L-amino acids or a mixture of D- and L-amino acids joined by peptide bonds. A polypeptide is comprised of approximately twenty, standard naturally occurring amino acids, although natural and synthetic amino acids which are not members of the standard twenty amino acids may also be used. The standard twenty amino acids include alanine (Ala, A), arginine (Arg, R), asparagine (Asn, N), aspartic acid (Asp, D), cysteine (Cys, C), glutamine (Gln, Q), glutamic acid (Glu, E), glycine (Gly, G), histidine, (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V). The terms “polypeptide sequence” or “amino acid sequence” are an alphabetical representation of a polypeptide molecule.

Conservative substitutions of amino acids in proteins and polypeptides are known in the art. For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the polypeptides provided herein.

Substantial changes in protein function or immunological identity are made by selecting substitutions that are less conservative, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

A “variant” refers to a molecule substantially similar in structure. Thus, in one embodiment, a variant refers to a protein whose amino acid sequence is similar to a reference amino acid sequence, but does not have 100% identity with the respective reference sequence. The variant protein has an altered sequence in which one or more of the amino acids in the reference sequence is deleted or substituted, or one or more amino acids are inserted into the sequence of the reference amino acid sequence. As a result of the alterations, the variant protein has an amino acid sequence which is at least 60%, 70%, 75%, 80%, 85%, 90%, or 95% identical to the reference sequence. For example, variant sequences which are at least 95% identical have no more than 5 alterations, i.e. any combination of deletions, insertions or substitutions, per 100 amino acids of the reference sequence.

The term “complementary” refers to the topological compatibility or matching together of interacting surfaces of a probe molecule and its target. Thus, the target and its probe can be described as complementary, and furthermore, the contact surface characteristics are complementary to each other.

The term “hybridization” refers to a process of establishing a non-covalent, sequence-specific interaction between two or more complementary strands of nucleic acids into a single hybrid, which in the case of two strands is referred to as a duplex.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

Reference will now be made in detail to specific aspects of the disclosed materials, compounds, compositions, articles, and methods, examples of which are illustrated in the accompanying Examples and Figures.

### Single Chain Trimer Nucleic Acid

Described herein are single chain trimer nucleic acid sequences encoding a first T cell epitope, a β2-microglobulin, and a MHC class I heavy chain sequence.

In some embodiments, the single chain trimer nucleic acid can include nucleic acid sequences selected from any of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 498, or SEQ ID NO: 500 and homologs having at least 90% (e.g. 90%, 91%, 92%, 93%, 94%, 95%, 96%, 97%, 98%, 99%, 99.8%, or 99.9%) identity with a nucleic acid sequence of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 498, or SEQ ID NO: 500.

In some embodiments, the single chain trimer nucleic acid can encode an amino acid selected from any one of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 497, or SEQ ID NO: 499 and homologs having at least 90% (e.g. 90%, 91%, 92%, 93%, 94%, 95%, 96%, 97%, 98%, 99%, 99.8%, or 99.9%) identity with an amino acid sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 497, or SEQ ID NO: 499.

In some embodiments, the first T cell epitope can include an epitope listed in Table 1 to Table 3 or homologs having at least 90% (e.g. 90%, 91%, 92%, 93%, 94%, 95%, 96%, 97%, 98%, 99%, 99.8%, or 99.9%) identity with an epitope of Table 2.

In some embodiments, the first T cell epitope can include an epitope listed in Table 2 or homologs having at least 90% (e.g. 90%, 91%, 92%, 93%, 94%, 95%, 96%, 97%, 98%, 99%, 99.8%, or 99.9%) identity with an epitope of Table 2.

In some embodiments, the single chain trimer nucleic acid can include a nucleic acid encoding an amino to carboxy terminal order T cell epitope, the β2 microglobulin, and the MHC heavy chain sequence.

Table 1 lists DNA and protein sequences for exemplary cancer vaccines constructs. Table 1 also includes SEQ ID NO: 21 and SEQ ID NO: 22, SEQ ID NO: 23, SEQ ID NO: 24, SEQ ID NO: 25, SEQ ID NO. 26 universal CD4 T cell epitope.

SEQ ID NO: 21 and SEQ ID NO: 22, SEQ ID NO: 23, SEQ ID NO: 24, SEQ ID NO: 25, SEQ ID NO: 26, or an epitope listed in Table 5 can be a universal CD4 T cell epitope able to activate preexisting memory CD4 T cell, which can effectively promote antitumoral activity of CD8 T cells.

TABLE 1
DNA and protein sequences in constructs
Table 1. DNA and Protein Sequences in Constructs

| | SEQ ID | |
|---|---|---|
| Name | NO: | Sequence |
| CV1-KR/G12C-A0201; | 1 | MSRSVALAVLALLSLSGLEAKLVVVGACGVGCGASGGGGSGGGGSIQRTPKI |
| protein sequence | | QVYSRHPAENGKSNFLNCYVSGFHPSDIEVDLLKNGERIEKVEHSDLSFSKD |
| | | WSFYLLYYTEFTPTEKDEYACRVNHVTLSQPKIVKWDRDMGGGGSGGGGSGG |
| | | GGSGGGGSGSGSHSMRYFFTSVSRPGRGEPRFIAVGYVDDTQFVREDSDAAS |
| | | QRMEPRAPWIEQEGPEYWDGETRKVKAHSQTHRVDLGTLRGCYNQSEAGSHT |
| | | VQRMYGCDVGSDWRFLRGYHQYAYDGKDYIALKEDLRSWTAADMAAQTTKHK |
| | | WEAAHVAEQLRAYLEGTCVEWLRRYLENGKETLQRTDAPKTHMTHHAVSDHE |
| | | ATLRCWALSFYPAEITLTWQRDGEDQTQDTELVETRPAGDGTFQKWAAVVVP |
| | | SGQEQRYTCHVQHEGLPKPLTLRWEPSSQPTIPIVGIIAGLVLFGAVITGAV |
| | | VAAVMWRRKSSDRKGGSYSQAASSDSAQGSDVSLTACKVATNFSLLKQAGDV |
| | | EENPGPMKWVTFISLLFLESSAYSILMQYIKANSKFIGIPMGLPQSIALSSL |
| | | MVAQPMGLPAKFVAAWTLKAAA |
| CV1-KR/G12C-A0201; | 2 | ATGTCTCGCTCCGTGGCCTTAGCTGTGCTCGCGCTACTCTCTCTTTCTGGCC |
| DNA sequence | | TGGAGGCTAAACTTGTGGTAGTTGGAGCTTGCGGCGTAGGCTGCGGCGC |
| | | CTCTGGCGGCGGAGGATCCGGAGGCGGAGGAAGCATCCAGCGTACTCCA |
| | | AAGATTCAGGTTTACTCACGTCATCCAGCAGAGAATGGAAAGTCAAATTT |
| | | CCTGAATTGCTATGTGTCTGGGTTTCATCCATCCGACATTGAAGTTGACTT |
| | | ACTGAAGAATGGAGAGAGAATTGAAAAAGTGGAGCATTCAGACTTGTCTT |
| | | TCAGCAAGGACTGGTCTTTCTATCTCTTGTACTACACTGAATTCACCCCCAC |
| | | TGAAAAAGATGAGTATGCCTGCCGTGTGAACCATGTGACTTTGTCACAGC |
| | | CCAAGATAGTTAAGTGGGATCGAGACATGGGCGGAGGCGGCTCTGGCGG |
| | | AGGAGGCAGCGGCGGTGGCGGCAGCGGCGGCGGCGGCTCTGGCAGCGG |
| | | CTCTCACTCCATGAGGTATTTCTTCACATCCGTGTCCCGGCCCGGCCGCGG |
| | | GGAGCCCCGCTTCATCGCAGTGGGCTACGTGGACGACACGCAGTTCGTGC |
| | | GGTTCGACAGCGACGCCGCGAGCCAGAGGATGGAGCCGCGGGCGCCGT |
| | | GGATAGAGCAGGAGGGTCCGGAGTATTGGGACGGGGAGACACGGAAAG |
| | | TGAAGGCCCACTCACAGACTCACCGAGTGGACCTGGGGACCCTGCGCGG |
| | | CTGCTACAACCAGAGCGAGGCCGGTTCTCACACCGTCCAGAGGATGTATG |
| | | GCTGCGACGTGGGGTCGGACTGGCGCTTCCTCCGCGGGTACCACCAGTAC |
| | | GCCTACGACGGCAAGGATTACATCGCCCTGAAAGAGGACCTGCGCTCTTG |
| | | GACCGCGGCGGACATGGCAGCTCAGACCACCAAGCACAAGTGGGAGGCG |
| | | GCCCATGTGGCGGAGCAGTTGAGAGCCTACCTGGAGGGCACGTGCGTGG |
| | | AGTGGCTCCGCAGATACCTGGAGAACGGGAAGGAGACGCTGCAGCGCAC |
| | | GGACGCCCCCAAAACGCATATGACTCACCACGCTGTCTCTGACCATGAAG |
| | | CCACCCTGAGGTGCTGGGCCCTGAGCTTCTACCCTGCGGAGATCACACTG |
| | | ACCTGGCAGCGGGATGGGGAGGACCAGACCCAGGACACGGAGCTCGTG |
| | | GAGACCAGGCCTGCAGGGGATGGAACCTTCCAGAAGTGGGCGGCTGTGG |
| | | TGGTGCCTTCTGGACAGGAGCAGAGATACACCTGCCATGTGCAGCATGAG |
| | | GGTTTGCCCAAGCCCCTCACCCTGAGATGGGAGCCGTCTTCCCAGCCCACC |
| | | ATCCCCATCGTGGGCATCATTGCTGGCCTGGTTCTCTTTGGAGCTGTGATC |
| | | ACTGGAGCTGTGGTCGCTGCTGTGATGTGGAGGAGGAAGAGCTCAGATA |
| | | GAAAAGGAGGGAGCTACTCTCAGGCTGCAAGCAGTGACAGTGCCCAGGG |
| | | CTCTGATGTGTCTCTCACAGCTTGTAAAGTGGCTACCAACTTCAGCCTGCT |
| | | GAAGCAGGCTGGCGATGTGGAAGAAAACCCTGGCCCAATGAAGTGGGTA |
| | | ACCTTTATTTCCCTTCTTTTTCTCTTTAGCTCGGCTTATTCCATACTAATGCA |
| | | ATACATAAAAGCAAACAGCAAATTCATAGGAATACCAATGGGACTACCAC |
| | | AAAGCATAGCACTAAGCAGCCTAATGGTAGCACAACCAATGGGACTACCA |
| | | GCAAAATTCGTAGCAGCATGGACACTAAAAGCAGCAGCATGATAG |
| CV2-KR/G12C-A0301; | 3 | MSRSVALAVLALLSLSGLEAVVGACGVGKGCGASGGGGGGGGSIQREPKIQ |
| protein sequence | | VYSRHPAENGKSNFLNCYVSGFHPSDIEVDLLKNGERIEKVEHSDLSFSKDW |
| | | SFYLLYYTEFTPTEKDEYACRVNHVTLSQPKIVKWDRDMGGGGSGGGGSGGG |
| | | GSGGGGSGSGSHSMRYFFTSVSRPGRGEPRFIAVGYVDDTQFVREDSDAASQ |
| | | RMEPRAPWIEQEGPEYWDQETRNVKAQSQTDRVDLGTLRGCYNQSEAGSHTI |
| | | QIMYGCDVGSDGRFLRGYRQDAYDGKDYIALNEDLRSWTAADMAAQITKRKW |
| | | EAAHEAEQLRAYLDGTCVEWLRRYLENGKETLQRTDPPKTHMTHHPISDHEA |
| | | TLRCWALGFYPAEITLTWQRDGEDQTQDTELVETRPAGDGTFQKWAAVVVPS |
| | | GEEQRYTCHVQHEGLPKPLTLRWELSSQPTIPIVGIIAGLVLIGAVITGAVV |
| | | AAVMWRRKSSDRKGGSYTQAASSDSAQGSDVSLTACKVATNFSLLKQAGQVE |
| | | ENPGPMKWVTFISLLELESSAYSILMQYIKANSKFIGIPMGLPQSIALSSLM |
| | | VAQPMGLPAKEVAAWTLKAZA |
| CV2-KR/G12C-A0301; | 4 | ATGTCTCGCTCCGTGGCCTTAGCTGTGCTCGCGCTACTCTCTCTTTCEGGCC |
| DNA sequence | | TGGAGGCTGTAGTTGGAGCTTGTGGCGTAGGCAAGGGCTGCGGCGCCECTGG |
| | | CGGCGGAGGATCCGGAGGCGGAGGAAGCATCCAGCGTACTCCAAAGATTCAG |
| | | GTTTACTCACGTCATCCAGCAGAGAATGGAAAGTCAAATTTCCTGAATTGCT |
| | | ATGTGTCTGGGTTTCATCCATCCGACATTGAAGTTGACTTACTGAAGAATGG |
| | | AGAGAGAATTGAAAAAGTGGAGCATTCAGACTTGTCTTTCAGCAAGGACTGG |
| | | TCTTTCTATCTCTTGTACTACACTGAATTCACCCCCACTGAAAAAGATGAGT |
| | | ATGCCTGCCGTGTGAACCATGTGACTTTGTCACAGCCCAAGATAGTTAAGTG |
| | | GGATCGAGACATGGGCGGAGGCGGCTCTGGCGGAGGAGGCAGCGGCGGTGGC |
| | | GGCAGCGGCGGCGGCGGCTCTGGCAGCGGCTCCCACTCCATGAGGTAETTCT |
| | | TCACATCCGTGTCCCGGCCCGGCCGCGGGGAGCCCCGCTTCATCGCCGTGGG |
| | | CTACGTGGACGACACGCAGITCGTGCGGITCGACAGCGACGCCGCGAGCCAG |
| | | AGGATGGAGCCGCGGGCGCCGTGGATAGAGCAGGAGGGGCCGGAGTATTGGG |
| | | ACCAGGAGACACGGAATGTGAAGGCCCAGTCACAGACTGACCGAGTGGACCT |
| | | GGGGACCCTGCGCGGCTGCTACAACCAGAGCGAGGCCGGTTCTCACACCATC |
| | | CAGATAATGTATGGCTGCGACGTGGGGTCGGACGGGCGCTTCCTCCGCGGGT |
| | | ACCGGCAGGACGCCTACGACGGCAAGGATTACATCGCCCTGAACGAGGACCT |
| | | GCGCTCTTGGACCGCGGCGGACATGGCGGCTCAGATCACCAAGCGCAAGTGG |
| | | GAGGCGGCCCATGAGGCGGAGCAGTTGAGAGCCTACCTGGATGGCACGTGCG |
| | | TGGAGTGGCTCCGCAGATACCTGGAGAACGGGAAGGAGACGCTGCAGCGCAC |
| | | GGACCCCCCCAAGACACATATGACCCACCACCCCATCTCTGACCATGAGGCC |
| | | ACCCTGAGGTGCTGGGCCCTGGGCTTCTACCCTGCGGAGATCACACTGACCT |
| | | GGCAGCGGGATGGGGAGGACCAGACCCAGGACACGGAGCTCGTGGAGACCAG |
| | | GCCTGCAGGGGATGGAACCTTCCAGAAGTGGGCGGCTGTGGTGGTGCCTTCT |
| | | GGAGAGGAGCAGAGATACACCTGCCATGTGCAGCATGAGGGTCTGCCCAAGC |
| | | CCCTCACCCTGAGATGGGAGCTGTCTTCCCAGCCCACCATCCCCATCGTGGG |
| | | CATCATTGCTGGCCTGGTTCTCCTTGGAGCTGTGATCACTGGAGCTGTGGTC |
| | | GCTGCCGTGATGTGGAGGAGGAAGAGCTCAGATAGAAAAGGAGGGAGITACA |
| | | CTCAGGCTGCAAGCAGTGACAGTGCCCAGGGCTCTGATGTGTCCCTCACAGC |
| | | TTGTAAAGTGGCTACCAACTTCAGCCTGCTGAAGCAGGCTGGCGATGEGGAA |
| | | GAAAACCCTGGCCCAATGAAGTGGGTAACCTTTATTTCCCTTCTTTT_CTCT |
| | | TTAGCTCGGCTTATTCCATACTAATGCAATACATAAAAGCAAACAGCAAATT |
| | | CATAGGAATACCAATGGGACTACCACAAAGCATAGCACTAAGCAGCCZAATG |
| | | GTAGCACAACCAATGGGACTACCAGCAAAATTCGTAGCAGCATGGACACTAA |
| | | AAGCAGCAGCATGATAG |
| CV3-KR/G12C-A1101; | 5 | MSRSVALAVLALLSLSGLEAVVGACGVGKGCGASGGGGSGGGGSIQRTPKIQ |
| protein sequence | | VYSRHPAENGKSNFLNCYVSGFHPSDIEVDLLKNGERIEKVEHSDLSFSKDW |
| | | SFYLLYYTEFTPTEKDEYACRVNHVTLSQPKIVKWDRDMGGGGSGGGGSGGG |
| | | GSGGGGSGSGSHSMRYFYTSVSRPGRGEPRFIAVGYVDDTQFVREDSDAASQ |
| | | RMEPRAPWIEQEGPEYWDQETRNVKAQSQTDRVDLGTLRGCYNQSEDGSHTI |
| | | QIMYGCDVGPDGRLLRGYRQDAYDGKDYIALNEDLRSWTAADMAAQISKRKW |
| | | EAAHAAECQRAYLEGRCVEWLRRYLENGKETLQRTDPPKTHMTHHPISDHEA |
| | | TLRCWALGFYPAEITLTWQRDGEDQTQDTELVETRPAGDGTFQKWAAVVVPS |
| | | GEEQRYTCHVQHEGLPKPLTLRWELSSQPTIPIVGIIAGLVLLGAVLGAVV |
| | | AAVMWRRKSSDRKGGSYTQAASSDSAQGSDVSLTACKVATNFSLLKQAGDVE |
| | | ENPGPMKWVTFISLLELESSAYSILMQYIKANSKFIGIPMGLPQSIALSSLM |
| | | VAQPMGLPAKFVAAWTLKAAA |
| CV3-KR/G12C-A1101; | 6 | ATGTCTCGCTCCGTGGCCTTAGCTGTGCTCGCGCTACTCTCTCTTTCTGGCC |
| DNA sequence | | TGGAGGCTGTAGTTGGAGCTTGTGGCGTAGGCAAGGGCTGCGGCGCCECTGG |
| | | CGGCGGAGGATCCGGAGGCGGAGGAAGCATCCAGCGTACTCCAAAGASTCAG |
| | | GTTTACTCACGTCATCCAGCAGAGAATGGAAAGTCAAATTTCCTGAASTGCT |
| | | ATGTGTCTGGGTTTCATCCATCCGACATTGAAGTTGACTTACTGAAGAATGG |
| | | AGAGAGAATTGAAAAAGTGGAGCATTCAGACTTGTCTTTCAGCAAGGACTGG |
| | | TCTTTCTATCTCTTGTACTACACTGAATTCACCCCCACTGAAAAAGA-GAGT |
| | | ATGCCTGCCGTGTGAACCATGTGACTTTGTCACAGCCCAAGATAGTTAAGTG |
| | | GGATCGAGACATGGGCGGAGGCGGCTCTGGCGGAGGAGGCAGCGGCGGTGGC |
| | | GGCAGCGGCGGCGGCGGCTCTGGCAGCGGCTCCCACTCCATGAGGTASTTCT |
| | | ACACCTCCGTGTCCCGGCCCGGCCGCGGGGAGCCCCGCTTCATCGCCGTGGG |
| | | CTACGTGGACGACACGCAGTTCGTGCGGTTCGACAGCGACGCCGCGAGCCAG |
| | | AGGATGGAGCCGCGGGCGCCGTGGATAGAGCAGGAGGGGCCGGAGTATTGGG |
| | | ACCAGGAGACACGGAATGTGAAGGCCCAGTCACAGACTGACCGAGTGGACCT |
| | | GGGGACCCTGCGCGGCTGCTACAACCAGAGCGAGGACGGTTCTCACACCATC |
| | | CAGATAATGTATGGCTGCGACGTGGGGCCGGACGGGCGCTTACTCCGCGGGT |
| | | ACCGGCAGGACGCCTACGACGGCAAGGATTACATCGCCCTGAACGAGGACCT |
| | | GCGCTCTTGGACCGCGGCGGACATGGCAGCTCAGATCACCAAGCGCAAGTGG |
| | | GAGGCGGCCCATGCGGCGGAGCAGCAGAGAGCCTACCTGGAGGGCCGGTGCG |
| | | TGGAGTGGCTCCGCAGATACCTGGAGAACGGGAAGGAGACGCTGCAGCGCAC |
| | | GGACCCCCCCAAGACACATATGACCCACCACCCCATCTCTGACCATGAGGCC |
| | | ACCCTGAGGTGCTGGGCCCTGGGCTTCTACCCTGCGGAGATCACACTGACCT |
| | | GGCAGCGGGATGGGGAGGACCAGACCCAGGACACGGAGCTCGTGGAGACCAG |
| | | GCCTGCAGGGGATGGAACCTTCCAGAAGTGGGCGGCTGTGGTGGTGCCTTCT |
| | | GGAGAGGAGCAGAGATACACCTGCCATGTGCAGCATGAGGGTCTGCCCAAGC |
| | | CCCTCACCCTGAGATGGGAGCTGTCTTCCCAGCCCACCATCCCCATCGTGGG |
| | | CATCATTGCTGGCCTGGTTCTCCTTGGAGCTGTGATCACTGGAGCTGIGGTC |
| | | GCTGCCGTGATGTGGAGGAGGAAGAGCTCAGATAGAAAAGGAGGGAGTTACA |
| | | CTCAGGCTGCAAGCAGTGACAGTGCCCAGGGCTCTGATGTGTCTCTCACAGC |
| | | TTGTAAAGTGGCTACCAACTTCAGCCTGCTGAAGCAGGCTGGCGATGEGGAA |
| | | GAAAACCCTGGCCCAATGAAGTGGGTAACCTTTATTTCCCTTCTTTTTCTCT |
| | | TTAGCTCGGCTTATTCCATACTAATGCAATACATAAAAGCAAACAGCAAATT |
| | | CATAGGAATACCAATGGGACTACCACAAAGCATAGCACTAAGCAGCCZAATG |
| | | GTAGCACAACCAATGGGACTACCAGCAAAATTCGTAGCAGCATGGACACTAA |
| | | AAGCAGCAGCATGATAG |
| CV4-KR/G12C-B0702; | 7 | MSRSVALAVLALLSLSGLEAGACGVGKSAGCGASGGGGSGGGGSIQRTPKIQ |
| protein sequence | | VYSRHPAENGKSNELNCYVSGFHPSDIEVDLLKNGERIEKVEHSDLSFSKDW |
| | | SFYLLYYTEFTPTEKDEYACRVNHVTLSQPKIVKWDRDMGGGGSGGGGSGGG |
| | | GSGGGGSGSGSHSMRYFYTSVSRPGRGEPRFTSVGYVDDTQFVREDSDAASP |
| | | REEPRAPWIEQEGPEYWDRNTQIYKAQAQTDRESLRNLRGCYNQSEAGSHTL |
| | | QSMYGCDVGPDGRLLRGHDQLAYDGKDYIALNEDLRSWTAADTAAQITQRKW |
| | | EAAREAECRRAYLEGECVEWLRRYLENGKDKLERADPPKTHVTHHPISDHEA |
| | | TLRCWALGFYPAEITLTWQRDGEDQTQDTELVETRPAGDRTFQKWAAVVVPS |
| | | GEEQRYTCHVQHEGLPKPLTLRWEPSSQSTVPIVGIVAGLAVLAVVVIGAVV |
| | | AAVMCRRKSSGGKGGSYSQAACSDSAQGSDVSLTAATNFSLLKQAGDVEENP |
| | | GPMKWVTFISLLFLESSAYSILMQYIKANSKFIGIPMGLPQSIALSSLMVAQ |
| | | PMGLPAKFVAAWTLKAAA |
| CV4-KR/G12C-B0702; | 8 | ATGTCTCGCTCCGTGGCCTTAGCTGTGCTCGCGCTACTCTCTCTTTCTGGCC |
| DNA sequence | | TGGAGGCTGGAGCTTGCGGCGTAGGCAAGAGTGCCGGCTGCGGCGCCTCTGG |
| | | CGGCGGAGGATCCGGAGGCGGAGGAAGCATCCAGCGTACTCCAAAGASTCAG |
| | | GTTTACTCACGTCATCCAGCAGAGAATGGAAAGTCAAATTTCCTGAASTGCT |
| | | ATGTGTCTGGGTTTCATCCATCCGACATTGAAGTTGACTTACTGAAGAATGG |
| | | AGAGAGAATTGAAAAAGTGGAGCATTCAGACTTGTCTTTCAGCAAGGACTGG |
| | | TCTTTCTATCTCTTGTACTACACTGAATTCACCCCCACTGAAAAAGATGAGT |
| | | ATGCCTGCCGTGTGAACCATGTGACTTTGTCACAGCCCAAGATAGTTAAGTG |
| | | GGATCGAGACATGGGCGGAGGCGGCTCTGGCGGAGGAGGCAGCGGCGGTGGC |
| | | GGCAGCGGCGGCGGCGGCTCTGGCAGCGGCTCCCACTCCATGAGGTATTTCT |
| | | ACACCTCCGTGTCCCGGCCCGGCCGCGGGGAGCCCCGCTTCATCTCAGTGGG |
| | | CTACGTGGACGACACCCAGTTCGTGAGGTTCGACAGCGACGCCGCGAGTCCG |
| | | AGAGAGGAGCCGCGGGCGCCGTGGATAGAGCAGGAGGGGCCGGAGTATTGGG |
| | | ACCGGAACACACAGATCTACAAGGCCCAGGCACAGACTGACCGAGAGAGCCT |
| | | GCGGAACCTGCGCGGCTGCTACAACCAGAGCGAGGCCGGGTCTCACACCCTC |
| | | CAGAGCATGTACGGCTGCGACGTGGGGCCGGACGGGCGCCTCCTCCGCGGGC |
| | | ATGACCAGTTAGCCTACGACGGCAAGGATTACATCGCCCTGAACGAGGACCT |
| | | GCGCTCCTGGACCGCCGCGGACACGGCGGCTCAGATCACCCAGCGCAAGTGG |
| | | GAGGCGGCCCGTGAGGCGGAGCAGCGGAGAGCCTACCTGGAGGGCGAGTGCG |
| | | TGGAGTGGCTCCGCAGATACCTGGAGAACGGGAAGGACAAGCTGGAGCGCGC |
| | | TGACCCCCCAAAGACACACGTGACCCACCACCCCATCTCTGACCATGAGGCC |
| | | ACCCTGAGGTGCTGGGCCCTGGGTTTCTACCCTGCGGAGATCACACTGACCT |
| | | GGCAGCGGGATGGCGAGGACCAAACTCAGGACACTGAGCTTGTGGAGACCAG |
| | | ACCAGCAGGAGATAGAACCTTCCAGAAGTGGGCAGCTGTGGTGGTGCCTTCT |
| | | GGAGAAGAGCAGAGATACACATGCCATGTACAGCATGAGGGGCTGCCGAAGC |
| | | CCCTCACCCTGAGATGGGAGCCGTCTTCCCAGTCCACCGTCCCCATCGTGGG |
| | | CATTGTTGCTGGCCTGGCTGTCCTAGCAGTIGTGGTCATCGGAGCTG=GGTC |
| | | GCTGCTGTGATGTGTAGGAGGAAGAGTTCAGGTGGAAAAGGAGGGAGCTACT |
| | | CTCAGGCTGCGTGCAGCGACAGTGCCCAGGGCTCTGATGTGTCTCTCACAGC |
| | | TGCTACCAACTTCAGCCTGCTGAAGCAGGCTGGCGATGTGGAAGAAAACCCT |
| | | GGCCCAATGAAGTGGGTAACCTTTATTTCCCTTCTTTTTCTCTTTAGCTCGG |
| | | CTTATTCCATACTAATGCAATACATAAAAGCAAACAGCAAATTCATAGGAAT |
| | | ACCAATGGGACTACCACAAAGCATAGCACTAAGCAGCCTAATGGTAGCACAA |
| | | CCAATGGGACTACCAGCAAAATTCGTAGCAGCATGGACACTAAAAGCAGCAG |
| | | CATGATAG |
| CV5-KR/G12D-A0201; | 9 | MSRSVALAVLALLSLSGLEAKLVVVGADGVGCGASGGGGSGGGGSIQRTPKI |
| protein sequence | | QVYSRHPAENGKSNFLNCYVSGFHPSDIEVDLLKNGERIEKVEHSDLSESKD |
| | | WSFYLLYYTEFTPTEKDEYACRVNHVTLSQPKIVKWDRDMGGGGSGGGGSGG |
| | | GGSGGGGSGSGSHSMRYFFTSVSRPGRGEPRFIAVGYVDDTQFVREDSDAAS |
| | | QRMEPRAPWIEQEGPEYWDGETRKVKAHSQTHRVDLGTLRGCYNQSEAGSHT |
| | | VQRMYGCDVGSDWRFLRGYHQYAYDGKDYIALKEDLRSWTAADMAAQTTKHK |
| | | WEAAHVAEQLRAYLEGTCVEWLRRYLENGKETLQRTDAPKTHMTHHAVSDHE |
| | | ATLRCWALSFYPAEITLTWQRDGEDQTQDTELVETRPAGDGTFQKWAAVVVP |
| | | SGQEQRYTCHVQHEGLPKPLTLRWEPSSQPTIPIVGIIAGLVLFGAVITGAV |
| | | VAAVMWRRKSSDRKGGSYSQAASSDSAQGSDVSLTACKVATNFSLLKQAGDV |
| | | EENPGPMKWVTFISLLFLESSAYSILMQYIKANSKFIGIPMGLPQSIALSSL |
| | | MVAQPMGLPAKFVAAWTLKAAA |
| CV5-KR/G12D-A0201; | 10 | ATGTCTCGCTCCGTGGCCTTAGCTGTGCTCGCGCTACTCTCTCTTTCTGGCC |
| DNA sequence | | TGGAGGCTAAACTTGTGGTAGTTGGAGCTGACGGCGTAGGCTGCGGCGCCTC |
| | | TGGCGGCGGAGGATCCGGAGGCGGAGGAAGCATCCAGCGTACTCCAAAGATT |
| | | CAGGTTTACTCACGTCATCCAGCAGAGAATGGAAAGTCAAATTTCCTGAATT |
| | | GCTATGTGTCTGGGTTTCATCCATCCGACATTGAAGTTGACTTACTGAAGAA |
| | | TGGAGAGAGAATTGAAAAAGTGGAGCATTCAGACTTGTCTTTCAGCAAGGAC |
| | | TGGTCTTTCTATCTCTTGTACTACACTGAATTCACCCCCACTGAAAAAGATG |
| | | AGTATGCCTGCCGTGTGAACCATGTGACTTTGTCACAGCCCAAGATAGTTAA |
| | | GTGGGATCGAGACATGGGCGGAGGCGGCTCTGGCGGAGGAGGCAGCGGCGGT |
| | | GGCGGCAGCGGCGGCGGCGGCTCTGGCAGCGGCTCTCACTCCATGAGGTATT |
| | | TCTTCACATCCGTGTCCCGGCCCGGCCGCGGGGAGCCCCGCTTCATCGCAGT |
| | | GGGCTACGTGGACGACACGCAGTTCGTGCGGTTCGACAGCGACGCCGCGAGC |
| | | CAGAGGATGGAGCCGCGGGCGCCGTGGATAGAGCAGGAGGGTCCGGAGTATT |
| | | GGGACGGGGAGACACGGAAAGTGAAGGCCCACTCACAGACTCACCGAGTGGA |
| | | CCTGGGGACCCTGCGCGGCTGCTACAACCAGAGCGAGGCCGGTTCTCACACC |
| | | GTCCAGAGGATGTATGGCTGCGACGTGGGGTCGGACTGGCGCTTCCTCCGCG |
| | | GGTACCACCAGTACGCCTACGACGGCAAGGATTACATCGCCCTGAAAGAGGA |
| | | CCTGCGCTCTTGGACCGCGGCGGACATGGCAGCTCAGACCACCAAGCACAAG |
| | | TGGGAGGCGGCCCATGTGGCGGAGCAGTTGAGAGCCTACCTGGAGGGCACGT |
| | | GCGTGGAGTGGCTCCGCAGATACCTGGAGAACGGGAAGGAGACGCTGCAGCG |
| | | CACGGACGCCCCCAAAACGCATATGACTCACCACGCTGTCTCTGACCATGAA |
| | | GCCACCCTGAGGTGCTGGGCCCTGAGCTTCTACCCTGCGGAGATCACACTGA |
| | | CCTGGCAGCGGGATGGGGAGGACCAGACCCAGGACACGGAGCTCGTGGAGAC |
| | | CAGGCCTGCAGGGGATGGAACCTTCCAGAAGTGGGCGGCTGTGGTGGGCCT |
| | | TCTGGACAGGAGCAGAGATACACCTGCCATGTGCAGCATGAGGGTTTGCCCA |
| | | AGCCCCTCACCCTGAGATGGGAGCCGTCTTCCCAGCCCACCATCCCCATCGT |
| | | GGGCATCATTGCTGGCCTGGTTCTCTTTGGAGCTGTGATCACTGGAGCTGTG |
| | | GTCGCTGCTGTGATGTGGAGGAGGAAGAGCTCAGATAGAAAAGGAGGGAGCT |
| | | ACTCTCAGGCTGCAAGCAGTGACAGTGCCCAGGGCTCTGATGTGTCTCTCAC |
| | | AGCTTGTAAAGTGGCTACCAACTTCAGCCTGCTGAAGCAGGCTGGCGATGTG |
| | | GAAGAAAACCCTGGCCCAATGAAGTGGGTAACCTTTATTTCCCTTCTTTTTC |
| | | TCTTTAGCTCGGCTTATTCCATACTAATGCAATACATAAAAGCAAACAGCAA |
| | | ATTCATAGGAATACCAATGGGACTACCACAAAGCATAGCACTAAGCAGCCTA |
| | | ATGGTAGCACAACCAATGGGACTACCAGCAAAATTCGTAGCAGCATGGACAC |
| | | TAAAAGCAGCAGCATGATAG |
| CV6-KR/G12D-A0301; | 11 | MSRSVALAVLALLSLSGLEAVVGADGVGKGCGASGGGGSGGGGSIQRTPKIQ |
| protein sequence | | VYSRHPAENGKSNELNCYVSGFHPSDIEVDLLKNGERIEKVEHSDLSFSKDW |
| | | SFYLLYYTEFTPTEKDEYACRVNHVTLSQPKIVKWDRDMGGGGSGGGGSGGG |
| | | GSGGGGSGSGSHSMRYFFTSVSRPGRGEPRFIAVGYVDDTQFVREDSDAASQ |
| | | RMEPRAPWIEQEGPEYWDQETRNVKAQSQTDRVDLGTLRGCYNQSEAGSHTI |
| | | QIMYGCDVGSDGRFLRGYRQDAYDGKDYIALNEDLRSWTAADMAAQITKRKW |
| | | EAAHEAEQLRAYLDGTCVEWLRRYLENGKETLQRTDPPKTHMTHHPISDHEA |
| | | TLRCWALGFYPAEITLTWQRDGEDQTQDTELVETRPAGDGTFQKWAAVVVPS |
| | | GEEQRYTCHVQHEGLPKPLTLRWELSSQPTIPIVGIIAGLVLLGAVISGAVV |
| | | AAVMWRRKSSDRKGGSYTQAASSDSAQGSDVSLTACKVATNFSLLKQAGDVE |
| | | ENPGPMKWVTFISLLELESSAYSILMQYIKANSKFIGIPMGLPQSIALSSLM |
| | | VAQPMGLPAKFVAAWTLKAAA |
| CV6-KR/G12D-A0301; | 12 | ATGTCTCGCTCCGTGGCCTTAGCTGTGCTCGCGCTACTCTCTCTTTCEGGCC |
| DNA sequence | | TGGAGGCTGTAGTTGGAGCTGATGGCGTAGGCAAGGGCTGCGGCGCCTCTGG |
| | | CGGCGGAGGATCCGGAGGCGGAGGAAGCATCCAGCGTACTCCAAAGATTCAG |
| | | GTTTACTCACGTCATCCAGCAGAGAATGGAAAGTCAAATTTCCTGAATTGCT |
| | | ATGTGTCTGGGTTTCATCCATCCGACATTGAAGTTGACTTACTGAAGAATGG |
| | | AGAGAGAATTGAAAAAGTGGAGCATTCAGACTTGTCTTTCAGCAAGGACTGG |
| | | TCTTTCTATCTCTTGTACTACACTGAATTCACCCCCACTGAAAAAGATGAGT |
| | | ATGCCTGCCGTGTGAACCATGTGACTITGTCACAGCCCAAGATAGTTAAGTG |
| | | GGATCGAGACATGGGCGGAGGCGGCTCTGGCGGAGGAGGCAGCGGCGGTGGC |
| | | GGCAGCGGCGGCGGCGGCTCTGGCAGCGGCTCCCACTCCATGAGGTATTTCT |
| | | TCACATCCGTGTCCCGGCCCGGCCGCGGGGAGCCCCGCTTCATCGCCGTGGG |
| | | CTACGTGGACGACACGCAGTTCGTGCGGTTCGACAGCGACGCCGCGAGCCAG |
| | | AGGATGGAGCCGCGGGCGCCGTGGATAGAGCAGGAGGGGCCGGAGTATTGGG |
| | | ACCAGGAGACACGGAATGTGAAGGCCCAGTCACAGACTGACCGAGTGGACCT |
| | | GGGGACCCTGCGCGGCTGCTACAACCAGAGCGAGGCCGGTTCTCACACCATC |
| | | CAGATAATGTATGGCTGCGACGTGGGGTCGGACGGGCGCTTCCTCCGCGGGT |
| | | ACCGGCAGGACGCCTACGACGGCAAGGATTACATCGCCCTGAACGAGGACCT |
| | | GCGCTCTTGGACCGCGGCGGACATGGCGGCTCAGATCACCAAGCGCAAGTGG |
| | | GAGGCGGCCCATGAGGCGGAGCAGTTGAGAGCCTACCTGGATGGCACGTGCG |
| | | TGGAGTGGCTCCGCAGATACCTGGAGAACGGGAAGGAGACGCTGCAGCGCAC |
| | | GGACCCCCCCAAGACACATATGACCCACCACCCCATCTCTGACCATGAGGCC |
| | | ACCCTGAGGTGCTGGGCCCTGGGCTTCTACCCTGCGGAGATCACACTGACCT |
| | | GGCAGCGGGATGGGGAGGACCAGACCCAGGACACGGAGCTCGTGGAGACCAG |
| | | GCCTGCAGGGGATGGAACCTTCCAGAAGTGGGCGGCTGTGGTGGTGCCTTCT |
| | | GGAGAGGAGCAGAGATACACCTGCCATGTGCAGCATGAGGGTCTGCCCAAGC |
| | | CCCTCACCCTGAGATGGGAGCTGTCTTCCCAGCCCACCATCCCCATCGTGGG |
| | | CATCATTGCTGGCCTGGTTCTCCTTGGAGCTGTGATCACTGGAGCTGEGGTC |
| | | GCTGCCGTGATGTGGAGGAGGAAGAGCTCAGATAGAAAAGGAGGGAGETACA |
| | | CTCAGGCTGCAAGCAGTGACAGTGCCCAGGGCTCTGATGTGTCCCTCACAGC |
| | | TTGTAAAGTGGCTACCAACTTCAGCCTGCTGAAGCAGGCTGGCGATGEGGAA |
| | | GAAAACCCTGGCCCAATGAAGTGGGTAACCTTTATTTCCCTTCTTTTTCTCT |
| | | TTAGCTCGGCTTATTCCATACTAATGCAATACATAAAAGCAAACAGCAAATT |
| | | CATAGGAATACCAATGGGACTACCACAAAGCATAGCACTAAGCAGCCTAATG |
| | | GTAGCACAACCAATGGGACTACCAGCAAAATTCGTAGCAGCATGGACACTAA |
| | | AAGCAGCAGCATGATAG |
| CV7-KR/G12D-A1101; | 13 | MSRSVALAVLALLSLSGLEAVVGADGVGKGCGASGGGGSGGGGSIQREPKIQ |
| protein sequence | | VYSRHPAENGKSNFLNCYVSGFHPSDIEVDLLKNGERIEKVEHSDLSFSKDW |
| | | SFYLLYYTEFTPTEKDEYACRVNHVTLSQPKIVKWDRDMGGGGSGGGGSGGG |
| | | GSGGGGSGSGSHSMRYFYTSVSRPGRGEPRFIAVGYVDDTQFVREDSDAASQ |
| | | RMEPRAPWIEQEGPEYWDQETRNVKAQSQTDRVDLGTLRGCYNQSEDGSHTI |
| | | QIMYGCDVGPDGRLLRGYRQDAYDGKDYIALNEDLRSWTAADMAAQITKRKW |
| | | EAAHAAEQQRAYLEGRCVEWLRRYLENGKETLQRTDPPKTHMTHHPISDHEA |
| | | TLRCWALGFYPAEITLTWQRDGEDQTQDTELVETRPAGDGTFQKWAAVVVPS |
| | | GEEQRYTCHVQHEGLPKPLTLRWELSSQPTIPIVGIIAGLVLIGAVITGAVV |
| | | AAVMWRRKSSDRKGGSYTQAASSDSAQGSDVSLTACKVATNESLLKQAGDVE |
| | | ENPGPMKWVTFISLLELESSAYSILMQYIKANSKFIGIPMGLPQSIALSSLM |
| | | VAQPMGLPAKFVAAWTLKAAA |
| CV7-KR/G12D-A1101; | 14 | ATGTCTCGCTCCGTGGCCTTAGCTGTGCTCGCGCTACTCTCTCTTTCEGGCC |
| DNA sequence | | TGGAGGCTGTAGTTGGAGCTGATGGCGTAGGCAAGGGCTGCGGCGCCTCTGG |
| | | CGGCGGAGGATCCGGAGGCGGAGGAAGCATCCAGCGTACTCCAAAGASTCAG |
| | | GITTACTCACGTCATCCAGCAGAGAATGGAAAGTCAAATTTCCTGAASTGCT |
| | | ATGTGTCTGGGTTTCATCCATCCGACATTGAAGTTGACTTACTGAAGAATGG |
| | | AGAGAGAATTGAAAAAGTGGAGCATTCAGACTTGTCTTTCAGCAAGGACTGG |
| | | TCTTTCTATCTCTTGTACTACACTGAATTCACCCCCACTGAAAAAGATGAGT |
| | | ATGCCTGCCGTGTGAACCATGTGACTTTGTCACAGCCCAAGATAGTTAAGTG |
| | | GGATCGAGACATGGGCGGAGGCGGCTCTGGCGGAGGAGGCAGCGGCGGTGGC |
| | | GGCAGCGGCGGCGGCGGCTCTGGCAGCGGCTCCCACTCCATGAGGTATTTCT |
| | | ACACCTCCGTGTCCCGGCCCGGCCGCGGGGAGCCCCGCTTCATCGCCGTGGG |
| | | CTACGTGGACGACACGCAGTTCGTGCGGTTCGACAGCGACGCCGCGAGCCAG |
| | | AGGATGGAGCCGCGGGCGCCGTGGATAGAGCAGGAGGGGCCGGAGTASTGGG |
| | | ACCAGGAGACACGGAATGTGAAGGCCCAGTCACAGACTGACCGAGTGGACCT |
| | | GGGGACCCTGCGCGGCTGCTACAACCAGAGCGAGGACGGTTCTCACACCATC |
| | | CAGATAATGTATGGCTGCGACGTGGGGCCGGACGGGCGCTTACTCCGCGGGT |
| | | ACCGGCAGGACGCCTACGACGGCAAGGATTACATCGCCCTGAACGAGGACCT |
| | | GCGCTCTTGGACCGCGGCGGACATGGCAGCTCAGATCACCAAGCGCAAGTGG |
| | | GAGGCGGCCCATGCGGCGGAGCAGCAGAGAGCCTACCTGGAGGGCCGGTGCG |
| | | TGGAGTGGCTCCGCAGATACCTGGAGAACGGGAAGGAGACGCTGCAGCGCAC |
| | | GGACCCCCCCAAGACACATATGACCCACCACCCCATCTCTGACCATGAGGCC |
| | | ACCCTGAGGTGCTGGGCCCTGGGCTTCTACCCTGCGGAGATCACACTGACCT |
| | | GGCAGCGGGATGGGGAGGACCAGACCCAGGACACGGAGCTCGTGGAGACCAG |
| | | GCCTGCAGGGGATGGAACCTTCCAGAAGTGGGCGGCTGTGGTGGTGCCTTCT |
| | | GGAGAGGAGCAGAGATACACCTGCCATGTGCAGCATGAGGGTCTGCCCAAGC |
| | | CCCTCACCCTGAGATGGGAGCTGTCTTCCCAGCCCACCATCCCCATCGTGGG |
| | | CATCATTGCTGGCCTGGTTCTCCTTGGAGCTGTGATCACTGGAGCTGTGGTC |
| | | GCTGCCGTGATGTGGAGGAGGAAGAGCTCAGATAGAAAAGGAGGGAGTTACA |
| | | CTCAGGCTGCAAGCAGTGACAGTGCCCAGGGCTCTGATGTGTCTCTCACAGC |
| | | TTGTAAAGTGGCTACCAACTTCAGCCTGCTGAAGCAGGCTGGCGATGTGGAA |
| | | GAAAACCCTGGCCCAATGAAGTGGGTAACCTTTATTTCCCTTCTTTTTCTCT |
| | | TTAGCTCGGCTTATTCCATACTAATGCAATACATAAAAGCAAACAGCAAATT |
| | | CATAGGAATACCAATGGGACTACCACAAAGCATAGCACTAAGCAGCCZAATG |
| | | GTAGCACAACCAATGGGACTACCAGCAAAATTCGTAGCAGCATGGACACTAA |
| | | AAGCAGCAGCATGATAG |
| CV8-KR/G12D-B0702; | 15 | MSRSVALAVLALLSLSGLEAGADGVGKSAGCGASGGGGSGGGGSIQRTPKIQ |
| protein sequence | | VYSRHPAENGKSNFLNCYVSGFHPSDIEVDLLKNGERIEKVEHSDLSFSKDW |
| | | SFYLLYYTEFTPTEKDEYACRVNHVTLSQPKIVKWDRDMGGGGSGGGGSGGG |
| | | GSGGGGSGSGSHSMRYFYTSVSRPGRGEPRFISVGYVDDTQFVREDSDAASP |
| | | REEPRAPWIEQEGPEYWDRNTQIYKAQAQTDRESLRNLRGCYNQSEAGSHTL |
| | | QSMYGCDVGPDGRLLRGHDQLAYDGKDYIALNEDLRSWTAADTAAQITQRKW |
| | | EAAREAECRRAYLEGECVEWLRRYLENGKDKLERADPPKTHVTHHPISDHEA |
| | | TLRCWALGFYPAEITLTWQRDGEDQTQDTELVETRPAGDRTFQKWAAVVVPS |
| | | GEEQRYTCHVQHEGLPKPLILRWEPSSQSTVPIVGIVAGLAVLAVVVIGAVV |
| | | AAVMCRRKSSGGKGGSYSQAACSDSAQGSDVSLTAATNFSLLKQAGDVEENP |
| | | GPMKWVTFISLLFLFSSAYSILMQYIKANSKFIGIPMGLPQSIALSSLMVAQ |
| | | PMGLPAKFVAAWTLKAAA |
| CV8-KR/G12D-B0702; | 16 | ATGTCTCGCTCCGTGGCCTTAGCTGTGCTCGCGCTACTCTCTCTTTCTGGCC |
| DNA sequence | | TGGAGGCTGGAGCTGATGGCGTAGGCAAGAGTGCCGGCTGCGGCGCCTCTGG |
| | | CGGCGGAGGATCCGGAGGCGGAGGAAGCATCCAGCGTACTCCAAAGASTCAG |
| | | GTTTACTCACGTCATCCAGCAGAGAATGGAAAGTCAAATTTCCTGAATGCT |
| | | ATGTGTCTGGGTTTCATCCATCCGACATTGAAGTTGACTTACTGAAGAATGG |
| | | AGAGAGAATTGAAAAAGTGGAGCATTCAGACTTGTCTTTCAGCAAGGACTGG |
| | | TCTTTCTATCTCTTGTACTACACTGAATTCACCCCCACTGAAAAAGAGAGT |
| | | ATGCCTGCCGTGTGAACCATGTGACTTTGTCACAGCCCAAGATAGTTAAGTG |
| | | GGATCGAGACATGGGCGGAGGCGGCTCTGGCGGAGGAGGCAGCGGCGGTGGC |
| | | GGCAGCGGCGGCGGCGGCTCTGGCAGCGGCTCCCACTCCATGAGGTATTTCT |
| | | ACACCTCCGTGTCCCGGCCCGGCCGCGGGGAGCCCCGCTTCATCTCAGTGGG |
| | | CTACGTGGACGACACCCAGTTCGTGAGGTTCGACAGCGACGCCGCGAGTCCG |
| | | AGAGAGGAGCCGCGGGCGCCGTGGATAGAGCAGGAGGGGCCGGAGTATTGGG |
| | | ACCGGAACACACAGATCTACAAGGCCCAGGCACAGACTGACCGAGAGAGCCT |
| | | GCGGAACCTGCGCGGCTGCTACAACCAGAGCGAGGCCGGGTCTCACACCCTC |
| | | CAGAGCATGTACGGCTGCGACGTGGGGCCGGACGGGCGCCTCCTCCGCGGGC |
| | | ATGACCAGTTAGCCTACGACGGCAAGGATTACATCGCCCTGAACGAGGACCT |
| | | GCGCTCCTGGACCGCCGCGGACACGGCGGCTCAGATCACCCAGCGCAAGTGG |
| | | GAGGCGGCCCGTGAGGCGGAGCAGCGGAGAGCCTACCTGGAGGGCGAGTGCG |
| | | TGGAGTGGCTCCGCAGATACCTGGAGAACGGGAAGGACAAGCTGGAGCGCGC |
| | | TGACCCCCCAAAGACACACGTGACCCACCACCCCATCTCTGACCATGAGGCC |
| | | ACCCTGAGGTGCTGGGCCCTGGGTTTCTACCCTGCGGAGATCACACTGACCT |
| | | GGCAGCGGGATGGCGAGGACCAAACTCAGGACACTGAGCTTGTGGAGACCAG |
| | | ACCAGCAGGAGATAGAACCTTCCAGAAGTGGGCAGCTGTGGTGGTGCCTTCT |
| | | GGAGAAGAGCAGAGATACACATGCCATGTACAGCATGAGGGGCTGCCGAAGC |
| | | CCCTCACCCTGAGATGGGAGCCGTCTTCCCAGTCCACCGTCCCCATCGTGGG |
| | | CATTGTTGCTGGCCTGGCTGTCCTAGCAGTTGTGGTCATCGGAGCTGEGGTC |
| | | GCTGCTGTGATGTGTAGGAGGAAGAGTTCAGGTGGAAAAGGAGGGAGCTACT |
| | | CTCAGGCTGCGTGCAGCGACAGTGCCCAGGGCTCTGATGTGTCTCTCACAGC |
| | | TGCTACCAACTTCAGCCTGCTGAAGCAGGCTGGCGATGTGGAAGAAAACCCT |
| | | GGCCCAATGAAGTGGGTAACCTTTATTTCCCTTCTTTTTCTCTTTAGCTCGG |
| | | CTTATTCCATACTAATGCAATACATAAAAGCAAACAGCAAATTCATAGGAAT |
| | | ACCAATGGGACTACCACAAAGCATAGCACTAAGCAGCCTAATGGTAGCACAA |
| | | CCAATGGGACTACCAGCAAAATTCGTAGCAGCATGGACACTAAAAGCAGCAG |
| | | CATGATAG |
| CV9-HPV/E6-29- | 17 | MSRSVALAVLALLSLSGLEATIHDIILECVGCGASGGGGSGGGGSIQRTPKI |
| A0201; Protein | | QVYSRHPAENGKSNFLNCYVSGFHPSDIEVDLLKNGERIEKVEHSDLSESKD |
| sequence | | WSFYLLYYTEFTPTEKDEYACRVNHVTLSQPKIVKWDRDMGGGGSGGGGSGG |
| | | GGSGGGGSGSGSHSMRYFFTSVSRPGRGEPRFIAVGYVDDTQFVREDSDAAS |
| | | QRMEPRAPWIEQEGPEYWDGETRKVKAHSQTHRVDLGTLRGCYNQSEAGSHT |
| | | VQRMYGCDVGSDWRFLRGYHQYAYDGKDYIALKEDLRSWTAADMAAQTTKHK |
| | | WEAAHVAEQLRAYLEGTCVEWLRRYLENGKETLQRTDAPKTHMTHHAVSDHE |
| | | ATLRCWALSFYPAEITLTWQRDGEDQTQDTELVETRPAGDGTFQKWAAVVVP |
| | | SGQEQRYTCHVQHEGLPKPLTLRWEPSSQPTIPIVGIIAGLVLFGAVETGAV |
| | | VAAVMWRRKSSDRKGGSYSQAASSDSAQGSDVSLTACKVATNFSLLKQAGDV |
| | | EENPGPMKWVTFISLLFLESSAYSILMQYIKANSKFIGIPMGLPQSIALSSL |
| | | MVAQPMGLPAKFVAAWTLKAAA |
| CV9-HPV/E6-29- | 18 | ATGAGCAGAAGCGTGGCCCTGGCCGTGCTGGCCCTGCTGAGCCTGAGCGGCC |
| A0201; DNA sequence | | TGGAGGCCACCATCCACGACATCATCCTGGAATGCGTGGGATGCGGCGCTAG |
| | | CGGCGGGGGCGGAAGCGGTGGCGGAGGTAGCATTCAGAGAACCCCCAAGATC |
| | | CAAGTGTACAGCAGACACCCTGCCGAAAACGGAAAGAGCAACTTCCTGAACT |
| | | GCTACGTGAGCGGCTTCCACCCTAGCGACATCGAGGTGGACCTGCTGAAGAA |
| | | CGGCGAGAGAATCGAGAAGGTGGAGCACAGCGACCTGAGCTTCAGCAAGGAC |
| | | TGGAGCTTCTACCTGCTGTACTACACCGAGTTCACCCCCACCGAGAAGGACG |
| | | AGTACGCCTGCAGAGTGAACCACGTGACCCTGTCTCAGCCCAAGATCGTGAA |
| | | GTGGGACCGGGACATGGGGGGCGGAGGCTCGGGTGGCGGTGGCAGCGGCGGT |
| | | GGGGGTAGTGGGGGTGGCGGCAGCGGAAGTGGTAGCCACAGCATGAGATACT |
| | | TCTTCACAAGCGTGAGCAGACCCGGCAGAGGCGAGCCTAGATTCATCGCCGT |
| | | GGGCTACGTGGACGACACACAGTTCGTGAGATTCGACAGCGACGCCGCTTCT |
| | | CAGAGAATGGAGCCTAGAGCCCCCTGGATCGAGCAAGAGGGCCCCGAGTACT |
| | | GGGACGGCGAGACAAGAAAGGTGAAGGCCCACTCTCAGACCCACAGAGTGGA |
| | | CCTGGGCACCCTGAGAGGCTGCTACAATCAGAGCGAGGCCGGCAGCCACACC |
| | | GTGCAGAGAATGTACGGCTGCGACGTGGGCAGCGACTGGAGATTCCTGAGAG |
| | | GCTACCATCAGTACGCCTACGACGGCAAGGACTACATCGCCCTGAAGGAGGA |
| | | CCTGAGAAGCTGGACCGCCGCCGACATGGCCGCCCAAACCACCAAACACAAG |
| | | TGGGAGGCCGCCCACGTGGCCGAGCAGCTGAGAGCCTACCTGGAGGGCACCT |
| | | GCGTGGAGTGGCTGAGAAGATACCTGGAAAACGGCAAAGAGACGCTGCAGAG |
| | | AACCGACGCCCCCAAGACCCACATGACCCACCACGCCGTGAGCGACCACGAG |
| | | GCCACCCTGAGATGCTGGGCCCTGAGCTTCTACCCCGCAGAGATCACGCTGA |
| | | CCTGGCAGAGAGACGGCGAGGATCAGACCCAAGACACCGAGCTGGTGGAGAC |
| | | AAGACCGGCCGGCGATGGCACATTTCAGAAATGGGCCGCCGTTGTGGIGCCT |
| | | AGCGGCCAAGAGCAGAGATACACCTGCCACGTGCAGCACGAGGGCCTGCCCA |
| | | AGCCCCTGACCCTGAGATGGGAGCCTAGCTCTCAGCCCACCATCCCCATCGT |
| | | GGGCATCATCGCCGGCCTGGTGCTGTTCGGCGCCGTGATCACCGGCGCCGTG |
| | | GTGGCCGCCGTGATGTGGAGAAGAAAGAGCAGCGACCGCAAAGGGGGCAGCT |
| | | ATAGCCAAGCAGCTAGCTCTGACAGCGCCCAAGGCAGCGACGTGAGCCTGAC |
| | | CGCCTGCAAGGTGTAG |
| CV10-HPV/E7-11- | 19 | MSRSVALAVLALLSLSGLEAYMLDLQPETGCGASGGGGSGGGGSIQREPKIQ |
| A0201; Protein | | VYSRHPAENGKSNFLNCYVSGFHPSDIEVDLLKNGERIEKVEHSDLSFSKDW |
| sequence | | SFYLLYYTEFTPTEKDEYACRVNHVTLSQPKIVKWDRDMGGGGSGGGGSGGG |
| | | GSGGGGSGSGSHSMRYFFTSVSRPGRGEPRFIAVGYVDDTQFVREDSDAASQ |
| | | RMEPRAPWIEQEGPEYWDGETRKVKAHSQTHRVDLGTLRGCYNQSEAGSHTV |
| | | QRMYGCDVGSDWRFLRGYHQYAYDGKDYIALKEDLRSWTAADMAAQTEKHKW |
| | | EAAHVAECLRAYLEGTCVEWLRRYLENGKETLQRTDAPKTHMTHHAVSDHEA |
| | | TLRCWALSFYPAEITLTWQRDGEDQTQDTELVETRPAGDGTFQKWAAVVVPS |
| | | GQEQRYTCHVQHEGLPKPLILRWEPSSQPTIPIVGIIAGLVLFGAVITGAVV |
| | | AAVMWRRKSSDRKGGSYSQAASSDSAQGSDVSLTACKVATNFSLLKQAGDVE |
| | | ENPGPMKWVTFISLLFLESSAYSILMQYIKANSKFIGIPMGLPQSIALSSLM |
| | | VAQPMGLPAKFVAAWTLKAAA |
| CV10-HPV/E7-11- | 20 | ATGTCTCGCTCCGTGGCCTTAGCTGTGCTCGCGCTACTCTCTCTTTCTGGCC |
| A0201; DNA sequence | | TGGAGGCTtatatgttagatttgcaaccagagacaGGCTGCGGCGCCTCTGG |
| | | CGGCGGAGGATCCGGAGGCGGAGGAAGCATCCAGCGTACTCCAAAGASTCAG |
| | | GTTTACTCACGTCATCCAGCAGAGAATGGAAAGTCAAATTTCCTGAASTGCT |
| | | ATGTGTCTGGGTTTCATCCATCCGACATTGAAGTTGACTTACTGAAGAATGG |
| | | AGAGAGAATTGAAAAAGTGGAGCATTCAGACTTGTCTTTCAGCAAGGACTGG |
| | | TCTTTCTATCTCTTGTACTACACTGAATTCACCCCCACTGAAAAAGATGAGT |
| | | ATGCCTGCCGTGTGAACCATGTGACTTTGTCACAGCCCAAGATAGTTAAGTG |
| | | GGATCGAGACATGGGCGGAGGCGGCTCTGGCGGAGGAGGCAGCGGCGGTGGC |
| | | GGCAGCGGCGGCGGCGGCTCTGGCAGCggctctcactccatgaggtatttct |
| | | tcacatccgtgtcccggcccggccgcggggagccccgcttcatcgcagtggg |
| | | ctacgtggacgacacgcagttcgtgcggttcgacagcgacgccgcgagccag |
| | | aggatggagccgcgggcgccgtggatagagcaggagggtccggagtattggg |
| | | acggggagacacggaaagtgaaggcccactcacagactcaccgagtggacct |
| | | ggggaccctgcgcggcTGCtacaaccagagcgaggccggttctcacaccgtc |
| | | cagaggatgtatggctgcgacgtggggtcggactggcgcttcctccgcgggt |
| | | accaccagtacgcctacgacggcaaggattacatcgccctgaaagaggacct |
| | | gcgctcttggaccgcggcggacatggcagctcagaccaccaagcacaagtgg |
| | | gaggcggcccatgtggcggagcagttgagagcctacctggagggcacgtgcg |
| | | tggagtggctccgcagatacctggagaacgggaaggagacgctgcagcgcac |
| | | ggacgcccccaaaacgcatatgactcaccacgctgtctctgaccatgaagcc |
| | | accctgaggtgctgggccctgagcttctaccctgcggagatcacactgacct |
| | | ggcagcgggatggggaggaccagacccaggacacggagctcgtggagaccag |
| | | gcctgcaggggatggaaccttccagaagtgggcggctgtggtggtgccttct |
| | | ggacaggagcagagatacacctgccatgtgcagcatgagggtttgcccaagc |
| | | ccctcaccctgagatgggagccgtcttcccagcccaccatccccatcgtggg |
| | | catcattgctggcctggttctctttggagctgtgatcactggagctgtggtc |
| | | gctgctgtgatgtggaggaggaagagctcagatagaaaaggagggagctact |
| | | ctcaggctgcaagcagtgacagtgcccagggctctgatgtgtctctcacagc |
| | | ttgtaaagtgGCTACCAACTTCAGCCTGCTGAAGCAGGCTGGCGATGEGGAA |
| | | GAAAACCCTGGCCCAatgaagtgggtaacctttatttcccttctttttctct |
| | | ttagctcggcttattccATACTAATGCAATACATAAAAGCAAACAGCAAATT |
| | | CATAGGAATACCAATGGGACTACCACAAAGCATAGCACTAAGCAGCCTAATG |
| | | GTAGCACAACCAATGGGACTACCAGCAAAATTCGTAGCAGCATGGACACTAA |
| | | AAGCAGCAGCATGATAG |
| CV11-HPV/E6-E7-S2; | 497 | MHQKRTAMVQDPQERPRKLPQLCTELQTTIHDIILECVYCKQQLLRREVYDE |
| Protein sequence | | AFRDLCIVYRDGNPYAVGDKCLKFYSKISEYRHYCYSLYGTTLEQQYNKPLC |
| | | DLLIRCINCQKPLCPEEKQRHLDKKQRFHNTRGRWTGRCMSCCRSSRERRET |
| | | QLMPGDTPTLHEYMLDLQPETTDLYGYGQLNDSSEEEDEIDGPAGQAEPDRA |
| | | HYNIVTECCKCDSTLRLCVQSTHVDIRTLEDLLMGTLGIVCPICSQKPLGDI |
| | | AARDLICAQKENGLTVLPPLLTDEMIAQYTSALLAGTITSGWTFGAGAALQI |
| | | PFAMQMAYRENGIGVTQNVLYENQKLIANQFNSAIGKICDSLSSTASALGKL |
| | | QDVVNQNAQALNTLVKQLSSNFGAISSVLNDILSRLDPPEAEVQIDRLITGR |
| | | LQSLQTYVTQQLIRAAEIRASANLAATKMSECVLGQ |
| CV11-HPV/E6-E7-S2; | 498 | ATGCACCAGAAACGGACGGCCATGGTCCAGGATCCGCAGGAGAGGCCEAGGA |
| DNA sequence | | AGCTTCCACAACTCTGTACTGAGTTACAGACAACCATCCATGACATAATACT |
| | | GGAATGTGTATACTGCAAGCAGCAGCTACTGAGACGGGAAGTCTACGACTTC |
| | | GCTTTTCGGGACCTATGTATCGTGTATAGAGACGGTAACCCCTACGCGGTGG |
| | | GAGATAAGTGCCTGAAGTTCTATTCGAAGATCAGTGAGTACCGTCACTACTG |
| | | TTACAGCCTCTACGGTACAACCCTGGAACAGCAATACAATAAACCCCEGTGT |
| | | GACCTTCTCATCCGGTGCATCAATTGCCAGAAGCCTCTCTGTCCAGAGGAAA |
| | | AACAGCGACATCTGGACAAGAAACAAAGGTTCCACAATACTCGAGGGCGCTG |
| | | GACGGGGAGATGCATGTCATGCTGCAGATCATCCCGCACTAGACGCGAAACC |
| | | CAGCTCATGCCCGGCGACACCCCTACTTTGCACGAGTATATGCTGGASTTAC |
| | | AACCTGAGACAACAGATCTTTATGGGTACGGACAACTGAACGACTCTAGCGA |
| | | GGAAGAAGATGAGATTGATGGCCCAGCCGGCCAGGCCGAGCCGGATAGGGCA |
| | | CATTATAACATTGTTACCTTTTGCTGTAAATGtGACAGTACACTGCGCTTGT |
| | | GCGTGCAGAGCACCCACGTGGATATCAGGACATTGGAGGACCTCCTGATGGG |
| | | CACCCTGGGAATTGTCTGCCCCATTTGTTCCCAGAAACCACTCGGCGACATA |
| | | GCCGCCCGGGACCTGATCTGCGCCCAGAAGTTCAACGGCCTGACCGTGCTGC |
| | | CTCCCCTGCTGACCGACGAGATGATCGCCCAGTACACCAGCGCCCTGTTAGC |
| | | CGGAACaATCACCAGCGGCTGGACTTTCGGCGCTGGAGCCGCTCTGCAGATC |
| | | CCCTTCGCtATGCAGATGGCCTACCGGTTCAACGGCATCGGCGTGACCCAGA |
| | | ACGTGCTGTACGAGAACCAGAAGCTGATCGCCAACCAGITCAACAGCGCCAT |
| | | CGGCAAGATCCAGGACAGCCTGAGCAGCACCGCTAGCGCCCTGGGCAAGCTG |
| | | CAGGACGTGGTGAACCAGAACGCCCAGGCCCTGAACACCCTGGTGAAGCAGC |
| | | TGAGCAGCAACTTCGGCGCCATCAGCAGCGTGCTGAACGACATCCTGAGCCG |
| | | GCTGGACCCTCCCGAGGCCGAGGTGCAGATCGACCGGCTGATCACTGGCCGG |
| | | CTGCAGAGCCTGCAGACCTACGTGACCCAGCAGCTGATCCGGGCCGCCGAGA |
| | | TTCGGGCCAGCGCCAACCTGGCCGCCACCAAGATGAGCGAGTGCGTGCTGGG |
| | | CCAGTAA |
| CV12-HLA A0201; | 499 | MSRSVALAVLALLSLSGLEAGCGASGGGGSGGGGSIQRTPKIQVYSRHPAEN |
| Protein sequence | | GKSNFLNCYVSGFHPSDIEVDLLKNGERIEKVEHSDLSFSKDWSFYLLYYTE |
| | | FTPTEKDEYACRVNHVTLSQPKIVKWDRDMGGGGSGGGGSGGGGSGGGGSGS |
| | | GSHSMRYFFTSVSRPGRGEPRFIAVGYVDDTQFVRFDSDAASQRMEPRAPWI |
| | | EQEGPEYWDGETRKVKAHSQTHRVDLGTLRGCYNQSEAGSHTVQRMYGCDVG |
| | | SDWRFLRGYHQYAYDGKDYIALKEDLRSWTAADMAAQTTKHKWEAAHVAEQL |
| | | RAYLEGTCVEWLRRYLENGKETLQRTDAPKTHMTHHAVSDHEATLRCWALSE |
| | | YPAEITLTWQRDGEDQTQDTELVETRPAGDGTFQKWAAVVVPSGQEQRYTCH |
| | | VQHEGLPKPLTLRWEPSSQPTIPIVGIIAGLVLFGAVITGAVVAAVMWRRKS |
| | | SDRKGGSYSQAASSDSAQGSDVSLTACKV |
| CV12-HLA A0201; | 500 | ATGTCTCGCTCCGTGGCCTTAGCTGTGCTCGCGCTACTCTCTCTTTCEGGCC |
| DNA sequence | | TGGAGGCTGGCTGCGGCGCCTCTGGCGGCGGAGGATCCGGAGGCGGAGGAAG |
| | | CATCCAGCGTACTCCAAAGATTCAGGTTTACTCACGTCATCCAGCAGAGAAT |
| | | GGAAAGTCAAATTTCCTGAATTGCTATGTGTCTGGGTTTCATCCATCCGACA |
| | | TTGAAGTTGACTTACTGAAGAATGGAGAGAGAATTGAAAAAGTGGAGCATTC |
| | | AGACTTGTCTTTCAGCAAGGACTGGTCTTTCTATCTCTTGTACTACACTGAG |
| | | TTCACCCCCACTGAAAAAGATGAGTATGCCTGCCGTGTGAACCATGTGACTT |
| | | TGTCACAGCCCAAGATAGTTAAGTGGGATCGAGACATGGGCGGAGGCGGCTC |
| | | TGGCGGAGGAGGCAGCGGCGGTGGCGGCAGCGGCGGCGGCGGCTCTGGCAGC |
| | | ggctctcactccatgaggtatttcttcacatccgtgtcccggcccggccgcg |
| | | gggagccccgcttcatcgcagtgggctacgtggacgacacgcagttcgtgcg |
| | | gttcgacagcgacgccgcgagccagaggatggagccgcgggcgccgtggata |
| | | gagcaggagggtccggagtattgggacggggagacacggaaagtgaaggccc |
| | | actcacagactcaccgagtggacctggggaccctgcgcggcTGCtacaacca |
| | | gagcgaggccggttctcacaccgtccagaggatgtatggctgcgacgtgggg |
| | | tcggactggcgcttcctccgcgggtaccaccagtacgcctacgacggcaagg |
| | | attacatcgccctgaaagaggacctgcgctcttggaccgcggcggacatggc |
| | | agctcagaccaccaagcacaagtgggaggcggcccatgtggcggagcagttg |
| | | agagcctacctggagggcacgtgcgtggagtggctccgcagatacctggaga |
| | | acgggaaggagacgctgcagcgcacggacgcccccaaaacgcatatgactca |
| | | ccacgctgtctctgaccatgaagccaccctgaggtgctgggccctgagcttc |
| | | taccctgcggagatcacactgacctggcagcgggatggggaggaccagaccc |
| | | aggacacggagctcgtggagaccaggcctgcaggggatggaaccttccagaa |
| | | gtgggcggctgtggtggtgccttctggacaggagcagagatacacctgccat |
| | | gtgcagcatgagggtttgcccaagcccctcaccctgagatgggagccgtctt |
| | | cccagcccaccatccccatcgtgggcatcattgctggcctggttctctttgg |
| | | agctgtgatcactggagctgtggtcgctgctgtgatgtggaggaggAAAagc |
| | | tcagatagaaaaggagggagctactctcaggctgcaagcagtgacagtgccc |
| | | agggctctgatgtgtctctcacagcttgtaaagtgtga |
| Universal CD4 T | 21 | TLMQYTKANSKFTGT |
| epitopes-Tetanus; | | |
| Protein sequence | | |
| Universal CD4 T | 22 | ATACTAATGCAATACATAAAAGCAAACAGCAAATTCATAGGAATA |
| epitopes-Tetanus; | | |
| DNA sequence | | |
| Universal CD4 T | 23 | QSTALSSLMVAQ |
| epitopes-Diphtheria; | | |
| Protein sequence | | |
| Universal CD4 T | 24 | CAAAGCATAGCACTAAGCAGCCTAATGGTAGCACAA |
| epitopes-Diphtheria; | | |
| DNA sequence | | |
| Universal CD4 T | 25 | AKFVAAWTLKAAA |
| epitopes-PADRE | | |
| peptide; Protein | | |
| sequence | | |
| Universal CD4 T | 26 | GCAAAATTCGTAGCAGCATGGACACTAAAAGCAGCAGCA |
| epitopes-PADRE | | |
| peptide; DNA | | |
| sequence | | |

TABLE 2
List of T cell epitopes from human mutated gene.

| epitope | SEQ ID NO. | Gene | mutation | HLA type |
|---|---|---|---|---|
| KLVVVGADGV | 27 | KRAS | G12D | A*02:01 |
| KLVVVGACGV | 28 | KRAS | G12C | A*02:01 |
| GADGVGKSA | 29 | KRAS | G12D | B*07:02/C*08:02 |
| GADGVGKSAL | 30 | KRAS | G12D | B*07:02/C*08:02 |
| GACGVGKSA | 31 | KRAS | G12C | B*07:02/C*08:02 |
| GACGVGKSAL | 32 | KRAS | G12C | B*07:02/C*08:02 |
| GARGVGKSA | 33 | KRAS | G12R | B*07:02/C*08:02 |
| GARGVGKSAL | 34 | KRAS | G12R | B*07:02/C*08:02 |
| GAVGVGKSA | 35 | KRAS | G12V | B*07:02/C*08:02 |
| GAVGVGKSAL | 36 | KRAS | G12V | B*07:02/C*08:02 |
| VVGADGVGK | 37 | KRAS | G12D | A*11:01/A*03:01 |
| VVVGADGVGK | 38 | KRAS | G12D | A*11:01/A*03:01 |
| VVGAVGVGK | 39 | KRAS | G12V | A*11:01/A*03:01 |
| VVVGAVGVGK | 40 | KRAS | G12V | A*11:01/A*03:01 |
| VVGARGVGK | 41 | KRAS | G12R | A*11:01/A*03:01 |
| VVVGARGVGK | 42 | KRAS | G12R | A*11:01/A*03:01 |
| VVGACGVGK | 43 | KRAS | G12C | A*11:01/A*03:01 |
| VVVGACGVGK | 44 | KRAS | G12C | A*11:01/A*03:01 |
| KLVVVGAVGV | 45 | KRAS | G12V | A*02:01 |
| KLVVVGARGV | 46 | KRAS | G12R | A *02:01 |
| GAGDVGKSALTI | 47 | KRAS | G13D | A*02:01 |
| ILDTAGREEY | 48 | KRAS | Q61R | A01:01 |
| LATEKSRWSG | 49 | BRAF | V600E | A*02:01 |
| LATEKSRWS | 50 | BRAF | V600E | A*02:01 |
| ALHGGWTTK | 51 | PIK3CA | H1047L | A*03:01 |
| LHGGWTTKM | 52 | PIK3CA | H1047L | B*15:18 |
| ARHGGWTTKM | 53 | PIK3CA | H1047R | C*07:01/C*07:02 |
| STRDPLSEITK | 54 | PIK3CA | E545K | A*03:01 |
| GWVKPIIIGH | 55 | IDH1 | R132H | A*02:01 |
| HMTEVVRHC | 56 | TP53 | R175H | A*02:01 |
| SSCMGGMNWR | 57 | TP53 | R248W | A*68:01 |
| VVPCEPPEV | 58 | TP53 | Y220C | A*02:01 |
| LRMVRGTQV | 59 | ERBB3 | V104M | C*07:02 |
| LRMVRGTQVY | 60 | ERBB3 | V104M | C*07:02 |
| RMVRGTQVY | 61 | ERBB3 | V104M | B*15:18 |
| LHVLMGHVAAV | 62 | FBXW7 | R505G | A *02:01 |
| HVLMGHVAAV | 63 | FBXW7 | R505G | A*02:01 |
| VLMGHVAAV | 64 | FBXW7 | R505G | A*02:01 |
| FLAEHEYGL | 65 | KIAA1429 | D1358E | A*02:01 |
| YLVYQMLKV | 66 | MAPK13 | G137V | A*02:01 |
| MIDSKTAEM | 67 | DSG3 | 1444M | B*67 |
| ALPASERGWK | 68 | C3orf70 | S6L | B*40:01 |
| SYTHIQYLF | 69 | SH2D3C | Q129R | A*34:01 |
| LTFRDVAIEF | 70 | ZNF468 | Q19E | C*12:03 |
| HTKDIFNVK | 71 | ADAM29 | R399H | A*34:01 |
| MASFRKLML | 72 | LUZP4 | T8M | B*67:01 |
| FPNLPHLSF | 73 | MAGEC3 | R9H | C*07:02 |
| FPNLPHLSF | 74 | MAGEC3 | R9H | B*67:01 |
| MPLFPNLPHL | 75 | MAGEC3 | R9H | B*15:18 |
| SPIEIGLFI | 76 | OTOA | T195M | A*34:01 |
| SRHNKALKL | 77 | OTOA | T195M | C*07:02 |
| FPKLTKNML | 78 | PRSS55 | A236V | B*15:18 |
| MFPKLTKNM | 79 | PRSS55 | A236V | B*15:18 |
| MFPKLTKNML | 80 | PRSS55 | A236V | A *34:01 |
| IPALSARDL | 81 | SAGE1 | M193L | A*26:01 |
| LINMAATPI | 82 | SAGE1 | M193L | B*15:18 |
| MAATPIPAL | 83 | SAGE1 | M193L | B*15:18 |
| SARDLYATV | 84 | SAGE1 | M193L | B*15:18 |
| TPIPALSARDL | 85 | SAGE1 | M193L | A*26:01 |
| IVKNDLIAK | 86 | SPAG9 | E283K | B*40:01 |
| KVDKLTCEK | 87 | SPAG9 | E283K | A*03:01 |
| SVMKLCLIMV | 88 | AKAP4 | A712V | A*02:01 |
| TPAMEGAVA | 89 | ARX | V508M | B*35:01 |
| LLRQPTPAM | 90 | ARX | V508M | C*03:03 |
| MALLLVLFLV | 91 | CRISP2 | P5L | A*02:01 |
| LLLVLFLVTV | 92 | CRISP2 | P5L | A*02:01 |
| ALLLVLFLV | 93 | CRISP2 | P5L | A*02:01 |
| LLVLFLVTV | 94 | CRISP2 | P5L | A*02:01 |
| VLFLVTVLL | 95 | CRISP2 | P5L | A*02:01 |
| FLVTVLLPS | 96 | CRISP2 | P5L | A*02:01 |
| MALLLVLFL | 97 | CRISP2 | P5L | C*03:03 |
| LVLFLVTVL | 98 | CRISP2 | P5L | C*03:03 |
| KVWVQGHYL | 99 | MAGEC2 | R287Q | A*02:01 |
| WVQGHYLEY | 100 | MAGEC2 | R287Q | B*35:01 |
| EVPHSSPPY | 101 | MAGEC2 | R287Q | B*35:01 |
| VPHSSPPYY | 102 | MAGEC2 | R287Q | B*35:01 |
| FLNSGGDFL | 103 | AASDH | S589F | A*02:01 |
| MVIFWVYSV | 104 | ABCA2 | S715F | A*02:01 |
| FLVSLDHSM | 105 | ACOT8 | M252L | A*02:01 |
| LLLIVLLLTV | 106 | ACP2 | F393L | A*02:01 |
| YVFLCPDSSV | 107 | ADCY9 | S938F | A*02:01 |
| YLIHSSMSL | 108 | AKR1C3 | P119S | A*02:01 |
| FLLKSDGFFI | 109 | AKT2 | S34F | A*02:01 |
| VLATDQNFSL | 110 | ALKBH1 | P354S | A*02:01 |
| WIYEQLPEV | 111 | ALMS1 | R4120W | A*02:01 |
| FMALLSNLA | 112 | ARID1B | S2118F | A*02:01 |
| FLMKIQLSKV | 113 | ATG2B | E1609K | A*02:01 |
| KLWALSLAHA | 114 | ATP6V0A2 | R755K | A*02:01 |
| FLIITDFFL | 115 | ATXN10 | L159F | A*02:01 |
| ALAQLQQGV | 116 | BCAR1 | R255Q | A*02:01 |
| SISSCIPFV | 117 | C11orf74 | P63S | A*02:01 |
| KLMESSSVV | 118 | CDCA2 | P815L | A*02:01 |
| FMFYCAHWRT | 119 | CEPT1 | Q199R | A*02:01 |
| VLDEIGIKI | 120 | CHMP2B | E132K | A*02:01 |
| GMAGVAAPL | 121 | CHST2 | P150L | A*02:01 |
| FLLLLLLLV | 122 | CLMP | S2F | A*02:01 |
| YQIGWDRFCV | 123 | COL12A1 | T1055R | A*02:01 |
| ALLENMERL | 124 | COPZ1 | G97R | A*02:01 |
| SLADTRTTSV | 125 | CRYBG3 | A2862T | A*02:01 |
| TILELSDEV | 126 | CTNS | P73S | A*02:01 |
| KLFSENVEL | 127 | CYP2R1 | E212K | A*02:01 |
| SLGRRSLPGV | 128 | DLST | P25S | A*02:01 |
| ALLDRYFKA | 129 | DNPH1 | E159K | A*02:01 |
| ALFRHKVGYV | 130 | DOHH | E208K | A*02:01 |
| FILSITVIV | 131 | DPY19L3 | V424I | A*02:01 |
| SLQDAVFQGV | 132 | DSP | S2077F | A*02:01 |
| MQFFNFSWRV | 133 | ECE1 | R524Q | A*02:01 |
| FAFTVKFYL | 134 | EPB41L1 | P115L | A*02:01 |
| VMTSFSSEL | 135 | FAM171B | P645S | A*02:01 |
| GLCPRLVLV | 136 | FANCG | P243L | A*02:01 |
| VINDILFWL | 137 | FARS2 | S368L | A*02:01 |
| LLYYAVVKV | 138 | FAT1 | P2085L | A*02:01 |
| GLLDAGAIV | 139 | FURIN | M434I | A*02:01 |
| IIYYFVCYSI | 140 | FZD6 | S238F | A*02:01 |
| ILVKAVVMTPV | 141 | GAK | R578K | A*02:01 |
| FVTDAVPFPI | 142 | HSPH1 | S393F | A*02:01 |
| SLSGGFVAEL | 143 | KLF4 | P195L | A*02:01 |
| FLNYCSSPL | 144 | LTN1 | K1279N | A*02:01 |
| FLSELQISYV | 145 | MAP4K1 | S108F | A*02:01 |
| FLEMVQTLPL | 146 | MED13 | P1679L | A*02:01 |
| VQFCLTIPL | 147 | MED14 | P1344L | A*02:01 |
| LLLEMAPWFA | 148 | MFSD10 | P199L | A*02:01 |
| LLLDTDMLM | 149 | MLXIP | P270L | A*02:01 |
| VLPEENIQEV | 150 | MRGBP | I111N | A*02:01 |
| ALDSYKHKV | 151 | MTOR | P896S | A*02:01 |
| YCFELLFMV | 152 | MYCBP2 | S4018F | A*02:01 |
| VLQKLLMGV | 153 | MYCBPAP | E606K | A*02:01 |
| SLLDNIIGV | 154 | MYO1B | M14I | A*02:01 |
| LMSQGQMMV | 155 | NCOA6 | P657S | A*02:01 |
| SLMELDKFL | 156 | NRBP2 | F365L | A*02:01 |
| FLVKYLGHV | 157 | NUMB | P40L | A*02:01 |
| HMNSSLLFL | 158 | NVL | S19F | A*02:01 |
| KLHLFLEHV | 159 | AMPD2 | E494K | A*02:01 |
| TLAPSSHSL | 160 | ATN1 | P356S | A*02:01 |
| LLVHLFLEL | 161 | BEND3 | P583L | A*02:01 |
| ILYCDMCNL | 162 | BRPF1 | F292Y | A*02:01 |
| FVLSKLVHNV | 163 | C14orf93 | L303F | A*02:01 |
| TLDGFFFRV | 164 | C17orf58 | P8L | A*02:01 |
| SLVGQHILYV | 165 | CAD | S1928Y | A*02:01 |
| SLGAWRLYLL | 166 | CCDC28A | P19S | A*02:01 |
| FTFAQPPPA | 167 | CDK5R1 | S96F | A*02:01 |
| YLLDWLSVC | 168 | CHST15 | F468C | A*02:01 |
| GLYSTFPPV | 169 | DALRD3 | P427S | A*02:01 |
| LLFSATFEDFV | 170 | DDX19B | S129F | A*02:01 |
| FIAPPVYFNI | 171 | ELMSAN1 | S625F | A*02:01 |
| ALASALTSV | 172 | ESRP2 | P655L | A*02:01 |
| ALLDIIRFL | 173 | FADS2 | S429F | A*02:01 |
| SLYPAHPAL | 174 | FKBP15 | H413Y | A*02:01 |
| YLPEELVAL | 175 | FLII | A42V | A*02:01 |
| YLKAWRAPV | 176 | FM04 | G519R | A*02:01 |
| GLDDVSLSV | 177 | IQGAP3 | P560S | A*02:01 |
| FTDAGFHFA | 178 | ITGB1 | S277F | A*02:01 |
| LMFAIISGL | 179 | RAPGEF2 | S806L | A*02:01 |
| IIASFSSGV | 180 | RBBP5 | 1313F | A*02:01 |
| ILDGEMPQV | 181 | SEC23IP | P436L | A*02:01 |
| GSWNIIFFV | 182 | SLC25A30 | P274S | A*02:01 |
| KLIWAALREL | 183 | SMTN | R665W | A*02:01 |
| IMFMTPGSFL | 184 | SNX19 | L387F | A*02:01 |
| ALAFHSVEPV | 185 | STRN3 | P406S | A*02:01 |
| WLANGDTLCV | 186 | TBL2 | R162C | A*02:01 |
| FADTLLIVFV | 187 | TMCO1 | I65V | A*02:01 |
| VLLSGLVLLI | 188 | TMEM125 | V126I | A*02:01 |
| SLPAKSGLYPV | 189 | TRAPPC9 | E639K | A*02:01 |
| ILIEGLEFM | 190 | TRIAP1 | P58L | A*02:01 |
| RLPEMGFNV | 191 | TRRAP | S702F | A*02:01 |
| HLVIKVFWRV | 192 | UBE3C | P991L | A*02:01 |
| GLVEISWFFL | 193 | URGCP | P310L | A*02:01 |
| KLDLWSLPSV | 194 | USP15 | P800S | A*02:01 |
| VLLGHLQWV | 195 | WSB2 | S194L | A*02:01 |
| FTLSKPHGV | 196 | ZNF33A | S288F | A*02:01 |
| KLNKFSVFNI | 197 | KIAA1958 | P400S | A*02:01 |
| ITISQLFLV | 198 | KIF23 | S334F | A*02:01 |
| YLVFLPRLHV | 199 | LIG3 | G76R | A*02:01 |
| VQDEFFESV | 200 | LNPEP | S349F | A*02:01 |
| KMFHYTAIL | 201 | LPHN3 | H1049Y | A*02:01 |
| SLDLDFNPLL | 202 | MROH7 | S214F | A*02:01 |
| VLIAGITPT | 203 | MTHFD1L | V418I | A*02:01 |
| FMFTWCFCF | 204 | MYADM | S68F | A*02:01 |
| GIIEWMFAV | 205 | NID1 | G495E | A*02:01 |
| ILQEHLIPL | 206 | NLRP1 | P614L | A*02:01 |
| RLYHGTLRAL | 207 | NPHP4 | P159L | A*02:01 |
| SLPTSFPTL | 208 | NUP214 | P1463S | A*02:01 |
| YLTGVDIFL | 209 | PADI1 | S118F | A*02:01 |
| YLFFSALAHL | 210 | PAQR7 | S119F | A*02:01 |
| TLTENIIIPL | 211 | PKP2 | F491L | A*02:01 |
| ALPQAFPTV | 212 | PLEKHG4 | S635F | A*02:01 |
| MLISENSRFEV | 213 | PTK7 | D463N | A*02:01 |

TABLE 3
List of T cell epitopes from viral gene.

| | SEQ | | | |
|---|---|---|---|---|
| epitope | ID NO: | Virus | location | HLA |
| TIHDIILECV | 214 | HPV16 | E6 29-38 | A0201 |
| YMLDLQPET | 215 | HPV16 | E7 11-19 | A0201 |
| HDIILECV | 216 | HPV16 | E6 31-38 | B4002 |
| YMLDLQPETT | 217 | HPV | E7 11-20 | A0201 |
| TLGIVCPI | 218 | HPV | E7 86-93 | A0201 |
| KLPQLCTEV | 219 | HPV-16 | E6 (aa 11- | A*02 |
| (KL9V) b | | | 19) | |
| TIHDIILEC | 220 | HPV-16 | E6 (aa 29- | B*48 |
| (TIC9) | | | 37) | |
| KISEYRHYC | 221 | HPV-16 | E6 (aa 72- | A*02 |
| (KIC9) | | | 80) | |
| QLYNKPLCDV | 222 | HPV-16 | E6 (aa 90- | A*02 |
| (QLV10) b | | | 99) | |
| TLHEYMLDL | 223 | HPV-16 | E7 (aa 7-15) | A*02/B*48 |
| (YLL9) | | | | |
| CDSTLRLCV | 224 | HPV-16 | E7 (aa 61- | A*24 |
| (CDV9) | | | 69) | |
| LCVQSTHVDI | 225 | HPV-16 | E7 (aa 67- | A*24 |
| (LCI10) | | | 76) | |
| RTLEDLLMGV | 226 | HPV-16 | E7 (aa 77- | A*02 |
| (RTV10) b | | | 86) | |
| LEDLLMGTL | 227 | HPV-16 | E7 (aa 79- | B*60 |
| (LEL9) | | | 87) | |
| LLMGTLGIV | 228 | HPV-16 | E7 (aa 82- | A*02 |
| (LLV9) | | | 90) | |
| KYTFWEVNL | 229 | HPV16 | L1 443-452 | A*24:02/A*23:01 |
| YTFWEVNLK | 230 | HPV16 | L1 444-453 | A*68:01/A*11:01 |
| ILEDWNFGL | 231 | HPV16 | L1 398-408 | A*02:01/A*02:06 |
| DSLFFYLRR | 232 | HPV16 | L1 244-253 | A*33:01 |
| PPLELINTV | 233 | HPV16 | L1 186-195 | B*51:01 |
| YVARTNIYY | 234 | HPV16 | L1 27-36 | A*26:01/A*01:01/A* |
| | | | | 30:02/B*35:01/ |
| | | | | B*15:01 |
| VGHPYFPIK | 235 | HPV16 | L1 45-54 | A*30:01 |
| FVTVVDTTR | 236 | HPV16 | L1 330-339 | A*68:01 |
| RLLAVGHPY | 237 | HPV16 | L1 41-50 | A*32:01/B*15:01/A* |
| | | | | 30:02 |
| SAYAANAGV | 238 | HPV16 | L1 133-142 | A*68:02 |
| LLAVGHPYF | 239 | HPV16 | L1 42-51 | B*15:01 |
| FFYLRREQM | 240 | HPV16 | L1 247-256 | B*08:01 |
| EEYDLQFIF | 241 | HPV16 | L1 368-397 | B*44:03/B*44:02/B* |
| | | | | 40:01 |
| KFGFPDTSF | 242 | HPV16 | L1 82-91 | A*23:01/A*24:02 |
| TSQAIACQK | 243 | HPV16 | L1 422-431 | A*11:01 |
| VEVGRGQPL | 244 | HPV16 | L1 105-114 | B*40:01 |
| FGFPDTSFY | 245 | HPV16 | L1 83-92 | B*35:01/A*30:02/A* |
| | | | | 01:01 |
| ISTSETTYK | 246 | HPV16 | L1 348-357 | A*11:01 |
| LFLIHTHAR | 247 | HPV16 | E5 71-80 | A*33:01 |
| FLIHTHARF | 248 | HPV16 | E5 72-81 | B*15:01 |
| TYTSLIILV | 249 | HPV16 | E5 38-47 | A*24:02/A*23:01 |
| NIRGRWTGR | 250 | HPV16 | E6 134-143 | A*33:01 |
| AFRDLCIVY | 251 | HPV16 | E6 53-62 | A*30:02 |
| ISEYRHYCY | 252 | HPV16 | E6 80-89 | A*01:01 |
| DPQERPRKL | 253 | HPV16 | E6 11-20 | B*08:01/B*51:01 |
| CYSLYGTTL | 254 | HPV16 | E6 87-96 | A*24:02 |
| LQPETTDLY | 255 | HPV16 | E7 15-24 | B*15:01 |
| RAHYNIVTF | 256 | HPV16 | E7 49-58 | A*32:01/B*15:01/B* |
| | | | | 58:01/B*57:01/B*35: |
| | | | | 01 |
| EPDRAHYNI | 257 | HPV16 | E7 46-55 | B*51:01/B*53:01 |
| LLMGTLGIV | 258 | HPV16 | E7 82-91 | A*02:03 |
| MLLLHIHAI | 259 | HPV 18 | E5 (aa61-70) | A*02:01/A*02:03/A* |
| | | | | 02:06/B*08:01, |
| YAWVLVFVY | 260 | HPV 18 | E5 (aa31-40) | B*35:01 |
| KLPDLCTEL | 261 | HPV 18 | E6 (aa 13- | A*02 |
| (KLL9) | | | 21) | |
| KTVLELTEV | 262 | HPV 18 | E6 (aa 36- | A*02 |
| (KTV9) | | | 44) | |
| TVLELTEVF | 263 | HPV 18 | E6 (aa 37- | B*35:01/B*53:01, |
| | | | 45) | |
| LELTEVFEF | 264 | HPV 18 | E6 (aa 39- | B*44:03/B*44:02, |
| | | | 47) | |
| ELTEVFEFA | 265 | HPV 18 | E6 (aa 40- | A*68:02 |
| | | | 49) | |
| FEFAFKDLF | 266 | HPV 18 | E6 (aa 45- | B*40:01/B*44:03, |
| | | | 51) | |
| ELTEVFEFA | 267 | HPV 18 | E6 (aa 50- | A*02 |
| (ELA9) | | | 58) | |
| FAFRDLCIVY | 268 | HPV18 | E6 52-61 | B*35/B*57 |
| VVYRDSIPH | 269 | HPV 18 | E6 (aa 54- | A*11 |
| (VVH9) | | | 62) | |
| KCIDFYSRI | 270 | HPV 18 | E6 (aa 67- | A*02 |
| (KCI9) | | | 75) | |
| DFYSRIREL | 271 | HPV 18 | E6 (aa 70- | B*08:01 |
| | | | 79) | |
| SVYGDTLEK | 272 | HPV 18 | E6 (aa 84- | A*11 |
| (SVK9) | | | 92) | |
| LEKLTNTGL | 273 | HPV 18 | E6 (aa 90- | B*40:01 |
| | | | 99) | |
| RFHNIAGHY | 274 | HPV 18 | E6 (aa 126- | A*30:02 |
| | | | 135) | |
| TLQDIVLHL | 275 | HPV 18 | E7 (aa 7-15) | A*02 |
| (TLL9) | | | | |
| DDLRAFQQLF | 276 | HPV 18 | E7 (aa 81- | A*02/A*11/A*24/A* |
| LNTLS | | | 95) | 33 |
| (DDS15) c | | | | |
| FQQLFLNTL | 277 | HPV 18 | E7 (aa 86- | A*02 |
| (FQI9) | | | 94) | |
| QLFLNTLSFV | 278 | HPV 18 | E7 (aa 88- | A*02 |
| (QLV10) | | | 97) | |
| LFLNTLSFVCPWCA | 279 | HPV 18 | E7 (aa 89- | A*02/A*11/A*24/A* |
| S (LFS15) c | | | 103) | 33 |
| NLVPMVATV | 281 | CMV | pp65 495- | A0201 |
| | | | 503 | |
| TPRVTGGGA | 282 | CMV | pp65 417- | B0702 |
| | | | 425 | |
| PRVTGGGAM | 283 | CMV | pp65 418- | B0702 |
| | | | 426 | |
| FPTKDVALR | 284 | CMV | pp65 188- | A6801 |
| | | | 196 | |
| FIAGNSAYEYV | 285 | CMV | GB: 619- | A2 |
| | | | 628 | |
| SDEEEAIVAYTL | 286 | CMV | IE1: 378- | B18 |
| | | | 389 | |
| GVLDAVWRV | 287 | CMV | US8 74-82 | A*02 |
| ALWDVALLEV | 288 | CMV | UL150A | A*02 |
| | | | 152-161 | |
| TLIVNLVEV | 289 | CMV | UL100 200- | A*02 |
| | | | 208 | |
| GLFAVENFL | 290 | CMV | UL44 259- | A*02 |
| | | | 267 | |
| YPRPPGSGL | 291 | CMV | UL16 162- | B*07 |
| | | | 170 | |
| SPRDAWIVL | 292 | CMV | TRS1 166- | B*07 |
| | | | 174 | |
| SPSRDRFVQL | 293 | CMV | UL52 349- | B*07 |
| | | | 357 | |
| RPWKPGQRV | 294 | CMV | UL23 22-30 | B*07 |
| SPRHLYISL | 295 | CMV | UL46 76-84 | B*07 |
| SENGNLQVTY | 296 | CMV | UL112/UL11 | B*44 |
| | | | 3 125-134 | |
| HETGVYQMW | 297 | CMV | UL117 358- | B*44 |
| | | | 366 | |
| YADPFFLKY | 298 | CMV | UL105 715- | A*01 |
| | | | 723 | |
| VTEHDTLLY | 299 | CMV | UL44 245- | A*01 |
| | | | 253 | |
| RTDPATLTAY | 300 | CMV | UL69 569- | A*01 |
| | | | 578 | |
| ITEIALDRY | 301 | CMV | US28 122- | A*01 |
| | | | 130 | |
| NTDFRVLELY | 302 | CMV | UL55 657- | A*01 |
| | | | 665 | |
| GLYTQPRWK | 303 | CMV | UL77 228- | A*03 |
| | | | 236 | |
| RVKNRPIYR | 304 | CMV | UL57 790- | A*03 |
| | | | 798 | |
| LPHERHREL | 305 | CMV | UL34 180- | B*08 |
| | | | 188 | |
| LPYPRGYTL | 306 | CMV | UL26 61-69 | B*08 |
| YLVRRPMTI | 307 | CMV | UL13 465- | B*08 |
| | | | 473 | |
| LPLKMLNI | 308 | CMV | UL83 116- | B*51 |
| | | | 123 | |
| LPYPRGYTL | 309 | CMV | UL38 156- | B*51 |
| | | | 164 | |
| FPVEVRSHV | 310 | CMV | UL26 61-69 | B*51 |
| DARSRIHNV | 311 | CMV | UL56 503- | B*51 |
| | | | 511 | |
| VLEETSVML | 312 | CMV | IE-1 | A*0201 |
| KLGGALQAK | 313 | CMV | IE-1 | A*0301 |
| QYDPVAALF | 314 | CMV | pp65 | A*2301 |
| AYAQKIFKI | 315 | CMV | pp65 | A*2301 |
| VYALPLKML | 316 | CMV | pp65 | A*2402 |
| RPHERNGFTVL | 317 | CMV | pp65 | B*0702 |
| TPRVTGGGAM | 318 | CMV | pp65 | B*0702 |
| ELKRKMIYM | 319 | CMV | IE-1 | B*0801 |
| ELNRKMIYM | 320 | CMV | IE-1 | B*0801 |
| ELRRKMMYM | 321 | CMV | IE-1 | B*0801 |
| QIKVRVDMV | 322 | CMV | IE-1 | B*0801 |
| IPSINVHHY | 323 | CMV | pp65 | B*3501 |
| DTPLIPLTIF | 324 | EBV | EBNA 2 | A*02 |
| FLRGRAYGL | 325 | EBV | EBNA 3a | B8 |
| EENLLDFVRF | 326 | EBV | EBNA 3c | B44 |
| RPPIFIRRL | 327 | EBV | EBNA 3 A: | B7 |
| | | | 379-387 | |
| RLRAEAQVK | 328 | EBV | EBNA 3 A: | A3 |
| | | | 603-611 | |
| IVTDFSVIK | 329 | EBV | EBNA 3 B: | A11 |
| | | | 416-424 | |
| EGGVGWRHW | 330 | EBV | EBNA 3 C: | B44 |
| | | | 163-171 | |
| QPRAPIRPI | 331 | EBV | EBNA 3 C: | B7 |
| | | | 881-891 | |
| FRLGRAYGL | 332 | EBV | EBNA 3 A: | B8 |
| | | | 325-333 | |
| YPLHEQHGM | 333 | EBV | EBNA 3 A: | B35 |
| | | | 458-466 | |
| CLGGLLTMV | 334 | EBV | LMP-2: 426- | A2 |
| | | | 434 | |
| GLCTLVAML | 335 | EBV | BMLF-1: | A2 |
| | | | 280-288 | |
| RAKFKQLL | 336 | EBV | BZLF-1: | B8 |
| | | | 190-197 | |
| PRSPTVFYNIPPMPL | 337 | EBV | EBNA 2 | B18 |
| RGIKEHVIQNAFRK | 338 | EBV | EBNA 3c | A24 |
| A | | | | |
| YVLDHLIVV | 339 | EBV | BRLFI | A2.1 |
| TLDYKPLSV | 340 | EBV | BMRF1 | A2.1 |
| QAKWRLQTL | 341 | EBV | EBNA3A | B8 |
| FLDKGTYTL | 342 | EBV | BALF4 | A*0201 |
| GLCTLVAML | 343 | EBV | BMLF1259- | A*0201 |
| | | | 267 | |
| TLDYKPLSV | 344 | EBV | BMRF1 | A*0201 |
| YVLDHLIVV | 345 | EBV | BRLF1109- | A*0201 |
| | | | 117 | |
| LLDFVRFMGV | 346 | EBV | EBNA 3B | A*0201 |
| YLLEMLWRL | 347 | EBV | LMP-1 | A*0201 |
| YLQQNWWTL | 348 | EBV | LMP1159- | A*0201 |
| | | | 167 | |
| CLGGLLTMV | 349 | EBV | LMP-2A | A*0201 |
| FLYALALLL | 350 | EBV | LMP-2A | A*0201 |
| RLRAEAQVK | 351 | EBV | EMNA 3A | A*0301 |
| AVFDRKSDAK | 352 | EBV | EBNA 3B | A*1101 |
| IVTDESVIK | 353 | EBV | EBNA 3B | A*1101 |
| DYCNVLNKEF | 354 | EBV | BRLF1 | A*2402 |
| TYGPVFMCL | 355 | EBV | LMP-2 | A*2402 |
| IACPIVMRY | 356 | EBV | BRLF1 | A*2902 |
| IVTDFSVIK | 357 | EBV | EBNA 3B | A*6801 |
| RPQGGSRPEFVKL | 358 | EBV | BMRF1 | B*0702 |
| RPPIFIRRL | 359 | EBV | EBNA 3A | B*0702 |
| QPRAPIRPI | 360 | EBV | EBNA 6 | B*0702 |
| RAKFKQLL | 361 | EBV | BZLF1 | B*0801 |
| FLRGRAYGL | 362 | EBV | EBNA 3A | B*0801 |
| EPLPQGQLTAY | 363 | EBV | BZLF1 | B*3501 |
| HPVAEADYFEY | 364 | EBV | EBNA 1 | B*3501 |
| HPVGDADYFEY | 365 | EBV | EBNA 1 | B*3501 |
| HPVGEADYFEY | 366 | EBV | EBNA 1 | B*3501 |
| HPVGQADYFEY | 367 | EBV | EBNA 1 | B*3501 |
| YPLHEQHGM | 368 | EBV | EBNA 3A | B*3501 |
| AVLDGLLSL | 369 | HTLV | bZIP factor | A*0201 |
| GLLSLEEEL | 370 | HTLV | bZIP factor | A*0201 |
| LLFGYPVYV | 371 | HTLV | Tax | A*0201 |
| SFHSLHLLF | 372 | HTLV | tax | A*2402 |
| LPVSCPEDL | 373 | HTLV | bZIP factor | B*0702 |
| VLGGCRHKL | 374 | HBV | HBX 133- | A*02:01 |
| | | | 141 | |
| HLSLRGLPV | 375 | HBV | HBX 52-60 | A*02:01 |
| GGCRHKLVC | 376 | HBV | HBX 135- | A*11:01 |
| | | | 143 | |
| VLHKRTLGL | 377 | HBV | HBX 92-100 | A*02:01 |
| KVLHKRTLGL | 378 | HBV | HBX 91-100 | A*02:01 |
| GLSAMSTTDL | 379 | HBV | HBX 99-108 | A*02 |
| AMSTTDLEA | 380 | HBV | HBX 102- | A*02:01 |
| | | | 110 | |
| VLCLRPVGA | 381 | HBV | HBX 15-23 | A*02:01 |
| QLDPARDVL | 382 | HBV | HBX 8-16 | A*02:01 |
| CLFKDWEEL | 383 | HBV | HBX 115- | A*02:01 |
| | | | 123 | |
| NAHQVLPKV | 384 | HBV | HBX 84-92 | A*02:01 |
| TLGLAAMST | 385 | HBV | HBX 97-105 | A*02:01 |
| KLVCSPAPC | 386 | HBV | HBX 140- | A*02:01 |
| | | | 148 | |
| VVPTDHGAHL | 387 | HBV | HBX 44-53 | A*02:01 |
| SITEVECFL | 388 | polyomaviruses | JCV-VP1- | A*02:01 |
| | | (JCV/BKV) | p36-44 | |
| ILMWEAVTL | 389 | polyomaviruses | JCV-VP1- | A*02:01 |
| | | (JCV/BKV) | p100-108 | |
| AITEVECFL | 390 | polyomaviruses | BKV-VP1- | A*02:01 |
| | | (JCV/BKV) | p44-52 | |
| LLMWEAVTV | 391 | polyomaviruses | BKV-VP1- | A*02:01 |
| | | (JCV/BKV) | p108-116 | |
| IGVTSLMNV | 392 | polyomaviruses | OP50 | A*02:01 |
| | | (JCV/BKV) | | |
| VARIPLPNL | 393 | polyomaviruses | OP20 | A*02:01 |
| | | (JCV/BKV) | | |
| TEVIGVTSL | 394 | polyomaviruses | OP28 | A*02:01 |
| | | (JCV/BKV) | | |
| WVPDPTRNE | 395 | polyomaviruses | OP52 | A*02:01 |
| | | (JCV/BKV) | | |
| ENTRYEGTL | 396 | polyomaviruses | OP52 | A*02:01 |
| | | (JCV/BKV) | | |
| GMFTNRSGS | 397 | polyomaviruses | OP65 | A*02:01 |
| | | (JCV/BKV) | | |
| KNPYPISFL | 398 | polyomaviruses | OP72 | A*02:01 |
| | | (JCV/BKV) | | |
| PISFLLTDL | 399 | polyomaviruses | OP73 | A*02:01 |
| | | (JCV/BKV) | | |
| PMYGMDAQV | 400 | polyomaviruses | OP78 | A*02:01 |
| | | (JCV/BKV) | | |
| GMDAQVEEV | 401 | polyomaviruses | OP79 | A*02:01 |
| | | (JCV/BKV) | | |
| ELPGDPDMM | 402 | polyomaviruses | OP83 | A*02:01 |
| | | (JCV/BKV) | | |
| ILMWEAVTL | 403 | polyomaviruses | p100 | A*02:01 |
| | | (JCV/BKV) | | |
| DPDEHLRGF | 404 | polyomaviruses | OP12 | B7 |
| | | (JCV/BKV) | | |
| DPDEHLRGF | 405 | polyomaviruses | OP13 | B7 |
| | | (JCV/BKV) | | |
| SDSPNRDML | 406 | polyomaviruses | OP18 | B7 |
| | | (JCV/BKV) | | |
| EDLTCGNIL | 407 | polyomaviruses | OP23 | B7 |
| | | (JCV/BKV) | | |
| RTKYPDGTI | 408 | polyomaviruses | OP40 | B7 |
| | | (JCV/BKV) | | |
| ENTRYFGTL | 409 | polyomaviruses | OP53 | B7 |
| | | (JCV/BKV) | | |
| ITNTATTVL | 410 | polyomaviruses | OP57 | B7 |
| | | (JCV/BKV) | | |
| GPLCKGDNL | 411 | polyomaviruses | p244 | B7 |
| | | (JCV/BKV) | | |
| TPRVDGQPM | 412 | polyomaviruses | OP76 | B7 |
| | | (JCV/BKV) | | |
| NPYPISFLL | 413 | polyomaviruses | OP72 | B7 |
| | | (JCV/BKV) | | |
| NPYPISFLL | 414 | polyomaviruses | OP72 | B7 |
| | | (JCV/BKV) | | |

In some embodiments, the MHC class I heavy chain can include an HLA-A1:01, HLA-A2:01, HLA-A2:03, HLA-A2:06, HLA-A2:07, HLA-A3:01, HLA-A9, HLA-A10, HLA-A11:01, HLA-A23:01, HLA-A24:02, HLA-A25:01, HLA-A26:01, HLA-A29:02, HLA-A30:01, HLA-A30:02, HLA-A31:01, HLA-A32:02, HLA-A33:03, HLA-A34:01, HLA-A34:02, HLA-A68:01, HLA-A68:02, HLA-A74:01, HLA-B5, HLA-B7:02, HLA-B8:01, HLA-B-12, HLA-B13:01, HLA-B14:02, HLA-B15:01, HLA-B15:02, HLA-B15:03, HLA-B15:18, HLA-B18:01, HLA-B35:01, HLA-B38:02, HLA-B40:01, HLA-B40:02, HLA-B42:01, HLA-B44:02, HLA-B44:03, HLA-B45:01, HLA-B46:01, HLA-B49:01, HLA-B51:01, HLA-B52:01, HLA-B53:01, HLA-B54:01, HLA-B55:02, HLA-B57:01, HLA-B58:01, HLA-B67:01, HLA-C1:02, HLA-C2:02, HLA-C3:02, HLA-C3:03, HLA-C3:04, HLA-C4:01, HLA-C5:01, HLA-C6:02, HLA-C7:01, HLA-C7:02, HLA-C8:01, HLA-C8:02, HLA-C12:03, HLA-C14:02, HLA-C16:01, HLA-C17:01, or HLA-C18:01 heavy chain.

Table 4 is a list of top HLA alleles in North America.

HLA alleles
A*01:01
A*02:01
A*02:03
A*02:06
A*02:07
A*03:01
A*11:01
A*23:01
A*24:02
A*25:01
A*26:01
A*29:02
A*30:01
A*30:02
A*31:01
A*32:01
A*33:03
A*34:02
A*68:01
A*68:02
A*74:01
B*07:02
B*08:01
B*13:01
B*14:02
B*15:01
B*15:02
B*15:03
B*18:01
B*35:01
B*38:02
B*40:01
B*40:02
B*42:01
B*44:02
B*44:03
B*45:01
B*46:01
B*49:01
B*51:01
B*52:01
B*53:01
B*54:01
B*55:02
B*57:01
B*58:01
C*01:02
C*02:02
C*03:02
C*03:03
C*03:04
C*04:01
C*05:01
C*06:02
C*07:01
C*07:02
C*08:01
C*08:02
C*12:03
C*14:02
C*16:01
C*17:01
C*18:01

In some embodiments, the single chain trimer nucleic acid can further include a second T cell epitope. In some embodiments, the second T cell epitope can be a MHC class II restricted epitope. In some embodiments, the second T cell epitope comprises SEQ ID NO: 21 and SEQ ID NO: 22, SEQ ID NO: 23, SEQ ID NO: 24, SEQ ID NO: 25, SEQ ID NO: 26, or an epitope listed in Table 5 or a homolog having at least 90% (e.g. 90%, 91%, 92%, 93%, 94%, 95%, 96%, 97%, 98%, 99%, 99.8%, or 99.9%) identity with a sequence selected from SEQ ID NO. 21 SEQ ID NO: 22, SEQ ID NO: 23, SEQ ID NO: 24, SEQ ID NO: 25, SEQ ID NO: 26 and an epitope listed in Table 5.

Table 5 is a list of top MHC class II restricted epitopes (universal CD4 T cell epitope sequences).

| | SEQ | | | |
|---|---|---|---|---|
| | ID | | | |
| Epitope | NO: | Gene | origin | HLA type |
| AKFVAAWTLKAAA | 415 | PADRE | | pan DR cpitopc |
| | | peptide | | |
| SFIEDLLFNKVTLAD | 416 | S816- | SARS- | broadly recognized and present |
| | | 830 | CoV-2 | by common HLA II alleles |
| KPSKRSFIEDLLFNKVTLAD | 417 | S811- | SARS- | broadly recognized and present |
| A | | 831 | CoV-2 | by common HLA II alleles |
| GKLQDVVNQNAQALNTLVK | 418 | S946- | SARS- | broadly recognized and present |
| QL | | 966 | CoV-2 | by common HLA II alleles |
| KVEAEVQIDRLITGRLQSLQ | 419 | S986- | SARS- | broadly recognized and present |
| T | | 1006 | CoV-2 | by common HLA II alleles |
| DSKVGGNYNYLYRLFRK | 420 | S442- | SARS- | DRB1*11:01 |
| | | 458 | CoV-2 | |
| SKRSFIEDLLFNKVTLA | 421 | S813- | SARS- | DPA1*01:03/DPB1*04:01 |
| | | 829 | CoV-2 | |
| ASFSTFKCYGVSPTKLN | 422 | S372- | SARS- | DRB1*15:02 |
| | | 388 | CoV-2 | |
| VSMTKTSVDCTMYICGD | 423 | S729- | SARS- | DQA1*02:01/DQB1*02: 02 |
| | | 745 | CoV-2 | |
| SKRSFIEDLLFNKVTLA | 424 | S729- | SARS- | DPA1*01:03/DPB1*04:01 |
| | | 745 | CoV-2 | |
| SKRSFIEDLLFNKVTLA | 425 | S813- | SARS- | DPA1*01:03/DPB1*04:01 |
| | | 829 | CoV-2 | |
| REFVFKNIDGYFKIYSK | 426 | S190- | SARS- | DRB5*01:01 |
| | | 206 | CoV-2 | |
| SNLLLQYGSFCTQLNRA | 427 | S750- | SARS- | DRB1*15:01 |
| | | 766 | CoV-2 | |
| SKRSFIEDLLFNKVTLA | 428 | S813- | SARS- | DPA1*02:01/DPB1 |
| | | 829 | CoV-2 | |
| YAWNRKRISNCVADYSV | 429 | S351- | SARS- | DRB3*02:02 |
| | | 367 | CoV-2 | |
| VLYQDVNCTEVPVAIHA | 430 | S610- | SARS- | DRB1*13:01 |
| | | 626 | CoV-2 | |
| GAEHVNNSYECDIPIGA | 431 | S652- | SARS- | DQA1*02:01/DQB1*02: |
| | | 668 | CoV-2 | |
| SPRRARSVASQSIIA YT | 432 | S680 | SARS- | DQA1*01:03/DQB1*06: |
| | | 696 | CoV-2 | |
| PFNDGVYFASTEKSNII | 433 | S85- | SARS- | DRB3*02:02 |
| | | 101 | CoV-2 | |
| NIIRGWIFGTTLDSKTQ | 434 | S99- | SARS- | DPA1*01:03/DPB1*06:01 |
| | | 115 | CoV-2 | |
| VNNATNVVIKVCEFQFC | 435 | S120- | SARS- | DRB3*02:02 |
| | | 136 | CoV-2 | |
| QFCNDPFLGVYYHKNNK | 436 | S134- | SARS- | DRB1*11:01 |
| | | 150 | CoV-2 | |
| YAWNRKRISNCVADYSV | 437 | S351- | SARS- | DRB3*02:02, DRB4*01:03 |
| | | 367 | CoV-2 | |
| ASFSTFKCYGVSPTKLN | 438 | S372- | SARS- | DPA1*01:03/DPB1*06:01 |
| | | 388 | CoV-2 | |
| KLNDLCFTNVYADSFVI | 439 | S386- | SARS- | DPA1*02:01/DPB1*14: |
| | | 402 | CoV-2 | |
| FVIRGDEVRQIAPGQTG | 440 | S400- | SARS- | DQA1*05:05/DQB1*03: |
| | | 416 | CoV-2 | |
| ENQKLIANQFNSAIGKI | 441 | S918- | SARS- | DRB3*02:02 16 |
| | | 934 | CoV-2 | |
| NTLVKQLSSNFGAISSV | 442 | S960- | SARS- | DRB3*02:02 |
| | | 976 | CoV-2 | |
| LPLVSSQCVNLTTRTQL | 443 | S8--24 | SARS- | DRB4*01:01 |
| | | | CoV-2 | |
| VYYPDKVFRSSVLHSTQ | 444 | S36-52 | SARS- | DPA1*01:03/DPB1*04: |
| | | | CoV-2 | |
| SANNCTFEYVSQPFLMD | 445 | S162- | SARS- | DPA1*01:03/DPB1*04: |
| | | 178 | CoV-2 | |
| REFVFKNIDGYFKIYSK | 446 | S190- | SARS- | DQA1*01:01/DQB1*05: |
| | | 206 | CoV-2 | |
| PTNFTISVTTEILPVSM | 447 | S715- | SARS- | DRB1*07:01 |
| | | 731 | CoV-2 | |
| VSMTKTSVDCTMYICGD | 448 | S729- | SARS- | DQA1*02:01/DQB1*02: |
| | | 745 | CoV-2 | |
| SNLLLQYGSFCTQLNRA | 449 | S750- | SARS- | DRB1*15:01 |
| | | 766 | CoV-2 | |
| SKRSFIEDLLFNKVTLA | 450 | S813- | SARS- | DPA1*01:03/DPB1*04:01 |
| | | 829 | CoV-2 | |
| SNLLLQYGSFCTQLNRA | 451 | S750- | SARS- | HLA-DRB1*15:01 |
| | | 766 | CoV-2 | |
| SKRSFIEDLLFNKVTLA | 452 | S813- | SARS- | DPA1*01:03/DPB1*04:01 |
| | | 829 | CoV-2 | |
| LGVYYHKNNKSWMESEF | 453 | S141- | SARS- | DRB3*02:02 |
| | | 157 | CoV-2 | |
| SPRRARSVASQSIIAYT | 454 | S680- | SARS- | DQA1*02:01/DQB1*03: 03 |
| | | 696 | CoV-2 | |
| VSMTKTSVDCTMYICGD | 455 | S729- | SARS- | DQA1*02:01/DQB1*03: 03 |
| | | 745 | CoV-2 | |
| NCTFEYVSQPFLMDL | 456 | S165- | SARS- | DRB1*04:05/DQA1*02:01/DQ |
| | | 179 | CoV-2 | B1*02:02/DQA1*03:02/DQB1 |
| | | | | *02:02/DQA1*05:01/DQB1* |
| | | | | 02:01 |
| NFSQILPDPSKPSKR | 457 | S801- | SARS- | DRB1*03:01/DRB1*04:01 |
| | | 815 | CoV-2 | |
| RSFIEDLLFNKVT | 458 | S815- | SARS- | DPA1*01:03/DPB1*04:01 |
| | | 827 | CoV-2 | |
| HWFVTQRNFYEPQII | 459 | S1101- | SARS- | DRB1*04:05/DQA1*01:01/DQ |
| | | 1115 | CoV-2 | B1*05:01 |
| AALALLLLDRLNQLE | 460 | N217- | SARS- | DRB1*03:01/DRB1*11:01/DR |
| | | 231 | CoV-2 | B1*13:01/DQA1*01:01/DQB1* |
| | | | | 05:01 |
| RGDSTITSQDVANAVVGYG | 461 | VP2P2 | Rhinovirus | broadly recognized and present |
| V | | | | by common HLA II alleles |
| SDRIIQITRGDSTITSQDVA | 462 | VP2P2 | Rhinovirus | broadly recognized and present |
| | | | | by common HLA II alleles |
| PRFSLPFLSIASAYYMFYDG | 463 | VP1P23 | Rhinovirus | broadly recognized and present |
| | | | | by common HLA II alleles |
| VPYVNAVPMDSMVRHNNW | 464 | VP2P26 | Rhinovirus | broadly recognized and present |
| SL | | | | by common HLA II alleles |
| TSKGWWWKLPDALKDMGI | 465 | VP2P10 | Rhinovirus | broadly recognized and present |
| FG | | | | by common HLA II alleles |
| PHQFINLRSNNSATLIVPYV | 466 | VP2P24 | Rhinovirus | broadly recognized and present |
| | | | | by common HLA II alleles |
| SNNSATLIVPYVNAVPMDS | 467 | VP2P25 | Rhinovirus | broadly recognized and present |
| M | | | | by common HLA II alleles |
| QSGTNASVFWQHGQPFPRES | 468 | VP1P21 | Rhinovirus | broadly recognized and present |
| | | | | by common HLA II alleles |
| NEKQPSDDNWLNFDGTLLG | 469 | VP2P21 | Rhinovirus | broadly recognized and present |
| N | | | | by common HLA II alleles |
| HIVMQYMYVPPGAPIPTTRD | 470 | VP1P18 | Rhinovirus | broadly recognized and present |
| | | | | by common HLA II alleles |
| PKYVKQNTLKLAT | 471 | HA306- | Influenza | HLA-DRA1*01:01/HLA- |
| | | 318, | A | DRB1*04:01 /HLA- |
| | | | | DRA1*01:01/HLA- |
| | | | | DRB1*01:01 (HLA-DR1) |
| SGPLKAEIAQRLED* | 472 | M1 | Influenza | broadly recognized and present |
| | | | A | by common HLA II alleles |
| GLIYNRMGAVTTEV** | 473 | M1 | Influenza | broadly recognized and present |
| | | | A | by common HLA II alleles |
| QARQMVQAMRTIGTHP | 474 | M1 | Influenza | broadly recognized and present |
| | | | A | by common HLA II alleles |
| DPFRLLQNSQVFS | 475 | NP | Influenza | broadly recognized and present |
| | | | A | by common HLA II alleles |
| LPFERATIMAAFT | 476 | NP | Influenza | broadly recognized and present |
| | | | A | by common HLA II alleles |
| PTFSVQRNLPFER | 477 | NP | Influenza | broadly recognized and present |
| | | | A | by common HLA II alleles |
| NVVRKMMTNSQDT | 478 | PB1 | Influenza | broadly recognized and present |
| | | | A | by common HLA II alleles |
| IMFSNKMARLGKG | 479 | PB1 | Influenza | broadly recognized and present |
| | | | A | by common HLA II alleles |
| GMFNMLSTVLGVS | 480 | PB1 | Influenza | broadly recognized and present |
| | | | A | by common HLA II alleles |
| SIFYHAGSSRLLTVG | 481 | L1 93- | HPV18 | HLA-DRB1*09:01/HLA- |
| | | 107 | | DRB1*07:01 |
| YPLGRKFLVQAGLRRKPT | 482 | L1 524- | HPV18 | HLA-DRB5*01:01 |
| | | 541 | | |
| ATAFTVYVFCFLLPML | 483 | E5 47- | HPV18 | HLA- |
| | | 62 | | DPA1*01:03/DPB1*02:01 |
| RTNIYYHAGTSRLLA | 484 | L1 30- | HPV16 | HLA-DRB1*09:01 |
| | | 44 | | |
| ADVMTYIHSMNSTILED | 485 | L1 385- | HPV16 | HLA-DRB1*04:05/HLA- |
| | | 401 | | DRB1*09:01/HLA- |
| | | | | DRB3*02:02 |
| VYIPLFLIHTHARFLIT | 486 | E5 67- | HPV16 | HLA-DRB1*07:01 |
| | | 83 | | |
| IDKISDVSTIVPYIGPALNI | 487 | 632 | TTD | broadly recognized and present |
| | | 651 | | by common HLA II alleles |
| ILMQYIKANSKFIGI | 488 | 827-841 | TTD | broadly recognized and present |
| | | | | by common HLA II alleles |
| NNFTVSFWLRVPKVSASHLE | 489 | 950- | TTD | broadly recognized and present |
| T | | 969 | | by common HLA II alleles |
| PVFAGANYAAWAVNVAQVI | 490 | 271- | DTD | broadly recognized and present |
| | | 290 | | by common HLA II alleles |
| VHHNTEEIVAQSIALSSLMV | 491 | 321- | DTD | broadly recognized and present |
| | | 340 | | by common HLA II alleles |
| QSIALSSLMVAQAIPLVGEL | 492 | 331- | DTD | broadly recognized and present |
| | | 350 | | by common HLA II alleles |
| QSIALSSLMVAQ | 493 | 331-342 | DTD | broadly recognized and present |
| | | | | by common HLA II alleles |
| VDIGFAAYNFVESIINLFQV | 494 | 351- | DTD | broadly recognized and present |
| | | 370 | | by common HLA II alleles |
| QGESGHDIKITAENTPLPIA | 495 | 411- | DTD | broadly recognized and present |
| | | 430 | | by common HLA II alleles |
| GVLLPTIPGKLDVNKSKTHI | 496 | 431- | DTD | broadly recognized and present |
| | | 450 | | by common HLA II alleles |

In some embodiments, the single chain trimer nucleic acid can further include a flexible first linker between the first T cell epitope and the β2 microglobulin and second linker between the β2 microglobulin and the MHC class I heavy chain sequence.

In some embodiments, the nucleic acid can include plasmid DNA, minicircle DNA, microRNA, mRNA, self-amplifying RNA, circle RNA, DNA launched self-amplifying RNA, or viral vector. In some embodiments, the T cell epitope can include a viral, bacterial, parasitic, or cancer T cell epitope. In some embodiments, the T cell epitope can include a T cell epitope of tumor-specific antigens (TSA), Tumor-associated antigens (TAA), or viral-derived cancer antigens

### Compositions

Described herein are compositions including a single chain trimer nucleic acid encoding a first T cell epitope, a β2-microglobulin, and a MHC class I heavy chain sequence.

In some embodiments, the single chain trimer nucleic acid can include nucleic acid sequences selected from any of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 498, or SEQ ID NO: 500 and homologs having at least 90% (e.g. 90%, 91%, 92%, 93%, 94%, 95%, 96%, 97%, 98%, 99%, 99.8%, or 99.9%) identity with a nucleic acid sequence of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 498, or SEQ ID NO: 500.

In some embodiments, the single chain trimer nucleic acid can encode an amino acid selected from any one of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 497, or SEQ ID NO: 499 and homologs having at least 90% (e.g. 90%, 91%, 92%, 93%, 94%, 95%, 96%, 97%, 98%, 99%, 99.8%, or 99.9%) identity with an amino acid sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 497, or SEQ ID NO: 499.

In some embodiments, the first T cell epitope can include an epitope listed in Table 1 to Table 3 or homologs having at least 90% (e.g. 90%, 91%, 92%, 93%, 94%, 95%, 96%, 97%, 98%, 99%, 99.8%, or 99.9%) identity with an epitope of Table 1 to Table 3.

In some embodiments, the first T cell epitope can include an epitope listed in Table 2 or homologs having at least 90% (e.g. 90%, 91%, 92%, 93%, 94%, 95%, 96%, 97%, 98%, 99%, 99.8%, or 99.9%) identity with an epitope of Table 2.

In some embodiments, the single chain trimer nucleic acid can include a nucleic acid encoding an amino to carboxy terminal order T cell epitope, the β2 microglobulin, and the MHC heavy chain sequence.

In some embodiments, the single chain trimer nucleic acid can further include a second T cell epitope. In some embodiments, the second T cell epitope can be a MHC class II restricted epitope. In some embodiments, the second T cell epitope comprises SEQ ID NO: 21 and SEQ ID NO: 22, SEQ ID NO: 23, SEQ ID NO: 24, SEQ ID NO: 25, SEQ ID NO: 26, or an epitope listed in Table 5 or a homolog having at least 90% (e.g. 90%, 91%, 92%, 93%, 94%, 95%, 96%, 97%, 98%, 99%, 99.8%, or 99.9%) identity with a sequence selected from SEQ ID NO. 21 SEQ ID NO: 22, SEQ ID NO: 23, SEQ ID NO: 24, SEQ ID NO: 25, SEQ ID NO: 26 and an epitope listed in Table 5.

In some embodiments, the single chain trimer nucleic acid can further include a flexible first linker between the first T cell epitope and the β2 microglobulin and second linker between the β2 microglobulin and the MHC class I heavy chain sequence.

Suitable MHC class I heavy chain can include, but are not limited to an HLA-A1:01, HLA-A2:01, HLA-A2:03, HLA-A2:06, HLA-A2:07, HLA-A3:01, HLA-A9, HLA-A10, HLA-A11:01, HLA-A23:01, HLA-A24:02, HLA-A25:01, HLA-A26:01, HLA-A29:02, HLA-A30:01, HLA-A30:02, HLA-A31:01, HLA-A32:02, HLA-A33:03, HLA-A34:01, HLA-A34:02, HLA-A68:01, HLA-A68:02, HLA-A74:01, HLA-B5, HLA-B7:02, HLA-B8:01, HLA-B-12, HLA-B13:01, HLA-B14:02, HLA-B15:01, HLA-B15:02, HLA-B15:03, HLA-B15:18, HLA-B18:01, HLA-B35:01, HLA-B38:02, HLA-B40:01, HLA-B40:02, HLA-B42:01, HLA-B44:02, HLA-B44:03, HLA-B45:01, HLA-B46:01, HLA-B49:01, HLA-B51:01, HLA-B52:01, HLA-B53:01, HLA-B54:01, HLA-B55:02, HLA-B57:01, HLA-B58:01, HLA-B67:01, HLA-C1:02, HLA-C2:02, HLA-C3:02, HLA-C3:03, HLA-C3:04, HLA-C4:01, HLA-C5:01, HLA-C6:02, HLA-C7:01, HLA-C7:02, HLA-C8:01, HLA-C8:02, HLA-C12:03, HLA-C14:02, HLA-C16:01, HLA-C17:01, or HLA-C18:01 heavy chain.

Suitable MHC class II heavy chain can include, but are not limited to HLA-DP, HLA-DQ, or HLA-DR.

In some embodiments, the compositions can further include a pharmaceutically acceptable carrier. In some aspects, disclosed herein is a pharmaceutical composition can a single chain trimer nucleic acid (e.g., mRNA) encoding a first T cell epitope, a β2-microglobulin, and a MHC heavy chain sequence; and a pharmaceutically acceptable carrier. In some embodiments, the composition can be a nanoparticle, a lipid nanoparticle dispersion, a liposomal formulation, a lipid emulsion, vaccine, vector, or any combination thereof.

In some embodiments, the pharmaceutical composition can include the lipid nanoparticle including a single chain trimer nucleic acid (e.g., mRNA) encoding a first T cell epitope, a β2-microglobulin, and a MHC class I heavy chain sequence.

Described herein are also cells including compositions described herein. In some embodiments, the cell can include a dendritic cell, B cell, macrophage, or other cells derived from a subject that can be used to for immunotherapy. In some embodiments, the T cell epitope can include a viral, bacterial, parasitic, or cancer T cell epitope. In some embodiments, the T cell epitope can include a T cell epitope of tumor-specific antigens (TSA), Tumor-associated antigens (TAA), or viral-derived cancer antigens

### Delivery Methods

Suitable nucleic acid delivery vehicles are well known in the art and can include, but are not limited to lipid-based (e.g., a liposome formulation, lipoplexes, or lipid nanoparticles (LNP)), viral-based, or physical methods such as injection, microinjection, electroporation, ultrasound, gene gun, hydrodynamic applications, or any combination thereof.

### Nanoparticles

Described herein are nanoparticles including a single chain trimer nucleic acid (e.g., mRNA) encoding a first T cell epitope, a β2-microglobulin, and a MHC class I heavy chain sequence.

In some embodiments, the nanoparticle can be a lipid nanoparticle. In some embodiments, the nanoparticles can be a lipid-polycation complex, referred to as a cationic lipid nanoparticle. As a non-limiting example, the polycation may include a cationic peptide or a polypeptide such as, but not limited to, polylysine, polyornithine and/or polyarginine. In some embodiments, the lipid nanoparticle can include a non-cationic lipid such as, but not limited to, cholesterol or dioleoyl phosphatidylethanolamine (DOPE).

In some embodiments, described herein are lipid nanoparticles including a single chain trimer nucleic acid (e.g., mRNA) encoding a first T cell epitope, a β2-microglobulin, and a MHC class I heavy chain sequence.

A lipid nanoparticle formulation may be influenced by, but not limited to, the selection of the cationic lipid component, the degree of cationic lipid saturation, the nature of the PEGylation, ratio of all components and biophysical parameters such as size. In one example by Semple et al. (Nature Biotech. 2010 28:172-176), the lipid nanoparticle can further include 57% cationic lipid, 7% dipalmitoylphosphatidylcholine, 34% cholesterol, and 1.5% PEG-c-DMA. As another example, changing the composition of the cationic lipid can more effectively deliver siRNA to various antigen presenting cells (Basha et al. Mol Ther. 2011 19:2186-2200).

In some embodiments, lipid nanoparticle formulations may comprise 35 to 45% cationic lipid, 40% to 50% cationic lipid, 50% to 60% cationic lipid and/or 55% to 65% cationic lipid. In some embodiments, the ratio of lipid to nucleic acid (e.g., mRNA) in lipid nanoparticles may be 5:1 to 20:1, 10:1 to 25:1, 15:1 to 30:1 and/or at least 30:1.

Lipid nanoparticle formulations typically comprise a lipid, in particular, an ionizable cationic lipid, and further comprise a neutral lipid, a sterol and a molecule capable of reducing particle aggregation, for example a PEG or PEG-modified lipid.

In some embodiments, a lipid nanoparticle formulation comprises at least one ionizable lipid, cationic lipid, or any combination thereof; a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of 20-60% cationic lipid:5-25% neutral lipid:25-55% sterol; 0.5-15% PEG-lipid.

In some embodiments, a lipid nanoparticle formulation includes 25% to 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy) heptadecanedioate (L319), e.g., 35 to 65%, 45 to 65%, 60%, 57.5%, 50% or 40% on a molar basis.

In some embodiments, lipid nanoparticle formulations include 25-75% of a cationic lipid, 0.5-15% of the neutral lipid, 5-50% of the sterol, and 0.5-20% of the PEG or PEG-modified lipid on a molar basis.

In some embodiments, lipid nanoparticle formulations include 35-65% of a cationic lipid, 3-12% of the neutral lipid, 15-45% of the sterol, and 0.5-10% of the PEG or PEG-modified lipid on a molar basis.

In some embodiments, lipid nanoparticle formulations include 45-65% of a cationic lipid, 5-10% of the neutral lipid, 25-40% of the sterol, and 0.5-10% of the PEG or PEG-modified lipid on a molar basis.

In some embodiments, lipid nanoparticle formulations include 60% of a cationic lipid, 7.5% of the neutral lipid, 31% of the sterol, and 1.5% of the PEG or PEG-modified lipid on a molar basis.

In some embodiments, lipid nanoparticle formulations include 50% of a cationic lipid, 10% of the neutral lipid, 38.5% of the sterol, and 1.5% of the PEG or PEG-modified lipid on a molar basis.

In some embodiments, lipid nanoparticle formulations include 50% of a cationic lipid, 10% of the neutral lipid, 35% of the sterol, 4.5% or 5% of the PEG or PEG-modified lipid, and 0.5% of the targeting lipid on a molar basis.

In some embodiments, lipid nanoparticle formulations include 40% of a cationic lipid, 15% of the neutral lipid, 40% of the sterol, and 5% of the PEG or PEG-modified lipid on a molar basis.

In some embodiments, lipid nanoparticle formulations include 57.2% of a cationic lipid, 7.1% of the neutral lipid, 34.3% of the sterol, and 1.4% of the PEG or PEG-modified lipid on a molar basis.

In some embodiments, lipid nanoparticle formulations include 57.5% of a cationic lipid selected from the PEG lipid is PEG-cDMA (PEG-CDMA is further discussed in Reyes et al. (J. Controlled Release, 107, 276-287 (2005), the contents of which are herein incorporated by reference in their entirety), 7.5% of the neutral lipid, 31.5% of the sterol, and 3.5% of the PEG or PEG-modified lipid on a molar basis.

In some embodiments, lipid nanoparticle formulations including a lipid mixture in molar ratios of 20-70% cationic lipid: 5-45% neutral lipid: 20-55% cholesterol: 0.5-15% PEG-modified lipid. In some embodiments, lipid nanoparticle formulations including a lipid mixture in a molar ratio of 20-60% cationic lipid:5-25% neutral lipid:25-55% cholesterol: 0.5-15% PEG-modified lipid.

In some embodiments, the molar lipid ratio is 50/10/38.5/1.5 (mol % cationic lipid/neutral lipid, e.g., DSPC/Chol/PEG-modified lipid, e.g., PEG-DMG, PEG-DSG or PEG-DPG), 57.2/7.1/34.3/1.4 (mol % cationic lipid/neutral lipid, e.g., DPPC/Chol/PEG-modified lipid, e.g., PEG-cDMA), 40/15/40/5 (mol % cationic lipid/neutral lipid, e.g., DSPC/Chol/PEG-modified lipid, e.g., PEG-DMG), 50/10/35/4.5/0.5 (mol % cationic lipid/neutral lipid, e.g., DSPC/Chol/PEG-modified lipid, e.g., PEG-DSG), 50/10/35/5 (cationic lipid/neutral lipid, e.g., DSPC/Chol/PEG-modified lipid, e.g., PEG-DMG), 40/10/40/10 (mol % cationic lipid/neutral lipid, e.g., DSPC/Chol/PEG-modified lipid, e.g., PEG-DMG or PEG-cDMA), 35/15/40/10 (mol % cationic lipid/neutral lipid, e.g., DSPC/Chol/PEG-modified lipid, e.g., PEG-DMG or PEG-cDMA) or 52/13/30/5 (mol % cationic lipid/neutral lipid, e.g., DSPC/Chol/PEG-modified lipid, e.g., PEG-DMG or PEG-cDMA).

Non-limiting examples of lipid nanoparticle compositions and methods of making them are described, for example, in Semple et al. (2010) Nat. Biotechnol. 28:172-176; Jayarama et al. (2012), Angew. Chem. Int. Ed., 51:8529-8533; and Maier et al. (2013) Molecular Therapy 21, 1570-1578 (the contents of each of which are incorporated herein by reference in their entirety).

In some embodiments, lipid nanoparticle formulations may comprise a cationic lipid, a PEG lipid and a structural lipid and optionally comprise a non-cationic lipid. As a non-limiting example, a lipid nanoparticle may comprise 40-60% of cationic lipid, 5-15% of a non-cationic lipid, 1-2% of a PEG lipid and 30-50% of a structural lipid. As another non-limiting example, the lipid nanoparticle may comprise 50% cationic lipid, 10% non-cationic lipid, 1.5% PEG lipid and 38.5% structural lipid. As yet another non-limiting example, a lipid nanoparticle may comprise 55% cationic lipid, 10% non-cationic lipid, 2.5% PEG lipid and 32.5% structural lipid.

In some embodiments, the lipid nanoparticle formulations described herein may be 4 component lipid nanoparticles. The lipid nanoparticle may comprise a cationic lipid, a non-cationic lipid, a PEG lipid and a structural lipid. As a non-limiting example, the lipid nanoparticle may comprise 40-60% of cationic lipid, 5-15% of a non-cationic lipid, 1-2% of a PEG lipid and 30-50% of a structural lipid. As another non-limiting example, the lipid nanoparticle may comprise 50% cationic lipid, 10% non-cationic lipid, 1.5% PEG lipid and 38.5% structural lipid. As yet another non-limiting example, the lipid nanoparticle may comprise 55% cationic lipid, 10% non-cationic lipid, 2.5% PEG lipid and 32.5% structural lipid.

In some embodiments, the lipid nanoparticle formulations described herein may comprise a cationic lipid, a non-cationic lipid, a PEG lipid and a structural lipid. As a non-limiting example, the lipid nanoparticle comprises 50% of the cationic lipid DLin-KC2-DMA, 10% of the non-cationic lipid DSPC, 1.5% of the PEG lipid PEG-DOMG and 38.5% of the structural lipid cholesterol. As a non-limiting example, the lipid nanoparticle comprises 50% of the cationic lipid DLin-MC3-DMA, 10% of the non-cationic lipid DSPC, 1.5% of the PEG lipid PEG-DOMG and 38.5% of the structural lipid cholesterol. As a non-limiting example, the lipid nanoparticle comprises 50% of the cationic lipid DLin-MC3-DMA, 10% of the non-cationic lipid DSPC, 1.5% of the PEG lipid PEG-DMG and 38.5% of the structural lipid cholesterol. As yet another non-limiting example, the lipid nanoparticle comprises 55% of the cationic lipid L319, 10% of the non-cationic lipid DSPC, 2.5% of the PEG lipid PEG-DMG and 32.5% of the structural lipid cholesterol.

In some embodiments, the nanoparticles may be such as those described in U.S. Pat. No. 10,933,127, the contents of which is herein incorporated by reference in its entirety. In some embodiments, the nanoparticles may be such as those described in U.S. Pat. No. 10,933,127, for example, nanoparticles including a compound according to Formula (I), (Ia), (II), (IIa), (IIb), (IIc), (IId), or (IIe) described from column 101 to column 187.

In some embodiments, a nanoparticle (e.g., a lipid nanoparticle) has a mean diameter of 10-500 nm, 20-400 nm, 30-300 nm, 40-200 nm. In some embodiments, a nanoparticle (e.g., a lipid nanoparticle) has a mean diameter of 50-150 nm, 50-200 nm, 80-100 nm, or 80-200 nm.

The lipid nanoparticles (LNP) described herein may be made in a sterile environment.

In some embodiments, the LNP formulation may be formulated in a nanoparticle such as a nucleic acid-lipid particle. As a non-limiting example, the lipid particle may comprise one or more active agents or therapeutic agents; one or more cationic lipids comprising from 50 mol % to 85 mol % of the total lipid present in the particle; one or more non-cationic lipids comprising from 13 mol % to 49.5 mol % of the total lipid present in the particle; and one or more conjugated lipids that inhibit aggregation of particles comprising from 0.5 mol % to 2 mol % of the total lipid present in the particle.

The nanoparticle formulations may comprise a phosphate conjugate. The phosphate conjugate may increase in vivo circulation times and/or increase the targeted delivery of the nanoparticle. As a non-limiting example, the phosphate conjugates may include a compound of any one of the formulas described in International Application No. WO2013033438, the contents of which are herein incorporated by reference in its entirety.

The nanoparticle formulation may comprise a polymer conjugate. The polymer conjugate may be a water soluble conjugate. The polymer conjugate may have a structure as described in U.S. patent application No. 20130059360, the contents of which are herein incorporated by reference in its entirety. In some embodiments, polymer conjugates with the polynucleotides of the present disclosure may be made using the methods and/or segmented polymeric reagents described in U.S. patent application No. 20130072709, the contents of which are herein incorporated by reference in its entirety. In some embodiments, the polymer conjugate may have pendant side groups comprising ring moieties such as, but not limited to, the polymer conjugates described in U.S. Patent Publication No. US20130196948, the contents which are herein incorporated by reference in its entirety.

The nanoparticle formulations may comprise a conjugate to enhance the delivery of nanoparticles of the present disclosure in a subject.

In some embodiments, compositions comprising the polynucleotides of the present disclosure and a conjugate that may have a degradable linkage. Non-limiting examples of conjugates include an aromatic moiety comprising an ionizable hydrogen atom, a spacer moiety, and a water-soluble polymer. As a non-limiting example, pharmaceutical compositions comprising a conjugate with a degradable linkage and methods for delivering such pharmaceutical compositions are described in U.S. Patent Publication No. US20130184443, the contents of which are herein incorporated by reference in their entirety.

In some embodiments, lipid nanoparticles can include 20% to 80% of an ionizable lipid, cationic lipid, or any combination thereof; greater than 0% to 5% polyethylene glycol-lipid; greater than 0% to 20% helper lipids; 20% to 80% sterol; and a single chain trimer nucleic acid encoding a first T cell epitope, a β2-microglobulin, and a MHC class I heavy chain sequence encapsulated in the lipid nanoparticle.

In some embodiments, lipid nanoparticles can include 20% to 80% of an ionizable lipid, cationic lipid, or any combination thereof; greater than 0% to 5% polyethylene glycol-lipid; greater than 0% to 20% helper lipids; 20% to 80% sterol; and a single chain trimer nucleic acid (e.g., mRNA) encoding a first T cell epitope, a β2-microglobulin, and a MHC class I heavy chain sequence encapsulated in the lipid nanoparticle.

In one embodiment, the nanoparticle can include from 40% to 60% of an ionizable lipid, cationic lipid, or any combination thereof; from 1% to 2% polyethylene glycol-lipid; from 8% to 12% helper lipids; from 35% to 40% sterol; and a single chain trimer nucleic acid (e.g., mRNA) encoding a first T cell epitope, a β2-microglobulin, and a MHC class I heavy chain sequence encapsulated in the lipid nanoparticle.

In one embodiment, the nanoparticle can include from 40% to 60% of an ionizable lipid, cationic lipid, or any combination thereof; from 1% to 2% polyethylene glycol-lipid; from 8% to 12% helper lipids; from 35% to 40% sterol; and a single chain trimer nucleic acid (e.g., mRNA) encoding a first T cell epitope, a β2-microglobulin, and a MHC class I heavy chain sequence encapsulated in the lipid nanoparticle.

In some embodiments, the single chain trimer nucleic acid can include nucleic acid sequences selected from any of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 498, or SEQ ID NO: 500 and homologs having at least 90% (e.g. 90%, 91%, 92%, 93%, 94%, 95%, 96%, 97%, 98%, 99%, 99.8%, or 99.9%) identity with a nucleic acid sequence of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, or SEQ ID NO: 20, SEQ ID NO: 498, or SEQ ID NO: 500.

In some embodiments, the single chain trimer nucleic acid can encode an amino acid selected from any one of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 497, or SEQ ID NO: 499 and homologs having at least 90% (e.g. 90%, 91%, 92%, 93%, 94%, 95%, 96%, 97%, 98%, 99%, 99.8%, or 99.9%) identity with an amino acid sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 497, or SEQ ID NO: 499.

In some embodiments, the first T cell epitope can include an epitope listed in Table 1 to Table 3 or homologs having at least 90% (e.g. 90%, 91%, 92%, 93%, 94%, 95%, 96%, 97%, 98%, 99%, 99.8%, or 99.9%) identity with an epitope of Table 1 to Table 3.

In some embodiments, the first T cell epitope can include an epitope listed in Table 2 or homologs having at least 90% (e.g. 90%, 91%, 92%, 93%, 94%, 95%, 96%, 97%, 98%, 99%, 99.8%, or 99.9%) identity with an epitope of Table 2.

In some embodiments, the single chain trimer nucleic acid can include a nucleic acid encoding an amino to carboxy terminal order T cell epitope, the β2 microglobulin, and the MHC heavy chain sequence.

In some embodiments, the single chain trimer nucleic acid can further include a second T cell epitope. In some embodiments, the second T cell epitope can be a MHC class II restricted epitope. In some embodiments, the second T cell epitope comprises SEQ ID NO: 21 and SEQ ID NO: 22, SEQ ID NO: 23, SEQ ID NO: 24, SEQ ID NO: 25, SEQ ID NO: 26, or an epitope listed in Table 5 or a homolog having at least 90% (e.g. 90%, 91%, 92%, 93%, 94%, 95%, 96%, 97%, 98%, 99%, 99.8%, or 99.9%) identity with a sequence selected from SEQ ID NO. 21 SEQ ID NO: 22, SEQ ID NO: 23, SEQ ID NO: 24, SEQ ID NO: 25, SEQ ID NO: 26 and an epitope listed in Table 5.

In some embodiments, the single chain trimer nucleic acid can further include a flexible first linker between the first T cell epitope and the β2 microglobulin and second linker between the β2 microglobulin and the MHC class I heavy chain sequence.

Suitable MHC class I heavy chain can include, but are not limited to an HLA-A1:01, HLA-A2:01, HLA-A2:03, HLA-A2:06, HLA-A2:07, HLA-A3:01, HLA-A9, HLA-A10, HLA-A11:01, HLA-A23:01, HLA-A24:02, HLA-A25:01, HLA-A26:01, HLA-A29:02, HLA-A30:01, HLA-A30:02, HLA-A31:01, HLA-A32:02, HLA-A33:03, HLA-A34:01, HLA-A34:02, HLA-A68:01, HLA-A68:02, HLA-A74:01, HLA-B5, HLA-B7:02, HLA-B8:01, HLA-B-12, HLA-B13:01, HLA-B14:02, HLA-B15:01, HLA-B15:02, HLA-B15:03, HLA-B15:18, HLA-B18:01, HLA-B35:01, HLA-B38:02, HLA-B40:01, HLA-B40:02, HLA-B42:01, HLA-B44:02, HLA-B44:03, HLA-B45:01, HLA-B46:01, HLA-B49:01, HLA-B51:01, HLA-B52:01, HLA-B53:01, HLA-B54:01, HLA-B55:02, HLA-B57:01, HLA-B58:01, HLA-B67:01, HLA-C1:02, HLA-C2:02, HLA-C3:02, HLA-C3:03, HLA-C3:04, HLA-C4:01, HLA-C5:01, HLA-C6:02, HLA-C7:01, HLA-C7:02, HLA-C8:01, HLA-C8:02, HLA-C12:03, HLA-C14:02, HLA-C16:01, HLA-C17:01, or HLA-C18:01 heavy chain.

Suitable MHC class II heavy chain can include, but are not limited to HLA-DP, HLA-DQ, or HLA-DR.

In some embodiments, the cell can include a dendritic cell, B cell, macrophage, or other cells derived from a subject that can be used to for immunotherapy. In some embodiments, the T cell epitope can include a viral, bacterial, parasitic, or cancer T cell epitope. In some embodiments, the T cell epitope can include a T cell epitope of tumor-specific antigens (TSA), Tumor-associated antigens (TAA), or viral-derived cancer antigens

### Neutral Lipid

In some embodiments, a lipid nanoparticle formulation includes 0.5% to 15% on a molar basis of the neutral lipid, e.g., 3 to 12%, 5 to 10% or 15%, 10%, or 7.5% on a molar basis. Examples of neutral lipids include, without limitation, DSPC, POPC, DPPC, DOPE and SM. In some embodiments, the formulation includes 5% to 50% on a molar basis of the sterol (e.g., 15 to 45%, 20 to 40%, 40%, 38.5%, 35%, or 31% on a molar basis. A non-limiting example of a sterol is cholesterol. In some embodiments, a lipid nanoparticle formulation includes 0.5% to 20% on a molar basis of the PEG or PEG-modified lipid (e.g., 0.5 to 10%, 0.5 to 5%, 1.5%, 0.5%, 1.5%, 3.5%, or 5% on a molar basis. In some embodiments, a PEG or PEG modified lipid comprises a PEG molecule of an average molecular weight of 2,000 Da. In some embodiments, a PEG or PEG modified lipid comprises a PEG molecule of an average molecular weight of less than 2,000, for example around 1,500 Da, around 1,000 Da, or around 500 Da. Non-limiting examples of PEG-modified lipids include PEG-distearoyl glycerol (PEG-DMG) (also referred herein as PEG-C14 or C14-PEG), PEG-cDMA (further discussed in Reyes et al. J. Controlled Release, 107, 276-287 (2005) the contents of which are herein incorporated by reference in their entirety).

### Zwitterionic Lipids

In some embodiments, the composition may be encapsulated in, linked to and/or associated with zwitterionic lipids. Non-limiting examples of zwitterionic lipids and methods of using zwitterionic lipids are described in U.S. Patent Publication No. US20130216607, the contents of which are herein incorporated by reference in their entirety. In some aspects, the zwitterionic lipids may be used in the liposomes and lipid nanoparticles described herein.

### Cationic Lipids

Suitable cationic lipids may include, but are not limited to, 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof. As a non-limiting example, the cationic lipid may be selected from (20Z,23Z)-N,N-dimethylnonacosa-20,23-dien-10-amine, (17Z,20Z)-N,N-dimemylhexacosa-17,20-dien-9-amine, (1Z,19Z)-N5N-dimethylpentacosa-16, 19-dien-8-amine, (13Z,16Z)-N,N-dimethyldocosa-13,16-dien-5-amine, (12Z,15Z)-N,N-dimethylhenicosa-12,15-dien-4-amine, (14Z,17Z)-N,N-dimethyltricosa-14,17-dien-6-amine, (15Z,18Z)-N,N-dimethyltetracosa-15,18-dien-7-amine, (18Z,21Z)-N,N-dimethylheptacosa-18,21-dien-10-amine, (15Z,18Z)-N,N-dimethyltetracosa-15,18-dien-5-amine, (14Z,17Z)-N,N-dimethyltricosa-14,17-dien-4-amine, (19Z,22Z)-N,N-dimeihyloctacosa-19,22-dien-9-amine, (18Z,21 Z)-N,N-dimethylheptacosa-18,21-dien-8 amine, (17Z,20Z)-N,N-dimethylhexacosa-17,20-dien-7-amine, (16Z,19Z)-N,N-dimethylpentacosa-16,19-dien-6-amine, (22Z,25Z)-N,N-dimethylhentriaconta-22,25-dien-10-amine, (21 Z,24Z)-N,N-dimethyltriaconta-21,24-dien-9-amine, (18Z)-N,N-dimetylheptacos-18-en-10-amine, (17Z)-N,N-dimethylhexacos-17-en-9-amine, (19Z,22Z)-N,N-dimethyloctacosa-19,22-dien-7-amine, N,N-dimethylheptacosan-10-amine, (20Z,23Z)-N-ethyl-N-methylnonacosa-20,23-dien-10-amine, 1-[(11Z,14Z)-1-nonylicosa-11,14-dien-1-yl]pyrrolidine, (20Z)-N,N-dimethylheptacos-20-en-10-amine, (15Z)-N,N-dimethyl eptacos-15-en-10-amine, (14Z)-N,N-dimethylnonacos-14-en-10-amine, (17Z)-N,N-dimethylnonacos-17-en-10-amine, (24Z)-N,N-dimethyltritriacont-24-en-10-amine, (20Z)-N,N-dimethylnonacos-20-en-10-amine, (22Z)-N,N-dimethylhentriacont-22-en-10-amine, (16Z)-N,N-dimethylpentacos-16-en-8-amine, (12Z,15Z)-N,N-dimethyl-2-nonylhenicosa-12,15-dien-1-amine, (13Z,16Z)-N,N-dimethyl-3-nonyldocosa-13,16-dien-1 amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-N,N-dimethyl-21-[(1S,2R)-2-octylcyclopropyl]nonadecan-10-amine, octylcyclopropyl]henicosan-10-amine, N,N-dimethyl-1-[(1S,2S)-2-{[(1R,2R)-2-pentylcyclopropyl]methyl}cyclopropyl]nonadecan-10-amine,N,N-dimethyl-1-[(1S,2R)-2-octylcyclopropyl]hexadecan-8-amine, N,N-dimethyl-[(1R,2S)-2-undecyIcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-R1S,2R)-2-octylcyclopropyllpentadecan-8-amine, R—N,N-dimethyl-1-[(9Z,12Z)-octadeca-9,12-dien-1-yloxy]-3-(octyloxy) propan-2-amine, S—N,N-dimethyl-1-[(9Z,12Z)-octadeca-9,12-dien-1-yloxy]-3-(octyloxy) propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-R9Z,12Z)-octadeca-9,12-dien-1-yloxylpropan-2-amine, (2S)-1-(heptyloxy)-N,N-dimethyl-3-R9Z,12Z)-octadeca-9,12-dien-1-yloxylpropan-2-amine, N,N-dimethyl-1-(nonyloxy)-3-R9Z,12Z)-octadeca-9,12-dien-1-yloxylpropan-2-amine, N,N-dimethyl-1-[(9Z)-octadec-9-en-1-yloxy]-3-(octyloxy) propan-2-amine; (2S)—N,N-dimethyl-1-[(6Z,9Z,12Z)-octadeca-6,9,12-trien-1-yloxy]-3-(octyloxy) propan-2-amine, (2S)-1-[(11Z,14Z)-icosa-11,14-dien-1-yloxy]-N,N-dimethyl-3-(pentyloxy) propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy) propan-2-amine, 1-[(13Z,16Z)-docosa-13,16-dien-1-yloxy]-N,N-dimethyl-3-(octyloxy) propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy) propan-2-amine, 1-[(9Z)-hexadec-9-en-1-yloxy]-N,N-dimethyl-3-(octyloxy) propan-2-amine, (2R)—N,N-dimethyl-H (1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-R9Z,12Z)-octadeca-9,12-dien-1-yloxylpropan-2-amine, N,N-dimethyl-1-(octyloxy)-3-({8-R1S,25)-2-{[(1R,2R)-2-pentylcyclopropyl]methyl}cyclopropyl]octyl}oxy) propan-2-amine, N,N-dimethyl-1-1 [8-(2-oclylcyclopropyl) octyl]oxy}-3-(octyloxy) propan-2-amine and (11E,20Z,23Z)-N,N-dimethylnonacosa-11,20,2-trien-10-amine or a pharmaceutically acceptable salt or stereoisomer thereof. In some embodiments, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in U.S. Patent Publication No. US20130150625, herein incorporated by reference in its entirety. In some embodiments, the nanoparticles described herein may include an amine cationic lipid such as those described in International Patent Application No. WO2013059496, the contents of which are herein incorporated by reference in their entirety. In some embodiments, the cationic lipids may have an amino-amine or an amino-amide moiety.

In some embodiments, the cationic lipid may be a low molecular weight cationic lipid such as those described in U.S. patent application No. 20130090372, the contents of which are herein incorporated by reference in their entirety.

### Ionizable Lipid

Exemplary ionizable lipids are described in the US patent publications Nos. U.S. 2016/0311759, U.S. 2015/0376115, U.S. 2016/0151284, U.S. 2017/0210697, U.S. 2015/0140070, U.S. 2013/0178541, U.S. 2013/0303587, U.S. 2015/0141678, U.S. 2015/0239926, U.S. 2016/0376224, U.S. 2017/0119904, U.S. 2012/0149894, U.S. 2015/0057373, U.S. 2013/0090372, U.S. 2013/0274523, U.S. 2013/0274504, U.S. 2013/0274504, U.S. 2009/0023673, U.S. 2012/0128760, U.S. 2010/03241240, U.S. 2014/0200257, U.S. 2015/0203446, U.S. 2018/0005363, U.S. 2014/0308304, U.S. 2013/0338210, U.S. 2012/0101148, U.S. 2012/0027796, U.S. 2012/0058144, U.S. 2013/0323269, U.S. 2011/0117125, U.S. 2011/0256175, U.S. 2012/0202871, U.S. 2011/0076335, U.S. 2006/0083780, U.S. 2013/0123338, U.S. 2015/0064242, U.S. 2006/0051405, U.S. 2013/0065939, U.S. 2006/0008910, U.S. 2003/0022649, U.S. 2010/0130588, U.S. 2013/0116307, U.S. 2010/0062967, U.S. 2013/0202684, U.S. 2014/0141070, U.S. 2014/0255472, U.S. 2014/0039032, U.S. 2018/0028664, U.S. 2016/0317458, U.S. 2013/0195920, U.S. 2022/0062175, U.S. 2021/0121411, U.S. 2022/0009878, U.S. 2022/0040325, U.S. 2012/61657480, U.S. 2016/0074514, U.S. 2013/0330401, U.S. 2019/0185410, U.S. 2012/61617468, U.S. 2019/0032087, U.S. 2015/62184188, U.S. 2019/0127318, U.S. 2021/0002813, U.S. 2020/0345641, U.S. 2014/61944336, U.S. 2012/61657480, U.S. 2021/0059953, U.S. 2022/0162521, U.S. 2022/0235377, U.S. 2018/0085474, U.S. 2018/0000953, U.S. 2020/0129445, U.S. 2021/0145982, U.S. 2021/0378980, U.S. 2020/0254086, U.S. 2021/0346306, and U.S. 2018/0000953, the contents of all of which are incorporated herein by reference in their entirety.

In some embodiments, the nanoparticle comprises an ionizable lipid in a molar ratio of from 0% to 80%. In some embodiments, the ionizable lipid can be present in a molar ratio of at least 0%, (e.g., at least 5%, at least 10%, at least 20%, at least 30%, at least 40%, at least 50%, at least 60%, at least 70%, or at least 80%). In some embodiments, the ionizable lipid can be present in a molar ratio of 80% or less, (e.g., 80% or less, 70% or less, 60% or less, 50% or less, 40% or less, 30% or less, 20% or less, 10% or less, 5% or less, 1% or less, or 0.5% or less).

The ionizable lipid can be present in a molar ratio ranging from any of the minimum values described above to any of the maximum values described above. For example, in some embodiments, the ionizable lipid can be present in a molar ratio of from 0% to 80% (e.g., from greater than 0% to 80%, from greater than 0% to 70%, from greater than 0% to 60%, from greater than 0% to 50%, from greater than 0% to 40%, from greater than 0% to 30%, from greater than 0% to 20%, from greater than 0% to 10%, from greater than 0% to 5%, from greater than 0% to 1%, from greater than 0% to 0.5%, from 1% to 30%, from 1% to 20%, from 1% to 10%, from 1% to 5%, from 5% to 30%, from 5% to 20%, from 5% to 10%, from 10% to 30%, from 10% to 20%, from 20% to 30%, from 20% to 40%, or from 30% to 40%). Helper Lipid

In some embodiments, the nanoparticle comprises a helper lipid. In some embodiments, the helper lipid can be a non-cationic lipid. In some embodiments, the non-cationic lipid can include, but is not limited to, 1,2-dioleoyl-sn-glycero-3-phosphoethanolamine (DOPE), 1-palmitoyl-2-oleoyl-sn-glycero-3-phosphoethanolamine (POPE), 1,2-distearoyl-sn-glycero-3-phosphocholine (DSPC), 1-stearoyl-2-oleoyl-sn-glycero-3-phosphoethanolamine (SOPE), DPPC (1,2-dipalmitoyl-sn-glycero-3-phosphocholine), 1,2-dioleyl-sn-glycero-3-phosphotidylcholine (DOPC), 1,2-dipalmitoyl-sn-glycero-3-phosphoethanolamine (DPPE), 1,2-dimyristoyl-sn-glycero-3-phosphoethanolamine (DMPE), 1,2-dioleoyl-5/7-glycero-3-phospho-(1′-rac-glycerol) (DOPG), or combinations thereof. In one embodiment, the non-cationic lipid is 1,2-dioleoyl-sn-glycero-3-phosphoethanolamine (DOPE). In one embodiment, the non-cationic lipid is 1-palmitoyl-2-oleoyl-sn-glycero-3-phosphoethanolamine (POPE), In one embodiment, the non-cationic lipid is 1,2-distearoyl-sn-glycero-3-phosphocholine (DSPC). In one embodiment, the non-cationic lipid is 1-stearoyl-2-oleoyl-sn-glycero-3-phosphoethanolamine (SOPE). While several non-cationic lipids are described here, additional non-cationic lipids can be used in combination with the compounds disclosed herein.

In some embodiments, the nanoparticle comprises a helper lipid in a molar ratio of from 0% to 20%. In some embodiments, the nanoparticle comprises a polyethylene glycol-lipid in a molar ratio of about 0%, 0.25%, 0.5%, 0.75%, 1%, 1.5%, 2%, 3%, 4%, 5%, 10%, 15%, or 20%.

### Polyethylene Glycol-Lipid

In some embodiments, the nanoparticle includes a polyethylene glycol-lipid (PEG-lipid). PEG-lipid is incorporated to form a hydrophilic outer layer and stabilize the particles. Nonlimiting examples of polyethylene glycol-lipids include PEG-modified lipids such as PEG-modified phosphatidylethanolamines, PEG-modified phosphatidic acids, PEG-modified ceramides, PEG-modified dialkylamines, PEG-modified diacylglycerols, and PEG-modified dialkylglycerols. Representative polyethylene glycol-lipids include DMG-PEG, DLPE-PEGs, DMPE-PEGs, DPPC-PEGs, and DSPE-PEGs. In one embodiment, the polyethylene glycol-lipid is 1,2-dimyristoyl-sn-glycerol, methoxypolyethylene glycol (DMG-PEG). In one embodiment, the polyethylene glycol-lipid is 1,2-dimyristoyl-sn-glycerol, methoxypolyethylene glycol-2000 (DMG-PEG2000). DMG-PEGXXXX means 1,2-dimyristoyl-sn-glycerol, methoxypolyethylene glycol-XXXX, wherein XXXX signifies the molecular weight of the polyethylene glycol moiety, e.g. DMG-PEG2000 or DMG-PEG5000.

In some embodiments, the nanoparticle comprises a polyethylene glycol-lipid in a molar ratio of from 0% to 5%. In some embodiments, the nanoparticle comprises a polyethylene glycol-lipid in a molar ratio of about 0%, 0.25%, 0.5%, 0.75%, 1%, 1.5%, 2%, 3%, 4%, or 5%. In one embodiment, the nanoparticle comprises a polyethylene glycol-lipid in a molar ratio of 0.75%.

In some embodiments, the ratio of PEG in the lipid nanoparticle formulations may be increased or decreased and/or the carbon chain length of the PEG lipid may be modified from C14 to C18 to alter the pharmacokinetics and/or biodistribution of the lipid nanoparticle formulations. As a non-limiting example, lipid nanoparticle formulations may contain 0.5% to 3.0%, 1.0% to 3.5%, 1.5% to 4.0%, 2.0% to 4.5%, 2.5% to 5.0% and/or 3.0% to 5.0% of the lipid molar ratio of PEG-c-DOMG (R-3-[(ωthoxy-poly(ethyleneglycol) 2000) carbamoyl)]-1,2-dimyristyloxypropyl-3-amine) (also referred to herein as PEG-DOMG) as compared to the cationic lipid, DSPC and cholesterol. In some embodiments, the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol).

In some embodiments, the LNP formulations may contain PEG-c-DOMG at 3% lipid molar ratio. In some embodiments, the LNP formulations may contain PEG-c-DOMG at 1.5% lipid molar ratio.

In some embodiments, the pharmaceutical compositions may include at least one of the PEGylated lipids described in International Publication No. WO2012099755, the contents of which are herein incorporated by reference in their entirety.

In some embodiments, the LNP formulation may contain PEG-DMG 2000 (1,2-dimyristoyl-sn-glycero-3-phophoethanolamine-N-[methoxy (polyethylene glycol)-2000). In some embodiments, the LNP formulation may contain PEG-DMG 2000, a cationic lipid known in the art and at least one other component. In some embodiments, the LNP formulation may contain PEG-DMG 2000, a cationic lipid known in the art, DSPC and cholesterol. As a non-limiting example, the LNP formulation may contain PEG-DMG 2000, DLin-DMA, DSPC and cholesterol. As another non-limiting example the LNP formulation may contain PEG-DMG 2000, DLin-DMA, DSPC and cholesterol in a molar ratio of 2:40:10:48 (see e.g., Geall et al., Nonviral delivery of self-amplifying RNA (e.g., mRNA) vaccines, PNAS 2012; PMID: 22908294, the contents of each of which are herein incorporated by reference in their entirety).

### Sterol

In some embodiments, the nanoparticle includes a sterol. Sterols are well known to those skilled the art and generally refers to those compounds having a perhydrocyclopentanophenanthrene ring system and having one or more OH substituents. Examples of sterols include, but are not limited to, cholesterol, campesterol, ergosterol, sitosterol, and the like.

In some embodiments, the sterol is selected from a cholesterol-based lipid. In some embodiments, the one or more cholesterol-based lipids are selected from cholesterol, PEGylated cholesterol, DC-Choi (N,N-dimethyl-N-ethylcarboxamidocholesterol), 1,4-bis(3-N-oleylamino-propyl) piperazine, or combinations thereof.

The sterol can be used to tune the particle permeability and fluidity base on its function in cell membranes. In one embodiment, the sterol is cholesterol.

In some embodiments, the nanoparticle comprises a sterol in a molar ratio of from 20% to 80%. In some embodiments, the nanoparticle comprises a sterol in a molar ratio of 25%, 30%, 35%, 40%, 45%, or 50%. In one embodiment, the nanoparticle comprises a sterol in a molar ratio of 40%.

The nanoparticle formulations may be a carbohydrate nanoparticle comprising a carbohydrate carrier and a nucleic acid sequence (e.g., mRNA) described herein. As a non-limiting example, the carbohydrate carrier may include, but is not limited to, an anhydride-modified phytoglycogen or glycogen-type material, phtoglycogen octenyl succinate, phytoglycogen beta-dextrin, anhydride-modified phytoglycogen beta-dextrin. (See e.g., International Publication No. WO2012109121; the contents of which are herein incorporated by reference in their entirety).

Nanoparticle formulations of the present disclosure may be coated with a surfactant or polymer in order to improve the delivery of the particle. In some embodiments, the nanoparticle may be coated with a hydrophilic coating such as, but not limited to, PEG coatings and/or coatings that have a neutral surface charge. The hydrophilic coatings may help to deliver nanoparticles with larger payloads such as, but not limited to, RNA (e.g., mRNA) within the central nervous system. As a non-limiting example nanoparticles comprising a hydrophilic coating and methods of making such nanoparticles are described in U.S. Patent Publication No. US20130183244, the contents of which are herein incorporated by reference in their entirety.

In some embodiments, the lipid nanoparticles of the present disclosure may be hydrophilic polymer particles. Non-limiting examples of hydrophilic polymer particles and methods of making hydrophilic polymer particles are described in U.S. Patent Publication No. US20130210991, the contents of which are herein incorporated by reference in their entirety.

In some embodiments, the lipid nanoparticles of the present disclosure may be hydrophobic polymer particles.

In some embodiments, an immune response may be elicited by delivering a lipid nanoparticle which may include a nanospecies, a polymer and an immunogen. (U.S. Publication No. 20120189700 and International Publication No. WO2012099805; each of which is herein incorporated by reference in their entirety). The polymer may encapsulate the nanospecies or partially encapsulate the nanospecies. The immunogen may be a recombinant protein, a modified RNA and/or a polynucleotide described herein. In some embodiments, the lipid nanoparticle may be formulated for use in a vaccine such as, but not limited to, against a pathogen.

Lipid nanoparticles may be engineered to alter the surface properties of particles so the lipid nanoparticles may penetrate the mucosal barrier. Mucus is located on mucosal tissue such as, but not limited to, oral (e.g., the buccal and esophageal membranes and tonsil tissue), ophthalmic, gastrointestinal (e.g., stomach, small intestine, large intestine, colon, rectum), nasal, respiratory (e.g., nasal, pharyngeal, tracheal and bronchial membranes), genital (e.g., vaginal, cervical and urethral membranes). Nanoparticles larger than 10-200 nm which are preferred for higher drug encapsulation efficiency and the ability to provide the sustained delivery of a wide array of drugs have been thought to be too large to rapidly diffuse through mucosal barriers. Mucus is continuously secreted, shed, discarded or digested and recycled so most of the trapped particles may be removed from the mucosa tissue within seconds or within a few hours. Large polymeric nanoparticles (200 nm-500 nm in diameter) which have been coated densely with a low molecular weight polyethylene glycol (PEG) diffused through mucus only 4 to 6-fold lower than the same particles diffusing in water (Lai et al. PNAS 2007 104(5):1482-487; Lai et al. Adv Drug Deliv Rev. 2009 61(2): 158-171; each of which is herein incorporated by reference in their entirety). The transport of nanoparticles may be determined using rates of permeation and/or fluorescent microscopy techniques including, but not limited to, fluorescence recovery after photobleaching (FRAP) and high resolution multiple particle tracking (MPT). As a non-limiting example, compositions which can penetrate a mucosal barrier may be made as described in U.S. Pat. No. 8,241,670 or International Patent Publication No. WO2013110028, the contents of each of which are herein incorporated by reference in its entirety.

The lipid nanoparticle engineered to penetrate mucus may comprise a polymeric material (i.e. a polymeric core) and/or a polymer-vitamin conjugate and/or a tri-block co-polymer. The polymeric material may include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates. The polymeric material may be biodegradable and/or biocompatible. Non-limiting examples of biocompatible polymers are described in International Patent Publication No. WO2013116804, the contents of which are herein incorporated by reference in their entirety. The polymeric material may additionally be irradiated. As a non-limiting example, the polymeric material may be gamma irradiated (see e.g., International App. No. WO201282165, herein incorporated by reference in its entirety). Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho) esters, poly(butyric acid), poly(valeric acid), poly(lactide-co-caprolactone), PEG-PLGA-PEG and trimethylene carbonate, polyvinylpyrrolidone. The lipid nanoparticle may be coated or associated with a co-polymer such as, but not limited to, a block co-polymer (such as a branched polyether-polyamide block copolymer described in International Publication No. WO2013012476, herein incorporated by reference in its entirety), and (poly(ethylene glycol))-(poly(propylene oxide))-(poly(ethylene glycol)) triblock copolymer (see e.g., U.S. Publication 20120121718 and U.S. Publication 20100003337 and U.S. Pat. No. 8,263,665, the contents of each of which is herein incorporated by reference in their entirety). The co-polymer may be a polymer that is generally regarded as safe (GRAS) and the formation of the lipid nanoparticle may be in such a way that no new chemical entities are created. For example, the lipid nanoparticle may comprise poloxamers coating PLGA nanoparticles without forming new chemical entities which are still able to rapidly penetrate human mucus (Yang et al. Angew. Chem. Int. Ed. 2011 50:2597-2600; the contents of which are herein incorporated by reference in their entirety). A non-limiting scalable method to produce nanoparticles which can penetrate human mucus is described by Xu et al. (see, e.g., J Control Release 2013, 170(2):279-86; the contents of which are herein incorporated by reference in their entirety).

The vitamin of the polymer-vitamin conjugate may be vitamin E. The vitamin portion of the conjugate may be substituted with other suitable components such as, but not limited to, vitamin A, vitamin E, other vitamins, cholesterol, a hydrophobic moiety, or a hydrophobic component of other surfactants (e.g., sterol chains, fatty acids, hydrocarbon chains and alkylene oxide chains).

The lipid nanoparticle engineered to penetrate mucus may include surface altering agents such as, but not limited to, polynucleotides, anionic proteins (e.g., bovine serum albumin), surfactants (e.g., cationic surfactants such as for example dimethyldioctadecylammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin β4 dornase alfa, neltenexine, erdosteine) and various DNases including rhDNase. The surface altering agent may be embedded or enmeshed in the particle's surface or disposed (e.g., by coating, adsorption, covalent linkage, or other process) on the surface of the lipid nanoparticle. (see e.g., U.S. Publication 20100215580 and U.S. Publication 20080166414 and US20130164343; the contents of each of which are herein incorporated by reference in their entirety).

In some embodiments, the mucus penetrating lipid nanoparticles may comprise at least one polynucleotide described herein. The polynucleotide may be encapsulated in the lipid nanoparticle and/or disposed on the surface of the particle. The polynucleotide may be covalently coupled to the lipid nanoparticle. Formulations of mucus penetrating lipid nanoparticles may comprise a plurality of nanoparticles.

In some embodiments, the compositions can be formulated as a solid lipid nanoparticle. A solid lipid nanoparticle (SLN) may be spherical with an average diameter between 10 to 1000 nm. SLN possess a solid lipid core matrix that can solubilize lipophilic molecules and may be stabilized with surfactants and/or emulsifiers. In some embodiments, the lipid nanoparticle may be a self-assembly lipid-polymer nanoparticle (see Zhang et al., ACS Nano, 2008, 2 (8), pp 1696-1702; the contents of which are herein incorporated by reference in their entirety). As a non-limiting example, the SLN may be the SLN described in International Patent Publication No. WO2013105101, the contents of which are herein incorporated by reference in their entirety. As another non-limiting example, the SLN may be made by the methods or processes described in International Patent Publication No. WO2013105101, the contents of which are herein incorporated by reference in their entirety.

In some embodiments, the compositions of the present disclosure may be encapsulated in a nanoparticle. Nanoparticles may be formulated by methods described herein and known in the art such as, but not limited to, International Pub Nos. WO2010005740, WO2010030763, WO2010005721, WO2010005723, WO2012054923, U.S. Publication Nos. US20110262491, US20100104645, US20100087337, US20100068285, US20110274759, US20100068286, US20120288541, US20130123351 and US20130230567 and U.S. Pat. Nos. 8,206,747, 8,293,276, 8,318,208 and 8,318,211; the contents of each of which are herein incorporated by reference in their entirety. In some embodiments, polymer nanoparticles may be identified by the methods described in US Pub No. US20120140790, the contents of which are herein incorporated by reference in their entirety.

In some embodiments, the nanoparticle may be formulated for sustained release. As used herein, “sustained release” refers to a pharmaceutical composition or compound that conforms to a release rate over a specific period of time. The period of time may include, but is not limited to, hours, days, weeks, months and years. As a non-limiting example, the sustained release nanoparticle may comprise a polymer and nucleic acid sequence of the present disclosure (see International Pub No. 2010075072 and US Pub No. US20100216804, US20110217377 and US20120201859, the contents of each of which are incorporated herein by reference in their entirety). In another non-limiting example, the sustained release formulation may comprise agents which permit persistent bioavailability such as, but not limited to, crystals, macromolecular gels and/or particulate suspensions (see U.S. Patent Publication No US20130150295, the contents of each of which are incorporated herein by reference in their entirety).

In some embodiments, the nanoparticles of the present disclosure may comprise a polymeric matrix. As a non-limiting example, the nanoparticle may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

In some embodiments, the nanoparticle comprises a diblock copolymer. In some embodiments, the diblock copolymer may include PEG in combination with a polymer such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof. In yet another embodiment, the diblock copolymer may be a high-X diblock copolymer such as those described in International Patent Publication No. WO2013120052, the contents of which are incorporated herein by reference in their entirety.

As a non-limiting example, the nanoparticle comprises a PLGA-PEG block copolymer (see U.S. Publication No. US20120004293 and U.S. Pat. No. 8,236,330, each of which is herein incorporated by reference in their entirety). In another non-limiting example, the therapeutic nanoparticle is a stealth nanoparticle comprising a diblock copolymer of PEG and PLA or PEG and PLGA (see U.S. Pat. No. 8,246,968 and International Publication No. WO2012166923, the contents of each of which are herein incorporated by reference in their entirety). In yet another non-limiting example, the nanoparticle is a stealth nanoparticle or a target-specific stealth nanoparticle as described in U.S. Patent Publication No. US20130172406, the contents of which are herein incorporated by reference in their entirety.

In some embodiments, the nanoparticle may comprise a multiblock copolymer (see e.g., U.S. Pat. Nos. 8,263,665 and 8,287,910 and U.S. Patent Pub. No. US20130195987, the contents of each of which are herein incorporated by reference in their entirety).

In yet another non-limiting example, the lipid nanoparticle comprises the block copolymer PEG-PLGA-PEG (see e.g., the thermosensitive hydrogel (PEG-PLGA-PEG) was used as a TGF-betal gene delivery vehicle in Lee et al. Thermosensitive Hydrogel as a Tgf-β1 Gene Delivery Vehicle Enhances Diabetic Wound Healing. Pharmaceutical Research, 2003 20(12): 1995-2000; as a controlled gene delivery system in Li et al. Controlled Gene Delivery System Based on Thermosensitive Biodegradable Hydrogel. Pharmaceutical Research 2003 20(6): 884-888; and Chang et al., Non-ionic amphiphilic biodegradable PEG-PLGA-PEG copolymer enhances gene delivery efficiency in rat skeletal muscle. J Controlled Release. 2007 118:245-253, the contents of each of which are herein incorporated by reference in their entirety). The nucleic acid (e.g., mRNA) vaccines of the present disclosure may be formulated in lipid nanoparticles comprising the PEG-PLGA-PEG block copolymer.

In some embodiments, the nanoparticle may comprise a multiblock copolymer (see e.g., U.S. Pat. Nos. 8,263,665 and 8,287,910 and U.S. Patent Pub. No. US20130195987, the contents of each of which are herein incorporated by reference in their entirety).

In some embodiments, the block copolymers described herein may be included in a polyion complex comprising a non-polymeric micelle and the block copolymer. (see e.g., U.S. Publication No. 20120076836, the contents of which are herein incorporated by reference in their entirety).

In some embodiments, the nanoparticle may comprise at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In some embodiments, the nanoparticles may comprise at least one poly(vinyl ester) polymer. The poly(vinyl ester) polymer may be a copolymer such as a random copolymer. As a non-limiting example, the random copolymer may have a structure such as those described in International Application No. WO2013032829 or U.S. Patent Publication No US20130121954, the contents of each of which are herein incorporated by reference in their entirety. In some embodiments, the poly(vinyl ester) polymers may be conjugated to the polynucleotides described herein.

In some embodiments, the nanoparticle may include at least one diblock copolymer. The diblock copolymer may be, but it not limited to, a poly(lactic) acid-poly(ethylene)glycol copolymer (see, e.g., International Patent Publication No. WO2013044219, the contents of which are herein incorporated by reference in their entirety). As a non-limiting example, the nanoparticle may be used to treat cancer (see International publication No. WO2013044219, the contents of which are herein incorporated by reference in their entirety).

In some embodiments, the nanoparticles may include at least one cationic polymer described herein and/or known in the art.

In some embodiments, the nanoparticles may include at least one amine-containing polymer such as, but not limited to polylysine, polyethylene imine, poly(amidoamine) dendrimers, poly(beta-amino esters) (see, e.g., U.S. Pat. No. 8,287,849, the contents of which are herein incorporated by reference in their entirety) and combinations thereof.

In some embodiments, the nanoparticles may comprise at least one degradable polyester which may contain polycationic side chains. Degradeable polyesters include, but are not limited to, poly(serine ester), poly(L-lactide-co-L-lysine), poly(4-hydroxy-L-proline ester), and combinations thereof. In some embodiments, the degradable polyesters may include a PEG conjugation to form a PEGylated polymer.

In some embodiments, the compositions may be formulated in colloid nanocarriers as described in U.S. Patent Publication No. US20130197100, the contents of which are herein incorporated by reference in their entirety.

In some embodiments, the nanoparticle may be optimized for oral administration. The nanoparticle may comprise at least one cationic biopolymer such as, but not limited to, chitosan or a derivative thereof. As a non-limiting example, the nanoparticle may be formulated by the methods described in U.S. Publication No. 20120282343, the contents of which are herein incorporated by reference in their entirety.

In some embodiments, LNPs comprise the lipid KL52 (an amino-lipid disclosed in U.S. Application Publication No. 2012/0295832, the contents of which are herein incorporated by reference in their entirety. Activity and/or safety (as measured by examining one or more of ALT/AST, white blood cell count and cytokine induction, for example) of LNP administration may be improved by incorporation of such lipids. LNPs comprising KL52 may be administered intravenously and/or in one or more doses. In some embodiments, administration of LNPs comprising KL52 results in equal or improved mRNA and/or protein expression as compared to LNPs comprising MC3.

In some embodiments, the lipid nanoparticle may be a limit size lipid nanoparticle described in International Patent Publication No. WO2013059922, the contents of which are herein incorporated by reference in their entirety. The limit size lipid nanoparticle may comprise a lipid bilayer surrounding an aqueous core or a hydrophobic core; where the lipid bilayer may comprise a phospholipid such as, but not limited to, diacylphosphatidylcholine, a diacylphosphatidylethanolamine, a ceramide, a sphingomyelin, a dihydrosphingomyelin, a cephalin, a cerebroside, a C8-C20 fatty acid diacylphophatidylcholine, and 1-palmitoyl-2-oleoyl phosphatidylcholine (POPC). In some embodiments, the limit size lipid nanoparticle may comprise a polyethylene glycol-lipid such as, but not limited to, DLPE-PEG, DMPE-PEG, DPPC-PEG and DSPE-PEG.

In some embodiments, the compositions may be formulated in a nanoparticle comprising an inner core comprising a non-cellular material and an outer surface comprising a cellular membrane. The cellular membrane may be derived from a cell or a membrane derived from a virus. As a non-limiting example, the nanoparticle may be made by the methods described in International Patent Publication No. WO2013052167, the contents of which are herein incorporated by reference in their entirety. As another non-limiting example, the nanoparticle described in International Patent Publication No. WO2013052167, the contents of which are herein incorporated by reference in their entirety, may be used to deliver the compositions described herein.

In some embodiments, the compositions may be formulated in porous nanoparticle-supported lipid bilayers (protocells). Protocells are described in International Patent Publication No. WO2013056132, the contents of which are herein incorporated by reference in their entirety.

In some embodiments, the compositions described herein may be formulated in polymeric nanoparticles as described in or made by the methods described in U.S. Pat. Nos. 8,420,123 and 8,518,963 and European Patent No. EP2073848B1, the contents of each of which are herein incorporated by reference in their entirety. As a non-limiting example, the polymeric nanoparticle may have a high glass transition temperature such as the nanoparticles described in or nanoparticles made by the methods described in U.S. Pat. No. 8,518,963, the contents of which are herein incorporated by reference in their entirety. As another non-limiting example, the polymer nanoparticle for oral and parenteral formulations may be made by the methods described in European Patent No. EP2073848B1, the contents of which are herein incorporated by reference in their entirety.

In some embodiments, the compositions described herein may be formulated in nanoparticles used in imaging. The nanoparticles may be liposome nanoparticles such as those described in U.S. Patent Publication No US20130129636, herein incorporated by reference in its entirety. As a non-limiting example, the liposome may comprise gadolinium (III) 2-{4,7-bis-carboxymethyl-10-[(N,N-distearylamidomethyl-N′-amido-methyl]-1,4,7,10-tetra-azacyclododec-1-yl}-acetic acid and a neutral, fully saturated phospholipid component (see, e.g., U.S. Patent Publication No US20130129636, the contents of which are herein incorporated by reference in their entirety).

In some embodiments, the nanoparticles which may be used in the present disclosure are formed by the methods described in U.S. Patent Application No. US20130130348, the contents of which are herein incorporated by reference in their entirety.

In some embodiments, the compositions of the present disclosure may be formulated in a swellable nanoparticle. The swellable nanoparticle may be, but is not limited to, those described in U.S. Pat. No. 8,440,231, the contents of which are herein incorporated by reference in their entirety.

The compositions of the present disclosure may be formulated in polyanhydride nanoparticles such as, but not limited to, those described in U.S. Pat. No. 8,449,916, the contents of which are herein incorporated by reference in their entirety.

The nanoparticles and microparticles of the present disclosure may be geometrically engineered to modulate macrophage and/or the immune response. In some embodiments, the geometrically engineered particles may have varied shapes, sizes and/or surface charges in order to incorporate the polynucleotides of the present disclosure for targeted delivery. Other physical features the geometrically engineering particles may have include, but are not limited to, fenestrations, angled arms, asymmetry and surface roughness, charge which can alter the interactions with cells and tissues. As a non-limiting example, nanoparticles of the present disclosure may be made by the methods described in International Publication No WO2013082111, the contents of which are herein incorporated by reference in their entirety.

In some embodiments, the nanoparticles of the present disclosure may be water soluble nanoparticles such as, but not limited to, those described in International Publication No. WO2013090601, the contents of which are herein incorporated by reference in their entirety. The nanoparticles may be inorganic nanoparticles which have a compact and zwitterionic ligand in order to exhibit good water solubility. The nanoparticles may also have small hydrodynamic diameters (HD), stability with respect to time, pH, and salinity and a low level of non-specific protein binding.

In some embodiments, the nanoparticles of the present disclosure may be developed by the methods described in U.S. Patent Publication No. US20130172406, the contents of which are herein incorporated by reference in their entirety.

In some embodiments, the nanoparticles of the present disclosure are stealth nanoparticles or target-specific stealth nanoparticles such as, but not limited to, those described in U.S. Patent Publication No. US20130172406, the contents of which are herein incorporated by reference in their entirety. The nanoparticles of the present disclosure may be made by the methods described in U.S. Patent Publication No. US20130172406, the contents of which are herein incorporated by reference in their entirety.

In some embodiments, the stealth or target-specific stealth nanoparticles may comprise a polymeric matrix. The polymeric matrix may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates or combinations thereof.

In some embodiments, the nanoparticle may be a nanoparticle-nucleic acid hybrid structure having a high-density nucleic acid layer. As a non-limiting example, the nanoparticle-nucleic acid hybrid structure may made by the methods described in U.S. Patent Publication No. US20130171646, the contents of which are herein incorporated by reference in their entirety. The nanoparticle may comprise a nucleic acid such as, but not limited to, polynucleotides described herein and/or known in the art.

At least one of the nanoparticles of the present disclosure may be embedded in the core a nanostructure or coated with a low density porous 3-D structure or coating which is capable of carrying or associating with at least one payload within or on the surface of the nanostructure. Non-limiting examples of the nanostructures comprising at least one nanoparticle are described in International Patent Publication No. WO2013123523, the contents of which are herein incorporated by reference in their entirety.

In some embodiments, compositions may be delivered using smaller LNPs. Such particles may comprise a diameter from below 0.1 μm up to 100 nm such as, but not limited to, less than 0.1 μm, less than 1.0 μm, less than 5 μm, less than 10 μm, less than 15 μm, less than 20 μm, less than 25 μm, less than 30 μm, less than 35 μm, less than 40 μm, less than 50 μm, less than 55 μm, less than 60 μm, less than 65 μm, less than 70 μm, less than 75 μm, less than 80 μm, less than 85 μm, less than 90 μm, less than 95 μm, less than 100 μm, less than 125 μm, less than 150 μm, less than 175 μm, less than 200 μm, less than 225 μm, less than 250 μm, less than 275 μm, less than 300 μm, less than 325 μm, less than 350 μm, less than 375 μm, less than 400 μm, less than 425 μm, less than 450 μm, less than 475 μm, less than 500 μm, less than 525 μm, less than 550 μm, less than 575 μm, less than 600 μm, less than 625 μm, less than 650 μm, less than 675 μm, less than 700 μm, less than 725 μm, less than 750 μm, less than 775 μm, less than 800 μm, less than 825 μm, less than 850 μm, less than 875 μm, less than 900 μm, less than 925 μm, less than 950 μm, less than 975 μm, or less than 1000 μm.

In some embodiments, compositions may be delivered using smaller LNPs, which may comprise a diameter from 1 nm to 100 nm, from 1 nm to 10 nm, 1 nm to 20 nm, from 1 nm to 30 nm, from 1 nm to 40 nm, from 1 nm to 50 nm, from 1 nm to 60 nm, from 1 nm to 70 nm, from 1 nm to 80 nm, from 1 nm to 90 nm, from 5 nm to from 100 nm, from 5 nm to 10 nm, 5 nm to 20 nm, from 5 nm to 30 nm, from 5 nm to 40 nm, from 5 nm to 50 nm, from 5 nm to 60 nm, from 5 nm to 70 nm, from 5 nm to 80 nm, from 5 nm to 90 nm, from 10 to 50 nm, from 20 to 50 nm, from 30 to 50 nm, from 40 to 50 nm, from 20 to 60 nm, from 30 to 60 nm, from 40 to 60 nm, from 20 to 70 nm, from 30 to 70 nm, from 40 to 70 nm, from 50 to 70 nm, from 60 to 70 nm, from 20 to 80 nm, from 30 to 80 nm, from 40 to 80 nm, from 50 to 80 nm, from 60 to 80 nm, from 20 to 90 nm, from 30 to 90 nm, from 40 to 90 nm, from 50 to 90 nm, from 60 to 90 nm and/or from 70 to 90 nm.

In some embodiments, such LNPs are synthesized using methods comprising microfluidic mixers. Examples of microfluidic mixers may include, but are not limited to, a slit interdigital micromixer including, but not limited to those manufactured by Microinnova (Allerheiligen bei Wildon, Austria) and/or a staggered herringbone micromixer (SHM) (Zhigaltsev, I. V. et al., Bottom-up design and synthesis of limit size lipid nanoparticle systems with aqueous and triglyceride cores using millisecond microfluidic mixing have been published (Langmuir. 2012. 28:3633-40; Belliveau, N. M. et al., Microfluidic synthesis of highly potent limit-size lipid nanoparticles for in vivo delivery of siRNA. Molecular Therapy-Nucleic Acids. 2012. 1:e37; Chen, D. et al., Rapid discovery of potent siRNA-containing lipid nanoparticles enabled by controlled microfluidic formulation. J Am Chem Soc. 2012. 134(16):6948-51, the contents of each of which are herein incorporated by reference in their entirety). In some embodiments, methods of LNP generation comprising SHM, further comprise the mixing of at least two input streams wherein mixing occurs by microstructure-induced chaotic advection (MICA). According to this method, fluid streams flow through channels present in a herringbone pattern causing rotational flow and folding the fluids around each other. This method may also comprise a surface for fluid mixing wherein the surface changes orientations during fluid cycling. Methods of generating LNPs using SHM include those disclosed in U.S. Application Publication Nos. 2004/0262223 and 2012/0276209, the contents of each of which are herein incorporated by reference in their entirety.

In some embodiments, the compositions of the present disclosure may be formulated in lipid nanoparticles created using a micromixer such as, but not limited to, a Slit Interdigital Microstructured Mixer (SIMM-V2) or a Standard Slit Interdigital Micro Mixer (SSIMM) or Caterpillar (CPMM) or Impinging-jet (IJMM) from the Institut für Mikrotechnik Mainz GmbH, Mainz Germany).

In some embodiments, the compositions of the present disclosure may be formulated in lipid nanoparticles created using microfluidic technology (see, e.g., Whitesides, George M. The Origins and the Future of Microfluidics. Nature, 2006 442:368-373; and Abraham et al. Chaotic Mixer for Microchannels. Science, 2002 295:647-651; each of which is herein incorporated by reference in its entirety). As a non-limiting example, controlled microfluidic formulation includes a passive method for mixing streams of steady pressure-driven flows in micro channels at a low Reynolds number (see, e.g., Abraham et al. Chaotic Mixer for Microchannels. Science, 2002 295:647-651, the contents of which are herein incorporated by reference in their entirety).

In some embodiments, the compositions of the present disclosure may be formulated in lipid nanoparticles created using a micromixer chip such as, but not limited to, those from Harvard Apparatus (Holliston, Mass.) or Dolomite Microfluidics (Royston, UK). A micromixer chip can be used for rapid mixing of two or more fluid streams with a split and recombine mechanism.

In some embodiments, the compositions of the disclosure may be formulated in lipid nanoparticles having a diameter from 10 to 100 nm such as, but not limited to, 10 to 20 nm, 10 to 30 nm, 10 to 40 nm, 10 to 50 nm, 10 to 60 nm, 10 to 70 nm, 10 to 80 nm, 10 to 90 nm, 20 to 30 nm, 20 to 40 nm, 20 to 50 nm, 20 to 60 nm, 20 to 70 nm, 20 to 80 nm, 20 to 90 nm, 20 to 100 nm, 30 to 40 nm, 30 to 50 nm, 30 to 60 nm, 30 to 70 nm, 30 to 80 nm, 30 to 90 nm, 30 to 100 nm, 40 to 50 nm, 40 to 60 nm, 40 to 70 nm, 40 to 80 nm, about 40 to about 90 nm, about 40 to 100 nm, 50 to 60 nm, 50 to 70 nm, 50 to 80 nm, 50 to 90 nm, 50 to 100 nm, 60 to 70 nm, 60 to 80 nm, 60 to 90 nm, 60 to 100 nm, 70 to 80 nm, 70 to 90 nm, 70 to 100 nm, 80 to 90 nm, 80 to 100 nm, and/or 90 to 100 nm.

In some embodiments, the lipid nanoparticles may have a diameter from 10 to 500 nm.

In some embodiments, the lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

Described herein are also composition including an effective amount of a nanoparticle described herein and a pharmaceutically acceptable carrier. In some aspects, the pharmaceutical compositions can include a pharmaceutically acceptable carrier and a nanoparticle including a single chain trimer nucleic acid (e.g., mRNA) encoding a first T cell epitope, a β2-microglobulin, and a first MHC heavy chain sequence encapsulated in the nanoparticle. In some aspects, the pharmaceutical composition can include a pharmaceutically acceptable carrier and a lipid nanoparticle including a single chain trimer nucleic acid (e.g., mRNA) encoding a first T cell epitope, a β2-microglobulin, and a first MHC heavy chain sequence encapsulated in the lipid nanoparticle.

Described herein are also composition including an effective amount of a nanoparticle described herein and a pharmaceutically acceptable carrier. In some aspects, the pharmaceutical compositions can include a pharmaceutically acceptable carrier and a nanoparticle including a single chain nucleic acid dimer (e.g., mRNA) encoding a T cell epitope, and a primary MHC heavy chain sequence encapsulated in the nanoparticle. In some aspects, the pharmaceutical composition can include a pharmaceutically acceptable carrier and a lipid nanoparticle including a single chain nucleic acid dimer (e.g., mRNA) encoding a T cell epitope, and a primary MHC heavy chain sequence encapsulated in the lipid nanoparticle.

Liposomes, lipoplexes, or lipid nanoparticles may be used to improve the efficacy of polynucleotides directed protein production as these formulations may be able to increase cell transfection by the RNA (e.g., mRNA) polynucleotide; and/or increase the translation of encoded protein. One such example involves the use of lipid encapsulation to enable the effective systemic delivery of polyplex plasmid DNA (Heyes et al., Mol Ther. 2007 15:713-720; the contents of which are incorporated herein by reference in their entirety). The liposomes, lipoplexes, or lipid nanoparticles may also be used to increase the stability of the polynucleotide.

Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In some embodiments, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, Pa.).

In some embodiments, pharmaceutical compositions described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No US20130122104; all of which are incorporated herein in their entireties). The original manufacture method by Wheeler et al. was a detergent dialysis method, which was later improved by Jeffs et al. and is referred to as the spontaneous vesicle formation method. The liposome formulations are composed of 3 to 4 lipid components in addition to the polynucleotide. As an example a liposome can contain, but is not limited to, 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. As another example, certain liposome formulations may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al.

In some embodiments, liposome formulations may comprise from about 25.0% cholesterol to about 40.0% cholesterol, from about 30.0% cholesterol to about 45.0% cholesterol, from about 35.0% cholesterol to about 50.0% cholesterol and/or from about 48.5% cholesterol to about 60% cholesterol. In some embodiments, formulations may comprise a percentage of cholesterol selected from the group consisting of 28.5%, 31.5%, 33.5%, 36.5%, 37.0%, 38.5%, 39.0% and 43.5%. In some embodiments, formulations may comprise from about 5.0% to about 10.0% DSPC and/or from about 7.0% to about 15.0% DSPC.

In some embodiments, the compositions may be formulated in liposomes such as, but not limited to, DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713); herein incorporated by reference in its entirety) and hyaluronan-coated liposomes (Quiet Therapeutics, Israel).

In some embodiments, the compositions may be formulated in a lipid vesicle, which may have crosslinks between functionalized lipid bilayers.

In some embodiments, the compositions may be formulated in a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine. In some embodiments, the compositions may be formulated in a lipid-polycation complex, which may further include a non-cationic lipid such as, but not limited to, cholesterol or dioleoyl phosphatidylethanolamine (DOPE).

In some embodiments, the composition can be formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, Mass.), and polyethylenimine (PEI) or protamine-based targeted and non-targeted delivery of nucleic acids acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293 Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132, the contents of each of which are incorporated herein by reference in their entirety).

In some embodiments, such formulations may also be constructed or compositions altered such that they passively or actively are directed to different cell types in vivo, including but not limited to hepatocytes, immune cells, tumor cells, endothelial cells, antigen presenting cells, and leukocytes (Akinc et al. Mol Ther. 2010 18:1357-1364; Song et al., Nat Biotechnol. 2005 23:709-717; Judge et al., J Clin Invest. 2009 119:661-673; Kaufmann et al., Microvasc Res 2010 80:286-293; Santel et al., Gene Ther 2006 13:1222-1234; Santel et al., Gene Ther 2006 13:1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 2008 5:25-44; Peer et al., Science. 2008 319:627-630; Peer and Lieberman, Gene Ther. 2011 18:1127-1133, the contents of each of which are incorporated herein by reference in their entirety). One example of passive targeting of formulations to liver cells includes the DLin-DMA, DLin-KC2-DMA and DLin-MC3-DMA-based lipid nanoparticle formulations, which have been shown to bind to apolipoprotein E and promote binding and uptake of these formulations into hepatocytes in vivo (Akinc et al. Mol Ther. 2010 18:1357-1364, the contents of which are incorporated herein by reference in their entirety). Formulations can also be selectively targeted through expression of different ligands on their surface as exemplified by, but not limited by, folate, transferrin, N-acetylgalactosamine (GalNAc), and antibody targeted approaches (Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; Peer and Lieberman, Gene Ther. 2011 18:1127-1133, the contents of each of which are incorporated herein by reference in their entirety).

In some embodiments, the composition of the present disclosure can be formulated for controlled release and/or targeted delivery. As used herein, “controlled release” refers to a pharmaceutical composition or compound release profile that conforms to a particular pattern of release to effect a therapeutic outcome. In some embodiments, the compositions may be encapsulated into a delivery agent described herein and/or known in the art for controlled release and/or targeted delivery. As used herein, the term “encapsulate” means to enclose, surround or encase. As it relates to the formulation of the compounds of the disclosure, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or compound of the disclosure may be enclosed, surrounded or encased within the delivery agent. “Partially encapsulation” means that less than 10, 10, 20, 30, 40 50 or less of the pharmaceutical composition or compound of the disclosure may be enclosed, surrounded or encased within the delivery agent. Advantageously, encapsulation may be determined by measuring the escape or the activity of the pharmaceutical composition or compound of the disclosure using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the disclosure are encapsulated in the delivery agent.

In some embodiments, the controlled release formulation may include, but is not limited to, tri-block co-polymers. As a non-limiting example, the formulation may include two different types of tri-block co-polymers (U.S. Pat. Nos. 9,901,554 and 9,795,679, the contents of each of which are incorporated herein by reference in their entirety).

In some embodiments, the compositions may be encapsulated into a lipid nanoparticle or a rapidly eliminated lipid nanoparticle and the lipid nanoparticles or a rapidly eliminated lipid nanoparticle may then be encapsulated into a polymer, hydrogel and/or surgical sealant described herein and/or known in the art. As a non-limiting example, the polymer, hydrogel or surgical sealant may be PLGA, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.).

In some embodiments, the compositions described herein formulated for controlled release and/or targeted delivery may also include at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In some embodiments, the degradable polyesters may include a PEG conjugation to form a PEGylated polymer.

In some embodiments, the compositions described herein formulated for controlled release and/or targeted delivery may also include at least one PEG and/or PEG related polymer derivatives as described in U.S. Pat. No. 8,404,222, the contents of which are incorporated herein by reference in their entirety.

In some embodiments, the compositions described herein formulated for controlled release delivery may be the controlled release polymer system described in US20130130348, the contents of which are incorporated herein by reference in their entirety.

In some embodiments, the synthetic nanocarriers may be formulated for targeted release. In some embodiments, the synthetic nanocarrier is formulated to release the polynucleotides at a specified pH and/or after a desired time interval. As a non-limiting example, the synthetic nanoparticle may be formulated to release the RNA (e.g., mRNA) vaccines after 24 hours and/or at a pH of 4.5 (see International Publication Nos. WO2010138193 and WO2010138194 and U.S. Pub Nos. US20110020388 and US20110027217, each of which is herein incorporated by reference in their entireties).

In some embodiments, the synthetic nanocarriers may be formulated for controlled and/or sustained release of the nucleic acids described herein. As a non-limiting example, the synthetic nanocarriers for sustained release may be formulated by methods known in the art, described herein and/or as described in International Pub No. WO2010138192 and US Pub No. 20100303850, each of which is herein incorporated by reference in their entirety.

In some embodiments, the synthetic nanocarrier may be formulated for use as a vaccine. As a non-limiting example, the synthetic nanocarrier may include but is not limited to nanocarriers described in International Publication No. WO2011150264, WO2011150249, WO2012024621, WO201202629, WO2012024632 and U.S. Publication No. US20110293701, U.S. Publication No. US20110293723, US20120064110, US20120058153 and US20120058154 the contents of each of which are herein incorporated by reference in their entirety. The vaccine dosage form may be selected by methods described herein, known in the art and/or described in International Publication No. WO2011150258 and U.S. Publication No. US20120027806, the contents of each of which are herein incorporated by reference in their entirety).

In some embodiments, the compositions may be formulated in colloid nanocarriers as described in U.S. Patent Publication No. US20130197100, the contents of which are herein incorporated by reference in their entirety.

In some embodiments, the compositions of the disclosure may be formulated for delivery using the drug encapsulating microspheres described in International Patent Publication No. WO2013063468 or U.S. Pat. No. 8,440,614, the contents of each of which are herein incorporated by reference in their entirety. The microspheres may comprise a compound of the formula (I), (II), (III), (IV), (V) or (VI) as described in International Patent Publication No. WO2013063468, the contents of which are herein incorporated by reference in their entirety. In some embodiments, the amino acid, peptide, polypeptide, lipids (APPL) are useful in delivering the RNA (e.g., mRNA) polynucleotides of the disclosure to cells (see International Patent Publication No. WO2013063468, the contents of which are herein incorporated by reference in their entirety).

In some embodiments, the compositions may be delivered, localized and/or concentrated in a specific location using the delivery methods described in International Patent Publication No. WO2013063530, the contents of which are herein incorporated by reference in their entirety. As a non-limiting example, a subject may be administered an empty polymeric particle prior to, simultaneously with or after delivering the compositions described herein to the subject. The empty polymeric particle undergoes a change in volume once in contact with the subject and becomes lodged, embedded, immobilized or entrapped at a specific location in the subject.

In some embodiments, the compositions may be formulated in an active substance release system (see, e.g., U.S. Patent Publication No. US20130102545, the contents of which are herein incorporated by reference in their entirety). The active substance release system may comprise 1) at least one nanoparticle bonded to an oligonucleotide inhibitor strand which is hybridized with a catalytically active nucleic acid and 2) a compound bonded to at least one substrate molecule bonded to a therapeutically active substance (e.g., polynucleotides described herein), where the therapeutically active substance is released by the cleavage of the substrate molecule by the catalytically active nucleic acid.

In some embodiments the compositions may be associated with a cationic or polycationic compounds, including protamine, nucleoline, spermine or spermidine, or other cationic peptides or proteins, such as poly-L-lysine (PLL), polyarginine, basic polypeptides, cell penetrating peptides (CPPs), including HIV-binding peptides, HIV-1 Tat (HIV), Tat-derived peptides, Penetratin, VP²² derived or analog peptides, Pestivirus Ems, HSV, VP²² (Herpes simplex), MAP, KALA or protein transduction domains (PTDs), PpT620, prolin-rich peptides, arginine-rich peptides, lysine-rich peptides, MPG-peptide(s), Pep-1, L-oligomers, Calcitonin peptide(s), Antennapedia-derived peptides (particularly from Drosophila antennapedia), pAntp, p1s1, FGF, Lactoferrin, Transportan, Buforin-2, Bac715-24, SynB, SynB(1), pVEC, hCT-derived peptides, SAP, histones, cationic polysaccharides, for example chitosan, polybrene, cationic polymers, e.g. polyethyleneimine (PEI), cationic lipids, e.g. DOTMA: [1-(2,3-sioleyloxy)propyl)]-N,N,N-trimethylammonium chloride, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS: Dioctadecylamidoglicylspermin, DIMRI: Dimyristooxypropyl dimethyl hydroxyethyl ammonium bromide, DOTAP: dioleoyloxy-3-(trimethylammonio)propane, DC-6-14: O,O-ditetradecanoyl-N-.alpha.-trimethylammonioacetyl)diethanolamine chloride, CLIP 1: rac-[(2,3-dioctadecyloxypropyl)(2-hydroxyethyl)]-dimethylammonium chloride, CLIP6: rac-[2 (2,3-dihexadecyloxypropyloxymethyloxy)ethyl]-trimethylammonium, CLIP9: rac-[2 (2,3-dihexadecyloxypropyloxysuccinyloxy)ethyl]-trimethylammonium, oligofectamine, or cationic or polycationic polymers, e.g. modified polyaminoacids, such as beta-aminoacid-polymers or reversed polyamides, etc., modified polyethylenes, such as PVP (poly(N-ethyl-4-vinylpyridinium bromide)), etc., modified acrylates, such as pDMAEMA (poly(dimethylaminoethyl methylacrylate)), etc., modified amidoamines such as pAMAM (poly(amidoamine)), etc., modified polybetaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI: poly(ethyleneimine), poly(propyleneimine), etc., polyallylamine, sugar backbone based polymers, such as cyclodextrin based polymers, dextran based polymers, chitosan, etc., silan backbone based polymers, such as PMOXA-PDMS copolymers, etc., blockpolymers consisting of a combination of one or more cationic blocks (e.g. selected from a cationic polymer as mentioned above) and of one or more hydrophilic or hydrophobic blocks (e.g. polyethyleneglycole), etc.

In other embodiments, the composition is not associated with a cationic or polycationic compound.

### Vaccines

Described herein are vaccines including the compositions described herein. In some aspects, described herein are vaccines including a composition including a single chain trimer nucleic acid (e.g., mRNA) encoding a first T cell epitope, a β2-microglobulin, and a first MHC heavy chain sequence.

In some aspects, also described herein are vaccines including a composition including a single chain nucleic acid dimer (e.g., mRNA) encoding a T cell epitope, and a primary MHC heavy chain sequence.

In some embodiments, vaccines can be formulated in a nanoparticle described herein. In some embodiments, vaccines can be formulated in a lipid nanoparticle described herein. In some embodiments, vaccines can be formulated in a lipid-polycation complex described herein, referred to as a cationic lipid nanoparticle. The vaccines of the disclosure can be formulated using one or more liposomes, lipoplexes, or lipid nanoparticles described herein. In some embodiments, pharmaceutical compositions of vaccines can include liposomes described herein. In some embodiments, the vaccines may be formulated in a lipid vesicle, which may have crosslinks between functionalized lipid bilayers. In some embodiments, the vaccines may be formulated in a lipid-polycation complex.

In some embodiments, described herein are vaccines including a lipid nanoparticle including a single chain trimer nucleic acid (e.g., mRNA) encoding a first T cell epitope, a β2-microglobulin, and a first MHC heavy chain sequence. In some embodiments, also described herein are vaccines including a lipid nanoparticle including a single chain nucleic acid dimer (e.g., mRNA) encoding a T cell epitope, and a primary MHC heavy chain sequence.

### Methods of Administration

The compositions as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy (Polyethylene glycol)-1000], 1,2-Distearoyl-sn-glycero-3-Phosphoethanolamine-N-[Methoxy (Polyethylene glycol)-2000], and 1,2-Distearoyl-sn-glycero-3-Phosphoethanolamine-N-[Methoxy (Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly(meth)acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The compounds can be incorporated microparticles, nanoparticles, or combinations thereof that provide controlled release of the compounds and/or additional active agents. For example, the compounds can be incorporated into polymeric microparticles, which provide controlled release of the drug(s). Release of the drug(s) is controlled by diffusion of the drug(s) out of the microparticles and/or degradation of the polymeric particles by hydrolysis and/or enzymatic degradation. Suitable polymers include ethylcellulose and other natural or synthetic cellulose derivatives.

Polymers, which are slowly soluble and form a gel in an aqueous environment, such as hydroxypropyl methylcellulose or polyethylene oxide, may also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the compound can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30 to 300° C.

In some cases, it may be desirable to alter the rate of water penetration into the microparticles. To this end, rate-controlling (wicking) agents may be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins, which are water insoluble, such as zein, can also be used as materials for the formation of drug containing microparticles. Additionally, proteins, polysaccharides and combinations thereof, which are water-soluble, can be formulated with drug into microparticles and subsequently cross-linked to form an insoluble network. For example, cyclodextrins can be complexed with individual drug molecules and subsequently cross-linked.

Encapsulation or incorporation of drug into carrier materials to produce drug-containing microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. These processes are known in the art.

For some carrier materials it may be desirable to use a solvent evaporation technique to produce drug-containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, drug(s) in a particulate form is homogeneously dispersed in a water-insoluble or slowly water soluble material. To minimize the size of the drug particles within the composition, the drug powder itself may be milled to generate fine particles prior to formulation. The process of jet milling, known in the pharmaceutical art, can be used for this purpose. In some embodiments, drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water-soluble protein can be spray coated onto the microparticles and subsequently cross-linked by the one of the methods described above. Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

In certain embodiments, it may be desirable to provide continuous delivery of one or more compounds to a patient in need thereof. For intravenous or intraarterial routes, this can be accomplished using drip systems, such as by intravenous administration. For topical applications, repeated application can be done or a patch can be used to provide continuous administration of the compounds over an extended period of time.

The compounds described herein can be incorporated into injectable/implantable solid or semi-solid implants, such as polymeric implants. In one embodiment, the compounds are incorporated into a polymer that is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication require polymers having a melting point that is below the temperature at which the substance to be delivered and polymer degrade or become reactive. The device can also be prepared by solvent casting where the polymer is dissolved in a solvent and the drug dissolved or dispersed in the polymer solution and the solvent is then evaporated. Solvent processes require that the polymer be soluble in organic solvents. Another method is compression molding of a mixed powder of the polymer and the drug or polymer particles loaded with the active agent.

Alternatively, the compounds can be incorporated into a polymer matrix and molded, compressed, or extruded into a device that is a solid at room temperature. For example, the compounds can be incorporated into a biodegradable polymer, such as polyanhydrides, polyhydroalkanoic acids (PHAs), PLA, PGA, PLGA, polycaprolactone, polyesters, polyamides, polyorthoesters, polyphosphazenes, proteins and polysaccharides such as collagen, hyaluronic acid, albumin and gelatin, and combinations thereof and compressed into solid device, such as disks, wafers, or extruded into a device, such as rods.

The release of the compounds from the implant can be varied by selection of the polymer, the molecular weight of the polymer, and/or modification of the polymer to increase degradation, such as the formation of pores and/or incorporation of hydrolyzable linkages. Methods for modifying the properties of biodegradable polymers to vary the release profile of the compounds from the implant are well known in the art.

In some embodiments, the compounds or pharmaceutical compositions can be administered locally. In some embodiments, the compounds are incorporated in a delivery system such as gels, nanoparticles, microparticles, or implants such as (e.g., rods, discs, wafers, orthopedic implants) for sustained release. In some embodiments, the compounds can be administered using a local delivery implantable system comprising the compounds incorporated within a gel, nanoparticles, microparticles, or an implant. In some embodiments, the pharmaceutical compositions comprise a delivery system such as gels, nanoparticles, microparticles, or implants such as (e.g., rods, discs, wafers, orthopedic implants) for sustained release of paroxetine or a pharmaceutically acceptable salt or derivative thereof.

The active ingredient may be administered in such amounts, time, and route deemed necessary in order to achieve the desired result. The exact amount of the active ingredient will vary from subject to subject, depending on the species, age, and general condition of the subject, the severity of the infection, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intraarterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Compositions described herein are typically formulated in dosage unit form for ease of administration and uniformity of dosage. It will be understood, however, that the total daily usage of compositions may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, the compositions may be administered at dosage levels sufficient to deliver 0.0001 mg/kg to 100 mg/kg, 0.001 mg/kg to 0.05 mg/kg, 0.005 mg/kg to 0.05 mg/kg, 0.001 mg/kg to 0.005 mg/kg, 0.05 mg/kg to 0.5 mg/kg, 0.01 mg/kg to 50 mg/kg, 0.1 mg/kg to 40 mg/kg, 0.5 mg/kg to 30 mg/kg, 0.01 mg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic, diagnostic, prophylactic, or imaging effect (see, e.g., the range of unit doses described in International Publication No WO2013078199, the contents of which are herein incorporated by reference in their entirety). The desired dosage may be delivered three times a day, two times a day, once a day, every other day, every third day, every week, every two weeks, every three weeks, every four weeks, every 2 months, every three months, every 6 months, etc. In some embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. In exemplary embodiments, compositions may be administered at dosage levels sufficient to deliver 0.0005 mg/kg to 0.01 mg/kg, e.g., about 0.0005 mg/kg to about 0.0075 mg/kg, e.g., about 0.0005 mg/kg, about 0.001 mg/kg, about 0.002 mg/kg, about 0.003 mg/kg, about 0.004 mg/kg or about 0.005 mg/kg.

In some embodiments, the compositions may be administered once or twice (or more) at dosage levels sufficient to deliver 0.025 mg/kg to 0.250 mg/kg, 0.025 mg/kg to 0.500 mg/kg, 0.025 mg/kg to 0.750 mg/kg, or 0.025 mg/kg to 1.0 mg/kg.

In some embodiments, the compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.0100 mg, 0.025 mg, 0.050 mg, 0.075 mg, 0.100 mg, 0.125 mg, 0.150 mg, 0.175 mg, 0.200 mg, 0.225 mg, 0.250 mg, 0.275 mg, 0.300 mg, 0.325 mg, 0.350 mg, 0.375 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.775 mg, 0.800 mg, 0.825 mg, 0.850 mg, 0.875 mg, 0.900 mg, 0.925 mg, 0.950 mg, 0.975 mg, or 1.0 mg. Higher and lower dosages and frequency of administration are encompassed by the present disclosure. For example, a composition described herein may be administered three or four times.

In some embodiments, the compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.010 mg, 0.025 mg, 0.100 mg or 0.400 mg.

In some embodiments, the composition (e.g., vaccine) for use in a method of vaccinating a subject is administered to the subject as a single dosage of between 10 μg/kg and 400 μg/kg of the nucleic acid vaccine (in an effective amount to vaccinate the subject). In some embodiments, the composition (e.g., vaccine) for use in a method of vaccinating a subject is administered to the subject as a single dosage of between 10 μg and 400 μg of the nucleic acid vaccine (in an effective amount to vaccinate the subject). In some embodiments, a composition (e.g., vaccine) for use in a method of vaccinating a subject is administered to the subject as a single dosage of 25-1000 μg (e.g., a single dosage of mRNA encoding an infection agent and a mRNA encoding T-cell epitope, or an mRNA encoding an infection agent and at least one universal T-cell epitope). In some embodiments, a composition (e.g., vaccine) is administered to the subject as a single dosage of 25, 50, 100, 150, 200, 250, 300, 350, 400, 450, 500, 550, 600, 650, 700, 750, 800, 850, 900, 950 or 1000 μg. For example, a composition (e.g., vaccine) may be administered to a subject as a single dose of 25-100, 25-500, 50-100, 50-500, 50-1000, 100-500, 100-1000, 250-500, 250-1000, or 500-1000 μg. In some embodiments, a composition (e.g., vaccine) for use in a method of vaccinating a subject is administered to the subject as two dosages, the combination of which equals 25-1000 μg of the composition (e.g., vaccine).

A composition (e.g., vaccine pharmaceutical composition) described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

### Methods of Use

Described herein are methods of activating and/or expanding an antigen-presenting cell said method including co-culturing the antigen-presenting cell and a cell including the single chain trimer nucleic acid described herein, the compositions described herein, the nanoparticles described herein, the vaccines described herein, or the pharmaceutical composition described herein. In some embodiments, the method of activating and/or expanding an antigen-presenting cell can include co-culturing an antigen-presenting cell and a cell including the lipid nanoparticles described herein.

Described herein are also methods of treating a subject with a viral infection, bacterial infection, parasitic infection and/or a cancer said method including administering to the subject a therapeutically effective amount of including the single chain trimer nucleic acid described herein, the compositions described herein, the nanoparticles described herein, the vaccines described herein, the cells described herein, or the pharmaceutical composition described herein. In some embodiments, described are methods of treating a subject with a viral infection, bacterial infection, parasitic infection and/or a cancer said method including administering to the subject a therapeutically effective amount of a lipid nanoparticle described herein.

In some embodiments, described herein are methods of treating a subject with a cancer said method including administering to the subject a therapeutically effective amount of including the single chain trimer nucleic acid described herein, the compositions described herein, the nanoparticles described herein, the vaccines described herein, the cells described herein, or the pharmaceutical composition described herein. In some embodiments, described are methods of treating a subject with a cancer said method including administering to the subject a therapeutically effective amount of lipid nanoparticle described herein.

Described herein are also methods of treating a subject with a viral infection, bacterial infection, parasitic infection and/or a cancer said method including obtaining antigen-presenting cells from the subject; contacting the antigen-presenting cells from the subject with the single chain trimer nucleic acid described herein, the compositions described herein, the nanoparticles described herein, the vaccines described herein, or the pharmaceutical composition described herein thereby activating the antigen-presenting cells, and administering the activated antigen-presenting cells to the subject.

In some embodiments, described are methods of treating a subject with a viral infection, bacterial infection, parasitic infection and/or a cancer said method including obtaining antigen-presenting cells from the subject; contacting the antigen-presenting cells from the subject with a lipid nanoparticle described herein thereby activating the antigen-presenting cells, and administering the activated antigen-presenting cells to the subject.

In some embodiments, described herein are methods of treating a subject with a cancer said method including obtaining antigen-presenting cells from the subject; contacting the antigen-presenting cells from the subject including the single chain trimer nucleic acid described herein, the compositions described herein, the nanoparticles described herein, the vaccines described herein, the cells described herein, or the pharmaceutical composition described herein thereby activating the antigen-presenting cells, and administering the activated antigen-presenting cells to the subject.

In some embodiments, described are methods of treating a subject with a cancer said method including obtaining antigen-presenting cells from the subject; contacting the antigen-presenting cells from the subject with a lipid nanoparticle described herein thereby activating the antigen-presenting cells, and administering the activated antigen-presenting cells to the subject.

Described herein are also methods of preventing a subject with a viral infection, bacterial infection, parasitic infection and/or a cancer said method including administering to the subject a therapeutically effective amount of including the single chain trimer nucleic acid described herein, the compositions described herein, the nanoparticles described herein, the vaccines described herein, the cells described herein, or the pharmaceutical composition described herein. In some embodiments, described are methods of preventing a subject with a viral infection, bacterial infection, parasitic infection and/or a cancer said method including administering to the subject a therapeutically effective amount of a lipid nanoparticle described herein.

In some embodiments, described herein are methods of preventing a subject with a cancer said method including administering to the subject a therapeutically effective amount of including the single chain trimer nucleic acid described herein, the compositions described herein, the nanoparticles described herein, the vaccines described herein, the cells described herein, or the pharmaceutical composition described herein. In some embodiments, described are methods of preventing a subject with a cancer said method including administering to the subject a therapeutically effective amount of lipid nanoparticle described herein.

Described herein are also methods of preventing a subject with a viral infection, bacterial infection, parasitic infection and/or a cancer said method including obtaining antigen-presenting cells from the subject; contacting the antigen-presenting cells from the subject with including the single chain trimer nucleic acid described herein, the compositions described herein, the nanoparticles described herein, the vaccines described herein, the cells described herein, or the pharmaceutical composition described herein thereby activating the antigen-presenting cells, and administering the activated antigen-presenting cells to the subject.

In some embodiments, described are methods of preventing a subject with a viral infection, bacterial infection, parasitic infection and/or a cancer said method including obtaining antigen-presenting cells from the subject; contacting the antigen-presenting cells from the subject with a lipid nanoparticle described herein thereby activating the antigen-presenting cells, and administering the activated antigen-presenting cells to the subject.

In some embodiments, described are methods of preventing a subject with a cancer said method including obtaining antigen-presenting cells from the subject; contacting the antigen-presenting cells from the subject with including the single chain trimer nucleic acid described herein, the compositions described herein, the nanoparticles described herein, the vaccines described herein, the cells described herein, or the pharmaceutical composition described herein thereby activating the antigen-presenting cells, and administering the activated antigen-presenting cells to the subject.

In some embodiments, described are methods of preventing a subject with a cancer said method including obtaining antigen-presenting cells from the subject; contacting the antigen-presenting cells from the subject with a lipid nanoparticle described herein thereby activating the antigen-presenting cells, and administering the activated antigen-presenting cells to the subject.

In some embodiments, the antigen-presenting cell can be a dendritic cell, B cell, macrophage, or other cells derived from a subject that can be used to for immunotherapy.

In some cases, the cancer is a circulating cancer cell (circulating tumor cell). In some cases, the cancer is a metastatic cancer cell. In some embodiments, the compositions and methods described herein are used to treat both local and metastatic tumors. In some embodiments, the compositions and methods described herein are useful for treating or preventing metastasis or recurrence of a cancer. In some embodiments, the compositions and methods described herein are useful for the prevention of recurrence of excised solid tumors. In some embodiments, the compositions and methods described herein are useful for the prevention of metastasis of excised solid tumors.

In one aspect, the methods described herein are used to treat cancer, for example, melanoma, lung cancer (including lung adenocarcinoma, basal cell carcinoma, squamous cell carcinoma, large cell carcinoma, bronchioloalveolar carcinoma, bronchogenic carcinoma, non-small-cell carcinoma, small cell carcinoma, mesothelioma); breast cancer (including ductal carcinoma, lobular carcinoma, inflammatory breast cancer, clear cell carcinoma, mucinous carcinoma, serosal cavities breast carcinoma); colorectal cancer (colon cancer, rectal cancer, colorectal adenocarcinoma); anal cancer; pancreatic cancer (including pancreatic adenocarcinoma, islet cell carcinoma, neuroendocrine tumors); prostate cancer; prostate adenocarcinoma; ovarian carcinoma (ovarian epithelial carcinoma or surface epithelial-stromal tumor including serous tumor, endometrioid tumor and mucinous cystadenocarcinoma, sex-cord-stromal tumor); liver and bile duct carcinoma (including hepatocellular carcinoma, cholangiocarcinoma, hemangioma); esophageal carcinoma (including esophageal adenocarcinoma and squamous cell carcinoma); oral and oropharyngeal squamous cell carcinoma; salivary gland adenoid cystic carcinoma; bladder cancer; bladder carcinoma; carcinoma of the uterus (including endometrial adenocarcinoma, ocular, uterine papillary serous carcinoma, uterine clear-cell carcinoma, uterine sarcomas, leiomyosarcomas, mixed mullerian tumors); glioma, glioblastoma, medulloblastoma, and other tumors of the brain; kidney cancers (including renal cell carcinoma, clear cell carcinoma, Wilm's tumor); cancer of the head and neck (including squamous cell carcinomas); cancer of the stomach (gastric cancers, stomach adenocarcinoma, gastrointestinal stromal tumor); testicular cancer; germ cell tumor; neuroendocrine tumor; cervical cancer; carcinoids of the gastrointestinal tract, breast, and other organs; signet ring cell carcinoma; mesenchymal tumors including sarcomas, fibrosarcomas, haemangioma, angiomatosis, haemangiopericytoma, pseudoangiomatous stromal hyperplasia, myofibroblastoma, fibromatosis, inflammatory myofibroblastic tumor, lipoma, angiolipoma, granular cell tumor, neurofibroma, schwannoma, angiosarcoma, liposarcoma, rhabdomyosarcoma, osteosarcoma, leiomyoma, leiomysarcoma, skin, including melanoma, cervical, retinoblastoma, head and neck cancer, pancreatic, brain, thyroid, testicular, renal, bladder, soft tissue, adenal gland, urethra, cancers of the penis, myxosarcoma, chondrosarcoma, osteosarcoma, chordoma, malignant fibrous histiocytoma, lymphangiosarcoma, mesothelioma, squamous cell carcinoma; epidermoid carcinoma, malignant skin adnexal tumors, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma anaplastic; glioblastoma multiforme, neuroblastoma, medulloblastoma, malignant meningioma, malignant schwannoma, neurofibrosarcoma, parathyroid carcinoma, medullary carcinoma of thyroid, bronchial carcinoid, pheochromocytoma, Islet cell carcinoma, malignant carcinoid, malignant paraganglioma, melanoma, Merkel cell neoplasm, cystosarcoma phylloide, salivary cancers, thymic carcinomas, and cancers of the vagina among others.

In one embodiment, bacterial organisms that can cause bacterial infection can include, but are not limited to Streptococcus pneumoniae, Haemophilus influenzae, Staphylococcus aureus, Streptococcus pyrogenes, Corynebacterium diphtheriae, Listeria monocytogenes, Bacillus anthracis, Clostridium tetani, Clostridium botulinum, Clostridium perfringens, Neisseria meningitidis, Neisseria gonorrhoeae, Streptococcus mutans, Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptospirosis interrogans, Borrelia burgdorferi, Camphylobacter jejuni. Viruses that can cause viral infection include, but are not limited to Metapneumovirus such as human Metapneumovirus (hMPV), parainfluenza viruses such as human parainfluenza viruses (hPIV) types 1, 2, and 3 (hPIV1, hPIV2 and hPIV3, respectively), respiratory syncytial virus (RSV), measles virus (MeV), coronaviruses (e.g., MERS-COV, SARS-CoV, SARS-COV2, HCoV-OC43, HCOV-229E, HCoV-NL63, HCoV-NL, HCoV-NH, HCoV-HKU1), poxviruses (e.g., smallpox, monkeypox), influenza A and B, human immunodeficiency virus (HIV), varicella-zoster, herpes simplex 1 and 2, cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, and the like. Other organism that can cause infections include fungal, protozoan, and parasitic organisms such as Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis, Schistosoma mansoni, and the like.

In some embodiments, the methods can further include administering an additional active agent. In some embodiments, the additional active agent.

As used herein, an “active agent” refers to therapeutic agents, diagnostic agents, or prophylactic agents. As discussed herein, the therapeutic agents can be released from the disclosed compounds, compositions, and systems in a biologically active form.

It is further understood, that as used herein, the terms “therapeutic agents” refers to one or more therapeutic agents, active ingredients, or substances that can be used to treat a medical condition. Therapeutic agent includes any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins and minerals such as essential amino acids, calcium, iron, potassium, zinc, vitamin B12, and the like; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; an antimicrobial agents (including antibiotics, antiviral agents, antiparasitic, and anti-fungal agents), anti-inflammatory agents (including steroids and non-steroidal anti-inflammatory agents), anti-coagulant agents, ophthalmic agents, gastrointestinal drugs, antiplatelet agents, and antiseptic agents, steroidal agent, anti-neoplastic agent, anti-cancer agent, antigen, antibody (e.g., cetuximab, anti-CD24 antibody, panitumumab and bevacizumab), birth control agent, progestational agent, anti-cholinergic, nutritional agent, analgesics and analgesic combinations such as acetaminophen, acetylsalicylic acid, and the like; anesthetics such as lidocaine, xylocaine, and the like, anorexics such as dexadrine, phendimetrazine tartrate, and the like; anti-epileptics, local and general anesthetics, hypnotics, sedatives, antipsychotic agents, neuroleptic agents, antidepressants such as isocarboxazid, amoxapine, and the like; anxiolytics, antagonists, neuron blocking agents, anticholinergic and cholinomimetic agents, antimuscarinic and muscarinic agents, antiparkinsonian agents, anti-Alzheimer's agents, antiadrenergics, antiarrhythmics, antihypertensive agents, hormones such as insulin, progestins, estrogens, corticoids, glucocorticoids, androgens, and the like; and nutrients, antiarthritics such as methylprednisolone, ibuprofen, and the like; antiasthmatics such as terbutaline sulfate, theophylline, ephedrine, and the like; anticonvulsants such as phenyloin sodium, diazepam, and the like; antiallergenics, antihistamines such as diphenhydramine HCl, chlorpheniramine maleate, and the like; antinauseants, antineoplastics, antipruritics, antipyretics; antispasmodics such as belladonna alkaloids, dicyclomine hydrochloride, and the like; cardiovascular agents such as prazosin HCl, nitroglycerin, propranolol HCl, hydralazine HCl, pancrelipase, succinic acid dehydrogenase, and the like; vasoactive agent, cardiovascular preparations (including calcium channel blockers, beta-blockers, beta-agonists and antiarrythmics), antihypertensives, diuretics such as furosemide, spironolactone, and the like; vasodilators; central nervous system stimulants; cough and cold preparations; decongestants; diagnostics; bone growth stimulants and bone resorption inhibitors; muscle relaxants; psychostimulants; sedatives; tranquilizers such as thorazine, diazepam, chlorpromazine HCl, reserpine, chlordiazepoxide HCl, and the like; antiulcer drugs such as rantidine HCl, cimetidine HCl, and the like; anti-asthmatic agents, anti-diarrheals, anti-obesity agents, anti-thrombotic agents, anti-tussive agents, anti-uricemic agents, anti-anginal agents, appetite suppressants, expectorants, hyperglycemic agents, hypoglycemic agents, thyroid and anti-thyroid agents, tissue growth agents, uterine relaxants, immunomodulator, including, for example, cytokines, interleukins, interferon, colony stimulating factor, tumor necrosis factor, and the like; immunosuppressants such as rapamycin, tacrolimus, and the like; immunological agent; antigens, factors, growth factors, amino acids, peptides and proteins and fragments thereof (whether naturally occurring, chemically synthesized or recombinantly produced) such as LHRH, somatostatin, calcitonin, growth hormone, glucagon-like peptides, growth releasing factor, angiotensin, FSH, EGF, bone morphogenic protein (BMP), erythopoeitin (EPO), interferon, interleukin, collagen, fibrinogen, insulin, Factor VIII, Factor IX, Enbrel®, Rituxam®, Herceptin®, alpha-glucosidase, Cerazyme/Ceredose®, vasopressin, ACTH, human serum albumin, gamma globulin, structural proteins, blood product proteins, complex proteins, antigens or antigenic polypeptides, enzymes, antibodies, monoclonal antibodies, and the like; and nucleic acid molecules (polymeric forms of two or more nucleotides, polynucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. In certain embodiments of the present disclosure, the agent to be delivered may be a mixture of active agents.

Representative examples of antibiotics include amikacin, amoxicillin, ampicillin, atovaquone, azithromycin, aztreonam, bacitracin, carbenicillin, cefadroxil, cefazolin, cefdinir, cefditoren, cefepime, cefiderocol, cefoperazone, cefotetan, cefoxitin, cefotaxime, cefpodoxime, cefprozil, ceftaroline, ceftazidime, ceftibuten, ceftizoxime, ceftriaxone, chloramphenicol, colistimethate, cefuroxime, cephalexin, cephradine, cilastatin, cinoxacin, ciprofloxacin, clarithromycin, clindamycin, dalbavancin, dalfopristin, daptomycin, demeclocycline, dicloxacillin, doripenem, doxycycline, eravacycline, ertapenem, erythromycin, fidaxomicin, fosfomycin, gatifloxacin, gemifloxacin, gentamicin, imipenem, lefamulin, lincomycin, linezolid, lomefloxacin, loracarbef, meropenem, metronidazole, minocycline, moxifloxacin, nafcillin, nalidixic acid, neomycin, norfloxacin, ofloxacin, omadacycline, oritavancin, oxacillin, oxytetracycline, paromomycin, penicillin, pentamidine, piperacillin, plazomicin, quinupristin, rifaximin, sarecycline, secnidazole, sparfloxacin, spectinomycin, sulfamethoxazole, sulfisoxazole, tedizolid, telavancin, telithromycin, ticarcillin, tigecycline, tobramycin, trimethoprim, trovafloxacin, and vancomycin.

Representative examples of antiviral agents include, but are not limited to, abacavir, acyclovir, adefovir, amantadine, amprenavir, atazanavir, balavir, baloxavir marboxil, boceprevir, cidofovir, cobicistat, daclatasvir, darunavir, delavirdine, didanosine, docasanol, dolutegravir, doravirine, ecoliever, edoxudine, efavirenz, elvitegravir, emtricitabine, enfuvirtide, entecavir, etravirine, famciclovir, fomivirsen, fosamprenavir, forscarnet, fosnonet, famciclovir, favipravir, fomivirsen, foscavir, ganciclovir, ibacitabine, idoxuridine, indinavir, inosine, inosine pranobex, interferon type I, interferon type II, interferon type III, lamivudine, letermovir, letermovir, lopinavir, loviride, maraviroc, methisazone, moroxydine, nelfinavir, nevirapine, nitazoxanide, oseltamivir, peginterferon alfa-2a, peginterferon alfa-2b, penciclovir, peramivir, pleconaril, podophyllotoxin, pyramidine, raltegravir, remdesevir, ribavirin, rilpivirine, rimantadine, rintatolimod, molnupiravir, ritonavir, saquinavir, simeprevir, sofosbuvir, stavudine, tarabivirin, telaprevir, telbivudine, tenofovir alafenamide, tenofovir disoproxil, tenofovir, tipranavir, trifluridine, trizivir, tromantadine, umifenovir, valaciclovir, valganciclovir, vidarabine, zalcitabine, zanamivir, and zidovudine.

Representative examples of anticoagulant agents include, but are not limited to, heparin, warfarin, rivaroxaban, dabigatran, apixaban, edoxaban, enoxaparin, and fondaparinux.

Representative examples of antiplatelet agents include, but are not limited to, clopidogrel, ticagrelor, prasugrel, dipyridamole, dipyridamole/aspirin, ticlopidine, and eptifibatide.

Representative examples of antifungal agents include, but are not limited to, voriconazole, itraconazole, posaconazole, fluconazole, ketoconazole, clotrimazole, isavuconazonium, miconazole, caspofungin, anidulafungin, micafungin, griseofulvin, terbinafine, flucytosine, terbinafine, nystatin, and amphotericin b.

Representative examples of steroidal anti-inflammatory agents include, but are not limited to, hydrocortisone, dexamethasone, prednisolone, prednisone, triamcinolone, methylprednisolone, budesonide, betamethasone, cortisone, and deflazacort. Representative examples of non-steroidal anti-inflammatory drugs include ibuprofen, naproxen, ketoprofen, tolmetin, etodolac, fenoprofen, flurbiprofen, diclofenac, piroxicam, indomethacin, sulindax, meloxicam, nabumetone, oxaprozin, mefenamic acid, and diflunisal.

Other examples of active agents include chloroquine, hydrochloroquine, Pyridoxal phosphate, Vitamin D, and Vitamin C.

Representative examples of anticytokine or immunomodulatory agents, but are not limited to, tocilizumab, sarilumab, bevacizumab, fingolimod, imiquimod, and eculizumab.

Immunotherapeutic agent can include but are not limited to an anti-CD40 antibody, an anti-PDL1 antibody (e.g., atezolizumab, durvalumab, or avelumab), an anti-PD1 antibody, an anti-CTLA4 antibody, programmed death protein 1 (PD-1) inhibitor or programmed death protein ligand 1 or 2 inhibitor include, (e.g., nivolumab (BMS), pembrolizumab (Merck), pidilizumab (CureTech/Teva), AMP-244 (Amplimmune/GSK), BMS-936559 (BMS), and MEDI4736 (Roche/Genentech)), or a combination thereof.

Representative examples of contraceptives include, but are not limited to, progestins, estrogens, or any combination thereof. For example, suitable progestins include, but are not limited to, natural and synthetic compounds having progestational activity, such as, for example, progesterone, chlormadinone acetate, norethindrone, cyproterone acetate, norethindrone acetate, desogestrel, levonorgestrel, drospirenone, trimegestone, norgestrel, norgestimate, norelgestromin, etonogestrel, gestodene, and other natural and/or synthetic gestagens. For example suitable estrogens include, but are not limited to, natural and synthetic compounds having estrogenic activity, such as, for example, estradiol (17β-estradiol), 17α-estradiol, estriol, estrone, and their esters, such as the acetate, sulfate, valerate or benzoate esters of these compounds, including, for example, estradiol 17β-cypionate, estradiol 17-propionate, estradiol 3-benzoate, and piperazine estrone sulfate; ethinyl estradiol; conjugated estrogens (natural and synthetic); mestranol; agonistic anti-estrogens; and selective estrogen receptor modulators. Other examples of contraceptives include gonodotropin releasing hormone (GnRh) or anologs thereof such as deslorelin, avorelin, leuprolide, triptorelin, nafarelin, goserelin, buserelin, and fertirelin.

The term “steroid” refers to compounds belonging to or related to the following illustrative families of compounds: corticosteroids, mineralicosteroids, and sex steroids (including, for example, potentially androgenic or estrogenic or anti-androgenic and anti-estrogenic molecules). Included among these are, for example, prednisone, prednisolone, methyl-prednisolone, triamcinolone, fluocinolone, aldosterone, spironolactone, danazol (otherwise known as OPTINA), and others. In some embodiments, the therapeutic agent may comprise a steroid.

Exemplary cancer drugs or anti-cancer agents can include, but are not limited to, antimetabolite anti-cancer agents and antimitotic anti-cancer agents, and combinations thereof. Various antimetabolite and antimitotic anti-cancer agents, including single such agents or combinations of such agents, may be employed in the methods and compositions described herein.

Antimetabolic anti-cancer agents typically structurally resemble natural metabolites, which are involved in normal metabolic processes of cancer cells such as the synthesis of nucleic acids and proteins. The antimetabolites, however, differ enough from the natural metabolites such that they interfere with the metabolic processes of cancer cells. In the cell, antimetabolites are mistaken for the metabolites they resemble, and are processed by the cell in a manner analogous to the normal compounds. The presence of the “decoy” metabolites prevents the cells from carrying out vital functions and the cells are unable to grow and survive. For example, antimetabolites may exert cytotoxic activity by substituting these fraudulent nucleotides into cellular DNA, thereby disrupting cellular division, or by inhibition of critical cellular enzymes, which prevents replication of DNA.

In one aspect, therefore, the antimetabolite anti-cancer agent is a nucleotide or a nucleotide analog. In certain aspects, for example, the antimetabolite agent may comprise purine (e.g., guanine or adenosine) or analogs thereof, or pyrimidine (cytidine or thymidine) or analogs thereof, with or without an attached sugar moiety.

Suitable antimetabolite anti-cancer agents for use in the present disclosure may be generally classified according to the metabolic process they affect, and can include, but are not limited to, analogues and derivatives of folic acid, pyrimidines, purines, and cytidine. Thus, in one aspect, the antimetabolite agent(s) is selected from the group consisting of cytidine analogs, folic acid analogs, purine analogs, pyrimidine analogs, and combinations thereof.

In one particular aspect, for example, the antimetabolite agent is a cytidine analog. According to this aspect, for example, the cytidine analog may be selected from the group consisting of cytarabine (cytosine arabinodside), azacitidine (5-azacytidine), and salts, analogs, and derivatives thereof.

In another particular aspect, for example, the antimetabolite agent is a folic acid analog. Folic acid analogs or antifolates generally function by inhibiting dihydrofolate reductase (DHFR), an enzyme involved in the formation of nucleotides; when this enzyme is blocked, nucleotides are not formed, disrupting DNA replication and cell division. According to certain aspects, for example, the folic acid analog may be selected from the group consisting of denopterin, methotrexate (amethopterin), pemetrexed, pteropterin, raltitrexed, trimetrexate, and salts, analogs, and derivatives thereof.

In another particular aspect, for example, the antimetabolite agent is a purine analog. Purine-based antimetabolite agents function by inhibiting DNA synthesis, for example, by interfering with the production of purine containing nucleotides, adenine and guanine which halts DNA synthesis and thereby cell division. Purine analogs can also be incorporated into the DNA molecule itself during DNA synthesis, which can interfere with cell division. According to certain aspects, for example, the purine analog may be selected from the group consisting of acyclovir, allopurinol, 2-aminoadenosine, arabinosyl adenine (ara-A), azacitidine, azathiprine, 8-aza-adenosine, 8-fluoro-adenosine, 8-methoxy-adenosine, 8-oxo-adenosine, cladribine, deoxycoformycin, fludarabine, gancylovir, 8-aza-guanosine, 8-fluoro-guanosine, 8-methoxy-guanosine, 8-oxo-guanosine, guanosine diphosphate, guanosine diphosphate-beta-L-2-aminofucose, guanosine diphosphate-D-arabinose, guanosine diphosphate-2-fluorofucose, guanosine diphosphate fucose, mercaptopurine (6-MP), pentostatin, thiamiprine, thioguanine (6-TG), and salts, analogs, and derivatives thereof.

In yet another particular aspect, for example, the antimetabolite agent is a pyrimidine analog. Similar to the purine analogs discussed above, pyrimidine-based antimetabolite agents block the synthesis of pyrimidine-containing nucleotides (cytosine and thymine in DNA; cytosine and uracil in RNA). By acting as “decoys,” the pyrimidine-based compounds can prevent the production of nucleotides, and/or can be incorporated into a growing DNA chain and lead to its termination. According to certain aspects, for example, the pyrimidine analog may be selected from the group consisting of ancitabine, azacitidine, 6-azauridine, bromouracil (e.g., 5-bromouracil), capecitabine, carmofur, chlorouracil (e.g. 5-chlorouracil), cytarabine (cytosine arabinoside), cytosine, dideoxyuridine, 3′-azido-3′-deoxythymidine, 3′-dideoxycytidin-2′-ene, 3′-deoxy-3′-deoxythymidin-2′-ene, dihydrouracil, doxifluridine, enocitabine, floxuridine, 5-fluorocytosine, 2-fluorodeoxycytidine, 3-fluoro-3′-deoxythymidine, fluorouracil (e.g., 5-fluorouracil (also known as 5-FU), gemcitabine, 5-methylcytosine, 5-propynylcytosine, 5-propynylthymine, 5-propynyluracil, thymine, uracil, uridine, and salts, analogs, and derivatives thereof. In one aspect, the pyrimidine analog is other than 5-fluorouracil. In another aspect, the pyrimidine analog is gemcitabine or a salt thereof.

In certain aspects, the antimetabolite agent is selected from the group consisting of 5-fluorouracil, capecitabine, 6-mercaptopurine, methotrexate, gemcitabine, cytarabine, fludarabine, pemetrexed, and salts, analogs, derivatives, and combinations thereof. In other aspects, the antimetabolite agent is selected from the group consisting of capecitabine, 6-mercaptopurine, methotrexate, gemcitabine, cytarabine, fludarabine, pemetrexed, and salts, analogs, derivatives, and combinations thereof. In one particular aspect, the antimetabolite agent is other than 5-fluorouracil. In a particularly preferred aspect, the antimetabolite agent is gemcitabine or a salt or thereof (e.g., gemcitabine HCl (Gemzar®)).

Other antimetabolite anti-cancer agents may be selected from, but are not limited to, the group consisting of acanthifolic acid, aminothiadiazole, brequinar sodium, Ciba-Geigy CGP-30694, cyclopentyl cytosine, cytarabine phosphate stearate, cytarabine conjugates, Lilly DATHF, Merrel Dow DDFC, dezaguanine, dideoxycytidine, dideoxyguanosine, didox, Yoshitomi DMDC, Wellcome EHNA, Merck & Co. EX-015, fazarabine, fludarabine phosphate, N-(2′-furanidyl)-5-fluorouracil, Daiichi Seiyaku FO-152, 5-FU-fibrinogen, isopropyl pyrrolizine, Lilly LY-188011; Lilly LY-264618, methobenzaprim, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, tiazofurin, Erbamont TIF, tyrosine kinase inhibitors, Taiho UFT and uricytin, among others.

In one aspect, the antimitotic anti-cancer agent is a microtubule inhibitor or a microtubule stabilizer. In general, microtubule stabilizers, such as taxanes and epothilones, bind to the interior surface of the beta-microtubule chain and enhance microtubule assembly by promoting the nucleation and elongation phases of the polymerization reaction and by reducing the critical tubulin subunit concentration required for microtubules to assemble. Unlike mictrotubule inhibitors, such as the vinca alkaloids, which prevent microtubule assembly, the microtubule stabilizers, such as taxanes, decrease the lag time and dramatically shift the dynamic equilibrium between tubulin dimers and microtubule polymers towards polymerization. In one aspect, therefore, the microtubule stabilizer is a taxane or an epothilone. In another aspect, the microtubule inhibitor is a vinca alkaloid.

In some embodiments, the anti-cancer agent may comprise a taxane or derivative or analog thereof. The taxane may be a naturally derived compound or a related form, or may be a chemically synthesized compound or a derivative thereof, with antineoplastic properties. The taxanes are a family of terpenes, including, but not limited to paclitaxel (Taxol®) and docetaxel (Taxotere®), which are derived primarily from the Pacific yew tree, Taxus brevifolia, and which have activity against certain tumors, particularly breast and ovarian tumors. In one aspect, the taxane is docetaxel or paclitaxel. Paclitaxel is a preferred taxane and is considered an antimitotic agent that promotes the assembly of microtubules from tubulin dimers and stabilizes microtubules by preventing depolymerization. This stability results in the inhibition of the normal dynamic reorganization of the microtubule network that is essential for vital interphase and mitotic cellular functions.

Also included are a variety of known taxane derivatives, including both hydrophilic derivatives, and hydrophobic derivatives. Taxane derivatives include, but are not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; deoxygenated paclitaxel compounds such as those described in U.S. Pat. No. 5,440,056; and taxol derivatives described in U.S. Pat. No. 5,415,869. As noted above, it further includes prodrugs of paclitaxel including, but not limited to, those described in WO 98/58927; WO 98/13059; and U.S. Pat. No. 5,824,701. The taxane may also be a taxane conjugate such as, for example, paclitaxel-PEG, paclitaxel-dextran, paclitaxel-xylose, docetaxel-PEG, docetaxel-dextran, docetaxel-xylose, and the like. Other derivatives are mentioned in “Synthesis and Anticancer Activity of Taxol Derivatives,” D. G. I. Kingston et al., Studies in Organic Chemistry, vol. 26, entitled “New Trends in Natural Products Chemistry” (1986), Atta-ur-Rabman, P. W. le Quesne, Eds. (Elsevier, Amsterdam 1986), among other references. Each of these references is hereby incorporated by reference herein in its entirety.

Various taxanes may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267) (each of which is hereby incorporated by reference herein in its entirety), or obtained from a variety of commercial sources, including for example, Sigma-Aldrich Co., St. Louis, Mo.

Alternatively, the antimitotic anti-cancer agent can be a microtubule inhibitor; in one preferred aspect, the microtubule inhibitor is a vinca alkaloid. In general, the vinca alkaloids are mitotic spindle poisons. The vinca alkaloid agents act during mitosis when chromosomes are split and begin to migrate along the tubules of the mitosis spindle towards one of its poles, prior to cell separation. Under the action of these spindle poisons, the spindle becomes disorganized by the dispersion of chromosomes during mitosis, affecting cellular reproduction. According to certain aspects, for example, the vinca alkaloid is selected from the group consisting of vinblastine, vincristine, vindesine, vinorelbine, and salts, analogs, and derivatives thereof.

The antimitotic anti-cancer agent can also be an epothilone. In general, members of the epothilone class of compounds stabilize microtubule function according to mechanisms similar to those of the taxanes. Epothilones can also cause cell cycle arrest at the G2-M transition phase, leading to cytotoxicity and eventually apoptosis. Suitable epithiolones include epothilone A, epothilone B, epothilone C, epothilone D, epothilone E, and epothilone F, and salts, analogs, and derivatives thereof. One particular epothilone analog is an epothilone B analog, ixabepilone (Ixempra™).

In certain aspects, the antimitotic anti-cancer agent is selected from the group consisting of taxanes, epothilones, vinca alkaloids, and salts and combinations thereof. Thus, for example, in one aspect the antimitotic agent is a taxane. More preferably in this aspect the antimitotic agent is paclitaxel or docetaxel, still more preferably paclitaxel. In another aspect, the antimitotic agent is an epothilone (e.g., an epothilone B analog). In another aspect, the antimitotic agent is a vinca alkaloid.

Examples of cancer drugs that may be used in the present disclosure include, but are not limited to: thalidomide; platinum coordination complexes such as cisplatin (cis-DDP), oxaliplatin and carboplatin; anthracenediones such as mitoxantrone; substituted ureas such as hydroxyurea; methylhydrazine derivatives such as procarbazine (N-methylhydrazine, MIH); adrenocortical suppressants such as mitotane (o,p′-DDD) and aminoglutethimide; RXR agonists such as bexarotene; and tyrosine kinase inhibitors such as sunitimib and imatinib.

Examples of additional cancer drugs include alkylating agents, antimetabolites, natural products, hormones and antagonists, and miscellaneous agents. Alternate names are indicated in parentheses. Examples of alkylating agents include nitrogen mustards such as mechlorethamine, cyclophosphainide, ifosfamide, melphalan sarcolysin) and chlorambucil; ethylenimines and methylmelamines such as hexamethylmelamine and thiotepa; alkyl sulfonates such as busulfan; nitrosoureas such as carmustine (BCNU), semustine (methyl-CCNU), lomustine (CCNU) and streptozocin (streptozotocin); DNA synthesis antagonists such as estramustine phosphate; and triazines such as dacarbazine (DTIC, dimethyl-triazenoimidazolecarboxamide) and temozolomide. Examples of antimetabolites include folic acid analogs such as methotrexate (amethopterin); pyrimidine analogs such as fluorouracin (5-fluorouracil, 5-FU, SFU), floxuridine (fluorodeoxyuridine, FUdR), cytarabine (cytosine arabinoside) and gemcitabine; purine analogs such as mercaptopurine (6-mercaptopurine, 6-MP), thioguanine (6-thioguanine, TG) and pentostatin (2′-deoxycoformycin, deoxycoformycin), cladribine and fludarabine; and topoisomerase inhibitors such as amsacrine. Examples of natural products include vinca alkaloids such as vinblastine (VLB) and vincristine; taxanes such as paclitaxel, protein bound paclitaxel (Abraxane) and docetaxel (Taxotere); epipodophyllotoxins such as etoposide and teniposide; camptothecins such as topotecan and irinotecan; antibiotics such as dactinomycin (actinomycin D), daunorubicin (daunomycin, rubidomycin), doxorubicin, histrelin, bleomycin, mitomycin (mitomycin C), idarubicin, epirubicin; enzymes such as L-asparaginase; and biological response modifiers such as interferon alpha and interlelukin 2. Examples of hormones and antagonists include luteinising releasing hormone agonists such as buserelin; adrenocorticosteroids such as prednisone and related preparations; progestins such as hydroxyprogesterone caproate, rnedroxyprogesterone acetate and megestrol acetate; estrogens such as diethylstilbestrol and ethinyl estradiol and related preparations; estrogen antagonists such as tamoxifen and anastrozole; androgens such as testosterone propionate and fluoxymesterone and related preparations; androgen antagonists such as flutamide and bicalutamide; and gonadotropin-releasing hormone analogs such as leuprolide. Alternate names and trade-names of these and additional examples of cancer drugs, and their methods of use including dosing and administration regimens, will be known to a person versed in the art.

In some aspects, the anti-cancer agent may comprise a chemotherapeutic agent. Suitable chemotherapeutic agents include, but are not limited to, alkylating agents, antibiotic agents, antimetabolic agents, hormonal agents, plant-derived agents and their synthetic derivatives, anti-angiogenic agents, differentiation inducing agents, cell growth arrest inducing agents, apoptosis inducing agents, cytotoxic agents, agents affecting cell bioenergetics i.e., affecting cellular ATP levels and molecules/activities regulating these levels, biologic agents, e.g., monoclonal antibodies, kinase inhibitors and inhibitors of growth factors and their receptors, gene therapy agents, cell therapy, e.g., stem cells, or any combination thereof.

According to these aspects, the chemotherapeutic agent is selected from the group consisting of cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrexate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, erlotinib hydrochloride and combinations thereof. Each possibility represents a separate aspect of the invention.

Anti-neoplastic agent can be selected from the group consisting of Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Adrucil (Fluorouracil), Afatinib Dimaleate, Afinitor (Everolimus), Akynzeo (Netupitant and Palonosetron Hydrochloride), Aldara (Imiquimod), Aldesleukin, Alemtuzumab, Alimta (Pemetrexed Disodium), Aloxi (Palonosetron Hydrochloride), Ambochlorin (Chlorambucil), Amboclorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, BEACOPP, Becenum (Carmustine), Beleodaq (Belinostat), Belinostat, Bendamustine Hydrochloride, BEP, Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Busulfan, Busulfex (Busulfan), Cabazitaxel, Cabozantinib-S-Malate, CAF, Campath (Alemtuzumab), Camptosar (Irinotecan Hydrochloride), Capecitabine, CAPOX, Carboplatin, CARBOPLATIN-TAXOL, Carfilzomib, Carmubris (Carmustine), Carmustine, Carmustine Implant, Casodex (Bicalutamide), CeeNU (Lomustine), Ceritinib, Cerubidine (Daunorubicin Hydrochloride), Cervarix (Recombinant HPV Bivalent Vaccine), Cetuximab, Chlorambucil, CHLORAMBUCIL-PREDNISONE, CHOP, Cisplatin, Clafen (Cyclophosphamide), Clofarabine, Clofarex (Clofarabine), Clolar (Clofarabine), CMF, Cometriq (Cabozantinib-S-Malate), COPP, COPP-ABV, Cosmegen (Dactinomycin), Crizotinib, CVP, Cyclophosphamide, Cyfos (Ifosfamide), Cyramza (Ramucirumab), Cytarabine, Cytarabine, Liposomal, Cytosar-U (Cytarabine), Cytoxan (Cyclophosphamide), Dabrafenib, Dacarbazine, Dacogen (Decitabine), Dactinomycin, Dasatinib, Daunorubicin Hydrochloride, Decitabine, Degarelix, Denileukin Diftitox, Denosumab, DepoCyt (Liposomal Cytarabine), DepoFoam (Liposomal Cytarabine), Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Ifex (Ifosfamide), Ifosfamide, Ifosfamidum (Ifosfamide), Imatinib Mesylate, Imbruvica (Ibrutinib), Imiquimod, Inlyta (Axitinib), Interferon Alfa-2b, Recombinant, Intron A (Recombinant Interferon Alfa-2b), Iodine I 131 Tositumomab and Tositumomab, Ipilimumab, Iressa (Gefitinib), Irinotecan Hydrochloride, Istodax (Romidepsin), Ixabepilone, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Keytruda (Pembrolizumab), Kyprolis (Carfilzomib), Lanreotide Acetate, Lapatinib Ditosylate, Lenalidomide, Lenvatinib Mesylate, Lenvima (Lenvatinib Mesylate), Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Liposomal Cytarabine, Lomustine, Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Lynparza (Olaparib), Marqibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Netupitant and Palonosetron Hydrochloride, Neupogen (Filgrastim), Nexavar (Sorafenib Tosylate), Nilotinib, Nivolumab, Nolvadex (Tamoxifen Citrate), Nplate (Romiplostim), Obinutuzumab, Odomzo (Sonidegib), OEPA, Ofatumumab, OFF, Olaparib, Omacetaxine Mepesuccinate, Oncaspar (Pegaspargase), Ondansetron Hydrochloride, Ontak (Denileukin Diftitox), Opdivo (Nivolumab), OPPA, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, Pegaspargase, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, R-EPOCH, Revlimid (Lenalidomide), Rheumatrex (Methotrexate), Rituxan (Rituximab), Rituximab, Romidepsin, Romiplostim, Rubidomycin (Daunorubicin Hydrochloride), Ruxolitinib Phosphate, Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synovir (Thalidomide), Synribo (Omacetaxine Mepesuccinate), TAC, Tafinlar (Dabrafenib), Talc, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Thiotepa, Toposar (Etoposide), Topotecan Hydrochloride, Toremifene, Torisel (Temsirolimus), Tositumomab and Iodine I 131 Tositumomab, Totect (Dexrazoxane Hydrochloride), TPF, Trametinib, Trastuzumab, Treanda (Bendamustine Hydrochloride), Trisenox (Arsenic Trioxide), Tykerb (Lapatinib Ditosylate), Unituxin (Dinutuximab), Vandetanib, VAMP, Vectibix (Panitumumab), VeIP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Zaltrap (Ziv-Aflibercept), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and Zytiga (Abiraterone Acetate).

Growth factors useful as therapeutic agents include, but are not limited to, transforming growth factor-α (“TGF-α”), transforming growth factors (“TGF-β”), platelet-derived growth factors (“PDGF”), fibroblast growth factors (“FGF”), including FGF acidic isoforms 1 and 2, FGF basic form 2 and FGF 4, 8, 9 and 10, nerve growth factors (“NGF”) including NGF 2.5s, NGF 7.0s and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), granulocyte colony stimulating factor (G-CSF), insulin like growth factor (IGF) I and II, hepatocyte growth factor, glial neurotrophic growth factor (GDNF), stem cell factor (SCF), keratinocyte growth factor (KGF), transforming growth factors (TGF), including TGFs alpha, beta, beta1, beta2, beta3, skeletal growth factor, bone matrix derived growth factors, and bone derived growth factors and mixtures thereof.

Cytokines useful as anti-cancer agents can include, but are not limited to, cardiotrophin, stromal cell derived factor, macrophage derived chemokine (MDC), macrophage inflammatory proteins 1 alpha (MIP-1alpha), 2, 3 alpha, 3 beta, 4 and 5, interleukins (IL)-1, IL-2, IL-3, IL-4, IL-5, IL-6, IL-7, IL-8, IL-9, IL-10, IL-11, IL-12, IL-13, IL-15, IL-16, IL-17 and IL-18. TNF-α, and TNF-β, interferons (IFN) (e.g., interferon alpha, interferon alpha A, interferon beta, interferon gamma, interferon 1 alpha), growth factors (e.g., colony-stimulating factors (CSF), granulocyte colony-stimulating factor (G-CSF) (e.g., filgrastim (Neupogen®; Amgen)) hematopoietic growth factors (HGFs)), melanoma growth stimulatory activity (MGSA), monoclonal antibodies (mAb)-anti-CD40 antibody, an anti-PDL1 antibody (e.g., atezolizumab, durvalumab, or avelumab), an anti-PD1 antibody, an anti-CTLA4 antibody, programmed death protein 1 (PD-1) inhibitor or programmed death protein ligand 1 or 2 inhibitor include, (e.g., nivolumab (BMS), pembrolizumab (Merck), pidilizumab (CureTech/Teva), AMP-244 (Amplimmune/GSK), BMS-936559 (BMS), and MEDI4736 (Roche/Genentech)), or a combination thereof.

Immunoglobulins useful in the present disclosure include, but are not limited to, IgG, IgA, IgM, IgD, IgE, and mixtures thereof. Some preferred growth factors include VEGF (vascular endothelial growth factor), NGFs (nerve growth factors), PDGF-AA, PDGF-BB, PDGF-AB, FGFb, FGFa, and BGF.

Other molecules useful as anti-cancer agents include but are not limited to growth hormones, leptin, leukemia inhibitory factor (LIF), tumor necrosis factor alpha and beta, endostatin, thrombospondin, osteogenic protein-1, bone morphogenetic proteins 2 and 7, osteonectin, somatomedin-like peptide, osteocalcin.

Tumor antigens can be based on specific mutations (neoepitopes) and those expressed by cancer-germline genes (antigens common to tumors found in multiple patients, referred to herein as “traditional cancer antigens” or “shared cancer antigens”). In some embodiments, a traditional antigen is one that is known to be found in cancers or tumors generally or in a specific type of cancer or tumor. In some embodiments, a traditional cancer antigen is a non-mutated tumor antigen. In some embodiments, a traditional cancer antigen is a mutated tumor antigen. Tumor antigens can include, but are not limited to, CD2, CD19, CD20, CD22, CD27, CD33, CD37, CD38, CD40, CD44, CD47, CD52, CD56, CD70, CD79, CD137, 4-IBB, 5T4, AGS-5, AGS-16, Angiopoietin 2, B2M, B7.1, B7.2, B7DC, B7H1, B7H2, B7H3, BT-062, BTLA, CAIX, CTLA4, Cripto, ED-B, ErbB1, ErbB2, ErbB3, ErbB4, EGFL7, EpCAM, EphA2, EphA3, EphB2, FAP, Fibronectin, Folate Receptor, Ganglioside GM3, GD2, glucocorticoid-induced tumor necrosis factor receptor (GITR), gp100, gpA33, GPNMB, ICOS, IGFIR, Integrin av, Integrin αvβ, LAG-3, Lewis Y, Mesothelin, c-MET, MN Carbonic anhydrase IX, MUCI, MUC16, Nectin-4, NKGD2, NOTCH, OX40, OX40L, PD-1, PDL1, PSCA, PSMA, RANKL, ROR1, ROR2, SLC44A4, Syndecan-1, TACI, TAG-72, Tenascin, TIM3, TRAILR1, TRAILR2, VEGFR-1, VEGFR-2, VEGFR-3, cellular tumor antigen p53, abnormal products of ras, cancer antigen 15-3 (CA 15-3), cancer antigen 19-9 (CA 19-9), cancer antigen-125 (CA-125), cancer antigen 195 (CA 195), C219, cancer antigen 549 (CA 549), HER-2 or neu oncoprotein (C-erb B-2), MHC class II antigens (e.g., human leukocyte antigens (HLA)-A, HLA-B, HLA-C, HLA-A*01, HLA-A*02, or HLA-A*11), MHC class I antigens (e.g., human leukocyte antigens-DR, HLA-DQ, HLA-DP, HLA-DRB1*04, HLA-DRB1*07, or HLA-DRB1), oncofetal antigens (e.g., alphafetoprotein (AFP) and carcinoembryonic antigen (CEA)), cancer-testis antigens (eg. MAGEA1, MAGEA2, MAGEA3, MAGEA4, MAGEA5, MAGEA6, MAGEA8, MAGEA9, MAGEA10, MAGEA11, MAGEA12, BAGE, BAGE2, BAGE3, BAGE4, BAGE5, MAGEB1, MAGEB2, MAGEB5, MAGEB6, MAGEB3, MAGEB4, GAGE1, GAGE2A, GAGE3, GAGE4, GAGES, GAGE6, GAGE7, GAGE8, SSX1, SSX2, SSX2b, SSX3, SSX4, CTAG1B, LAGE-1b, CTAG2, MAGEC1, MAGEC3, SYCP1, BRDT, MAGEC2, SPANXA1, SPANXB1, SPANXC, SPANXD, SPANXN1, SPANXN2, SPANXN3, SPANXN4, SPANXN5, XAGE1D, XAGE1C, XAGE1B, XAGE1, XAGE2, XAGE3, XAGE-3b, XAGE-4/RP11-167P23.2, XAGE5, DDX43, SAGE1, ADAM2, PAGES, CT16.2, PAGE1, PAGE2, PAGE2B, PAGE3, PAGE4, LIPI, VENTXP1, IL13RA2, TSP50, CTAGE1, CTAGE-2, CTAGES, SPA17, ACRBP, CSAG1, CSAG2, DSCR8, MMA1b, DDX53, CTCFL, LUZP4, CASC5, TFDP3, JARID1B, LDHC, MORC1, DKKL1, SPO11, CRISP2, FMR1NB, FTHL17, NXF2, TAF7L, TDRD1, TDRD6, TDRD4, TEX15, FATE1, TPTE, CT45A1, CT45A2, CT45A3, CT45A4, CT45A5, CT45A6, HORMAD1, HORMAD2, CT47A1, CT47A2, CT47A3, CT47A4, CT47A5, CT47A6, CT47A7, CT47A8, CT47A9, CT47A10, CT47A11, CT47B1, SLCO6A1, TAG, LEMD1, HSPB9, CCDC110, ZNF165, SPACA3, CXorf48, THEG, ACTL8, NLRP4, COX6B2, LOC348120, CCDC33, LOC196993, PASD1, LOC647107, TULP2, CT66/AA884595, PRSS54, RBM46, CT69/BC040308, CT70/BI818097, SPINLW1, TSSK6, ADAM29, CCDC36, LOC440934, SYCE1, CPXCR1, TSPY3, TSGA10, HIWI, MIWI, PIWI, PIWIL2, ARMC3, AKAP3, Cxorf61, PBK, C21orf99, OIP5, CEP290, CABYR, SPAG9, MPHOSPH1, ROPN1, PLAC1, CALR3, PRM1, PRM2, CAGE1, TTK, LY6K, IMP-3, AKAP4, DPPA2, KIAA0100, DCAF12, SEMG1, POTED, POTEE, POTEA, POTEG, POTEB, POTEC, POTEH, GOLGAGL2 FA, CDCA1, PEPP2, OTOA, CCDC62, GPATCH2, CEP55, FAM46D, TEX14, CTNNA2, FAM133A, LOC130576, ANKRD45, ELOVL4, IGSF11, TMEFF1, TMEFF2, ARX, SPEF2, GPAT2, TMEM108, NOL4, PTPN20A, SPAG4, MAEL, RQCD1, PRAME, TEX101, SPATA19, ODF1, ODF2, ODF3, ODF4, ATAD2, ZNF645, MCAK, SPAG1, SPAG6, SPAG8, SPAG17, FBXO39, RGS22, cyclin A1, C15orf60, CCDC83, TEKT5, NR6A1, TMPRSS12, TPPP2, PRSS55, DMRT1, EDAG, NDR, DNAJB8, CSAG3B, CTAGIA, GAGE12B, GAGE12C, GAGE12D, GAGE12E, GAGE12F, GAGE12G, GAGE12H, GAGE12I, GAGE12J, GAGE13, LOC728137, MAGEA2B, MAGEA9B/LOC728269, NXF2B, SPANXA2, SPANXB2, SPANXE, SSX4B, SSX5, SSX6, SSX7, SSX9, TSPY1D, TSPY1E, TSPY1F, TSPY1G, TSPY1H, TSPY1I, TSPY2, XAGE1E, XAGE2B/CTD-2267G17.3, and/or variants thereof), oncogenic viral antigens (e.g., human papillomavirus (hpv) or hepatitis b), oncogenic bacterial antigens (e.g., Helicobacter pylori), and/or variants thereof.

Diagnostic agents include gases; metals; commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MRI include gadolinium chelates, as well as iron, magnesium, manganese, copper, and chromium. Examples of materials useful for CAT and x-ray imaging include iodine-based materials.

Vaccines may comprise isolated proteins or peptides, inactivated organisms and viruses, dead organisms and viruses, genetically altered organisms or viruses, cell extracts, and RNA encoding at least one antigenic polypeptide or an immunogenic fragment thereof (e.g., an immunogenic fragment capable of inducing an immune response to the antigenic polypeptide). Active agents may be combined with interleukins, interferon, cytokines, and adjuvants such as cholera toxin, alum, Freund's adjuvant, etc. Prophylactic agents can include antigens of bacterial organisms such as Streptococcus pneumoniae, Haemophilus influenzae, Staphylococcus aureus, Streptococcus pyrogenes, Corynebacterium diphtheriae, Listeria monocytogenes, Bacillus anthracis, Clostridium tetani, Clostridium botulinum, Clostridium perfringens, Neisseria meningitidis, Neisseria gonorrhoeae, Streptococcus mutans, Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptospirosis interrogans, Borrelia burgdorferi, Camphylobacter jejuni, and the like; antigens of viruses such as human Metapneumovirus (hMPV), human parainfluenza viruses (hPIV) types 1, 2, and 3 (hPIV1, hPIV2 and hPIV3, respectively), respiratory syncytial virus (RSV), measles virus (MeV), coronaviruses (e.g., MERS-COV, SARS-COV, SARS-COV2, HCoV-OC43, HCOV-229E, HCoV-NL63, HCOV-NL, HCoV-NH, HCoV-HKU1), poxviruses (e.g., smallpox, monkeypox), influenza A and B, HIV, varicella-zoster, herpes simplex 1 and 2, cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, and the like; antigens of fungal, protozoan, and parasitic organisms such as Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof.

The nucleic acids that can be introduced according to the methods herein include DNA, cDNA, and RNA sequences of all types. For example, the nucleic acid can be double stranded DNA, single-stranded DNA, complexed DNA, encapsulated DNA, naked RNA, encapsulated RNA, plasmid RNA, mini RNA, circular RNA, messenger RNA (mRNA), tRNA, short interfering RNA (siRNA), double stranded RNA (dsRNA), micro-RNA (miRNA), antisense RNA (asRNA), self-amplify mRNA (saRNA), guide RNA (gRNA), cRNA and combinations thereof. In some embodiments, the nucleic acid can include plasmid DNA, miniDNA, mRNA, self-amplifying RNA, or circular RNA. The nucleic acid can also be DNA constructs, such as expression vectors, expression vectors encoding a desired gene product (e.g., a gene product homologous or heterologous to the subject into which it is to be introduced), and the like.

In some embodiments, the nucleic acids (e.g., mRNA) may be used to induce a balanced immune response against an infection. In some embodiments, the nucleic acids (e.g., mRNA) may be used to induce a balanced immune response against viruses such as Metapneumovirus such as human Metapneumovirus (hMPV), parainfluenza viruses such as human parainfluenza viruses (hPIV) types 1, 2, and 3 (hPIV1, hPIV2 and hPIV3, respectively), respiratory syncytial virus (RSV), measles virus (MeV), coronaviruses (e.g., MERS-COV, SARS-COV, SARS-COV2, HCoV-OC43, HCoV-229E, HCoV-NL63, HCOV-NL, HCOV-NH, HCoV-HKU1), poxviruses (e.g., smallpox, monkeypox), influenza A and B, HIV, varicella-zoster, herpes simplex 1 and 2, cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, and the like. In some embodiments, the nucleic acid (e.g., mRNA) may be used to induce a balanced immune response against respiratory viruses. The term “respiratory viruses” refers herein to viruses causing respiratory diseases. For example, negative-sense, single-stranded RNA virus of the family Paramyxoviridae such as human Metapneumovirus (hMPV), human parainfluenza viruses (hPIV) types 1, 2, and 3 (hPIV1, hPIV2 and hPIV3, respectively), RSV, and Measles virus (MeV). Another example of respiratory viruses are coronaviruses. Coronaviruses are enveloped viruses with a positive-sense single-stranded RNA genome and with a nucleocapsid of helical symmetry. Coronaviruses are species of virus belonging to the subfamily Coronavirinae in the family Coronaviridae, in the order Nidovirales.

Representative examples of betacoronaviruses include, but are not limited to an embecovirus 1 (e.g., Betacoronavirus 1, Human coronavirus OC43, China Rattus coronavirus HKU24, Human coronavirus HKU1, Murine coronavirus), a hibecovirus (e.g., Bat Hp-betacoronavirus Zhejiang2013), a merbecovirus (e.g., Hedgehog coronavirus 1, Middle East respiratory syndrome-related coronavirus (MERS-COV), Pipistrellus bat coronavirus HKU5, Tylonycteris bat coronavirus HKU4), a nobecovirus (e.g., Rousettus bat coronavirus GCCDC1, Rousettus bat coronavirus HKU9), a sarbecovirus (e.g., severe acute respiratory syndrome coronavirus (SARS-COV), severe acute respiratory syndrome coronavirus 2 (SARS-COV-2).

Representative examples of gammacoronaviruses include, but are not limited to, a cegacovirus (e.g., Beluga whale coronavirus SQ1) and an Igacovirus (e.g., Avian coronavirus (IBV)).

Representative examples of deltacoronaviruses include, but are not limited to, an andecovirus (e.g., Wigeon coronavirus HKU20), a buldecovirus (e.g., Bulbul coronavirus HKU11, Porcine coronavirus HKU15 (PorCoV HKU15), Munia coronavirus HKU13, White-eye coronavirus HKU16), a herdecovirus (e.g., Night heron coronavirus HKU19), and a moordecovirus (e.g., Common moorhen coronavirus HKU21).

In some embodiments, the coronavirus is a human coronavirus. Representative examples of human coronaviruses include, but are not limited to, human coronavirus 229E (HCoV-229E), human coronavirus OC43 (HCoV-OC43), human coronavirus HKU1 (HCoV-HKU1), Human coronavirus NL63 (HCoV-NL63), severe acute respiratory syndrome coronavirus (SARS-COV), severe acute respiratory syndrome coronavirus 2 (SARS-COV-2), and Middle East respiratory syndrome-related coronavirus (MERS-COV).

In one embodiment, the coronavirus infection is an infection of the upper and/or lower respiratory tract. The “upper respiratory tract” includes the mouth, nose, sinus, middle ear, throat, larynx, and trachea. The “lower respiratory tract” includes the bronchial tubes (bronchi) and the lungs (bronchi, bronchioles and alveoli), as well as the interstitial tissue of the lungs.

In another embodiment, the coronavirus infection is an infection of the gastrointestinal tract. The “gastrointestinal tract” may include any area of the canal from the mouth to the anus, including the mouth, esophagus, stomach, and intestines. In yet another embodiment, the coronavirus infection is a renal infection.

Other indications associated with coronavirus infections are described in Gralinski & Baric, 2015, J. Pathol. 235:185-195 and Cavanagh, 2005, “Coronaviridae: a review of coronavirus and toroviruses”, Coronaviruses with Special Emphasis on First Insights Concerning SARS 1, ed. By A. Schmidt, M. H. Wolff and O. Weber, Birkhauser Verlag Baser, Switzerland, each of which is incorporated herein by reference in their entirety.

In some embodiments, the nucleic acids disclosed herein can include at least one chemically modified nucleotide. In some embodiments, the at least one chemically modified nucleotide comprises a chemically modified nucleobase, a chemically modified ribose, a chemically modified phosphodiester linkage, or a combination thereof.

In one embodiment, the at least one chemically modified nucleotide is a chemically modified nucleobase. In some embodiments, the mRNA can include a heterologous 5′ untranslated region (5′UTR). In some embodiments, the mRNA can include a heterologous 3′ untranslated region (3′UTR).

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically related may be substituted for the agents described herein while the same or similar results would be achieved. All such similar substitutes and modifications apparent to those skilled in the art are deemed to be within the spirit, scope and concept of the disclosure as defined by the appended claims.

By way of non-limiting illustration, examples of certain embodiments of the present disclosure are given below.

### EXAMPLES

To overcome the current challenges with cancer vaccines, we utilize lipid nanoparticle (LNP) delivery technology to deliver nucleic acids (DNA or RNA) to express single-chain trimer (SCT), which consists of peptide, β2-microglobulin (β2m), and MHC class I heavy chain to bypass antigen processing and result in stable presentation of the peptide on the surface of antigen-presenting cells, as well as a universal MHC II-restricted peptide to activate host memory CD4 T cells to enhance tumor-specific CD8 T cell immunity.

### Example 1: Kb-E6-SCT mRNA Induces a Robust Antigen-Specific CD8+ T Cell Immune Response and Demonstrates Potent Antitumor Activity in Animal Models

Design and Validation of Kb-E6-SCT mRNA

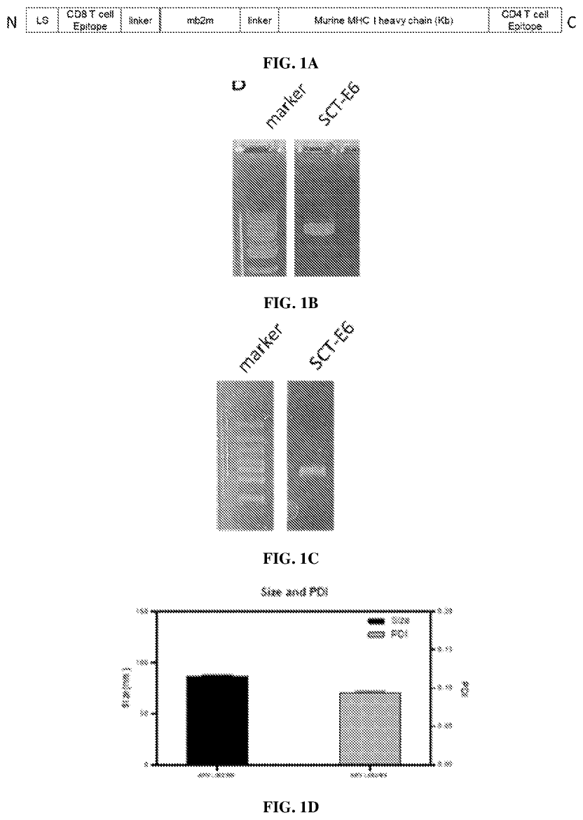

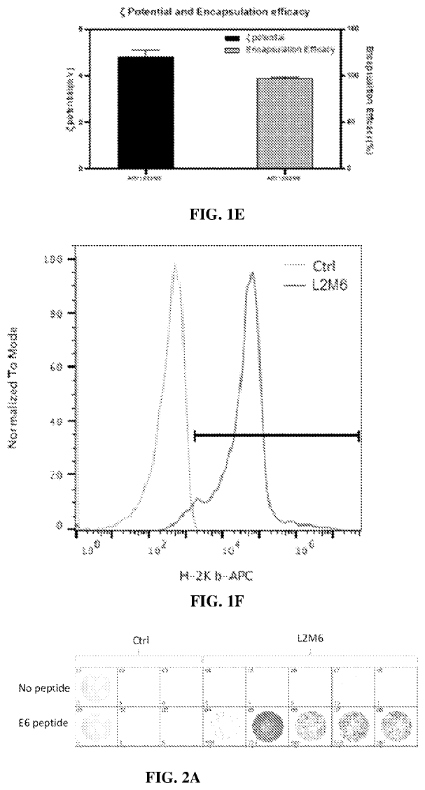

To establish the capability of mRNA encoding Kb-E6-SCT to enhance the efficacy of mRNA vaccines against HPV E6/E7-expressing tumors in animal models, we devised and expressed mRNA encoding SCT. The SCT comprises a peptide, β2m, and mouse MHC Class I, Kb. Kb-E6-SCT mRNA is illustrated in FIG. 1A and in vitro potency was studied, and results are shown in FIGS. 1B-1E. The linearized plasmid template is shown in FIG. 1B. The in vitro transcription of mRNA of SCT-E6 is shown in FIG. 1C. Results for the physicochemical characterization of LNP formulation and encapsulation efficacy of L2M6 are shown in FIGS. 1D-1E. FIG. 1D exhibits particle size and polydispersity index, and FIG. 1E demonstrates zeta potential and encapsulation efficiency. Additionally, FIG. 1F illustrates the expression of SCT-E6 in 293T cells post-transfection.

### Animal Studies

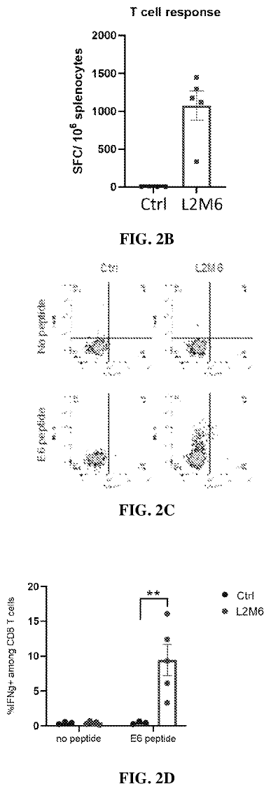

C57BL/6 mice were immunized i.m. with 1 μg mRNA of the indicated vaccine twice two weeks apart, 7 days after the last immunization, E6-CD8 T cell peptide was used to determine the E6 specific T cell population by ELISPOT and intracellular IFN-gamma staining. Results are shown in FIGS. 2A-2C.

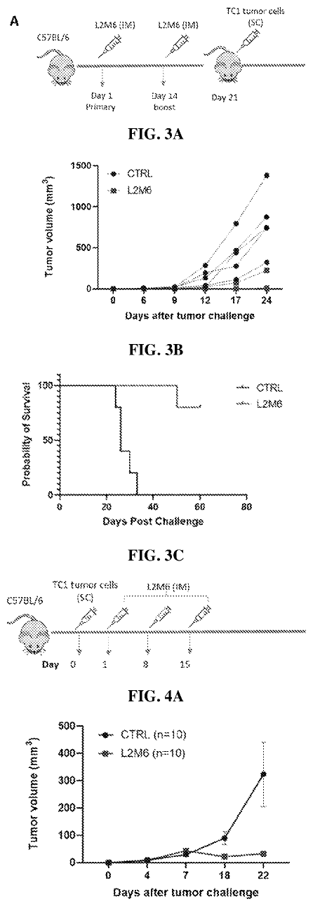

In vivo antitumor efficacy of prophylactic vaccination with mRNA-L2M6 was assayed. An illustration is shown in FIG. 3A of 5 C57BL/c mice immunized with 1 μg of mRNA/SCT-E6 intramuscular on day 1 and day 14, on day 21, 1×10⁵ TC1 tumor cells, a well-established cancer cell line derived from lung epithelial cells that have been transformed with oncogenic human papillomavirus (HPV) E6 and E7 genes, were injected subcutaneously. Tumor growth and survival were monitored. Results are shown in FIG. 3B and FIG. 3C.

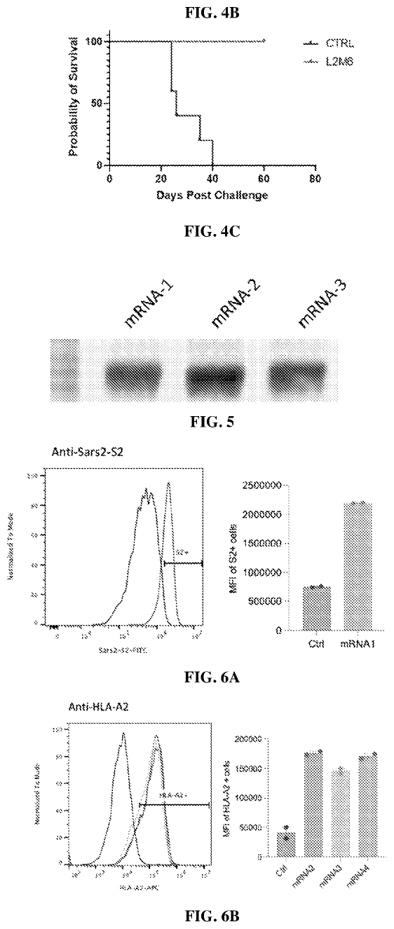

In vivo efficacy of therapeutic administration of mRNA-L2M6 vaccine was assayed. An illustration is shown in FIG. 4A of 10 female C57BL/6 mice injected with 2×10\hat{ }5 TC-1 tumor cells on the frank s.c., day 1, 8 and 15 after tumor challenge, mice received three doses of immunization of L2M6 or PBS. Tumor progression and long-term survival was monitored for each experimental group. Results are shown in FIG. 4B and FIG. 4C.

### Conclusion:

In conclusion, our study demonstrates that mice immunized with the Kb-E6-SCT mRNA vaccine exhibit heightened E6-specific CD8+ T cell immune responses. More significantly, our findings reveal a substantial enhancement in the antitumoral effects against E6-expressing tumor cells. This underscores the effectiveness of our strategy to bypass antigen processing and presentation mechanisms, highlighting its potential for significantly augmenting the potency of cancer vaccines.

### Example 2: A Therapeutic mRNA Vaccine Elicits a Broad and Specific CD8 T Cell Response Targeting HPV 16 E6 in HLA A2.1 Transgenic Mice

Design and Validation of Four mRNAs

1. HPV-E6-E7-S2-mRNA (mRNA-1): This DNA construct comprises HPV-16 E6, E7 and the Spike glycoprotein (subunit S2) of SARS-COV-2 (SEQ ID NO: 498), it encodes full-length HPV-16 E6/E7 protein (SEQ ID NO: 497), in which the known oncogenic transforming regions are inactivated, and truncated S2 protein, aiming to induce broad E6- and E7-specific T cell responses. Additionally, it stimulates S2-specific CD4 T cell responses, considering the widespread exposure to SARS-COV-2 infection or COVID-19 immunization globally, these S2-specific CD4 T cells can enhance the CD8 T cell antitumor functions.

2. HLA A0201 mRNA (mRNA-2): This DNA construct encodes HLA A0201 gene (SEQ ID NO: 500), facilitating its protein overexpression (SEQ ID NO: 499) to enhance the T cell immunity.

3. HLA A0201-E6-SCT mRNA (mRNA-3): This DNA construct encodes an immunodominant T cell epitope derived from E6 of HPV16, a β2-microglobulin, and HLA A0201 (SEQ ID NO: 17 and 18).

4. HLA A0201-E7-SCT mRNA (mRNA-4): A DNA construct encodes a immunodominant T cell epitope derived from E7 of HPV16, a β2-microglobulin, and HLA A0201 (SEQ ID NO: 19 and 20).

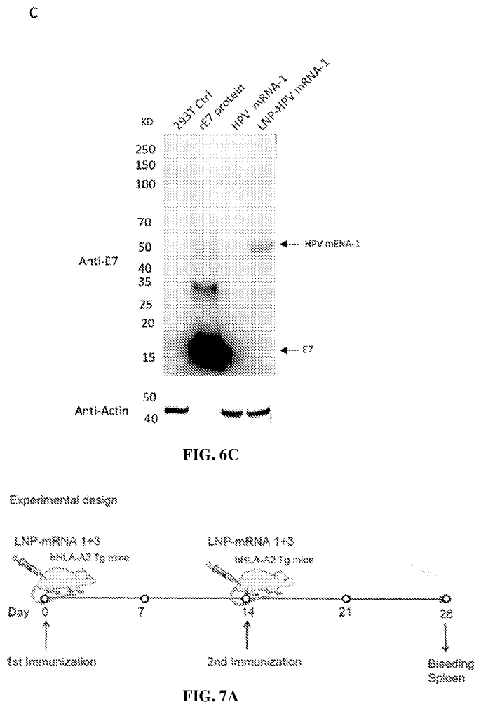

The mRNAs were transcribed from the indicated constructs and are illustrated in FIG. 5. FIGS. 6A-6C shows the in vitro validation of mRNA 1-4 after transfection into 293T cells.

### Animal Studies

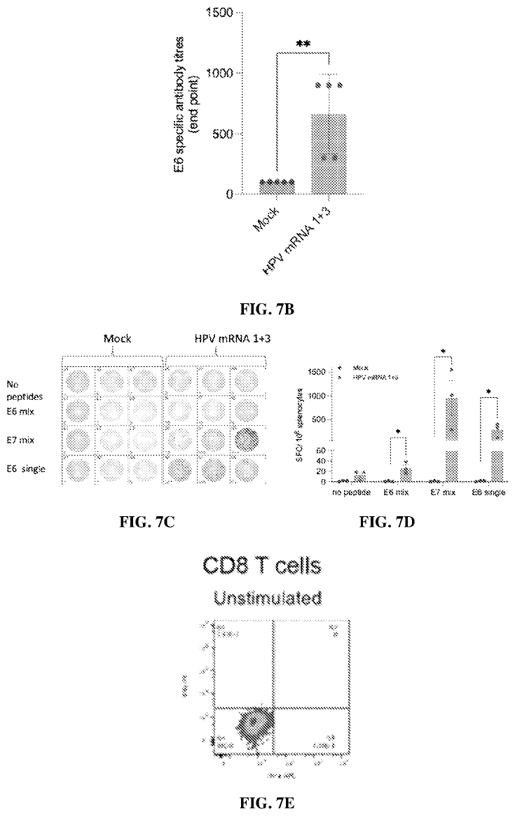

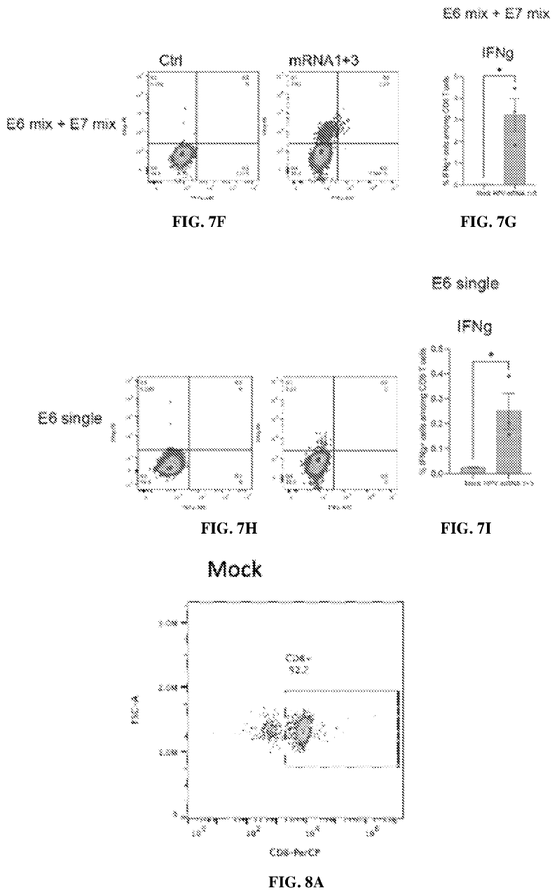

HLA-A2.1 transgenic mice (C57BL/6-Mcph1Tg(HLA-A2.1)1Enge/J, strain #003475) were purchased from Jackson Laboratory. The mice underwent intramuscular immunization with 1 μg of the specified vaccine on two occasions, two weeks apart. Fourteen days after the final immunization, spleens were harvested for analysis, FIG. 7A.

CD8+ T Cell Response Analysis: Antigen-specific T cell populations were determined using ELISPOT and intracellular IFN-gamma staining by stimulation with E6, E7 peptides, or E6-CD8 T cell peptide. Results are presented in FIGS. 7B-7D.

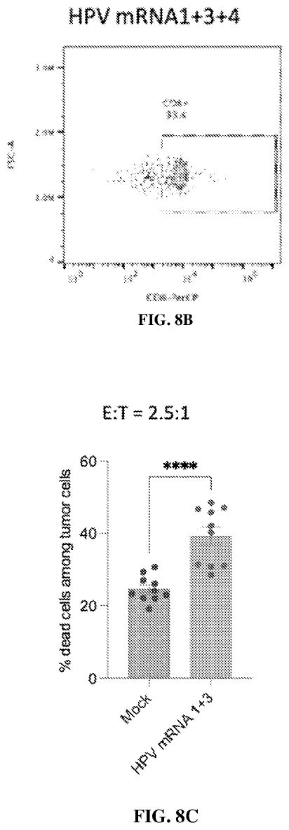

In Vitro CTL Killing Assays: CD8 T cell responses elicited by HPV-16 mRNA were further assessed through in vitro CTL killing assays, as depicted in FIGS. 8A-8B. Fourteen days post-two immunizations with mRNA 1 and mRNA-3, splenocytes were harvested from HLA-A2.1 transgenic mice. CD8+ T cells were isolated (FIG. 8A) and co-cultured with tumor cells (TC1/A2), which express HPV-16 E6/E7 and are transduced with HLA A2.1. Cell death was determined by FACS, as illustrated in FIG. 8B.

### Conclusion:

In conclusion, the vaccine utilizing mRNA encoding HPV 16 E6/E7/S2 and SCT-E6/E7 has demonstrated promising results in HLA A2.1 transgenic mice. The vaccine induced a robust and specific CD8 T cell response targeting HPV E6 and E7 antigens. Moreover, the activated CD8 T cells exhibited the ability to effectively eliminate HLA A2 positive E6/E7-expressing tumor cells in an ex vivo setting. These findings suggest that the SCT technology employed in this vaccine has the potential to enhance the clearance of established HPV-positive cancers in vivo.

What is claimed is:

1. A single chain trimer nucleic acid encoding a first T cell epitope, a β2-microglobulin, and a MHC class I heavy chain amino acid sequence.

2. The single chain trimer nucleic acid of claim 1, wherein the nucleic acid comprises plasmid DNA, minicircle DNA, microRNA, mRNA, self-amplifying RNA, circle RNA, DNA launched self-amplifying RNA, or viral vector.

3. The single chain trimer nucleic acid of claim 1 or 2, wherein the T cell epitope comprises a viral, bacterial, parasitic, or cancer T cell epitope.

4. The single chain trimer nucleic acid of any one of claims 1-3, wherein the T cell epitope comprises a tumor-specific antigens (TSA), Tumor-associated antigens (TAA), or viral-derived cancer antigens.

5. The single chain trimer nucleic acid of claim any of claims 1-4; wherein the single chain trimer nucleic acid comprises SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 498, or SEQ ID NO: 500.

6. The single chain trimer nucleic acid of any one of claims 1-5; wherein the first T cell epitope comprises an epitope listed in Table 2.

7. The single chain trimer nucleic acid of any one of claims 1-6, wherein the single chain trimer nucleic acid encodes a single chain trimer having SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 497, or SEQ ID NO: 499.

8. The single chain trimer nucleic acid of any of claims 1-7, wherein single chain trimer nucleic acid comprises a nucleic acid encoding an amino to carboxy terminal order T cell epitope, the β2 microglobulin, and the MHC heavy chain sequence.

9. The single chain trimer nucleic acid of any of claims 1-8, wherein the MHC class I heavy chain comprises an HLA-A1:01, HLA-A2:01, HLA-A2:03, HLA-A2:06, HLA-A2:07, HLA-A3:01, HLA-A9, HLA-A10, HLA-A11:01, HLA-A23:01, HLA-A24:02, HLA-A25:01, HLA-A26:01, HLA-A29:02, HLA-A30:01, HLA-A30:02, HLA-A31:01, HLA-A32:02, HLA-A33:03, HLA-A34:01, HLA-A34:02, HLA-A68:01, HLA-A68:02, HLA-A74:01, HLA-B5, HLA-B7:02, HLA-B8:01, HLA-B-12, HLA-B13:01, HLA-B14:02, HLA-B15:01, HLA-B15:02, HLA-B15:03, HLA-B15:18, HLA-B18:01, HLA-B35:01, HLA-B38:02, HLA-B40:01, HLA-B40:02, HLA-B42:01, HLA-B44:02, HLA-B44:03, HLA-B45:01, HLA-B46:01, HLA-B49:01, HLA-B51:01, HLA-B52:01, HLA-B53:01, HLA-B54:01, HLA-B55:02, HLA-B57:01, HLA-B58:01, HLA-B67:01, HLA-C1:02, HLA-C2:02, HLA-C3:02, HLA-C3:03, HLA-C3:04, HLA-C4:01, HLA-C5:01, HLA-C6:02, HLA-C7:01, HLA-C7:02, HLA-C8:01, HLA-C8:02, HLA-C12:03, HLA-C14:02, HLA-C16:01, HLA-C17:01, or HLA-C18:01 heavy chain.

10. The single chain trimer nucleic acid of any of claims 1-9, further comprising a second T cell epitope.

11. The single chain trimer nucleic acid of claim 10, wherein the second T cell epitope is a MHC class II restricted epitope.

12. The single chain trimer nucleic acid of claim 11, wherein the second T cell epitope comprises SEQ ID NO: 21, SEQ ID NO: 22, SEQ ID NO: 23, SEQ ID NO: 24, SEQ ID NO: 25, SEQ ID NO: 26, or an epitope listed in Table 5.

13. The single chain trimer nucleic acid of any of claims 1-12, wherein the single chain trimer nucleic acid further comprises a flexible first linker between the first T cell epitope and the β2 microglobulin and second linker between the β2 microglobulin and the MHC class I heavy chain sequence.

14. A composition comprising a single chain trimer nucleic acid encoding a first T cell epitope, a β2-microglobulin, and a MHC class I heavy chain sequence.

15. The composition of claim 14, wherein the nucleic acid comprises plasmid DNA, minicircle DNA, microRNA, mRNA, self-amplifying RNA, circle RNA, DNA launched self-amplifying RNA.

16. The composition of claim 14 or 15, wherein the T cell epitope comprises a viral, bacterial, parasitic, or cancer T cell epitope.

17. The composition of any one of claims 14-16, wherein the T cell epitope comprises a T cell epitope of tumor-specific antigens (TSA), Tumor-associated antigens (TAA), or viral-derived cancer antigens.

18. The composition of claim any of claims 14-17; wherein the single chain trimer nucleic acid comprises SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 498, or SEQ ID NO: 500.

19. The composition of claim any of claims 14-18; wherein the first T cell epitope comprises an epitope listed in Table 2.

20. The composition of any one of claims 14-19, wherein the single chain trimer nucleic acid encodes a single chain trimer having SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 497, or SEQ ID NO: 499.

21. The composition of any of claims 14-20, wherein single chain trimer nucleic acid comprises a nucleic acid encoding an amino to carboxy terminal order T cell epitope, the β2 microglobulin, and the MHC heavy chain sequence.

22. The composition of any of claims 14-21, wherein the MHC class I heavy chain comprises an HLA-A1:01, HLA-A2:01, HLA-A2:03, HLA-A2:06, HLA-A2:07, HLA-A3:01, HLA-A9, HLA-A10, HLA-A11:01, HLA-A23:01, HLA-A24:02, HLA-A25:01, HLA-A26:01, HLA-A29:02, HLA-A30:01, HLA-A30:02, HLA-A31:01, HLA-A32:02, HLA-A33:03, HLA-A34:01, HLA-A34:02, HLA-A68:01, HLA-A68:02, HLA-A74:01, HLA-B5, HLA-B7:02, HLA-B8:01, HLA-B-12, HLA-B13:01, HLA-B14:02, HLA-B15:01, HLA-B15:02, HLA-B15:03, HLA-B15:18, HLA-B18:01, HLA-B35:01, HLA-B38:02, HLA-B40:01, HLA-B40:02, HLA-B42:01, HLA-B44:02, HLA-B44:03, HLA-B45:01, HLA-B46:01, HLA-B49:01, HLA-B51:01, HLA-B52:01, HLA-B53:01, HLA-B54:01, HLA-B55:02, HLA-B57:01, HLA-B58:01, HLA-B67:01, HLA-C1:02, HLA-C2:02, HLA-C3:02, HLA-C3:03, HLA-C3:04, HLA-C4:01, HLA-C5:01, HLA-C6:02, HLA-C7:01, HLA-C7:02, HLA-C8:01, HLA-C8:02, HLA-C12:03, HLA-C14:02, HLA-C16:01, HLA-C17:01, or HLA-C18:01 heavy chain.

23. The composition any of claims 14-20, further comprising a second T cell epitope.

24. The composition of claim 23, wherein the second T cell epitope is a MHC class II restricted epitope.

25. The composition of claim 23, wherein the second T cell epitope comprises SEQ ID NO: 21, SEQ ID NO: 22, SEQ ID NO: 23, SEQ ID NO: 24, SEQ ID NO: 25, SEQ ID NO: 26, or an epitope listed in Table 5.

26. The composition of any of claims 14-25, wherein the single chain trimer nucleic acid further comprises a flexible first linker between the first T cell epitope and the β2 microglobulin and second linker between the β2 microglobulin and the MHC class I heavy chain sequence.

27. The composition of any one of claims 14-26, wherein the composition is a nanoparticle, a lipid nanoparticle dispersion, a liposomal formulation, a lipid emulsion, vaccine, vector, or any combination thereof.

28. The composition of any one of claims 14-27, wherein the composition is a lipid nanoparticle, wherein the lipid nanoparticle comprises 20% to 80% of ionizable lipid, cationic lipid, or any combination thereof; 0% to 5% pegylated lipids; 0% to 40% helper lipids; and 0% to 80% sterol.

29. A lipid nanoparticle comprising:
20% to 80% of ionizable lipid, cationic lipid, or any combination thereof;
0% to 5% pegylated lipids; 0% to 40% helper lipids;
0% to 80% sterol; and
a single chain trimer nucleic acid encoding a first T cell epitope, a β2-microglobulin,
and a MHC class I heavy chain sequence encapsulated in the lipid nanoparticle.

30. The lipid nanoparticle of claim 29, wherein the nucleic acid plasmid DNA, minicircle DNA, microRNA, mRNA, self-amplifying RNA, circle RNA, or DNA launched self-amplifying RNA.

31. The lipid nanoparticle of claim 29 or 30, wherein the T cell epitope comprises a viral, bacterial, parasitic, or cancer T cell epitope.

32. The lipid nanoparticle of any one of claims 29-31, wherein the T cell epitope comprises a T cell epitope of tumor-specific antigens (TSA), Tumor-associated antigens (TAA), or viral-derived cancer antigens

33. The lipid nanoparticle of claim any of claims 29-32; wherein the single chain trimer nucleic acid comprises SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 498, or SEQ ID NO: 500.

34. The lipid nanoparticle of claim any of claims 29-33; wherein the first T cell epitope comprises an epitope listed in Table 2.

35. The lipid nanoparticle of any one of claims 29-34, wherein the single chain trimer nucleic acid encodes a single chain trimer having SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 497, or SEQ ID NO: 499.

36. The lipid nanoparticle of any of claims 29-35, wherein single chain trimer nucleic acid comprises a nucleic acid encoding an amino to carboxy terminal order T cell epitope, the β2 microglobulin, and the MHC heavy chain sequence.

37. The lipid nanoparticle of any of claims 29-36, wherein the MHC class I heavy chain comprises an HLA-A1:01, HLA-A2:01, HLA-A2:03, HLA-A2:06, HLA-A2:07, HLA-A3:01, HLA-A9, HLA-A10, HLA-A11:01, HLA-A23:01, HLA-A24:02, HLA-A25:01, HLA-A26:01, HLA-A29:02, HLA-A30:01, HLA-A30:02, HLA-A31:01, HLA-A32:02, HLA-A33:03, HLA-A34:01, HLA-A34:02, HLA-A68:01, HLA-A68:02, HLA-A74:01, HLA-B5, HLA-B7:02, HLA-B8:01, HLA-B-12, HLA-B13:01, HLA-B14:02, HLA-B15:01, HLA-B15:02, HLA-B15:03, HLA-B15:18, HLA-B18:01, HLA-B35:01, HLA-B38:02, HLA-B40:01, HLA-B40:02, HLA-B42:01, HLA-B44:02, HLA-B44:03, HLA-B45:01, HLA-B46:01, HLA-B49:01, HLA-B51:01, HLA-B52:01, HLA-B53:01, HLA-B54:01, HLA-B55:02, HLA-B57:01, HLA-B58:01, HLA-B67:01, HLA-C1:02, HLA-C2:02, HLA-C3:02, HLA-C3:03, HLA-C3:04, HLA-C4:01, HLA-C5:01, HLA-C6:02, HLA-C7:01, HLA-C7:02, HLA-C8:01, HLA-C8:02, HLA-C12:03, HLA-C14:02, HLA-C16:01, HLA-C17:01, or HLA-C18:01 heavy chain.

38. The lipid nanoparticle any of claims 29-37, further comprising a second T cell epitope.

39. The lipid nanoparticle of claim 38, wherein the second T cell epitope is a MHC class II restricted epitope.

40. The lipid nanoparticle of claim 38, wherein the second T cell epitope comprises SEQ ID NO: 21, SEQ ID NO: 22, SEQ ID NO: 23, SEQ ID NO: 24, SEQ ID NO: 25, SEQ ID NO: 26, or an epitope listed in Table 5.

41. The lipid nanoparticle of any of claims 29-40, wherein the single chain trimer nucleic acid further comprises a flexible first linker between the first T cell epitope and the β2 microglobulin and second linker between the β2 microglobulin and the MHC class I heavy chain sequence.

42. A pharmaceutical composition comprising the single chain trimer nucleic acid of any one of claims 1-13, the composition of any of claims 14-28, or the lipid nanoparticle of any one of claims 29-41.

43. A cell comprising the single chain trimer nucleic acid of any one of claims 1-13, the composition of any of claims 14-28, or the lipid nanoparticle of any one of claims 29-41, or the pharmaceutical composition of claim 42.

44. The cell of claim 37, wherein the cell comprises a dendritic cell, B cell, macrophage, or other cells derived from a subject that can be used to for immunotherapy.

45. A method of activating and/or expanding an antigen presenting cell said method comprising co-culturing the antigen-presenting cell and a cell comprising a single chain trimer nucleic acid of any one of claims 1-13, the composition of any of claims 14-28, or the lipid nanoparticle of any one of claims 29-41, or the pharmaceutical composition of claim 42.

46. A method of treating a subject with a viral infection, bacterial infection, parasitic infection and/or a cancer said method comprising administering to the subject a therapeutically effective amount of the single chain trimer nucleic acid of any one of claims 1-13, the composition of any of claims 14-28, or the lipid nanoparticle of any one of claims 29-41, the pharmaceutical composition of claim 42, or the cell of any one of claims 43-44.

47. A method of treating a subject with a viral infection, bacterial infection, parasitic infection and/or a cancer said method comprising obtaining antigen-presenting cells from the subject; contacting the antigen-presenting cells from the subject with the single chain trimer nucleic acid of any one of claims 1-13, the composition of any of claims 14-28, or the lipid nanoparticle of any one of claims 29-41, or the pharmaceutical composition of claim 42 thereby activating the antigen-presenting cells, and administering the activated cells to the subject.

48. The method of claim 45 or claim 47, wherein the antigen-presenting cell comprises a dendritic cell, B cell, macrophage, or other cells derived from a subject that can be used to for immunotherapy.
